(19) United States
(12) Patent Application Publication
(10) Pub. No.: US 2025/0177513 A1
(43) Pub. Date: Jun. 5, 2025

(54) RABIES G PROTEIN AND USES THEREOF

(71) Applicant: Icosavax, Inc., Seattle, WA (US)

(72) Inventors: ANDREW FELDHAUS, Seattle, WA (US); DOUGLAS HOLTZMAN, Seattle, WA (US)

(21) Appl. No.: 19/037,942

(22) Filed: Jan. 27, 2025

Related U.S. Application Data
(63) Continuation of application No. PCT/US23/71904, filed on Aug. 9, 2023.
(60) Provisional application No. 63/371,148, filed on Aug. 11, 2022.

(51) Int. Cl. A61K 39/205; A61K 39/00; A61P 37/04; C07K 14/005; C12N 7/00

(52) U.S. Cl. CPC A61K 39/205; A61P 37/04; C07K 14/005; C12N 7/00; A61K 2039/5258; A61K 2039/575; C12N 2760/20122; C12N 2760/20123; C12N 2760/20134; C12N 2760/20171

(57) ABSTRACT

Provided are polypeptide comprising a rabies G protein ectodomain. The rabies G protein ectodomain may have a deletion of the fusion loop domain. Further provided are nanoparticle vaccines for rabies virus. Further provided are pharmaceutical compositions, methods of manufacturing, and methods of use, e.g., immunizing a subject to generate a protective immune response to rabies virus.

### CROSS-REFERENCE TO RELATED APPLICATION

This application is a continuation of PCT Application No. PCT/US23/71904, filed Aug. 9, 2023, which claims the benefit of U.S. Provisional Patent Application No. 63/371,148, filed Aug. 11, 2022. The contents of each of which is incorporated by reference herein in its entirety.

### INCORPORATION BY REFERENCE OF SEQUENCE LISTING

This application contains a Sequence Listing which has been submitted in .XML format via EFS-WEB and is hereby incorporated by reference in its entirety. Said .XML copy, created on Aug. 7, 2023, is named 061291-507001WO_SeqList_ST26.xml and is 236 kilobytes in size.

### FIELD

The disclosure relates generally to vaccines for rabies virus.

### BACKGROUND

Rabies virus (RV) is a lethal viral pathogen that is transmitted to humans primarily via the bites of infected dogs. Estimates suggest that each year rabies kills >55,000 individuals, primarily children in Asia and Africa. Worldwide annual direct expenditures on rabies interventions, including antibodies for post-exposure prophylaxis, are over US $1B with total economic impact of rabies estimated to be >US $8B. As rabies is endemic in wild animals (foxes, skunks, ferrets, bats, and others) there is no possibility of eradication of the pathogen.

RV is a lyssavirus in the family of Rhabdoviridae. Lyssaviruses have a 12-kb non-segmented RNA genome that encodes five viral proteins: a nucleoprotein (N), a phosphoprotein (P), a matrix protein (M), a glycoprotein (G) and an RNA-dependent RNA polymerase (or large protein, L). RV is composed of two structural and functional units: an internal helical nucleocapsid and an external envelope. The nucleocapsid consists of a ribonucleoprotein complex comprising the genomic RNA and tightly bound N protein and the L and P proteins. The lipid envelope is derived from the host cytoplasmic membrane during budding. G protein spikes, composed of trimers of glycosylated ectodomains, allow the virus to bind to host cell receptors and facilitate fusion of the viral and host membranes. The M protein forms oligomers that bind to the outside of the nucleocapsid, giving rigidity to the virion structure and providing a binding platform for the viral G protein, which is the known target for protective immunity. The G protein is a class III viral fusion protein.

Vaccines against rabies have been available for over a hundred years, however their cost and complex immunization schedules reduce access. Despite international momentum to eliminate rabies deaths with available tools, current vaccines are suboptimal for controlling infections and disease due to RV. Limitations of the current products include poor yield and purity of product, lack of thermostability, sensitivity to freeze-thaw, and difficult regimens, including multiple doses delivered through intradermal immunization.

There is therefore a need for an improved vaccine against rabies.

### SUMMARY

In one aspect, the disclosure provides a polypeptide, comprising a rabies G protein ectodomain, wherein the rabies G protein ectodomain comprises a deletion of a fusion loop domain of the rabies G protein ectodomain, wherein the deleted fusion loop domain is about residue 70 to about residue 200 of the rabies G protein ectodomain, numbered according to SEQ ID NO: 53.

In variations, the disclosure provides polypeptide, comprising a rabies G protein ectodomain, wherein the rabies G protein ectodomain comprises a deletion of a fusion loop domain of the rabies G protein ectodomain, wherein the deleted fusion loop domain is about residue 50 to about residue 180, about residue 70 to about residue 180, about residue 80 to about residue 180, about residue 90 to about residue 180, or about residue 100 to about residue 180; about residue 50 to about residue 190, about residue 70 to about residue 190, about residue 80 to about residue 190, about residue 90 to about residue 190, or about residue 100 to about residue 190; about residue 50 to about residue 200, about residue 70 to about residue 200, about residue 80 to about residue 200, about residue 90 to about residue 200, or about residue 100 to about residue 200; about residue 50 to about residue 210, about residue 70 to about residue 210, about residue 80 to about residue 210, about residue 90 to about residue 210, or about residue 100 to about residue 210; about residue 50 to about residue 220, about residue 70 to about residue 220, about residue 80 to about residue 220, about residue 90 to about residue 220, or about residue 100 to about residue 220 of the rabies G protein ectodomain, numbered according to SEQ ID NO: 53.

In some embodiments of the polypeptide, the deleted fusion loop domain is residue 66 to residue 207 of the rabies G protein ectodomain, numbered according to SEQ ID NO: 53.

In some embodiments of the polypeptide, the rabies G protein ectodomain comprises a first polypeptide segment linked to a second polypeptide segment, wherein the first polypeptide segment shares at least 70%, at least 80%, at least 85%, at least 90%, at least 91%, at least 92%, at least 93%, at least 94%, at least 95%, at least 96%, at least 97%, at least 98%, at least 99%, or 100% identity to residues 20-65 of SEQ ID NO: 53; and wherein the second polypeptide segment shares at least 70%, at least 80%, at least 85%, at least 90%, at least 91%, at least 92%, at least 93%, at least 94%, at least 95%, at least 96%, at least 97%, at least 98%, at least 99%, or 100% identity to residues 208-417 of SEQ ID NO: 53.

In some embodiments of the polypeptide, the polypeptide is a fusion protein comprising, in N to C terminal order, the first polypeptide segment, a polypeptide linker, and the second polypeptide segment.

In some embodiments of the polypeptide, the rabies G protein ectodomain shares at least 70%, at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs: 77-81.

In some embodiments of the polypeptide, the polypeptide comprises a multimerization domain.

In some embodiments of the polypeptide, the multimerization domain is a trimerization domain.

In some embodiments of the polypeptide, the multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to FoldOn (SEQ ID NO: 58).

In some embodiments of the polypeptide, the multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to a polypeptide sequence selected from SEQ ID NOs: 1, 4, 5, 7, 9, 18, 19, 21, 24, 25, 26, 29, 30, 31, 34, 36, 37, 39, 42, 43, 44, 45, 46, 47, 48, 49, 50, and 51, wherein the interface residues identified in Table 3 are conserved.

In some embodiments of the polypeptide, the multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50A (SEQ ID NO: 7).

In some embodiments of the polypeptide, the multimerization domain is I53-50A-Δcys (SEQ ID NO: 67).

In some embodiments of the polypeptide, the polypeptide shares at least 70%, at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs: 93-96.

In some embodiments of the polypeptide, the multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-dn5B (SEQ ID NO: 75).

In some embodiments of the polypeptide, the polypeptide shares at least 70%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 101 or SEQ ID NO: 102.

In some embodiments of the polypeptide, the multimerization domain is a ferritin polypeptide capable of forming a ferritin particle.

In some embodiments of the polypeptide, the polypeptide shares at least 70%, at least 80%, at least 85%, at least 90%, at least 91%, at least 92%, at least 93%, at least 94%, at least 95%, at least 96%, at least 97%, at least 98%, at least 99%, or 100% identity to Construct F-ferritin (SEQ ID NO:103).

In some embodiments of the polypeptide, the multimerization domain is a particle-forming domain.

In another aspect, the disclosure provides a nanoparticle, comprising a polypeptide according to any embodiment. In some embodiments, the polypeptide comprises the rabies G protein ectodomain according to any embodiment.

In some embodiments of the nanoparticle, the nanoparticle is a protein-based virus-like particle (pbVLP) or nanostructure.

In some embodiments of the nanoparticle, the nanoparticle lacks a lipid component.

In some embodiments of the nanoparticle, the nanoparticle comprises a lipid component.

In some embodiments of the nanoparticle, the nanoparticle comprises a second polypeptide component.

In some embodiments of the nanoparticle, the nanoparticle comprises a second polypeptide component and the first polypeptide component, and wherein the nanoparticle is a self-assembling nanoparticle comprising the first and second polypeptide components symmetrically arranged with point group symmetry.

In some embodiments of the nanoparticle, (a) the first polypeptide component comprises any polypeptide of the disclosure, and the first polypeptide component forms a first homomeric complex via the multimerization domain of the polypetide; (b) the second polypeptide component comprises a second multimerization domain, and the second polypeptide component forms a second homomeric complex via the second multimerization domain of the polypetide; (c) the first homomeric complex and the second homomeric complex assemble to form the nanoparticle with point-group symmetry; (d) the nanoparticle lacks other polypeptide components; and/or (e) the nanoparticle lacks lipid components.

In some embodiments of the nanoparticle, the nanoparticle has icosahedral symmetry.

In some embodiments of the nanoparticle, the first multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50A (SEQ ID NO: 7) or I53-50A ΔCys (SEQ ID NO: 67); and the second multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50B (SEQ ID NO: 8) or to I53-50B.4PosT1 (SEQ ID NO: 34).

In some embodiments of the nanoparticle, the first multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-dn5B (SEQ ID NO: 75); and the second multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-dn5A (SEQ ID NO: 74).

In some embodiments of the nanoparticle, the first polypeptide component shares at least 70%, at least 80%, at least 85%, at least 90%, at least 91%, at least 92%, at least 93%, at least 94%, at least 95%, at least 96%, at least 97%, at least 98%, at least 99%, or 100% identity to a polypeptide sequence selected from SEQ ID NOs: 93-96; and the second polypeptide component shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50B (SEQ ID NO: 8) or to I53-50B.4PosT1 (SEQ ID NO: 34).

In some embodiments of the nanoparticle, the first polypeptide component shares at least 70%, at least 80%, at least 85%, at least 90%, at least 91%, at least 92%, at least 93%, at least 94%, at least 95%, at least 96%, at least 97%, at least 98%, at least 99%, or 100% identity to a polypeptide sequence selected from SEQ ID NOs: 101-102; and the second polypeptide component shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-dn5A (SEQ ID NO: 74).

In another aspect, the disclosure provides a pharmaceutical composition for use as a vaccine, comprising any polypeptide or nanoparticle of the disclosure, and optionally one or more pharmaceutically acceptable excipients.

In some embodiments of the pharmaceutical composition, the pharmaceutical composition comprises at least one adjuvant.

In some embodiments of the pharmaceutical composition, the pharmaceutical composition comprises an oil-in-water emulsion.

In some embodiments of the pharmaceutical composition, the pharmaceutical composition comprises a squalene-based oil-in-water emulsion.

In some embodiments of the pharmaceutical composition, the pharmaceutical composition comprises a toll-like receptor (TLR) immunostimulant.

In some embodiments of the pharmaceutical composition, the pharmaceutical composition comprises squalene, SLA, GLA, R848, IMQ, 3M-052, CpG, saponin (QS21), or combinations thereof.

In another aspect, the disclosure provides a polynucleotide, comprising a polynucleotide sequence encoding any polypeptide or nanoparticle of the disclosure.

In another aspect, the disclosure provides a vector, comprising a polynucleotide comprising a polynucleotide sequence encoding any polypeptide or nanoparticle of the disclosure.

In another aspect, the disclosure provides a method of generating an immune response in a subject to a subject infected with rabies virus, comprising administering any polypeptide, nanoparticle, pharmaceutical composition, polynucleotide, or vector of the disclosure in an amount effective to generate an immune response.

In another aspect, the disclosure provides a method of immunizing a subject against infection by rabies to a subject infected with rabies virus, comprising administering any polypeptide, nanoparticle, pharmaceutical composition, polynucleotide, or vector of the disclosure in an amount effective to generate an immune response.

In another aspect, the disclosure provides a method of providing post-exposure prophylaxis to a subject infected with rabies virus, comprising administering any polypeptide, nanoparticle, pharmaceutical composition, polynucleotide, or vector of the disclosure in an amount effective to generate an immune response.

In some embodiments of the method, the immune response comprises a humoral immune response.

In some embodiments of the method, the immune response comprises a polyclonal antibody response against a rabies G protein.

In some embodiments of the method, the immune response comprises a neutralizing antibody response to rabies virus.

In some embodiments of the method, the method generates a protective immune response to rabies virus.

In some embodiments of the method, the method generates neutralizing antibodies to rabies virus.

In some embodiments of the method, the administering step comprises intramuscular injection or subcutaneous injection.

In some embodiments of the method, the method results in the production of Rabies-specific neutralizing antibodies in the subject in need thereof.

In some embodiments of the method, the method results in an increase in Rabies-specific neutralizing antibodies in the subject in need thereof, of at least about 2-fold, at least about 3-fold, at least about 4-fold, at least about 5-fold, at least about 10-fold, at least about 15-fold, at least about 20-fold, or at least about 25-fold increase compared to Rabies-specific neutralizing antibodies in the same subject prior to the administering step.

In some embodiments of the method, the method generates a neutralizing titer of at least 0.5 IU/mL in a rapid fluorescent focus inhibition test (RFFIT) and/or a fluorescent antibody virus neutralization (FAVN) test.

In some embodiments of the method, the subject is a non-human animal.

In some embodiments of the method, the subject is a companion animal.

In some embodiments of the method, the subject is a human.

In another aspect, the disclosure provides a host cell, comprising a polynucleotide comprising a polynucleotide sequence encoding any polypeptide or nanoparticle of the disclosure.

In another aspect, the disclosure provides a method of manufacturing a vaccine, comprising: culturing the host cell of the disclosure in a culture medium so that the host cell secretes a first polypeptide component into the culture media; purifying the first polypeptide component from the culture media; mixing the first polypeptide component with a second polypeptide component, wherein the first and second polypeptide components self-assemble to form a nanoparticle; and/or purifying the nanoparticle.

In another aspect, the disclosure provides a kit, comprising a polypeptide, nanoparticle, polynucleotide, vector, or pharmaceutical composition of the disclosure.

### BRIEF DESCRIPTION OF THE DRAWINGS

FIG. 1 is the diagram of the sequence of rabies G protein with secondary structure elements indicated.

FIG. 2 is a figure depicting one- and two-component nanoparticles (without the rabies G protein).

FIG. 3 is a diagram of a protein-based virus-like particle (pbVLP) having two components. I53-50A+I53-50B or I53-dn5B+I53-dn5A multimerization domains may be used to form VLPs. A VLP having icosahedral (I53) symmetry as shown.

FIG. 4 is an image of an SDS-PAGE analysis of a pbVLP (Construct F-CompA+CompB, I53-50 version).

FIG. 5 is a cryoelectron microgram of VLPs demonstrating that the VLPs form the predicted icosahedral particle.

FIG. 6 shows graphs of biolayer interferometry (BLI) with two neutralizing antibodies, D1-25 and 1112-1 (RD-Biotech), that recognize antigenic site III and II, respectively, of the rabies G protein, both without begin frozen (“pre-freeze”) or after a freeze-thaw (“post-freeze”).

FIG. 7 is a graph showing a potency ELISA assay with the two neutralizing antibodies (D1-25 and 1112-1 (RD-Biotech)). Construct F CompA expressed transiently in Expi293 (HEK) cells or CHO-K1 cells along with VLPs assembled with the CompAs were evaluated in the potency ELISA.

FIG. 8 is a graph of neutralizing titers in day 35 sera for the Construct C VLPs (AddaVax group) and ImRab3 group with geometric mean titers of 37.87 and 17.9 IU/ml respectively. Construct C VLPs formulated with Alhydrogel or Construct C-component induced low but detectable titers with geometric mean titers of 0.25 and 0.78 IU/ml respectively.

FIG. 9 is a graph showing that day 35 sera showed neutralizing titers were induced by Construct C (I53-50 and dn5), Construct B and Construct F at comparable levels and the titers of all animals were above the correlate of protection (0.5 IU/ml). The geometric mean titers induced by the VLPs ranged from 21.3 to 34.3 IU/ml and there were not statistically significant differences between the groups (Mann-Whitney test). The titers observed with the VLPs were comparable to those with ImRab3.

FIG. 10 is a graph showing that day 35 sera showed neutralizing geometric mean titers that range from 74.8 to 211 IU/ml for Construct C and Construct F. The titers of all Construct C and Construct F were not statistically significantly (Mann-Whitney test) between the groups. All animals (ICR mice) administered Construct C and Construct F had titers above the correlate of protection except for one animal in the 1 μg dose level for Construct F which was at background levels (<0.2 IU/ml). Titers observed with Construct C and Construct F were comparable to ImRab3 (geometric mean of 71.1) on day 35.

FIG. 11 is a graph showing that day 35 sera showed neutralizing titers for Construct C were above the correlate of protection with the 3 μg, 1 μg and 0.3 μg dosage in 17/18 animals (Syrian Golden hamsters) with geometric mean titers ranging from 2.2 to 6.7 IU/mL. The lowest Construct C dosage group, 0.1 μg, resulted in 2/6 animals with titers above the correlate of protection. The three highest doses (3 μg, 1 μg, and 0.3 μg) of Construct F resulted in comparable titers with all animals above the correlate of protection (18 animals) with geometric mean titers ranging from 4.8 to 5.7 IU/ml and the lowest dose with 3/6 animals above the correlate of protection.

FIG. 12 is a graph showing that day 35 sera showed neutralizing titers for Construct F I53-50 VLPs formulated with aqueous buffer, Alhydrogel, or AddaVax, Construct F dn5 VLPs, or Construct F-CompA. Construct F I53-50 VLPs formulated with AddaVax induced significantly higher titers.

FIG. 13 is a graph showing survival rate of animals administered either saline and challenged with rabies virus, immunized with Construct F VLPs, or RabAvert immunized.

FIG. 14 is a graph showing neutralizing antibody titers from day 34 sera from animals administered either saline, Construct F VLPs, or RabVert.

### SELECTED SEQUENCES IDENTIFIERS

| SEQ ID NO: | |
|---|---|
| 1-51 | Nanoparticle Polypeptide Sequences |
| 52 | N-terminal signal peptide |
| 53 | Ectodomain of rabies virus protein G |
| 54 | Ectodomain of rabies virus protein |
| | G without signal peptide |
| 55 | Ectodomain of rabies virus protein G, |
| | without fusion loop domain |
| 56 | Ectodomain of rabies virus protein G, |
| | first polypeptide segment |
| 57 | Ectodomain of rabies virus protein G, |
| | second polypeptide segment |
| 58 | Foldon domain of T4 fibritin |
| 59-63 | Gly/Ser linkers |
| 64 | CTD₄₂₃₋₄₄₁ |
| 65 | CTD₄₂₃₋₄₅₄ |
| 66 | CTD₄₂₃₋₄₅₉ |
| 67 | I53-50A-Δcys |
| 68-73 | T33 sequences |
| 74 | I53-dn5A |
| 75 | I53-dn5B |
| 76-81 | Ectodomain variants |
| 81-96 | I53-50A fusions |
| 97-102 | I53-dn5B fusions |
| 103 | Construct F ferritin |
| 104-170 | Rabies G Protein Sequences |
| 171-172 | 153_dn5A.1 and 153_dn5A.2 |
| 173 | I3-01 |
| 174 | I3-01 (M31) |
| 175-181 | 1WA3 sequences |
| 182-186 | Ferritin polypeptides |

### DETAILED DESCRIPTION

The present disclosure provides polypetides, nanoparticles, and related compositions useful in generating immune responses to the rabies G protein, as well as methods of making and using the same. Without being bound by theory, the rabies G protein is believed to comprise a fusion loop domain that includes two fusion loops and flanking sequences that together assume a complex tertiary structure. Deletion of this fusion loop domain may, at least in some cases, increase the expression, stability, and/or assembly competence of the rabies G protein. Moreover, as least in some cases, antigenic sites on the rabies G protein are preserved.

Epitopes for several neutralizing antibodies directed against the rabies G protein have been described. Antigenic site I comprises residues 226-231, site II is a discontinous epitope comprised of residues 34-42 and 198-200; site III consists of residues 226-231 (discontinous epitope 198-200 and 330-338) (Kuzmina et al. J Antivir Antiretrovir 5:22013(2013)); and site IV contains a critical residue 251 (Luo et al. Microbiol. Immunol. 39:693-702 (1995)). In addition, neutralizing antibody RG719 binds to residues 249-268 (Ni et al. Microbiol. Immunol. 39(9):693-702 (1995)). These are examples and not an exhaustive list of epitopes described for neutralization antibodies against rabies G protein.

The neutralizing epitopes are generally conformation epitopes so peptides corresponding to the epitopes do not bind the antibodies and thus do not induce neutralizing titers, although Dietzschold et al. J. Virol., 595-602 (1982), described generating neutralizing titers via immunization with three different CNBr-cleaved peptides of rabies G protein.

Unlike other glycoproteins (e.g., influenza HA), there is no proteolysis site but the rabies G protein contains two small loops (residues 91-97 and 139-145) that form a bipartite peptide involved in fusion.

### Polypeptides

The native sequence of the rabies G protein (GenBank P15199.2) is shown below with the signal sequence underlined and italicized and the transmembrane and intracellular portion underlined (SEQ ID NO: 53):

(SEQ ID NO: 53)

| | |
|---|---|
| 1 | MVPQVLLFVP LLGFSLCFGK FPIYTIPDEL GPWSPIDIHH |
| 41 | LSCPNNLVVE DEGCTNLSEF SYMELKVGYI SAIKVNGFTC |
| 81 | TGVVTEAETY TNFVGYVITT FKRKHFRPTP DACRAAYNWK |
| 121 | MAGDPRYEES LHNPYPDYHW LRTVRTTKES LIIISPSVTD |
| 161 | LDPYDKSLHS RGFPGGKCSG ITVSSTYCST NHDYTIWMPE |
| 201 | NPGPRTPCDI FTNSRGKRAS KGNKTCGFVD ERGLYKSLKG |
| 241 | ACRLKLCGVL GLRLMDGTWV AMQTSDETKW CPPDQLVNLH |
| 281 | DFRSDEIEHL VVEELVKKRE ECLDALESIM TTKSVSFRRL |
| 321 | SHLRKLVPGF GKAYTIFNKT LMEADAHYKS VRTWNEIIPS |
| 361 | KGCLKVGGRC HPHVNGVFFN GIILGPDGHV LIPEMQSSLL |
| 401 | QQHMELLKSS VIPLMHPLAD PSTVFKEGDE AEDFVEVHLP |
| 441 | DVYKQISGVD LGLPNWGKYV LMTAGAMIGL VLIFSLMTWC |
| 481 | RRANRPESKQ RSFGGTGRNV SVTSQSGKVI PSWESYRSGG |
| 521 | EIRL |

The native signal sequence is post-translationally cleaved when the protein is expressed. The native signal sequence may be replaced with another signal sequence for expression of the ectodomain, or in some embodiments no signal sequence is used. Accordingly, the native rabies G protein ectodomain has the following sequence:

(SEQ ID NO: 54)
K FPIYTIPDEL GPWSPIDIHH
LSCPNNLVVE DEGCTNLSEF SYMELKVGYI SAIKVNGFTC
TGVVTEAETY TNFVGYVTTT FKRKHFRPTP DACRAAYNWK
MAGDPRYEES LHNPYPDYHW LRTVRTTKES LIIISPSVTD
LDPYDKSLHS RGFPGGKCSG ITVSSTYCST NHDYTIWMPE
NPGPRTPCDI FTNSRGKRAS KGNKTCGFVD ERGLYKSLKG
ACRLKLCGVL GLRLMDGTWV AMQTSDETKW CPPDQLVNLH
DFRSDEIEHL VVEELVKKRE ECLDALESIM TTKSVSFRRL
SHLRKLVPGF GKAYTIFNKT LMEADAHYKS VRTWNEIIPS
KGCLKVGGRC HPHVNGVFFN GIILGPDGHV LIPEMQSSLL
QQHMELLKSS VIPLMHPLAD PSTVFKEGDE AEDFVEVHLP
DVYKQISGVD LGLPNWGKY

However, without being bound by theory, the C-terminus of the rabies G protein may be further C-terminally truncated to about residue 400, about residue 410, or about residue 420.

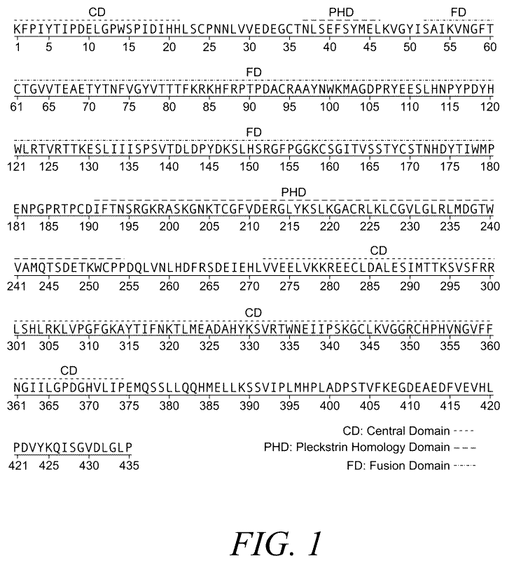

Without being bound by theory, FIG. 1 depicts the predicted secondary structure and disulfide bond pattern of the rabies G protein, numbered beginning at the N-terminus of the protein with the signal peptide removed. The fusion loop domain is considered to comprise about residue 70 to about residue 200 of the rabies G protein ectodomain, numbered according to SEQ ID NO: 53, or about residue 50 to about residue 180 of the rabies G protein ectodomain, numbered according to FIG. 1.

The present disclosure demonstrates expression of a rabies G protein antigen as a soluble trimer and as a component of a protein-based VLP. Without being bound by theory, the antigens described herein may be superior to known rabies G protein antigens in that they may have increased stability; may be expressed in higher yield; are less toxic to host cells; and/or may be better suited to display on virus-like particles including but not limited to protein-based virus-like particles.

In some embodiments, the rabies G protein ectodomain comprises one or more internal insertions and/or deletions. In particular, the rabies G protein ectodomain may comprise a deletion of part, all, or subtantially all of the fusion loop domain of the rabies G protein ectodomain. In some embodiments, the rabies G protein ectodomain comprises a substitution of one or more, two or more, or three or more amino acid.

In one aspect, the disclosure provides a polypeptide, comprising a rabies G protein ectodomain, wherein the rabies G protein ectodomain comprises a deletion of a fusion loop domain of the rabies G protein ectodomain, wherein the deleted fusion loop domain is about residue 70 to about residue 200 of the rabies G protein ectodomain, numbered according to SEQ ID NO: 53.

The deleted fusion loop domain may be about residue 50 to about residue 180, about residue 70 to about residue 180, about residue 80 to about residue 180, about residue 90 to about residue 180, or about residue 100 to about residue 180; about residue 50 to about residue 190, about residue 70 to about residue 190, about residue 80 to about residue 190, about residue 90 to about residue 190, or about residue 100 to about residue 190; about residue 50 to about residue 200, about residue 70 to about residue 200, about residue 80 to about residue 200, about residue 90 to about residue 200, or about residue 100 to about residue 200; about residue 50 to about residue 210, about residue 70 to about residue 210, about residue 80 to about residue 210, about residue 90 to about residue 210, or about residue 100 to about residue 210; about residue 50 to about residue 220, about residue 70 to about residue 220, about residue 80 to about residue 220, about residue 90 to about residue 220, or about residue 100 to about residue 220 of the rabies G protein ectodomain, numbered according to SEQ ID NO: 53.

In some embodiments of the polypeptide, the deleted fusion loop domain is residue 66 to residue 207 of the rabies G protein ectodomain, numbered according to SEQ ID NO: 53.

In some embodiments, the rabies G protein ectodomain is SEQ ID NO: 55, or a variant thereof:

(SEQ ID NO: 55)
K FPIYTIPDEL GPWSPIDIHH
LSCPNNLVVE DEGCTNLSEF SYMELXXXXX XXXXXXXXXX
XXXXXXXXXX XXXXXXXXXX XXXXXXXXXX XXXXXXXXXX
XXXXXXXXXX XXXXXXXXXX XXXXXXXXXX XXXXXXXXXX
XXXXXXXXXX XXXXXXXXXX XXXXXXXXXX XXXXXXXXXX
XXXXXXXCDI FTNSRGKRAS KGNKTCGFVD ERGLYKSLKG
ACRLKLCGVL GLRLMDGTWV AMQTSDETKW CPPDQLVNLH
DFRSDEIEHL VVEELVKKRE ECLDALESIM TTKSVSFRRL
SHLRKLVPGF GKAYTIFNKT LMEADAHYKS VRTWNEIIPS
KGCLKVGGRC HPHVNGVFFN GIILGPDGHV LIPEMQSSLL
QQHMELLKSS VIPLMHP

where X represents any amino acid or is absent (i.e., one or more of the X residues may be deleted to remove all or part of this loop in the predicted structure). Illustrative variants may share at least 70%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 55, across the full length of SEQ ID NO: 55 not including the segment representated by X. The polypeptide linker may be any suitable linker. A Gly-Ser linker may be used, such as the polypeptide sequence GSGSGSG. Other suitable linkers include Ser-Ser-Ile-Ser-Asn and Gly-Ser-Gly-Ser-Gly-Ser-Gly.

The deletion of the fusion loop domain may leave a first polypeptide and a second polypeptide segment, which may be linked by a polypeptide linker. In variations, the two polypeptide segments are co-expressed without a polypeptide linker. In further variations, two polypeptide segments are expressed separately and combined together. Chemical linking may be used to cause the two polypeptide segments to remain in complex.

For example, the rabies G protein ectodomain may comprise a first polypeptide segment that shares at least 70%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 56:

(SEQ ID NO: 56)
K FPIYTIPDEL GPWSPIDIHH
LSCPNNLVVE DEGCTNLSEF SYMEL

and/or a second polypeptide segment that shares at least 70%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 57:

(SEQ ID NO: 57)
CDI FTNSRGKRAS KGNKTCGFVD ERGLYKSLKG
ACRLKLCGVL GLRLMDGTWV AMQTSDETKW CPPDQLVNLH
DFRSDEIEHL VVEELVKKRE ECLDALESIM TTKSVSFRRL
SHLRKLVPGF GKAYTIFNKT LMEADAHYKS VRTWNEIIPS
KGCLKVGGRC HPHVNGVFFN GIILGPDGHV LIPEMQSSLL
QQHMELLKSS VIPLMHP

The linker may be any suitable linker. A Gly-Ser linker may be used, such as the polypeptide sequence GSGSGSG.

In some embodiments of the polypeptide, the rabies G protein ectodomain comprises a first polypeptide segment linked to a second polypeptide segment, wherein the first polypeptide segment shares at least 70%, at least 80%, at least 85%, at least 90%, at least 91%, at least 92%, at least 93%, at least 94%, at least 95%, at least 96%, at least 97%, at least 98%, at least 99%, or 100% identity to residues 20-65 of SEQ ID NO: 53; and wherein the second polypeptide segment shares at least 70%, at least 80%, at least 85%, at least 90%, at least 91%, at least 92%, at least 93%, at least 94%, at least 95%, at least 96%, at least 97%, at least 98%, at least 99%, or 100% identity to residues 208-417 of SEQ ID NO: 53.

In some embodiments of the polypeptide, the polypeptide is a fusion protein comprising, in N to C terminal order, the first polypeptide segment, a polypeptide linker, and the second polypeptide segment.

In some embodiments, the rabies G protein ectodomain comprises a deletion, truncation, or subsitution (e.g., with a linker) of amino acid residues G68 through F211, 170 through T212, S71 through T211, S71 through F211, A72 through P207, 173 through P207, K74 through P207, D137 through V144, L141 through V144, or P136 through K148, relative to the reference polypeptide sequence SEQ ID NO: 53. The polypeptide linker sequence, if present, may replace 1, 2, 3, 4, 5, 6, 7, 8, 9, 10, 11, 12 or more amino acid residues. The linker sequence may be shorter than the sequence replaced. For example, residues G68 through P207 may be replaced with a linker of 1, 2, 3, 4, 5, 6, 7, 8, 9, 10, 11, or 12 amino acid residues, optionally a series of glycine, serine, threonine, or alanine residues, or a series of glycine and serine residues (i.e., a glycine-serine linker).

The C-terminus of the polypeptide may be at various suitable residues in the ectodomin of the rabies G protein. In variations, the polypeptide may retain the transmembrane and/or intracellular segments of the rabies G protein. In some embodiments, the C-terminal residues are any residue in the predicted non-helical region (residues 355-445). In some embodiments, the rabies G protein ectodomain terminates at residue 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, or 461 of the native sequence (SEQ ID NO: 53).

In some embodiments, the C-terminal truncation is a deletion of residues 418 through the C terminus of the rabies G protein relative to reference sequence SEQ ID NO: 53, also referred to as a C-terminal truncation at residue 417.

In some embodiments, the rabies G protein ectodomain comprises a sequence that shares at least 70%, at least 80%, at least 85%, at least 90%, at least 91%, at least 92%, at least 93%, at least 94%, at least 95%, at least 96%, at least 97%, at least 98%, at least 99%, or 100% identity to TVFKEGDEAEDFVEVHLPD (SEQ ID NO: 64).

In some embodiments, the rabies G protein ectodomain comprises a sequence that shares at least 70%, at least 80%, at least 85%, at least 90%, at least 91%, at least 92%, at least 93%, at least 94%, at least 95%, at least 96%, at least 97%, at least 98%, at least 99%, or 100% identity to TVFKEGDEAEDFVEVHLPDVYKQISGVDLGLP (SEQ ID NO: 65).

In some embodiments, the rabies G protein ectodomain comprises a sequence that shares at least 70%, at least 80%, at least 85%, at least 90%, at least 91%, at least 92%, at least 93%, at least 94%, at least 95%, at least 96%, at least 97%, at least 98%, at least 99%, or 100% identity to TVFKEGDEAEDFVEVHLPDVYKQISGVDLGLPNWGKY (SEQ ID NO: 66).

Various further modifications of the rabies G protein ectodomain may be included. Using methods described in the Examples, modification of the amino acid sequence may be made and tested to determine which increase (or reduce) expression, the ability to make stable cell lines, protein stability, nanoparticle assembly, and/or antigenicity. For example, and without limitation, the cysteine (Cys) residues of the sequence may be removed by substitutions of an alternative residue, such as serine (Ser), glycine (Gly), or alanine (Ala).

Various rabies G proteins may be used, including but not limited to the ectodomains in Table 1.

TABLE 1
Rabies G Protein Sequences

| Ref. | Ectodomain |
|---|---|
| ASL04726.1 | KFPIYTIPDKLGPWSPIDIHHLSCPNNLVVEDEGCTNLSGFSYMELKVGYISAIK |
| | VNGFTCTGVVTEAETYTNFVGYVTTTFKRKHFRPIPDACRAAYNWKMAGDPRYEE |
| | SLQNPYPDYHWLRTVKTTKESLIIISPSVADLDPYDKSLHSRVFPGGKCLGITVS |
| | STYCSTNHDYTIWMPEEPRLGTSCDIFTSSKGKKASKGGKTCGFVDERGLYKSLK |
| | GACKLKLCGVLGLRLMDGTWVSIQTSDDTKWCPSDQLVNLHDFHSDEIEHLVVEE |
| | LVKKREECLDALESIMTTKSVSFRRLSYLRKLVPGEGKAYTIFNKTLMEADAHYK |
| | SVRTWNEIIPSKGCLKVRERCHPPVDGVFFNGIILGPDGNVLIPEMQSSLLQQHM |
| | ELLESSVIPLMHPLADPS (SEQ ID NO: 104) |
| AAB30931.1 | KFPIYTIPDTLGPWSPIDIHHLSCPNNLVVEDEGCTNLSGFSYMELKVGHISAIK |
| | VNGFTCTGVVTEAETYTNFVGYVTTTFKRKHFRPTPDACRAAYNWKMAGDPRYEE |
| | SLHSPYPDYHWLRTVKTTKESLVIISPSVADLDPYDNSLHSRVFPSGKCSGITVS |
| | SVYCSTNHDYTVWMPESLRLGTSCDIFTNSRGKRVSKGSKTCGFVDERGLYKSLK |
| | GACKLKLCGVRGLRLMDGTWVAMQTSNETKWCPPDQLVNLHDLRSDEIEHLVIEE |
| | LVKKREECLDALESIITTKSVSFRRLSYLRKLVPGFGKAYTIFNKTLMEAEAHYK |
| | SVRTWNEIIPSKGCLRVGGRCHPHVNGVFFNGIILGPDGHVLIPEMQSSLLQQHI |
| | ELLESSVIPLMHPLADPF (SEQ ID NO: 105) |
| CCH26353.1 | KFPIYTIPDKLGPWSPIDIHHLSCPNNLVVEDEGCTNLSGFSYMELKVGYISAIK |
| | VNGFTCTGVVTEAETYTNFVGYVTTTFKRKHFRPTPDACRAAYNWKMAGDPRYEE |
| | SLHNPYPDYHWLRTVKTTKESLVIISPSVADLDPYDKSLHSRVFPSGKCLGITIS |
| | STYCSTNHDYTIWMPENPRLGTSCDIFTNSRGKRASKGSKTCGFVDERGLYKSLK |
| | GACKLKLCGVLGLRLMDGTWVAMQTSDETKWCPPDQLVNLHDFRSDEIEHLVVEE |
| | LVKKREECLDALESIMTTKSVSFRRLSHLRKLVPGFGKAYTIFNKTLMEADAHYK |
| | SVRTWNEIIPSKGCLRVGGRCHPHVNGVFFNGIILGPDGHVLIPEMQSSLLQQHM |
| | ELLESSVIPLMHPLADPS (SEQ ID NO: 106) |
| AFR56627.1 | KFPIYTIPDKLGPWSPIDIHHLSCPNNLVVEDEGCTNLSGFSYMELKVGYISAIK |
| | TNGFTCTGVVTEAETYTNFVGYVTTTFKRKHFRPTPDACRAAYNWKMAGDPRYEE |
| | SLHNPYPDYHWLRTVKTTKESLVIISPSVADLDPYDRSLHSRVFPGGNCSGVAVS |
| | STYCSTNHDYTIWMPENPRLGMSCDIFTNSRGKRASKGSETCGFVDERGLYKSLK |
| | GACKLKLCGVLGLRLMDGTWVAMQTSNETKWCPPGQLVNLHDFRSDEIEHLVVEE |
| | LVKKREECLDALESIMTTKSVSFRRLSHLRKLVPGFGKAYTIFNKTLMEADAHYK |
| | SVRTWNEIIPSKGCLRVGGRCHPHVNGVFFNGIILGPDGNVLIPEMQSSLLQQHM |
| | ELLVSSVIPLMHPMADPS (SEQ ID NO: 107) |
| ABI47945.1 | KFPIYTIPTKLGPWSPIDIHHLSCPNNLVVEDEGCTNLSGFSYMELKVGRISAIK |
| | VNGFTCTGVVTEAETYTNFVGYVTTTFKRKHFRPMPGCMYSRVQLEDGRSPQIEE |
| | SLHNPYPDYHWLRTVRTTKESLIIISPSVTDLDPYDKSLHSRVFPGRKCSGITVS |
| | STYCSTNHDYTVWMPEILRLGTSCDIFTNSRGKRASKGSKTCGFVDERGLYKSLK |
| | GACKLKLCGVPGLRLMDGTWVAMQTSNETKWCPPGQLVNLHDLHSDEIEHLVVEE |
| | LVKKREECLDALESITTTKSVSFRRLSHLRKLVPGFGKAYTIFNKTLMEAEAHYK |
| | SVRTWNEIIPSKGCLRVGGGCHPHVNGVFFNGIILGPDGHVLIPEMQSSLLQQHI |
| | ELLESSVIPLMHPLADPF (SEQ ID NO: 108) |
| AHB86382.1 | KFPIYTIPDKLGPWSPIDIHHLSCPNNLVVEDEGCNSLSGFSYMELKVGYISAIK |
| | VNGFTCTGVVTEAETYTNFVGYVTTTFKRKHFRPMPDACRSAYNWKLAGDPRYEE |
| | SLHNPYPEYHWLRTVKTTKESLIVISPSVADLDPYDKSLHSRIFPNGKCSGMTVS |
| | STYCSTNHEYTIWMPEKVGLGTSCDIFTTSKGKRASKGGRTCGFVDERGLYKSLK |
| | GACKLKLCGVSGLRLMDGTWVAIQTSDETKWCSPDQLVNLHDLHSDEIEHLVVEE |
| | LVRKREECLDALESIMTTKSVSFRRLSHLRKLVPGFGKAYTIFNKTLMEADAHYK |
| | SVRTWNEIIPSKGCLRVGGKCHPHVNGVFFNGIILGPDGHVLIPEMQSSLLQQHI |
| | ELLESSVIPLMHPLADLS (SEQ ID NO: 109) |
| AEV43288.1 | KFPIYTIPDKLGPWSPIDIHHLSCPNNLVVEDEGCTNLSGFSYMELKVGYISAIK |
| | VNGFTCTGVVTEAETYTNFVGYVTTTFKRKHFRPTPDACRSAYNWKMAGDPRYEE |
| | SLHNPYPDYHWLRTVKTTKESVVIISPSVADLDPYDKSLHSRVFPRGKCSGITVS |
| | SAYCSTNHDYTIWMPENPRLGTSCDIFTNSRGKRASKGSKTCGFVDERGLYKSLK |
| | GACKLKLCGVLGLRLMDGTWVAIQTSNETKWCPPDQLVNLHDFHSDEIEHLVVEE |
| | LVKKREECLDALESIMTTKSVSFRRLSHLRKLVPGFGKAYTIFNKTLMEADAHYK |
| | SVRTWNEIIPSKGCLRVGGRCHPHVNGVFFNGIILGPDGHVLIPEMQSSLLQQHM |
| | ELLKSSVIPLMHPLADPS (SEQ ID NO: 110) |
| ADJ68075.1 | KFPIYTIPDKLGPWSPIDIHHLSCPNNLVVEDEGCTNLSGFSYMELKVGYISAIK |
| | VNGFTCTGVVTEAETYTNFVGYVTTTFKRKHFRPMPDACRAAYNWKMAGDPRYEE |
| | SLHNPYPDYHWLRTVKTSKESLVIISPSVADLDPYDKSLHSRVFPGGKCSGITVS |
| | STCCSTNHDYTIWMPENPRLGTSCDIFTNSRGKRASKGGKTCGFVDERGLYKSLK |
| | GACKMKLCGVLGLRLMDGTWVAIQTSDEIKWCSPDQLVNLHDFHSDEIEHLVVEE |
| | LVKKREECLDALETIMTTKSVSFRRLSHLRKLVPGFGKAYTIFNKTLMEADAHYK |
| | SIRTWNEIIPSKGCLRVGGRCHPHVNGVFFNGIILGPDGHVLIPEMQSSLLHQHM |
| | ELLESSVIPLMHPLADPS (SEQ ID NO: 111) |
| AAK11477.1 | KFPIYTIPDKLGPWSPIDIHHLSCPNNLVVEDEGCTNLSGFSYMELKVGYISAIK |
| | VNGFTCTGVVTEAETYTNFVGYVTTTFKRKHFRPTPDECRAAYNWKMAGDPRYEE |
| | SLHNPYPDYHWLRTVKTTKESLVIISPSVADLDPYDKSLHSRVFPSGKCSGITVS |
| | STYCSTNHDYTIWMPENPRLGTSCDIFTNSRGKRASKGSKTCGFVDERGLYKSLK |
| | GACKLKLCGVLGLKLMDGTWVAMQTSDETKWCPPDQLVNLHDLRSDEIEHLVVEE |
| | LVKKREECLDALESIMTTKSVSFRRLSHLRKLVPGFGKAYTIFNKTLMEADAHYK |
| | SVRTWNEIIPSKGCLKVEGRCHPHVNGVFFNGVILGPDGHVLIPEMQSSLLQQHM |
| | ELLESSVIPLMHPLADPS (SEQ ID NO: 112) |
| AAA69485.1 | KFPIYTIPDKLGPWSPIDIHHLSCPNNLVVEDEGCNSLSGFSYMELKVGYISAIK |
| | VNGFTCTGVVTEAETYTNFVGYVTTTFKRKHFCPMPDACRSAYNWKMAGDPRYEE |
| | SLHNPYPDYHWLRTVKTTKESLIIISPSVAELDPYDKSLHSRIFPGGKCTGLTVS |
| | STYCSTNHDYTIWMPEKAGLGTSCDIFTNSKGKRASKGGKTCGFVDERGLYKSLK |
| | GACKLKLCGVSGLRLMDGTWVAIQTLDETKWCSPDQLVNLHDFHSDELEHLVVEE |
| | LVRKREECLDALESIMTTKSVSFRRLSHLRKLVPGFGKAYTIFNKTLMEADAHYK |
| | SVRTWSEIIPSKGCLRVGGRCHPHVNGVFFNGIILGPDGHVLIPEMQSSLLQQHV |
| | ELLESSVIPLMHPLADPS (SEQ ID NO: 113) |
| AMM70629.1 | KFPIYTIPDKLGPWSPIDIHHLSCPNNLVVEDEGCTSLSGFSYMELKVGYISAIK |
| | VNGFTCTGVVTEAETYTNFVGYVTTTFKRKHFRPMSDACRAAYNWKVAGDPRYEE |
| | SLQNPYPDYHWLRTVKTTKESLIIISPSVADLDPYDKSLRSRIFPGGKCLGITVS |
| | STYCSTNHDYTIWMPEEARLGTSCDIFTNSKGKRASKGGKICGFVDERGLYKSLK |
| | GACRLKLCGVSGLRLMDGTWVAIQTSEETKWCPSDQLVNLHDFHSDEIEHLVVEE |
| | LVKKREECLDTLESIMTTKSVSFRRLSHLRKLVPGFGKAYTIFNKTLMEADAHYN |
| | SVRTWNEIIPSKGCLKVGGRCHPHVDGVFFNGIILGPDGNVLIPEMQSSLLQQHM |
| | ELLESSVIPLMHPLADPS (SEQ ID NO: 114) |
| AMM70625.1 | KFPIYTIPDKLGPWSPIDIHHLSCPNNLVVEDEGCTNLSGFSYMELKVGYISAIK |
| | VNGFTCTGVVTEAETYTNFVGYVTTTFRRKHFRPTPDACRTAYNWKMAGDPRYEE |
| | SLHNPYPDYHWLRTVKTTKESLVIISPSVADLDPYDRSLHSRVFPNGKCSGITVS |
| | STYCPTNHDYTIWMPENPRLGTSCDIFVNSRGKRASKGSKTCGFVDERGLYKSLK |
| | GACKLKLCGVLGLRLMDGTWVAMRTSDETKWCPPDQLVNIHDFHSDEIEHLVVEE |
| | LVKKREECLDALESIMTTKSVSFRRLSHLRKLVPGFGKAYTIFNKTLMEADAHYK |
| | SVRTWDEIIPSKGCLRVGGRCHPHVHGVFFNGIILGPDGHVLIPEMQSSLLQQHM |
| | ELLESSVIPLMHPLADPS (SEQ ID NO: 115) |
| AIL01055.1 | KFPIYTIPDKLGPWSPIDIHHLSCPNNLVVEDEECTSLSGFSYMELKVGYISAIK |
| | VNGFTCTGVVTEAETYTNFVGYVTTTFKRKHFRPTPDACRAAYNWKMAGDPRYEE |
| | SLHNPYPDYHWLRTVKTTKESLIIISPSVADLDPYDKSLHSRVFPGGKCLGITIS |
| | STYCSTNHDYTIWMPEEARLGTSCDIFANSRGKRASKGGRTCGFVDERGLYKSLK |
| | GACKLKLCGVPGLRLMDGTWVTIQTSNETKWCPPDRLVNLHDFHSDEIEHLVVEE |
| | LVKKREECLDALESIMTTKSVSFRRLSYLRKLVPGFGKAYTIFNKTLMEADAHYK |
| | SVRTWNEIIPSKGCLKVGGRCHPHVDGVFFNGIILGPDGNVLIPEMQSSLLQQHM |
| | ELLESSVIPLMHPLADPS (SEQ ID NO: 116) |
| AIL01053.1 | GFPIYTIPDKLGPWSPIDIHHLSCPNNLVVEDEGCTSLSGFSYMELKVGYISAIK |
| | VNGFTCTGVVTEAETYTNFVGYVTTTFKRKHFRPMPDSCRAAYNWKTAGDPRYEE |
| | SLQNPYPDYHWLRTVKTTKESLIIISPSVADLDPYDKSLHSRVFPGGKCLGITVS |
| | STYCSTNHDYTIWMPEEARLGTSCDIFTNSRGKRASKGGRTCGFVDERGLYKSLK |
| | GACKLKLCGVLGLRLMDGTWVTIHTSNETKWCPSDQLVNLHDFHSDEIEHLVVEE |
| | LVKKREECLDALESIMTTKSVSFRRLSHLRKLVPGFGKAYTIFNKTLMEADAHYK |
| | SIRTWNEIIPSKGCLKVGGRCHPHVDGVFFNGIILGPDGNVLIPEMQSSLLQQHV |
| | ELLESSVIPLMHPLADPS (SEQ ID NO: 117) |
| AIL01046.1 | KFPIYPIPDKLGPWSPIDIHHLSCPNNLVGEDEGCISLSGFSYMELKVGYISAIK |
| | VNGFTCTGVVTEAETYTNFVGYVTTTFKRKHFRPIPDACRAAYNWKIAGDPRYEE |
| | SLQNPYPDYHWLRTVKTTKESLIIISPSVADLDPYDKSLHSRLFPGGKCLGITIS |
| | STYCSTNHDYTIWMPEEPRLGTSCDIFTNSKGKKASKGGKVCGFVDERGLYKSLK |
| | GACKLKLCGVSGLRLMDGTWVTIQTSDETKWCPPDQLVNLHDFHSDEIEHLVVEE |
| | LVKKREECLDALESIMTTKSVSFRRLSYLRKLVPGFGKAYTIFNKTLMEADAHYK |
| | SVRTWNEIIPSKGCLKVSDRCHPHVDGVFFNGIILGPDGNVLIPEMQSSLLQQHM |
| | ELLESSVIPLMHPLADPS (SEQ ID NO: 118) |
| AIL01038.1 | KFPIYTIPDKLGPWSPIDIHHLSCPNNLVVEDEGCTSLSGFSYMELKVGYISAIK |
| | VNGFTCTGVVTEAETYTNFVGYVTTTFKRKHFRPMPDACRAAHDWKIAGDPRYED |
| | SLQNPYPDYHWLRTVKTTKESLVIISPSVAELDPYDKSLHSRVFPSGKCLGITVS |
| | STYCSTNHDYTIWMPVEARLGTSCDIFTNSRGKKASKGGRTCGFVDERGLYKSLK |
| | GACKLKLCGVPGLRLMDGTWVSIQTSDDIKWCPPDQLVNLHDFHSDEIEHLVVEE |
| | LIKKREGCLDALESIMTTKSVSFRRLSHLRKLVPGFGKAYTIFNNTLMEADAHYK |
| | SVRTWNEVIPSKGCLKVGGRCHPPVNGVFFNGIILGPDGKVLIPEMQSSLLQQHM |
| | ELLESSVIPLMHPLADPS (SEQ ID NO: 119) |
| AIL01037.1 | KFPIYTIPEKLGPWSPIDIHHLSCPNNLVVEDEGCTTLSGFSYIELKVGYISAIK |
| | VNGFTCTGVVTEAETYTNFVGYVTTTFKRKHFRPMPDACRAAYNWKIAGDPRYEE |
| | SLHNPYPDYHWLRTVKTTKESLVIISPSVADLDPYDKSLHSRVFPGGKCLGITIS |
| | STYCSTNHDYTIWMPEEARLGTSCDIFTNSRGKRASKGGRTCGFVDERGLYKSLK |
| | GACKLKLCGVPGLRLMDGTWVSIQASDETKWCSPDQLVNVHDFHSDEIEHLVVEE |
| | LVKRREECLDALESIVTTKSISFRRLSHLRKLVPGFGKAYTIFNKTLMEADAHYK |
| | SVRTWNEIIPSKGCLSVGGRCHPHVNGVFFNGIILGPDGNVLIPEMQSSLLQQHM |
| | ELLESSVIPLMHPLADPS (SEQ ID NO: 120) |
| AIL01032.1 | KFPIYTIPEKLGPWSPIDIHHLGCPNNLVVEDEGCDSLSGFSYMELKVGYISAIK |
| | VNGFTCTGVVTEAETYTNFVGYVTTTFKRKHFRPMPDACRAAYNWKTAGDPRYEE |
| | SLHNPYPDYHWLRTVKTTKESLIIISPSVADLDPYDKSLHSRVFPGGKCLGITVS |
| | STYCSTNHDYTIWMPEETRLGTSCDIFTNSRGKRASKGGKTCGFVDERGLYKSLK |
| | GACKLKLCGVPGLRLMDGTWVSIQTPDETKWCSPDQLVNLHDFHSDEIEHLVVEE |
| | LVKKREECLDALESIMTTKSVSFRRLSHLRKLVPGEGKAYTIFNKTLMEADAHYK |
| | SVRTWDEIIPSKGCLRVGGRCHPHVNGVFFNGIILGPDGHVLIPEMQSSLLQQHM |
| | ELLESSVIPLMHPVANPS (SEQ ID NO: 121) |
| AIL01031.1 | EFPIYTIPEKLGPWSPIDIHHLSCPNNLVVEDEGCNSLSGFSYMELKVGYISSIK |
| | VNGFTCTGVVTEAETYTNFVGYVTTTFKRKHFRPMPDACRAAYNWKMSGDPRYEE |
| | SLHNPYPDYHWLRTVKTTKESLVIISPSVADLDPYDKSLHSRVFSGGKCLGITIS |
| | STYCSTNHDYTLWMPEEARLGTSCDIFTNSKGKRASKGGKTCGFVDERGLYKSLK |
| | GACKLKLCGVPGLRLMDGTWVSIQTSDETKWCPPDQLVNIHDFHSDEIEHLVVEE |
| | LVKKREECLDALESIMTTKSVSFRRLSHLRKLVPGFGKAYTIFNKTLMEADAHYK |
| | SVRTWNEVIPSKGCLSVGGRCHPHVDGVFFNGIILGPDGSVLIPEMQSSLLQQHM |
| | ELLESSVIPLMHPLANPS (SEQ ID NO: 122) |
| AIL01029.1 | KFPIYTIPDKIGPWSPIDIHHLSCPNNLVVEDEGCTSLSGFSYMELKVGYISAIK |
| | VNGFTCTGVVTEAETYTNFVGYVTTTFKRKHFRPTPDACRAAYNWKMAGDPRYED |
| | SLQNPYPDYHWLRTVKTTKESLIIISPSVADLDPYDKSLHSRVFPSGKCLGITVS |
| | STYCSTNHDYTIWMPEEARLGTSCDIFTNSKGKRASKGGKTCGFVDERGLYKSLR |
| | GACKLKLCGVPGLRLMDGTWVSIQTTDETKWCPSDQLVNLHDFHSDEIEHLVVEE |
| | LVKKREECLDALESIMTTKSVSFRRLSHLRKLVPGFGKAYTIFNKTLMEADAHYK |
| | SVQTWNEIIPSKGCLKVGGRCHPHVDGVFFNGIILGPDGNVLIPEMQSSLLQQHM |
| | ELLESSVIPLMHPLADPS (SEQ ID NO: 123) |
| AIL01021.1 | EFPIYTIPDKLGPWSPIDIHHLSCPNNLVAENDGCTSLSGFSYMELKVGYISAIK |
| | VNGFTCTGVMTEAETYTNFVGYVTTTFKRKHFRPMPDACRAAHDWKIAGDPRYED |
| | SLQNPYPDYHWLRTVKTTKESLVIISPSVADLDPYDKSLHSRVFPSGKCLGITVS |
| | STYCSTNHDYTIWMPVEPRLGTSCDIFTNSRGKRASKGGRVCGFVDERGLYKSLK |
| | GACKLKLCGVPGIRLMDGTWVSIQTSEEIKWCPPDRLVNLHDFHSDEIEHLVVEE |
| | LIKRRENCLDALESIMTTKSVSFRRLSHLRKLVPGFGKAYTIFNKTLMEADAHYK |
| | SVKTWNEVIPSKGCLEVGGRCHPPVNGVFFNGIILGPDGNVLIPEMQSSLLQQHM |
| | ELLESSVIPLMHPLADPS (SEQ ID NO: 124) |
| AIL01017.1 | KFPIYTIPDKLGPWSPIDIHHLSCPNNLVVEDEGCTSLSGFSYMELKVGYISAIK |
| | VNGFTCTGVVTEAETYTNFVGYVTTTFKRKHFRPMPDACRAAHEWKMAGDPRYED |
| | SLQNPYPDYHWLRTVKTTKESLIIISPSVADLDPYDKSLHSRVFPGGNCLGITIS |
| | STYCSTNHDYTIWMPEEARLGTSCDIFTSSRGKRASKGGKICGFVDERGLYKSLK |
| | GACKLKLCGVPGLRLMDGTWVSIQTSDETKWCPPDQLVNVHDFHSDEIEHLIVEE |
| | LVKKREECLDALESIMTTKSVSFRRLSHLRKLVPGFGKAYTIFNKTLMEADAHYK |
| | SVRTWNEIIPSKGCLKVGGMCHPHVGGVFFNGIILGPDGTVLIPEMQSSLLQQHM |
| | ELLESSVIPLMHPLADPS (SEQ ID NO: 125) |
| AIL00998.1 | KFPIYTIPEKLGPWSPIDIHHLSCPNNLVVEDEGCDSLSGFSYMELKVGYISAIK |
| | VNGFTCTGVVTEAETYTNFVGYVTTTFKRKHFRPMPDACRAAYNWKTAGDPRYEE |
| | SLHNPYPDYHWLRTVKTTKESLIIISPSVADLDPYDKSLHSRVFPGGKCLGITVS |
| | STYCSTNHDYTIWMPEEARLGTSCDIFTNSKGKRASKGGRTCGFVDERGLYKSLK |
| | GACKLKLCGVPGLRLMDGTWVAIQTPGETKWCSPDQLVNLHDFRSDEIEHLVVEE |
| | LIKKREECLDALESIMTTKSVSFRRLSNLRKLVPGFGKAYTIFNKTLMEADAHYK |
| | SVRTWDEIIPSKGCLRVGGRCHPHVNGVFFNGIILGPDGHVLIPEMQSSLLQQHM |
| | ELLESSVIPLIHPLADPS (SEQ ID NO: 126) |
| AIL00989.1 | KFPIYTIPEKLGPWSPIDIHHLSCPNNLVVEDEGCNSLSGFSYMELKVGYISAIK |
| | VNGFTCTGVVTEAETYTNFVGYVTTTFKRKHFRPMPDACRAAYNWKTAGDPRYEE |
| | SLHNPYPDYHWLRTVKTTKESLIIISPSVADLDPYDKSLHSRIFPGGKCLGITIS |
| | STYCSTNHDYTIWMPEEARLGTSCDIFTNSKGKRASKGGRTCGFVDERGLYKSLK |
| | GACKLKLCGVPGLRLMDGTWVAIQTPNETKWCSPDQLVNVHDFRSDEIEHLVVEE |
| | LIRKREECLDALESIMTTKSVSFRRLSNLRKLVPGFGKAYTIFNKTLMEADAHYK |
| | SVRTWNEIIPSRGCLKVGGRCHPHVNGVFFNGIILGPDGHILIPEMQSSLLQQHM |
| | ELLESSVIPLIHPLADPS (SEQ ID NO: 127) |
| AHB86369.1 | KFPIYTIPDKLGPWSPIDIHHLSCPNNLVVEDEGCNSLSGISYMELKVGYISAIK |
| | VNGFTCTGVVTEAETYTNFVGYVTTTFKRKHFHPMPDACRSAYNWKLAGDPRYEE |
| | SLHNPYPDYHWLRTVKTTKESLIIISPSVADLDPYDKSLHSRIFPSGKCSGMTIS |
| | STYCSTNHEYTIWMPEKAGLGTSCDIFTTSKGKRASKGGKTCGFVDERGLYKSLK |
| | GACKLKLCGVSGLRLMDGTWVAIQTSEETKWCPPDQLVNLHDLHSDEIEHLVVEE |
| | LVKKREECLDALESIMTTKSVSFRRLSHLRKLVPGFGKAYTIFNKTLMEADAHYK |
| | SIRTWNEIIPSKGCLRVGGKCHPHVDGVFFNGIILGPDGHVLIPEMQSSLLQQHM |
| | ELLESSVIPLMHPLADLS (SEQ ID NO: 128) |
| AGN94548.1 | KFPIYTIPDKLGPWSPIDIHHLSCPNNLVVEDEGCNSLSGFSYMELKVGYISAIK |
| | VNGFTCTGVVTEAETYTNFVGYVTTTFKRKHFRPVPDACRSAYNWKLAGDPRYEE |
| | SLHNPYPDYHWLRTVKTTKESLIIISPSVADLDPYDKSLHSRIFPDGKCSGITVS |
| | STHCSTNHDYTIWMPEKAGLGTSCDIFINSRGKRASKGGRTCGFVDERGLYKSLK |
| | GACKLKLCGVPGLRLMDGTWAAIQTADETKWCSPDQLVNLHDFHSDEIEHLVVED |
| | LVKKREECLDALESIMTTKSVSFRRLSHLRKLVPGFGKAYTIFNKTLMEADAHYK |
| | SVRTWNEIIPSKGCLRVGGRCHPHVNGVFFNGIILGPDGHVLIPEMQSSFLQQHM |
| | ELLESSVIPLMHPLADPS (SEQ ID NO: 129) |
| AGN94533.1 | KFPIYTIPDKLGPWSPIDIHHLSCPNNLVVEDEGCTNLSGFSYMELKVGYISAIK |
| | VNGFTCTGVVTEAETYTNFVGYVTTTERRKHFRPTPDACRAAYNWKMAGDPRYEE |
| | SLHNPYPDYHWLRTVKTTKESLVIISPSVADLDPYDKSLHSRVFPGGKCSGITVS |
| | STYCSTNHDYTIWMPENPRLGTSCDIFVNSRGKRASKGGKTCGFVDERGLYKSLK |
| | GACKLKLCGVPGLRLMDGTWVAIQTSDETKWCPPDQLVNLHDFRSDEIEHLVVEE |
| | LVKKREECLDALESIMTTKSVSFRRLSHLRKLVPGFGKAYTIFNKTLMEADAHYK |
| | SVRTWNEIIPSKGCLRVGGRCHPHVNGVFFNGIILGPDGHVLIPEMQSSLLQQHM |
| | ELLESSVIPLMHPLADPS (SEQ ID NO: 130) |
| AGN94484.1 | KFPIYTIPDKLGPWSPIDIHHLSCPNNLVAEDEGCTTLSGFSYMELKVGYISAIK |
| | VNGFTCTGVVTEAETYTNFIGYVTTTFKRKHFSPRPDACRTAYNWKITGDPRYEE |
| | SLQNPYPDYSWLRTVKTTKESLVIISPSVADLDPYDKSLHSRVFPGGRCLGITIY |
| | STYCSTNHDYTVWLPEEARLGTSCDIFVKSRGKRASKGGRTCGFVDERGLYRSLK |
| | GACKLKLCGVPGLRLVDGTWVTIQTSDETKWCPSEQLVNLHNFHSDEIEHLVVEE |
| | LVKKREECLDALESIMTTKSVSFRRLSHLRKLVPGFGKAYTIFNKTLMEADAHYT |
| | SVRTWNEIIPSKGCLKVGGKCHPHVGGVFFNGIILGPDGNILIPEMQSSLLQQHM |
| | ELLESSVIPLMHPLADPS (SEQ ID NO: 131) |
| AGN94472.1 | KFPIYTIPDKLGPWSPIDIHHLSCPNNLVVEDEGCNNLSGFSYMELKVGYISAIK |
| | VNGFTCTGVVTEAETYTNFVGYVTTTFKRKHFRPTPDACRAAYNWKIAGDPRYEE |
| | SLHNPYPDYHWLRTVKTTKESLVIISPSVADLDPYDKSLHSRVFPSGKCSGMTVS |
| | STCCSTNHDYTIWMPENPRLGTSCDIFTNSRGKRASKGSKTCGFVDERGLYKSLK |
| | GACKLKLCGVLGLRLMDGTWVAMQTSDEIKWCPPDQLVNLHDERSDEIEHLVVEE |
| | LVKKREECLDALESIMTTKSVSFRRLSHLRKLVPGFGKAYTIFNRTLMEADAHYK |
| | SVRTWNEVIPSKGCLRVGGRCHPHVNGVFFNGIILGPDGHVLIPEMQSSLLQQHM |
| | ELLESSVIPLMHPLANPS (SEQ ID NO: 132) |
| AGN94339.1 | KFPIYTIPDKLGPWSPIDIHHLSCPNNLVIEDEGCNNLSGFSYMELKVGYISAIK |
| | VNGFTCTGVVTEAETYTNFVGYVTTTFKRKHFRPMPDACRSAYNWKLAGDPRYEE |
| | SLHNPYPDYHWLRTVKTTKESLIVISPSVADLDPYDKSLHSRIFPGGKCSGMTVS |
| | STYCSTNHEYTIWMPEKPGLGTSCDIFATSKGKRASKGGRTCGFVDERGLYKSLK |
| | GACKLKLCGVSGLRLMDGTWVAIQTSDETKWCSPDQLVNLHDLHSDEIEHLVVEE |
| | LVRKREECLDALESIMTTKSVSFRRLSHLRKLVPGFGKAYTIFNKTLMEADAHYK |
| | SVRTWNEIIPSKGCLRVGGKCHPPVNGVFFNGIILGPDGHVLIPEMQSSLLQQHM |
| | ELLESSVIPLMHPLADPS (SEQ ID NO: 133) |
| AGN94293.1 | KFPIYTIPDKIGPWSPIDIHHLSCPNNLVVEDEGCTSLSGFSYMELKVGYISAIK |
| | VNGFTCTGVVTEAETYTNFVGYVTTTFKRKHFRPMPDACRAAYNWKMAGDPRYEE |
| | SLQNPYPDYNWLRTVKTTKESLIIISPSVADLDPYDKSLHSRVFPNGKCLGITVS |
| | STYCSTNHDYTIWMPEEARLGTSCDIFTNSKGKRASKGGKTCGFVDERGLYKSLR |
| | GACKLKLCGVPGLRLMDGTWVSIQTSDETKWCPPDQLVNVHDFHSDEIEHLVVEE |
| | LVRKREECLDALESIMTTKSVSFRRLSHLRKLVPGFGKAYTIFNKTLMEADAHYK |
| | SIQTWNEIIPSKGCLKVGGMCHPHLDGVFFNGIILGPDGNVLIPEMQSSLLQQHM |
| | ELLESSVIPLMHPLADPS (SEQ ID NO: 134) |
| AGN94153.1 | KFPLYTIPDKLGPWSPIDIHHLSCPNNLVVEDEGCTNLSGFSYMELKVGYISAIK |
| | VSGFTCTGVVTEAETYTNFVGYVTTTFKRRHFRPTPDACRAAYNWKMAGDPRYEE |
| | SLHNPYPDYHWLRTVKTTKESLVIISPSVTDLDPYDKSLHSRVFPGGKCSGITVS |
| | STYCSTNHDYTIWMPESPRLGTSCDIFTNSRGKRASKGSKTCGFVDERGLYKSLK |
| | GACKLKLCGVLGLRLMDGTWVAMQTSDETKWCLPDQLVNLHDFRSDEIEHLVVEE |
| | LVRKREECLDALESIMTTKSVSFRRLSHLRKLVPGFGKAYTIFNKTLMEADAHYK |
| | SIRTWNEIVPSKGCLRVGGRCHPHVNGVFFNGIILGPDGQVLIPEMQSSLLQQHM |
| | ELLESSVIPLMHPLADPS (SEQ ID NO: 135) |
| AGN94083.1 | KFPIYTIPDKLGPWSPIDIHHLSCPNNLVVEDEGCTNLSGFSYMELKVGYISAIK |
| | VNGFTCTGVVTEAETYTNFVGYVTTTFKRKHLRPAPDACRNAFNWKMAGDPRYEE |
| | SLHNPYPDYHWLRTVKTTKESLVIISPSVADLDPYDKSLHSRVFPSGKCSGITVS |
| | STYCSTNHDYTVWMPENPRLGTSCDIFTNSKGKRASKGSRTCGFVDERGLYKSLK |
| | GACKLKLCGVPGLRLTDGTWVAMQTSEETKWCPHDQLVNLHDFRLDEIEHLVVEE |
| | LVKKREECLDALESIMTTKSVSFRRLSHLRKLVPGFGKAYTIFNKTLMEADAHYK |
| | SVRTWNEIIPSKGCLKVGGSCHPHVNGVFFNGIILGPDGHVLIPEMQSSLLQQHM |
| | ELLESSVIPLMHPLADPS (SEQ ID NO: 136) |
| ALM97199.1 | KFPIYTIPDKLGPWSPIDIHHLSCPNNLVVEDEGCTNLSGFSYMELKVGYISAIK |
| | VNGFTCTGVVTEAETYTNFVGYVTTTFKRKHFRPTPDACRAAYNWKMAGDPRYEE |
| | SLHNPYPDYHWLRTVKTTKESLVVISPSVADLDPYDKSLHSRVFPSGKCSGITVS |
| | STYCSTNHDYTIWMPENPRLGTSCDIFVNSRGKRASKGSKTCGFVDERGLYKSLK |
| | GACKLKLCGVLGLRLMDGTWVAIQTSDETKWCPPDQLVNLHDFRSDEIEHLVVEE |
| | LVKKREECLDALESIMTTKSVSFRRLSHLRKLVPGFGKAYTIFNKTLMEADAHYK |
| | SVRTWNEIIPSKGCLRVGGRCHPHVNGVFFNGIILGPDGHVLIPEMQSSLLQQHM |
| | ELLESSVIPLMHPLADPS (SEQ ID NO: 137) |
| AFN24533.1 | KFPIYTIPEKLGPWSPIDIHHLSCPNNLVAEDEGCNSLSGFSYMELKVGYISAIK |
| | VNGFTCTGVVTEAETYTNFVGYVTTTFKRKHFRPTPDACRAAYNWKMAGDPRYEE |
| | SLHNPYPDYHWLRTVKTTKESLVIISPSVADLDPYDKSLHSRVFPDGKCLGITIS |
| | STYCSTNHDYTIWMPEEARLGTSCDIFTTSKGKRASKGDKICGFVDERGLYKSLK |
| | GACKLKLCGVLGLRLMDGTWVSIQTPGETKWCSPDQLVNLHDFHSDEIEHLVAEE |
| | LVKKREECLDALESIMTTKSVSFRRLSHLRKLVPGFGKAYTIFNKTLMEADAHYK |
| | SVRTWNEVIPSKGCLSVGGRCHPHVNGVFFNGIILGPDGSVLIPEMQSSLLQQHM |
| | ELLESSVIPLMHPLADPS (SEQ ID NO: 138) |
| AFN24525.1 | KFPIYTIPEKLGPWSPIDIHHLSCPNNLVVEDEGCNSLSGFSYMELKVGYISAIK |
| | VNGFTCTGVVTEAETYTNFVGYVTTTFKRKHFRPMPDACRAAYNWKMAGDPRYEE |
| | SLHNPYPDYHWLRTVKTTKESLVIISPSVADLDPYDKSLHSRVFPGGKCLGITIS |
| | STYCSTNHDYTIWMPEEARLGTSCDIFTNSKGKRASKGGRTCGFVDERGLYKSLK |
| | GACKLKLCGVPGLRLMDGTWVSIQTSEETKWCSSDQLVNIHDFRSDEIEHLVVEE |
| | LVKKREECLDALESIMTTKSVSFRRLSHLRKLVPGFGKAYTIFNNTLMEADAHYK |
| | SVKTWNEIIPSKGCLSVGGRCHPNVNGVFFNGIILGPDGSVLIPEMQSSLLQQHM |
| | ELLESSVIPLMHPLANPS (SEQ ID NO: 139) |
| AFN24383.1 | EFPIYTIPDKLGPWSPIDIHHLSCPNNLVVEDEGCTNLSGFSYMELKVGYISAIK |
| | VNGFTCTGVVTEAETYTNFVGYVTTTFKRKHFRPTPDACRAAYNWKMAGDPRYEE |
| | SLHNPYPDYHWLRTVKTTKESLVIISPSVADLDPYDKSLHSRVFPSGKCSGITVS |
| | STYCLTNHDYTIWMPENLRLGTSCDIFTNSRGKRASRGSKTCGFVDERGLYKSLK |
| | GACKLKLCGVLGLRLMDGTWVAMQTSDETKWCPSDQLVNLHDFRSDEIEHLVVEE |
| | LVKKREECLDAIESIMTTKSVSFRRLSHLRKLVPGFGKAYTIFNKTLMEADAHYK |
| | SVRTWNEIIPSKGCLRVGGRCHPHVNGVFFNGIILGPDGHVLIPEMQSSLLQQHV |
| | ELLESSVIPLMHPLADPS (SEQ ID NO: 140) |
| AFN24303.1 | KFPIYTIPDKLGPWSPIDIHHLSCPNNLVVEDEGCSNLSGFSYMELKVGYISAIK |
| | VDGFTCTGVVTEAETYTNFIGYVTTTFKRKHFRPMPDACRSAYNWKIAGDPRYEE |
| | SLHTPYPDYHWLRTVKTTKESLVIISPSVADLDPYDKSLHSMVFPNGKCSGITVY |
| | STYCSTNHDYTIWMPEKAGLGTSCDIFTNSKGKRASKGGRTCGFVDERGLYKSLR |
| | GACKLKLCGVPGLRLMDGTWATIQTPEETKWCSPDQLVNLHDFHSDEIEHLVVED |
| | LVKKREECLDTLESIMTTKSVSFRRLSHLRKLVPGFGKAYTIFNKTLMEADAHYK |
| | SVRTWNEIIPSKGCLRVGGRCHPHVNGVFFNGVIMGPDGHVLIPEMQSSLLQQHM |
| | ELLESSVIPLIHPLADPS (SEQ ID NO: 141) |
| AFN24193.1 | DFPIYTIPEKLGPWSPIDIHHLSCPNNLVVEDEGCSSLSGFSYMELKVGYISAIK |
| | VNGFTCTGVVTEAETYTNFVGYVTTTFKRKHFRPMPDACRAAYNWKTAGDPRYEE |
| | SLQNPYPDYHWLRTVKTTKESLVIISPSVADLDPYDKSLHSRVFSDGKCLGITIS |
| | STYCSTNHDYTIWMPEEARLGTSCDIFTTSRGKRASKGGKTCGFVDERGLYKSLK |
| | GACKLKLCGVSGLRLMDGTWVSIQAPDEAKWCSTDQLVNIHDIRSDEIEHLVVEE |
| | LVKKREECLDALESIMTTKSVSFRRLSHLRKLVPGFGKAYTIFNKTLMEADAHYK |
| | SVRTWSEVIPSKGCLSVGGRCHPHENGVFFNGIILGPDGSVLIPEMQSSLLQQHM |
| | ELLESSVIPLMHPLANPS (SEQ ID NO: 142) |
| AFN24083.1 | KFPIYTIPDKLGPWSPIDIHHLSCPNNLVVEDEGCTNLSGFSYMELKVGYISAIK |
| | VNGFTCTGVVTEAETYTNFIGYVTTVFKRKHSRPTPDACRAAYNWKMAGDPRYEE |
| | SLHNPYPDYHWLRTVKTTKESLVIISPSVADLDPYDKSLHSRVFPSGKCSGITVS |
| | STYCLTNHEYTIWMPENLRLGTSCDIFTNSRGKRASRGSKTCGFVDERGLYKSLK |
| | GACKLKLCGVLGLRLMDGTWVAMQTSDETKWCPPDQLVNLHDFRSDEIEHLVVEE |
| | LVKKREECLDAIESIMTTKSVSFRRLSHLRKLVPGFGKAYTIFNKTLMEADAHYK |
| | SVRTWNEIIPSKGCLRVGGRCHPHVKGVFFNGIILGPDGHVLIPEMQSSLLQQHM |
| | ELLESSIIPLMHPLADPS (SEQ ID NO: 143) |
| ABX46665.1 | KFPIYTIPDKLGPWSPIDIHHLSCPNNLVVEDEGCTNLSGFSYMELKVGYISAIK |
| | VNGFTCTGVVTEAETYTNFVGYVTTTFKRKHFRPTPDACRAAYNWKMAGDPRYEE |
| | SLHNPYPDYHWLRTVKTTKESLVIISPSVSDLDPYDKSLHSRVFPSGKCSGITVS |
| | STYCPTNHDYTIWMPENPRLGTSCDIFTNSRGKRASKGSKTCGFVDERGLYKSLK |
| | GACKLKLCGVLGLRLMDGTWAAIQTSDEAKWCPPDQLVNIHDFRSDEIEHLVVEE |
| | LVKKREECLDALESIMTTKSVSFRRLSHLRKLVPGFGKAYTIFNKTLMEADAHYK |
| | SVRTWNEIIPSKGCLRVGGRCHPHVNGVFFNGIILGPDGHVLIPEMQSSLLQQHM |
| | ELLESSVIPLMLPLADPS (SEQ ID NO: 144) |
| ABZ81200.1 | KFPIYTIPDKLGPWSPIDIHHLSCPNNLVVEDEGCTSLSGFSYMELKVGYISAIK |
| | VNGFTCTGVVTEAETYTNFVGYVTTTFKRKHFRPTPDACRAAYNWKVAGDPRYEE |
| | SLQNPYPDYHWLRTVKTTKESLIIISPSVADLDPYDKSLHSRVFPSGKCLGITIS |
| | STYCSTNHDYTIWMPEEARLGTSCDIFTNSKGKKASKGGRTCGFVDERGLYKSLK |
| | GACKLKLCGVPGLRLMDGTWVTIQTSDETKWCPPDQLVNLHDFHSDEIEHLVVEE |
| | LVKKREECLDALESIMTTKSVSFRRLSHLRKLVPGFGKAYTIFNKTLMEADAHYK |
| | SVRTWNEIIPSKGCLKVGGRCHPHVDGVFFNGIILGPDGNVLIPEMQSSLLQQHM |
| | ELLESSVIPLMHPLADPS (SEQ ID NO: 145) |
| AUT19580.1 | KFPIYTIPDKLGPWSPIDIHHLSCPNNLVVEDEGCTNLSGFSYMELKVGYISAIK |
| | VNGFTCTGVVTEAETYTNFVGYVTTTFKRKHLRPTPDACRAAYNWKMAGDPRYEE |
| | SLHNPYPDYHWLRTVKTTKESLVIISPSVADLDPYDKSLHSRVFPSGKCSGMTVS |
| | STYCSTNHDYTIWMPENPRLGTSCDIFTNSKGKRASKGSKICGFVDERGLYKSLK |
| | GACKLKLCGVTGLRLMDGTWVAMQTSDETKWCPPDQLVNLHDFRSDEIEHLVVEE |
| | LVKKREECLDALESIMITKSVSFRRLSHLRKLVPGFGKAYTIFNKTLMEADAHYK |
| | SVRTWNEIIPSKGCLRVGGRCHPHVNGVFFNGIILGPDGHVLIPEMQSSLLQQHM |
| | ELLESSVIPLMHPLADPS (SEQ ID NO: 146) |
| AUT19525.1 | KFPIYTIPDKLGPWSPIDIHHLSCPNNLVVEDEGCTNLSGFSYMELKVGYISAIK |
| | VNGFTCTGVVTEAETYTNFVGYVTTTFRRKHFRPTPDACRSAYNWKMAGDPRYEE |
| | SLHNPYPDYHWLRTVKTTKESLVIISPSVADLDPYDKSLHSRVFPNGKCSGLTVS |
| | STYCSTNHDYTIWMPENPRLGTSCDIFTNSRGKRASRGNKTCGFVDERGLYKSLK |
| | GACKLKLCGVLGLRLIDGTWVAMHTSDETKWCPPDQLVNLHDFRSDEIEHLVVEE |
| | LVKKREECLDALESIMTTKSVSFRRLSHLRKLVPGFGKAYTIFNKTLMEADAHYK |
| | SIRTWDEIVPSKGCLRVGGRCHPHVNGVFFNGIILGSDGHVLIPEMQSSLLQQHV |
| | ELLESSVVPLMHPLADPS (SEQ ID NO: 147) |
| AUT19520.1 | KFPIYTIPDKLGPWSPIDIHHLSCPNNLVIEDEGCTNLSGFSYMELKVGYISAIK |
| | VNGFTCTGVVTEAETYTNFVGYVTTTERRKHFRPTSDACRAAYNWKMAGDPRYEE |
| | SLHNPYPDYHWLRTVRTTKESLVIISPSVADLDPYDKSLHSRVFPNGRCSGLTVS |
| | STFCSTNHDYTIWMPENPRLGTSCDIFTNSRGKRASKGNRTCGFVDERGLYKSLK |
| | GACKLKLCGVLGLRLMDGTWVAMQTSEETKWCPPDQLVNLHDFRSDEIEHLVVEE |
| | LVKKREECLDALESIMTTKSVSFRRLSHLRKLVPGFGKAYTIFNKTLMEADAHYK |
| | SIRTWNEIIPSKGCLRVGGRCHPHVNGVFFNGIILGSDGHVLIPEMQSSLLQQHV |
| | ELLESSVVPLMHPLADPS (SEQ ID NO: 148) |
| ATP62005.1 | KFPIYTIPDKLGPWSPIDIHHLSCPNNLVVEDEGCTNLSEFSYVELKVGYILAIK |
| | MNGFTCTGVVTEAETYTNFVGYVTTTFKRKHFRPTPDACRAAYNWKMAGDPRYEE |
| | SLHNPYPDYHWLRTVRTTKESLVIISPSVADLDPYDRSLHSRVFPSGKCSGVAVS |
| | STYCSTNHDYTIWMPENPRLGMSCDIFTKSRGKRASKGSETCGFVDERGLYKSLK |
| | GACKLKLCGVPGLRLMDGTWVAMQTSNETKWCPPDQLVNLHDFRSDEIEHLVVEE |
| | LVRKREECLDALESIMTTKSVSFRRLSLLRKLVPGFGKAYTIFNKTLMEADAHYK |
| | SVRTWNEILPSKGCLRVGGRCHPHVNGVFFNGIILGPDGNVLIPEMQSSLLQQHM |
| | ELLESSVIPLVHPLADPS (SEQ ID NO: 149) |
| ASU55974.1 | KFPIYTIPDKLGPWSPIDIHHLSCPNNLVLEDEGCTNLSGFSYMELKVGYISAIK |
| | VSGFTCTGVVTEAETYTNFVGYVTTTFKRKHFRPTPDACRAAYNWKIAGDPRYEE |
| | SLHNPYPDYHWLRTVKTTKESLVIISPSVADLDPYDKSLHSRVFSREKCLGMTVS |
| | STYCSTNHDYTIWMPENPRLGTSCDIFTNSKGKRASRGSKTCGFVDERGLYKSLK |
| | GACKLKLCGVLGLRLVDGTWIAMQTSDEAKWCPSDQLVNLHDFRSDEIEHLVVEE |
| | LVKKREECLDALESIMTTKSVSFRRLSHLRKLVPGFGKAYTIFNKTLMEADAHYK |
| | SIRTWNEIIPSKGCLKVGGRCHPHVNGVFFNGIILGPDGHVLIPEMQSSLLQQHM |
| | ELLESSVIPLMHPLADPS (SEQ ID NO: 150) |
| AKN89688.1 | KFPIYTIPDKLGPWSPIDIHHLSCPNNLIVEDEGCSSLSGFSYMELKVGYISAIK |
| | VNGFTCTGVVTEAETYTNFVGYVTTTFKRKHFRPMPDACRAAYNWKMAGDPRYEE |
| | SLHNPYPDYHWLRTVKTTKESLIIISPSVADLDPYDKSLHSRVFPGGKCLGITIS |
| | STYCSTNHDYTIWMPEEPRLRTSCDIFTNSKGKRASKGGKMCGFVDERGLYKSLK |
| | GACKLKLCGVPGLRLMDGTWVSVQTPDETKWCSPDQLVNLHDFHSDEIEHLVVEE |
| | LVKKREECLDTLESIMTTKSVSFRRLSHLRKLVPGFGKAYTIFNKTLMEADAHYK |
| | SIQTWNEVIPSKGCLKVGGRCHPHVNGVFLNGIILGPDGNVLIPEMQSSLLQQHM |
| | ELLESSVIPLMHPLSDPS (SEQ ID NO: 151) |
| AKN89673.1 | KFPIYTIPDKLGPWSPIDIHHLSCPNNLIVEDEGCTSLSGLSYMELKVGYISAIK |
| | VNGFTCTGVVTEAETYTNFVGYVTTTFKRKHFRPMPDACRAAYNWKIAGDPRYEE |
| | SLQNPYPDSHWLRTVKTTKESLIIISPSVADLDPYDKSLRSRVFPGGKCLGITIS |
| | STYCSTNHDYTIWMPEEARLGTSCDIFTNSKGKRASKGGKICGFVDERGLYKSLK |
| | GACKLKLCGVSGLRLMDGTWVSIQIPDEIKWCPSDQLVNLHDFHSDEIEHLVVEE |
| | LVKKREECLDALESIMTTKSVSFRRLSHLRKLVPGFGKAYTIFNKTLMEADAHYN |
| | SVRTWNEIIPSKGCLKVGGRCHPHVDGVFFNGIIMGPDGSVLIPEMQSSLLQQHM |
| | ELLESSVIPLMHPLADPS (SEQ ID NO: 152) |
| AKN89668.1 | KFPVYTIPEKLGPWSPIDIHHLSCPNNLAVEDEGCNSLSGFSYMELKVGYISAIK |
| | VNGFTCTGVVTEAETYTNFVGYVTTTFKRKHFRPMPDACRAAYNWKMAGDPRYEE |
| | SLHNPYPDYHWLRTVTTTKESLIIISPSVANLDPYDKSLHSRVFPGGKCMGITVS |
| | STFCSTNHDYTIWMPENARLGTSCDIFTTSKGKKASKGGKICGFVDERGLYKSLK |
| | GACKLKLCGVSGLRLLDGTWVTIQTSDEIKWCPPDKLVNLHDFHSDEIEHLVVEE |
| | LVRKREECLDTLESIMTTKSVSFRRLSHLRKLVPGFGKAYTIFNKTLMEADAHYK |
| | SVNTWNEIIPSKGCLTVGGRCHPHVNGVFFNGIILGPDGHVLIPEMQSSLLQQHM |
| | ELLESSVIPLMHPLADPS (SEQ ID NO: 153) |
| AKN89653.1 | KFPIYTIPDKLGPWSPIDIHHLSCPNNLVVEDEGCTSLSGFSYMELKVGYISAIK |
| | VNGFTCTGVVTEAETYTNFVGYVTTTFKRKHFRPMPDACRAAHEWKTAGDPRYED |
| | SLQNPYPDYHWLRTVKTTKESLVIISPSVADLDPYDKSLHSRVFPGGKCLGVTIS |
| | STYCSTNHDYTIWMPEEAGLGTSCDIFTNSRGKRASKGGRICGFVDERGLYKSLK |
| | GACKLKLCGVPGLRLMDGTWVSIQAPDETKWCPPDQVVNLHDFHSDEIEHLVVEE |
| | LIKKREECLDALESIMTTKSVSFRRLSHLRKLVPGFGKAYTIFNKTLMEADAHYK |
| | SVRTWNEVVPSKGCLKVGGRCHSDVDGVFFNGIILGPDGNVLIPEMQSSLLQQHM |
| | ELLESSVIPLMHPLADPS (SEQ ID NO: 154) |
| AKN89648.1 | KFPIYTIPDKLGPWSPIDIHHLSCPNNLVVEDEGCTSLSGFSYMELKVGYISAIK |
| | VNGFTCTGVVTEAETYTNFVGYVTTTFKRRHERPMPDACRAAHNWKIAGDPRYED |
| | SLQNPYPDYHWLRTVKTTKESLIIISPSVADLDPYDKSLHSRVFPGGKCLGVTIS |
| | STYCSTNHDYTIWMPEEAGLGTSCDIFTNSRGKRASKGGKTCGFVDERGLYKSLK |
| | GSCKLKLCGVPGLRLIDGTWVSIQTPDETKWCPPDQVVNLHDFHSDEIEHLVVEE |
| | LIKKREECLDALESIMTTKSVSFRRLSHLRKLVPGFGKAYTIHNKTLMEADAHYK |
| | SVRTWNEIIPSKGCLRVGGRCHPNVDGVFFNGIILGPDGNVLIPEMQSSLLQQHM |
| | ELLESSVIPLMHPLADPS (SEQ ID NO: 155) |
| AEZ55919.1 | KFPIYTIPDKLGPWSPIDIHHLCCPNNLVAEEEGCTSLSGFSYMELKVGYISAIK |
| | VNGFTCTGVVTEAETYTNFVGYVTTTFKRKHFRPMPDACRAAYDWKMAGDPRYED |
| | SLQNPYPDYHWLRTVKTTKESLIIISPSVADLDPYDKSLHSRVFPGGKCLGITIS |
| | STYCSTNHDYTIWMPEEARLGTSCNIFTNSRGKKASKGGRTCGFVDERGLYKSLK |
| | GACKLKLCGVPGLRLMDGTWVSIQTSNETKWCPPDQLVNLHDFHSDEIEHLVVEE |
| | LVKKREECLDALESIMTTKSVSFRRLSHLRKLVPGFGKAYTIFNKTLMEADAHYK |
| | SVRTWNEIIPSKGCLKVGGRCHPHVDGVFFNGIILGPDGHVLIPEMQSSLLQQHM |
| | ELLESSVIPLIHPLADPS (SEQ ID NO: 156) |
| BAL49598.1 | KFPIYTIPDKLGPWSPIDIHHLSCPNNLVVEDEGCTNLSGFSYMELKVGYISAIK |
| | VNGFTCTGVVTEAETYTNFVGYVTTTFKRKHFRPTPDACRAAYNWKMAGDPRYEE |
| | SLHNPYPDYHWLRTVKTTKESLVIISPSVADLDPYDKSLHSRVFPSGKCLGITVS |
| | STYCSTNHDYTVWRPENPRLGTSCDIFTNSRGKRASKGSKTCGFVDERGWYKSWK |
| | GACKLKLCGVLGLRLIDGTWIAMQTSEETKWCPPDQLVNLHDFRSDEIEHLVVEE |
| | LVKKREECLDALESIMTTKSVSFRRLSHLRKLVPGFGKAYTIFNKTLMEADAHYK |
| | SIRTWNEIIPSKGCLRVGGKCHPHVNGVFFNGIILGPDGHVLIPEMQSSLLQQHM |
| | ELLESSVIPLMHPLADPY (SEQ ID NO: 157) |
| ADD84790.1 | KFPIYTIPDKLGPWSPIDLHHLSCPNNLVVEDEGCTNLSGFSYMELKVGYISAIK |
| | VNGFTCTGVVTEAETYTNFVGYVTTTFKRKHFRPTPDACRAAYNWKMAGDPRYEE |
| | SLHNPYPDYHWLRTVKTTKESLVIISPSVTDLDPYDKSLHSRVFPGGNCSGITVS |
| | STYCSTNHDYTIWMPENLRLGTSCDIFTHSRGKRASKGDKTCGFVDERGLYKSLK |
| | GACKLKLCGVLGLRLMDGTWVAMQTSDETKWCPPGQLVNLHDFRSDEIEHLVVEE |
| | LVKKREECLDALESIMTTKSVSFRRLSHLRKLVPGFGKAYTIFNKTLMEADAHYK |
| | SVQTWNEIIPSKGCLRVGERCHPHVNGVFFNGIILGSDGHVLIPEMQSSLLQQHM |
| | ELLESSVIPLMHPLADPS (SEQ ID NO: 158) |
| AAV85905.1 | KFPIYTIPDKLGPWSPIDIHHLSCPNNLVVEDEGCTNLSGFSYLELKVGHISAIK |
| | VNGFTCTGVVTEAETYTNFVGYVTTTFKRKHFRPTPDACRAAYNWKTAGDPRYEE |
| | SLQNPYPDYQWLRTVRTTKESLVIISPSAADLDPYDKSLHSRVFPSGKCSGITVS |
| | SVYCSTNHDYTIWMPENPRQGMSCDIFTNSRGKRASKERKTCGFVDERGLYKSLR |
| | GSCKLKLCGVLGLRLMDGTWVAMQTSNETKWCSPDQLVNLHDFHSDEIEHLVVEE |
| | LVKKREECLDALESIMTTKSVSFRRLSHLRKLVPGFGKAYTIINKTLMEAEAHYK |
| | SVRTWNEIVPSKGCLRVEGRCHPHVNGVFFNGIILGPDGHVLIPEMQSSLLQQHM |
| | ELLESSVIPLMHPLADPS (SEQ ID NO: 159) |
| ACR50749.1 | KFPIYTIPDKLGPWSPIDIHHLSCPNNLVVEDEGCTNLSGFSYMELKVGYISAIK |
| | VNGFTCTGVVTEAETYTNFVGYVTTTFRRKHFRPTPDACRAAYNWKMAGDPRYEE |
| | SLHNPYPDYHWLRTVKTTKESLVIISPSVADLDPYDKSLHSRVFLNGKCSGLTVS |
| | STYCSTNHDYSIWMPENPRLGTSCDIFVTSRGKRASKGSKTCGFVDERGLYKSLK |
| | GACKLKLCGVLGLRLIDGTWVAMQTSEEPKWCPPDQLVNLHNFRSDEIEHLVVEE |
| | LVKKREECLDALESIMTTKSVSFRRLSHLRKLVPGFGKAYTIFNKTLMEADAHYK |
| | SIRTWDEIIPSKGCLRVGGRCHPHVNGVFFNGIILGSDGHVLIPEMQSSLLQQHV |
| | ELLESSVIPLMHPLADPS (SEQ ID NO: 160) |
| YP_009094182.1 | DFPLYTIPEKIGPWTPIDLTHLSCPNNLLSEDDGCSSSSTFSYIELRTGYLTHQK |
| | VSGFTCTGVINEAVTYTNFVGYVTTTFKRKHFKPTASACRDAYHWKISGDPRYEE |
| | SLHTPYPDNSWLRTVTTTKESLLIISPSIVEMDIYSRSLHSPMFPTGRCYDFYKS |
| | TPSCLTNHDYTIWLPDDANVRLTCDIFVTSTGKKSMNGSKMCGFTDERGLYRTLK |
| | GACKLTLCGKPGLRLFDGTWISITRPEIVMWCSPNQLVNVHNNRVDEIEHLIVGD |
| | LIRRREECLDTLETVLMSKSVSFRRLSHFRKLVPGFGKAYTIANGSLMETNVHYK |
| | RVDRWEEILPSKGCLKLNDKCLNPENGVFFNGIIKGPDGQVLIPEMQSSLLKOHM |
| | DLLKASVFPLRHPLIDQT (SEQ ID NO: 161) |
| AMM70635.1 | KFPIYTIPDKLGPWSPIDIHHLSCPNNLVVEDEGCTTLTPESYMELKVGYITSIK |
| | VSGFTCTGVVTEAETYTNFVGYVTTTFRRRHFRPSVNSCRDAYNWKIAGDPRYEE |
| | SLHNPYPDSHWLRTVKTTKESLLIISPSVADMDAYDKKLYSKMFPNGKCSEISPG |
| | SPFCPTNHEYTIWMPESSNPGISCDIFTRSMGKKATKDGQLCGFVDERGLYKSLK |
| | GACRLRLCGISGLRLMDGSWVSLPQVNNSEWCSPDQLVNIHDFHSDEIEHLVADE |
| | LVKKREDCLDALETIIFTKSISFRRLSHLRKLVPGFGKAYTIINRTLMEAEAHYK |
| | SVREWKEIIPSKGCLKAGGRCYPHHNGIFFNGIILGPGGEILIPEMQSALLQQHI |
| | ELLESSVVPLKHPLADPS (SEQ ID NO: 162) |
| ADD84515.1 | DFPLYTIPEKIDSWTPIDMIHLSCPNNLLSGDDGCSTSTSFSYVELKTGYLTHQK |
| | VSGFTCTGVISEAVTYTNFVGYVTTTFKRKHFKPTVSACRDAFHWKISGDPRYEE |
| | SLHTPYPDSSWLRTVTTTKESLLIISPSIVEMDIYSRTLHSPMFPTGTCSRFYPS |
| | VPSCATNHDYTLWLPEDSNLSLICDIFVTSTGRKAMNGSKMCGFTDERGFYRTLR |
| | GACKLTLCGKPGLRLYDGTWVSFTRPEVHVWCSPNQLVNVHNNRIDELEHLIVDD |
| | LIQKREACLDTLETILMSKSISFRRLSHFRKLVPGYGKAYTILNGSLMETNVHYR |
| | RVDNWTDILPSKGCLKVGNKCLDPHKGVYFNGIIKGPDGHILIPEMQSGMLKQHI |
| | DLLKAAVFPLRHPLIDQS (SEQ ID NO: 163) |
| YP_007641390.1 | DFPLYTIPERLNPWTPIDLIHLSCPNNLLSDAEGCSETSSFTYVELKTGFLAHQK |
| | VPGFTCTGVINEAVTYTNFVGYVTTTFKRKHFKPTVSACRDAFNWKVSGDPRYEE |
| | SLHTPYPDNSWLRTVTTTKESLLIISPSIVEMDVYGRTLHSPMFPTGICSKFYPS |
| | IPSCATNHDYTLWLPEDPNLSLICDIFVTSTGRKAMNGSRMCGFTDERGFYRTLK |
| | GACKLTLCGKPGLRLYDGTWVSFTRPEIHVWCSPDQLVNVHNNRIDEIEHLIVDD |
| | LIRKREECLDTLETVLMSKSLSFRRLSHFRKLVPGYGKAYTILNGSLMETNVHYK |
| | RVDNWVDILPSKGCLKVNNKCMESDTGVFFNGIIKGPDGRILIPEMQSGLLKQHM |
| | DLLKAAVFPLRHPLIDKA (SEQ ID NO: 164) |
| AEE36617.1 | EFPLYTIPEKIEKWTPIDMIHLSCPNNLLSEEEGCNTESPLTYFELKSGYLAHQK |
| | VPGFTCTGVVTEAETYTNFVGYVTTTFKRKHFGPTVAACRDAYNWKVSGDPRYEE |
| | SLHTPYPDSSWLRTVTTTKESLLIISPSIVEMDVYGRTLHSPMFPSGICSKLYPS |
| | VPSCKTNHDYTLWLPEDPSLSLICDIFTSGSGRKAMNGSRICGFKDERGFYRSLK |
| | GACKLTLCGKPGIRLFDGTWVSFTRPEVHVWCTPNQLVNIHNDRLDEIEHLIVED |
| | IVKRREECLDTLETIFMSQSISFRRLSHFRKLVPGYGKAYTILNGSLMEANVYYK |
| | RVDKWADILPSKGCLKVGQQCMDPVNGVFFNGIIKGPDGQILIPEMQSEQLKQHM |
| | DLLKAAVFPLRHPLISQE (SEQ ID NO: 165) |
| AEE36616.1 | DFPLYTIPEKIGTWTPIDLIHLTCPNNLLSGDDGCSDTATFGYIELKTGYLTHQK |
| | VSGFTCTGVVNEAVTYTNFVGYVTTTFKRKHFKPTALACRDAFHWKISGDPRYEE |
| | SLHTPYPDNSWLRTVTTTKESLLIISPSIVEMDVYSRTLHSPMFPGGVCSKFYPS |
| | SPSCPTNHDYTLWLPEDANLSMACDIFVTSTGKKSMNGSRMCGFTDERGFYRTLK |
| | GACKLTLCGKPGLRLYDGTWVSFTRPEINVWCSPNQLVNVHNNRLDEIEHLIVGD |
| | LIRKREECLDTLETILMSKSISFRRLSHFRKLVPGYGKAYTIINGSLMETNVHYL |
| | RVDSWNDILPSKGCLKMNKQCVDSYKGVFFNGIIKGLDGHILIPEMQSSLLKQHM |
| | DLLKAAVEPLRHPLIDQN (SEQ ID NO: 166) |
| AAA67271.1 | EFPLYTIPEKIEKWTPIDMIHLSCPNNMLSEEEGCNTESPFTYFELKSGYLAHQK |
| | VPGFTCTGVVNEAETYTNFVGYVTTTFKRKHFKPTVAACRDAYNWKVSGDPRYEE |
| | SLHTPYPDSSWLRTVTTTKEALLIISPSIVEMDIYGRTLHSPMFPSGKCSKLYPS |
| | VPSCTTNHDYTLWLPEDSSLSLICDIFTSSSGQKAMNGSRICGFKDERGFYRSLK |
| | GSCKLTLCGKPGIRLFDGTWVSFTKPDVHVWCTPNQLVNIHNDRLDEVEHLIVDD |
| | IIKKREECLDTLETILMSQSVSFRRLSHFRKLVPGYGKAYTILNGSLMETNVYYK |
| | RVDRWADILPSRGCLKVGQQCMDPVKGVLFNGIIKGPDGQILIPEMQSEQLKQHM |
| | DLLKAAMFPLRHPLINRE (SEQ ID NO: 167) |
| ABZ81210.1 | EFPLYTIPEKIEKWTPIDMIHLSCPNNLLSEEEGCNTESPFTYFELKSGYLAHQK |
| | VPGFTCTGVVNEAETYTNFVGYVTTTFKRKHFMPTVAACRDAYNWKVSGDPRYEE |
| | SLHTPYPDSSWLRTVTTTKESLLIISPSIVEMDVYGRTLHSPMFPSGICSKLYPS |
| | VPSCKTNHDYTLWLPEDPSLSLICDIFTSSSGQKVMNGSRICGFRDERGFYRSLK |
| | GACKLTLCGRPGIRLFDGTWVSFTKPDIHVWCTPNQLVNIHNDRLDEIEHLIVED |
| | IIKKREECLDTLETILMSQSISFRRLSHFRKLVPGYGKAYTILNGSLMETNVYYK |
| | RVDKWTDILPSKGCLKVGQQCMEPVNGVLFNGIIKGPDGQILIPEMQSEQLKQHM |
| | DLLKAAVFPLRHPLIKRE (SEQ ID NO: 168) |
| ABZ81170.1 | DFPLYTIPEKIGPWTPIDLIHLSCPNNLQSEDEGCGTSSVFSYVELKTGYLTHQK |
| | VSGFTCTGVVNEAVTYTNFVGYVTTTFKRKHFKPTALACRDAYHWKISGDPRYEE |
| | SLHTPYPDNSWLRTVTTTKESLVIISPSIVEMDVYSRTLHSPMFPTGTCSRFYPS |
| | SPSCATNHDYTLWLPDDPNLSLACDIFVTSTGKKSMNGSRMCGFTDERGYYRTIK |
| | GACKLTLCGKPGLRLFDGTWISFTRPEVTTRCLPNQLVNIHNNRIDEVEHLIVED |
| | LIRKREECLDTLETVLMSKSISFRRLSHFRKLVPGYGKAYTILNGSLMETNVHYL |
| | KVDNWSEILPSKGCLKINNQCVAHYKGVFFNGIIKGPDGHILIPEMQSSLLKQHM |
| | DLLKAAVFPLRHPLIEPG (SEQ ID NO: 169) |
| AHZ89649.1 | KFPIYTIPDKLGPWSPIDIHHLSCPNNMVVEDEGCTNLSGFSYMELKVGYISAIK |
| | VNGFTCTGVVTEAETYTNFVGYVTTTFKRKHFRPVPDACRDAHNWKIAGDPRYEE |
| | SLHNPYPDYHWLRTVKTTKESLVIISPSVADLDPYDKSLHSSVFPGGKCSGITVS |
| | STYCSTNHDYTIWMPENPRLGTSCDIFTYSKGKRASKESKTCGFVDERGLYKSLK |
| | GACKLKLCGVPGLRLMDGTWVAIQTSDEVKWCPPDQLVNLHDLHSDELEHRVVEE |
| | LVKKREECLDALESIMTTKSVSFRRLSHLRKLVPGFGKAYTIFNKTLMEADAHYK |
| | SVRTWNEIIPSKGCLRVGGRCHPHVNGVFFNGIILGPDGHVLIPEMQSSLLQQHM |
| | ELLESSVIPLMHPLADPS (SEQ ID NO: 170) |

In some embodiments, the rabies G protein ectodomain shares at least 70%, at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs: 104-170.

Illustrative rabies G protein ectodomain sequences having fusion loop domain deletions are provided in Table 2.

TABLE 2

| | SEQ ID | |
|---|---|---|
| Construct | NO: | Sequence |
| A | 76 | MVPQVLLFVPLLGFSLCFGKFPIYTIPDELGPWSPIDIHHLSCPNNLVVEDE |
| | | GCTNLSEFSYMELKVGYISAIKVNGFTCTGVVTEAETYTNFVGYVTTTFKRK |
| | | HFRPTPDACRAAYNWKMAGDPRYEESLHNPYPDYHWLRTVRTTKESLIIISP |
| | | SVTDLDPYDKSLHSRGFPGGKCSGITVSSTYCSTNHDYTIWMPENPGPRTPC |
| | | DIFTNSRGKRASKGNKTCGFVDERGLYKSLKGACRLKLCGVLGLRLMDGTWV |
| | | AMQTSDETKWCPPDQLVNLHDFRSDEIEHLVVEELVKKREECLDALESIMTT |
| | | KSVSFRRLSHLRKLVPGFGKAYTIFNKTLMEADAHYKSVRTWNEIIPSKGCL |
| | | KVGGRCHPHVNGVFFNGIILGPDGHVLIPEMQSSLLQQHMELLKSSVIPLMH |
| | | PLADPSTVFKEGDEAEDFVEVHLPDVYKQISGVDLGLP |
| B | 77 | MELLILKANAITTILTAVTFCFASGKFPIYTIPDELGPWSPIDIHHLSCPNN |
| | | LVVEDEGCTNLSEFSYMELKVGYISAIKVNGFTCTGVVTSGSGRKHFRPTPD |
| | | ACRAAYNWKMAGDPRYEESLSGSGESLIIISPSVTDLDPYDKSLHSRGFPGG |
| | | KCSGITVSSTYCSTNHDYTIWMPENPGPRTPCDIFTNSRGKRASKGNKTCGF |
| | | VDERGLYKSLKGACRLKLCGVLGLRLMDGTWVAMQTSDETKWCPPDQLVNLH |
| | | DFRSDEIEHLVVEELVKKREECLDALESIMTTKSVSFRRLSHLRKLVPGFGK |
| | | AYTIFNKTLMEADAHYKSVRTWNEIIPSKGCLKVGGRCHPHVNGVFFNGIIL |
| | | GPDGHVLIPEMQSSLLQQHMELLKSSVIPLMHPLADPSTVFKEGDEAEDFVE |
| | | VHLPDVYKQISGVDLGLP |
| C | 78 | MVPQVLLFVPLLGFSLCFGKFPIYTIPDELGPWSPIDIHHLSCPNNLVVEDE |
| | | GCTNLSEFSYMELGSGSGSGCDIFTNSRGKRASKGNKTCGFVDERGLYKSLK |
| | | GACRLKLCGVLGLRLMDGTWVAMQTSDETKWCPPDQLVNLHDFRSDEIEPLV |
| | | VEELVKKREECLDALESIMTTKSVSFRRLSHLRKLVPGFGKAYTIFNKTLME |
| | | ADAHYKSVRTWNEIIPSKGCLKVGGRCHPHVNGVFFNGIILGPDGHVLIPEM |
| | | QSSLLQQHMELLKSSVIPLMHP |
| D | 79 | MVPQVLLFVPLLGFSLCFGKFPIYTIPDELGPWSPIDIHHLSCPNNLVVEDE |
| | | GCTNLSEFSYMELKVGSSIGKCDIFTNSRGKRASKGNKTCGFVDERGLYKSL |
| | | KGACRLKLCGVLGLRLMDGTWVAMQTSDETKWCPPDQLVNLHDFRSDEIEPL |
| | | VVEELVKKREECLDALESIMTTKSVSFRRLSHLRKLVPGFGKAYTIFNKTLM |
| | | EADAHYKSVRTWNEIIPSKGCLKVGGRCHPHVNGVFFNGIILGPDGHVLIPE |
| | | MQSSLLQQHMELLKSSVIPLMHPLADPSTVFKEGDEAEDFVEVHLPDVYKQI |
| | | SGVDLGLP |
| E | 80 | MVPQVLLFVPLLGFSLCFGKFPIYTIPDELGPWSPIDIHHLSCPNNLVVEDE |
| | | GCTNLSEFSYMELKVGIPGSNCDIFTNSRGKRASKGNKTCGFVDERGLYKSL |
| | | KGACRLKLCGVLGLRLMDGTWVAMQTSDETKWCPPDQLVNLHDFRSDEIEPL |
| | | VVEELVKKREECLDALESIMTTKSVSFRRLSHLRKLVPGFGKAYTIFNKTLM |
| | | EADAHYKSVRTWNEIIPSKGCLKVGGRCHPHVNGVFFNGIILGPDGHVLIPE |
| | | MQSSLLQQHMELLKSSVIPLMHP |
| F | 81 | MVPQVLLFVPLLGFSLCFGKFPIYTIPDELGPWSPIDIHHLSCPNNLVVEDE |
| | | GCTNLSEFSYMELGSGSGSGCDIFTNSRGKRASKGNKTCGFVDERGLYKSLK |
| | | GACRLKLCGVLGLRLMDGTWVAMQTSDETKWCPPDQLVNLHDFRSDEIEHLV |
| | | VEELVKKREECLDALESIMTTKSVSFRRLSHLRKLVPGFGKAYTIFNKTLME |
| | | ADAHYKSVRTWNEIIPSKGCLKVGGRCHPHVNGVFFNGIILGPDGHVLIPEM |
| | | QSSLLQQHMELLKSSVIPLMHP |

In some embodiments of the polypeptide, the rabies G protein ectodomain shares at least 70%, at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs: 77-81.

In some embodiments of the polypeptide, the polypeptide comprises a multimerization domain. In some embodiments of the polypeptide, the multimerization domain is a trimerization domain. In some embodiments of the polypeptide, the multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to FoldOn (SEQ ID NO: 58). In some embodiments of the polypeptide, the multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to a polypeptide sequence selected from SEQ ID NOs: 1, 4, 5, 7, 9, 18, 19, 21, 24, 25, 26, 29, 30, 31, 34, 36, 37, 39, 42, 43, 44, 45, 46, 47, 48, 49, 50, and 51, wherein the interface residues identified in Table 3 are conserved. In some embodiments of the polypeptide, the multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50A (SEQ ID NO: 7). In some embodiments of the polypeptide, the multimerization domain is I53-50A-Δcys (SEQ ID NO: 67). In some embodiments of the polypeptide, the polypeptide shares at least 70%, at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs: 93-96. In some embodiments of the polypeptide, the multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-dn5B (SEQ ID NO: 75). In some embodiments of the polypeptide, the polypeptide shares at least 70%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 101 or SEQ ID NO: 102. In some embodiments of the polypeptide, the multimerization domain is a ferritin polypeptide capable of forming a ferritin particle. In some embodiments of the polypeptide, the polypeptide shares at least 70%, at least 80%, at least 85%, at least 90%, at least 91%, at least 92%, at least 93%, at least 94%, at least 95%, at least 96%, at least 97%, at least 98%, at least 99%, or 100% identity to Construct F-ferritin (SEQ ID NO: 103). In some embodiments of the polypeptide, the multimerization domain is a particle-forming domain.

### Subunit Vaccines

The polypeptides of the disclosure may be used as subunit vaccines. For example, the polypeptide may comprise a trimerization domain, such as FoldOn or a GCN4 trimerization, or such as the I53-50A or I53-dn5B trimerization domains. In the absence of their partners (I53-50B and I53-dn5A, respectively) I53-50A or I53-dn5B trimerization domains may be used to generate solution trimers of the rabies G protein ectodomain. Such soluble trimers may be used as assay reagents (such as validation standards) or as subunit vaccines. For example, the disclosure provides a pharmaceutical composition comprising a polypeptide, the polypeptide comprising, in N- to C-terminal order, a rabies G protein ectodomain, wherein the rabies G protein ectodomain comprises a deletion of a fusion loop domain of the rabies G protein ectodomain; a polypeptide linker; and a trimerization domain, optionally selected from FoldOn, GCN4, I53-50A, or I53-dn5B trimerization domain.

### Nanoparticles

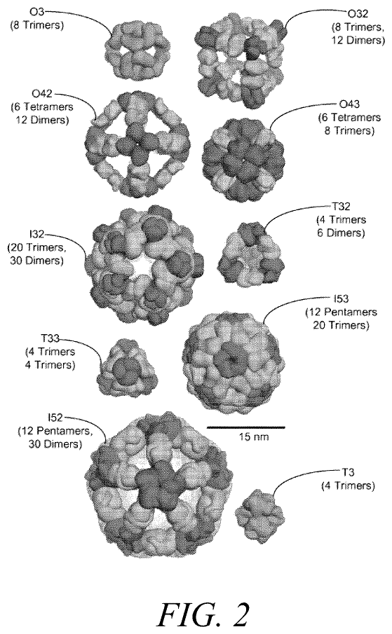

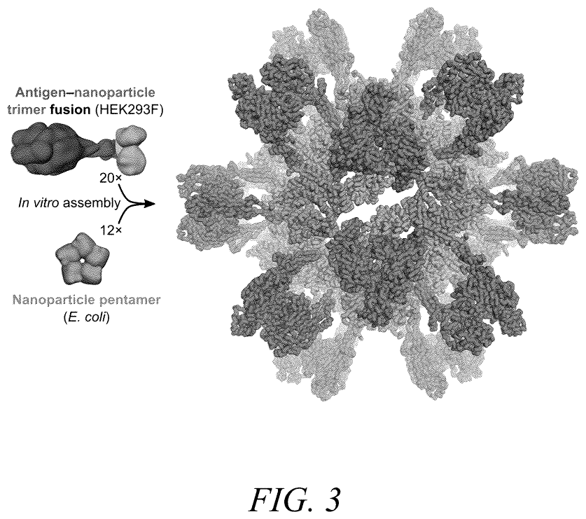

The present disclosure relates, in part, to polypeptides for use as a component of a nanoparticle. The nanoparticle may be a nanoparticle comprising a lipid component, or preferably a nanoparticle that is substantially free of or lacks a lipid component. For example, the nanoparticle may be a nanostructure of protein-based virus-like particle (pbVLP). Illustrative nanostructures are depicted in FIG. 2 without the rabies G protein shown. A further illustrative nanostructure or pbVLP is depcited in FIG. 3.

Nanoparticles of the present disclosure may display an antigen capable of eliciting immune responses to rabies virus. In some embodiments, the nanoparticles of the present disclosure are useful for preventing or decreasing the severity of infection with rabies. In particular, the nanoparticles of the disclosure display the ectodomain of the rabies G protein. The ectodomain may be attached to the core of the nanoparticle either non-covalently or covalently, including as a fusion protein or by other means disclosed herein. In some embodiments, a linker connects the ectodomain to a first polypeptide comprising a multimerization domain. The linker may be any chemical linkage including but not limited to a polypeptide used to form a N-terminal or C-terminal fusion of the ectodomain to the first polypeptide. The rabies G protein may optionally be displayed along a symmetry axis of the VLP. In some embodiments, the G protein is C-terminally linked to the first polypeptide comprising a trimerization domain. There may be an intervening trimerization tag (e.g., FoldOn tag).

### Protein-Based Virus-Like Particles

The nanoparticle of the disclosure may be protein-based virus-like particles (pbVLPs). The pbVLPs of the present invention may comprise multimeric protein assemblies adapted for display of the ectodomain of rabies G protein. The pbVLPs of the present invention comprise at least one polypeptide component, present in multiple copies in the pbVLP. The multimerization domain of the first polypeptide component may be derived from a naturally-occurring protein sequence by substitution of at least one amino acid residue. This first component may form the entire core of the pbVLP; or the core of the pbVLP may comprise a second polypeptide component, or a third, fourth, fifth and so on polypeptide component, such that the VLP comprises two, three, four, five, six, seven, or more components, each present in multiple copies. In some cases, the first polypeptide component, which comprises the polypeptide comprising the rabies G protein ectodomain, will form trimers related by 3-fold rotational symmetry and the second polypeptide component will form pentamers related by 5-fold rotational symmetry. In such cases, the VLP forms an “icosahedral particle” having 153 symmetry. Together these two polypeptide components are arranged such that the members of each homomeric complex are related to one another by symmetry operators. A general computational method for designing self-assembling protein materials, involving symmetrical docking of protein building blocks in a target symmetric architecture, is disclosed in U.S. Patent Pub. No. US 2015/0356240 A1.

The “core” of the pbVLP is used herein to describe the central portion of the pbVLP that links together the several copies of the rabies G protein ectodomain, displayed by the pbVLP. In some embodiments, the first component comprises a polypeptide comprising a rabies G protein ectodomain, a polypeptide linker, and a first multimerization domain. In other embodiments, the polypeptide comprising the rabies G protein ectodomain is non-covalently or covalently linked to the first multimerization domain. For example, an antibody or antigenic fragment thereof may be fused to the first multimerization domain and configured to bind the rabies G protein ectodomain, or a chemical tag on the ectodomain. A streptavidin-biotin (or neuravidin-biotin) system can be employed. Alternatively, various chemical linkers may be used. An advantage of designing a core to be a generic platform is that the one or more multimerization domains that comprise the core can be designed and optimized in advance and then tested with various ectodomains. It will be understood that in some cases, the same polypeptide may form a portion of the “core” and then extend outward as either an adaptor for attachment of the rabies G protein ectodomain or to include the ectodomain (i.e., a fusion protein). In embodiments of the present disclosure, the polypeptide comprises further polypeptide sequences in addition to the rabies G protein ectodomain. In certain embodiments, the ectodomain is glycosylated either natively or using alternative oligosaccharides (e.g., oligosaccharides specific to the host cell used to express the antigen).

The multimerization domain(s) drive self-assembly of the pbVLPs. In some cases, self-assembly may be further promoted by multimerization of the ectodomain even though the core would, in absence of the ectodomain, be independently capable of self-assembly. Without being bound by theory, rabies G protein forms a trimer in its native state. Display of the ectodomain on a VLP, at least in some embodiments, decreases the thermodynamic barrier for assembly and/or the equilibrium ratio of assembled to non-assembled VLP components. In some cases, the trimeric ectodomain placed along a 3-fold axis of the VLP promotes proper folding and conformation stability of the ectodomain and makes self-assembly of the VLP a cooperative process, in that the ectodomain is trimerized properly in part due to its display on a 3-fold axis of the core of the VLP, and the VLP is stabilized in its assembled form, at least in part, by non-covalent or covalent interactions amongst the trimer units. In some cases, introduction of mutations to the antigen or to the VLP components may optionally further stabilize assembly, in particular if cysteine residues are positioned to create intramolecular disulfide bonds. In some examples, a dimeric, trimeric, tetrameric, pentameric, or hexameric antigen is displayed upon a core designed to have a matching 2-fold, 3-fold, 4-fold, 5-fold, or 6-fold symmetry axis such that the core accommodates the arrangement of the multimeric antigen with the native symmetry of the antigen.

In some embodiments, a protein-based VLP can include a symmetric core. These include but are not limited to designed VLPs. For example, the protein ferritin has been used to generate a symmetric, protein-based VLP using naturally occurring ferritin sequences. Ferritin-based VLP are distinguished from designed VLPs in that no protein engineering is necessary to form a symmetric VLP from ferritin, other than fusing the viral protein (here a rabies G protein ectodomain) to the ferritin molecule. Protein design methods can be used to generate similar one- and two-component nanostructures based on template structures (e.g., structures deposited in the Protein Data Bank) or de novo (i.e., by computational design of new proteins having a desired structure but little or no homology to naturally occurring proteins). Such one- and two-component nanostructures can then be used as the core of a designed VLP. The terms “protein nanoparticle” or “nanoparticle” and the term “nanostructure” may be used to refer to protein-based VLPs as described herein.

The VLP may be a ferritin-based VLP. In some embodiments, the protein complex is a protein-based VLP (including ferritin, E2p, I3-01 and I3-01 variants) as described in U.S. Pat. Pub. No. US 2020/0009244 A1 and Int'l Pat. Pub. Nos. WO 2022/046583 A1 and WO 2021/210984 A1, the disclosures of which are incorporated by reference herein. The protein-based VLP may employ a variety of coupling techniques to attach an antigen to the VLP core, including but not limited to the SpyCatcher system described in, e.g., Escolano et al. Nature 570:468-473 (2019), He et al. Sci Adv. 7(12):eabf1591 (2021), and Tan et al. Nat. Commun. 12(1):542 (2021). The protein-based VLP may be a lumazine synthase nanoparticle as described, e.g., in Geng et al. PLoS Pathog. 17(9):e1009897 (2021). The protein-based VLP may be a ferritin nanoparticle as described, e.g., in Joyce et al. bioRxiv 2021.05.09.443331 and in U.S. Pat. Pub. No. US 2019/0330279 A1.

In the present disclosure, protein-based VLPs are distinguished from nanoparticle vaccines more generally, because the term “nanoparticle” vaccine has been used in the art to refer to protein-based or glycoprotein-based vaccines (see, e.g. U.S. Pat. No. 9,441,019), polymerized liposomes (see, e.g., U.S. Pat. No. 7,285,289), surfactant micelles (see, e.g., US Patent Pub. No. US 2004/0038406 A1), and synthetic biodegradable particles (see, e.g., U.S. Pat. No. 8,323,696). Use of the disclosed rabies G protein ectodomains in any of these formats is contemplated.

The protein-based VLPs of the present disclosure are distinguishable from VLPs that display the rabies G protein on the surface of a micelle particle containing a surfactant (e.g., NP-9); or alternatively, made by extracting antigenic proteins from live virus while retaining lipid constituents of the viral envelope. By contrast, the protein-based VLPs described herein are free of, or substantially free of, lipid and surfactants. Furthermore, the symmetric display of the rabies G protein on some embodiments of the protein-based VLPs of this disclosure may generate superior immune response to the G protein compared to other VLPs.

The term “virus-like particle” or “VLP” refers to a molecular assembly that resembles a virus but is non-infectious, and displays an antigenic protein, or antigenic fragment thereof, of a viral protein or glycoprotein. A “protein-based VLP” refers to a VLP formed from proteins or glycoproteins and is substantially free of other components (e.g., lipids). Protein-based VLPs may include post-translation modification and chemical modification, but are to be distinguished from micellar VLPs and VLPs formed by extraction of viral proteins from live or live inactivated virus preparations. The term “designed VLP” refers to a VLP comprising one or more polypeptides generated by computational protein design. Illustrative designed VLPs are VLPs that comprise nanostructures depicted in FIG. 2. The term “symmetric VLP” refers to a protein-based VLP with a symmetric core, such as shown in FIG. 2 and FIG. 3. These include but are not limited to designed VLPs.

The term “icosahedral particle” refers to a designed VLP having a core with icosahedral symmetry (e.g., the particles labeled I53 and I52 in Table 3). I53 refers to an icosahedral particle constructed from pentamers and trimers. I52 refers to an icosahedral particle constructed from pentamers and dimers. T33 refers to a tetrahedral particle constructed from two sets of trimers. T32 refers to a tetrahedral particle constructed from trimers and dimers.

The antigens may be attached to the core of the protein-based VLP either non-covalently or covalently, including as a fusion protein or by other means disclosed herein. Multimeric antigens may optionally be displayed along a symmetry axis of the VLP. Also provided are proteins and nucleic acid molecules encoding such proteins, formulations, and methods of use.

A non-limiting example of an embodiment is shown in FIG. 3, which depicts the G protein genetically fused to a multimerization domain of the VLP, which optionally is expressed recombinantly in a host cell (e.g., 293F cells); along with a pentameric homomeric complex of the second component, which is optionally expressed recombinantly in the same or a different host cell (e.g., E. coli cells). These two components self-assemble into a VLP displaying 20 G-protein trimers around an icosahedral core. In this embodiment, the core has a generic design. In some embodiments, the VLP is further linked to polypeptides or other agents capable of acting as an adjuvant. In some embodiments, the antigen (first component) and/or the second component comprises one or more T cell epitopes, optionally a T cell epitope of heterologous origin.

Other potential arrangements of polypeptides of the present disclosure are shown in FIG. 2. In some embodiments, the VLP is adapted for display of up to 8 trimers; 8 trimers and 12 dimers; 6 tetramers and 12 dimers; 6 tetramers and 8 trimers; 20 trimers and 30 dimers; 4 trimers and 6 dimers; 4 first trimers and 4 second trimers, or 8 trimers; 12 pentamers and 20 trimers; 12 pentamers and 30 dimers; or 4 trimers. In some cases, one of the symmetric axes is not used for antigen display, thus, in some embodiments the VLP is adapted for display of up to 8 trimers; 12 dimers; 6 tetramers; 20 trimers; 30 dimers; 4 trimers; 6 dimers; 8 trimers; or 12 pentamers. In some cases, monomeric antigens are displayed and thus, the VLP is adapted for display of up to 12, 24, 60, or 70 monomeric antigens. In some cases, the VLP comprises mixed pluralities of polypeptides such that otherwise identical polypeptides of the core of the VLP display different antigens or no antigen. Thus, depending on the ratio of polypeptides, the VLP is in some cases adapted for display of between 1 and 130 antigens (e.g., on the 152 particle) where each of the antigens displayed may be the same or may be different members of mixed population in proportion to any ratio chosen. The antigens may be co-expressed in a recombinant expression system and self-assembled before purification. Alternatively, the antigens may be expressed separately and then mixed together, either before or after purification from expression host and associated contaminants. In various embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more antigens are displayed. Non-limiting exemplary VLPs are provided in Bale et al. Science 353:389-94 (2016); Heinze et al. J. Phys. Chem B. 120:5945-5952 (2016); King et al. Nature 510:103-108 (2014); and King et al. Science 336:1171-71 (2012).

In some cases, the VLP is adapted to display the same antigen from two or more diverse strains of rabies. In non-limiting examples, the same VLP displays mixed populations of homotrimeric protein antigens or mixed heterotrimers of protein antigens from different strains of rabies. In further embodiments, particles may be made to display proteins from diverse sources, such as a rabies G protein ectodomain and one or more trimeric glycoproteins of HIV-1, HIV-2, EBV, CMV, RSV, influenza, Ebola, Marburg, Dengue, SARS, MERS, Hanta, or Zika virus. In some embodiments, the VLP comprises the trimeric glycoproteins of viruses that are related evolutionarily or in sequence identity to any of these exemplary viruses, including without limitation, a herpes virus, orthomyxovirus, paramyxovirus, pneumovirus, filovirus, flavivirus, reovirus, or retrovirus. In an embodiment, the VLP comprises the extracellular domain or domains of a transmembrane protein or glycoprotein, or an antigenic fragment thereof.

When mixed VLPs are made, it may be advantageous to ensure homomerization in a strain-specific manner rather than permit heterodimerization, such that, for example all strain 1 G proteins are displayed on one 3-fold axis of a T33 particle whereas all strain 2 G proteins are displayed on the other 3-fold axis of the T33 particle. This may be achieved by use a VLP comprising two or more pluralities of polypeptides as the core of the VLP with each plurality of polypeptides attached to a different antigen. Alternatively, a VLP may be engineered with one or more symmetry-breaking mutations, such as knob-in-hole mutations or intramolecular disulfide mutations, which have the effect of preventing trimer formation between the different antigens. In that case, the VLP displays multimeric antigens from different strains at symmetrically equivalent positions on the VLP, but each position on the VLP is occupied by homomers from the same strain, with only an insignificant proportion of inter-strain heteromeric antigens. In some cases, the antigen itself may be genetically engineered to prevent inter-strain heterodimerization. In an embodiment, the VLP is engineered to prevent heteromization of two antigenic proteins with conserved structure but divergent antigenicity, such as for example, a strain 1 G protein and strain 2 G protein, or a rabies G protein and a non-rabies antigenic protein. Furthermore, when mixed VLPs are made and the antigens are displayed as fusion proteins, the VLP will comprise three or more different proteins, as the fusion proteins will share identical (or equivalent) domains used to form the core of the VLP with different antigenic domains, one for each antigen displayed on the VLP.

### Attachment Modalities (Linkers)

The VLPs of the present disclosure display antigenic proteins in various ways including as gene fusion or by other means disclosed herein. As used herein, “linked to” or “attached to” denotes any means known in the art for causing two polypeptides to associate. The association may be direct or indirect, reversible or irreversible, weak or strong, covalent or non-covalent, and selective or nonselective. In some embodiments, a “polypeptide linker” is described which may refer to a linker between two parts of the G protein.

In some embodiments, attachment is achieved by genetic engineering to create an N- or C-terminus fusion of the rabies G protein ectodomain to the multimerization domain.

In some embodiments, attachment is achieved by post-translational covalent attachment of the rabies G protein ectodomain to the multimerization domain of the first component of the VLP. In some cases, chemical cross-linking is used to non-specifically attach the antigen to a VLP polypeptide. In some cases, chemical cross-linking is used to specifically attach the antigenic protein to a VLP polypeptide (e.g., to the first polypeptide or the second polypeptide). Various specific and non-specific cross-linking chemistries are known in the art, such as Click chemistry and other methods. In general, any cross-linking chemistry used to link two proteins may be adapted for use in the presently disclosed VLPs. In particular, chemistries used in creation of immunoconjugates or antibody drug conjugates may be used. In some cases, a VLP is created using a cleavable or non-cleavable linker. Processes and methods for conjugation of antigens to carriers are provided by, e.g., U.S. Patent Pub. No. US 2008/0145373 A1.

In an embodiment, attachment is achieved by non-covalent attachment to the VLP. In some cases, the rabies G protein ectodomain is engineered to be negatively charged on at least one surface and the core polypeptide is engineered to be positively charged on at least one surface, or positively and negatively charged, respectively. This promotes intermolecular association between the antigenic protein and the core polypeptide by electrostatic force. In some cases, shape complementarity is employed to cause linkage of antigen protein to core. Shape complementarity can be pre-existing or rationally designed. In an embodiment, the antigen is biotin-labeled and the polypeptide comprises a streptavidin, or vice versa. In an embodiment, streptavidin is displayed by gene fusion or otherwise as a tetramer on a 4-fold axis of the core and the biotin-labeled antigen is monomeric, dimeric, or tetrameric, permitting association to the core in a configuration appropriate for native multimerization of the antigen. In some cases, a protein-based adaptor is used to capture the antigenic protein. In some cases, the polypeptide is fused to a protein capable of binding a complementary protein, which is fused to the antigenic protein.

The immune reaction to rabies G may be controlled by altering the orientation of the ectodomain relative to the core. Depending on how the antigenic protein is attached to the core of the VLP, the antigenic protein may be displayed in various orientations. In some embodiments, the antigenic protein is displayed so that one or more known epitopes are oriented at or towards the distal end of the antigenic protein, such that these epitope(s) are preferentially accessible to the immune system. In some cases, the orientation will recapitulate the orientation of a viral protein with respect to the virus. Thus, in the case of rabies G protein, the antigenic protein (rabies G protein ectodomain) may be oriented so that the epitope identified in Marissen et al. (2005) J Virol. 79(8):4672-4678 is at the distal end of the protein. The choice of orientation may direct the immune system to one or the other epitope.

In some embodiments, epitope preference is control by other means, such as positioning of glycans on the VLP by addition or subtraction of the N-linked glycan sequence motif N-X-[T/S] at predetermined positions in the amino acid sequence of any of the polypeptides of the VLP including in the amino acid sequence of the antigenic protein.

In some cases, the epitopes found at intermediate distances from the proximal to the distal end will be preferred over epitopes more distally located depending on various considerations including but not limited to the overall geometry of the VLP, surface hydrophobicity, surface charge, and competitive binding of proteins endogenously present in the subject or proteins exogenously provided in the vaccine composition. The present disclosure encompasses all known methods of rational design of protein structure and the foregoing is not intended to be limiting.

### VLP Polypeptide Sequences

The polypeptides of the present disclosure may comprise various amino acids sequences. U.S. Patent Pub No. US 2015/0356240 A1 describes various methods for designing protein assemblies. As described in U.S. Patent Pub. No. US 2016/0122392 A1 and in International Patent Pub. No. WO 2014/124301 A1, the isolated polypeptides of SEQ ID NOS: 1-51 were designed for their ability to self-assemble in pairs to form VLPs, such as icosahedral particles. Further suitable VLPs are described in U.S. Patent Pub. No. US 2022/0072120 A1. The design method involved designing suitable interface residues for each member of the polypeptide pair that can be assembled to form the VLP. The VLPs so formed include symmetrically repeated, non-natural, non-covalent polypeptide-polypeptide interfaces that orient a first assembly and a second assembly into a VLP, such as one with an icosahedral symmetry. Thus, in one embodiment the first polypeptide and second polypeptide (that is, the two polypeptides of the core of the VLP) are selected from the group consisting of SEQ ID NOS: 1-51. In each case, an N-terminal methionine residue present in the full-length protein but typically removed to make a fusion is not included in the sequence. In various embodiments, one or more additional residues are deleted from the N-terminus and/or additional residues are added to the N-terminus (e.g., to form a helical extension).

TABLE 3

| | | | Identified |
|---|---|---|---|
| | Component | | interface |
| Name | Multimer | Amino Acid Sequence | residues |
| I53-34A | trimer | EGMDPLAVLAESRLLPLLTVRGGEDLAGLATVLELMG | I53-34A: |
| SEQ ID NO: 1 | | VGALEITLRTEKGLEALKALRKSGLLLGAGTVRSPKE | 28, 32, 36, 37, 186, |
| | | AEAALEAGAAFLVSPGLLEEVAALAQARGVPYLPGVL | 188, 191, 192, 195 |
| | | TPTEVERALALGLSALKFFPAEPFQGVRVLRAYAEVE | |
| | | PEVRFLPTGGIKEEHLPHYAALPNLLAVGGSWLLQGD | |
| | | LAAVMKKVKAAKALLSPQAPG | |
| I53-34B | pentamer | TKKVGIVDTTFARVDMAEAAIRTLKALSPNIKIIRKT | I53-34B: |
| SEQ ID NO: 2 | | VPGIKDLPVACKKLLEEEGCDIVMALGMPGKAEKDKV | 19, 20, 23, 24, 27, |
| | | CAHEASLGLMLAQLMTNKHIIEVFVHEDEAKDDDELD | 109, 113, 116, 117, |
| | | ILALVRAIEHAANVYYLLFKPEYLTRMAGKGLRQGRE | 120, 124, 148 |
| | | DAGPARE | |
| I53-40A | pentamer | TKKVGIVDTTFARVDMASAAILTLKMESPNIKIIRKT | I53-40A: |
| SEQ ID NO: 3 | | VPGIKDLPVACKKLLEEEGCDIVMALGMPGKAEKDKV | 20, 23, 24, 27, 28, |
| | | CAHEASLGLMLAQLMTNKHIIEVFVHEDEAKDDAELK | 109, 112, 113, 116, |
| | | ILAARRAIEHALNVYYLLFKPEYLTRMAGKGLRQGFE | 120, 124 |
| | | DAGPARE | |
| I53-40B | trimer | STINNQLKALKVIPVIAIDNAEDIIPLGKVLAENGLP | I53-40B: |
| SEQ ID NO: 4 | | AAEITFRSSAAVKAIMLLRSAQPEMLIGAGTILNGVQ | 47, 51, 54, 58, 74, 102 |
| | | ALAAKEAGATFVVSPGFNPNTVRACQIIGIDIVPGVN | |
| | | NPSTVEAALEMGLTTLKFFPAEASGGISMVKSLVGPY | |
| | | GDIRLMPTGGITPSNIDNYLAIPQVLACGGTWMVDKK | |
| | | LVTNGEWDEIARLTREIVEQVNP | |
| I53-47A | trimer | PIFTLNTNIKATDVPSDFLSLTSRLVGLILSKPGSYV | I53-47A: |
| SEQ ID NO: 5 | | AVHINTDQQLSFGGSTNPAAFGTLMSIGGIEPSKNRD | 22, 25, 29, 72, 79, 86, |
| | | HSAVLFDHLNAMLGIPKNRMYIHFVNLNGDDVGWNGT | 87 |
| | | TF | |
| I53-47B | pentamer | NQHSHKDYETVRIAVVRARWHADIVDACVEAFEIAMA | I53-47B: |
| SEQ ID NO: 6 | | AIGGDRFAVDVFDVPGAYEIPLHARTLAETGRYGAVL | 28, 31, 35, 36, 39, |
| | | GTAFVVNGGIYRHEFVASAVIDGMMNVOLSTGVPVLS | 131, 132, 135, 139, |
| | | AVLTPHRYRDSAEHHRFFAAHFAVKGVEAARACIEIL | 146 |
| | | AAREKIAA | |
| I53-50A | trimer | MEELFKKHKIVAVLRANSVEEAIEKAVAVFAGGVHLI | I53-50A: |
| SEQ ID NO: 7 | | EITFTVPDADTVIKALSVLKEKGAIIGAGTVTSVEQC | 25, 29, 33, 54, 57 |
| | | RKAVESGAEFIVSPHLDEEISQFCKEKGVFYMPGVMT | |
| | | PTELVKAMKLGHTILKLFPGEVVGPQFVKAMKGPFPN | |
| | | VKFVPTGGVNLDNVCEWFKAGVLAVGVGSALVKGTPD | |
| | | EVREKAKAFVEKIRGCTE | |
| I53-50B | pentamer | NQHSHKDYETVRIAVVRARWHAEIVDACVSAFEAAMA | I53-50B: |
| SEQ ID NO: 8 | | DIGGDRFAVDVFDVPGAYEIPLHARTLAETGRYGAVL | 24, 28, 36, 124, 125, |
| | | GTAFVVNGGIYRHEFVASAVIDGMMNVQLSTGVPVLS | 127, 128, 129, |
| | | AVLTPHRYRDSDAHTLLFLALFAVKGMEAARACVEIL | 131, 132, 133, 135, |
| | | AAREKIAA | 139 |
| I53-51A | trimer | FTKSGDDGNTNVINKRVGKDSPLVNFLGDLDELNSFI | I53-51A: |
| SEQ ID NO: 9 | | GFAISKIPWEDMKKDLERVQVELFEIGEDLSTQSSKK | 80, 83, 86, 87, 88, 90, |
| | | KIDESYVLWLLAATAIYRIESGPVKLFVIPGGSEEAS | 91, 94, 166, 172, 176 |
| | | VLHVTRSVARRVERNAVKYTKELPEINRMIIVYLNRL | |
| | | SSLLFAMALVANKRRNQSEKIYEIGKSW | |
| I53-51B | pentamer | NQHSHKDYETVRIAVVRARWHADIVDQCVRAFEEAMA | I53-51B: |
| SEQ ID NO: 10 | | DAGGDRFAVDVEDVPGAYEIPLHARTLAETGRYGAVL | 31, 35, 36, 40, 122, |
| | | GTAFVVNGGIYRHEFVASAVIDGMMNVQLSTGVPVLS | 124, 128, 131, 135, |
| | | AVLTPHRYRSSREHHEFFREHFMVKGVEAAAACITIL | 139, 143, 146, 147 |
| | | AAREKIAA | |
| I52-03A | pentamer | GHTKGPTPQQHDGSALRIGIVHARWNKTIIMPLLIGT | I52-03A: |
| SEQ ID NO: 11 | | IAKLLECGVKASNIVVQSVPGSWELPIAVQRLYSASQ | 28, 32, 36, 39, 44, 49 |
| | | LQTPSSGPSLSAGDLLGSSTTDLTALPTTTASSTGPF | |
| | | DALIAIGVLIKGETMHFEYIADSVSHGLMRVQLDTGV | |
| | | PVIFGVLTVLTDDQAKARAGVIEGSHNHGEDWGLAAV | |
| | | EMGVRRRDWAAGKTE | |
| I52-03B | dimer | YEVDHADVYDLFYLGRGKDYAAEASDIADLVRSRTPE | I52-03B: |
| SEQ ID NO: 12 | | ASSLLDVACGTGTHLEHFTKEFGDTAGLELSEDMLTH | 94, 115, 116, 206, 213 |
| | | ARKRLPDATLHQGDMRDFQLGRKFSAVVSMFSSVGYL | |
| | | KTVAELGAAVASFAEHLEPGGVVVVEPWWFPETFADG | |
| | | WVSADVVRRDGRTVARVSHSVREGNATRMEVHFTVAD | |
| | | PGKGVRHFSDVHLITLFHQREYEAAFMAAGLRVEYLE | |
| | | GGPSGRGLFVGVPA | |
| I52-32A | dimer | GMKEKFVLIITHGDFGKGLLSGAEVIIGKQENVHTVG | I52-32A: |
| SEQ ID NO: 13 | | LNLGDNIEKVAKEVMRIIIAKLAEDKEIIIVVDLFGG | 47, 49, 53, 54, 57, 58, |
| | | SPFNIALEMMKTFDVKVITGINMPMLVELLTSINVYD | 61, 83, 87, 88 |
| | | TTELLENISKIGKDGIKVIEKSSLKM | |
| I52-32B | pentamer | KYDGSKLRIGILHARWNLEIIAALVAGAIKRLQEFGV | I52-32B: |
| SEQ ID NO: 14 | | KAENIIIETVPGSFELPYGSKLFVEKQKRLGKPLDAI | 19, 20, 23, 30, 40 |
| | | IPIGVLIKGSTMHFEYICDSTTHQLMKLNFELGIPVI | |
| | | FGVLTCLTDEQAEARAGLIEGKMHNHGEDWGAAAVEM | |
| | | ATKFN | |
| I52-33A | pentamer | AVKGLGEVDQKYDGSKLRIGILHARWNRKIILALVAG | I52-33A: |
| SEQ ID NO: 15 | | AVLRLLEFGVKAENIIIETVPGSFELPYGSKLFVEKQ | 33, 41, 44, 50 |
| | | KRLGKPLDAIIPIGVLIKGSTMHFEYICDSTTHQLMK | |
| | | LNFELGIPVIFGVLTCLTDEQAEARAGLIEGKMHNHG | |
| | | EDWGAAAVEMATKFN | |
| I52-33B | dimer | GANWYLDNESSRLSFTSTKNADIAEVHRFLVLHGKVD | I52-33B: |
| SEQ ID NO: 16 | | PKGLAEVEVETESISTGIPLRDMLLRVLVFQVSKFPV | 61, 63, 66, 67, 72, |
| | | AQINAQLDMRPINNLAPGAQLELRLPLTVSLRGKSHS | 147, 148, 154, 155 |
| | | YNAELLATRLDERRFQVVTLEPLVIHAQDEDMVRAFN | |
| | | ALRLVAGLSAVSLSVPVGAVLIFTAR | |
| I32-06A | dimer | TDYIRDGSAIKALSFAIILAEADLRHIPQDLQRLAVR | I32-06A: |
| SEQ ID NO: 17 | | VIHACGMVDVANDLAFSEGAGKAGRNALLAGAPILCD | 9, 12, 13, 14, 20, 30, |
| | | ARMVAEGITRSRLPADNRVIYTLSDPSVPELAKKIGN | 33, 34 |
| | | TRSAAALDLWLPHIEGSIVAIGNAPTALFRLFELLDA | |
| | | GAPKPALIIGMPVGFVGAAESKDELAANSRGVPYVIV | |
| | | RGRRGGSAMTAAAVNALASERE | |
| I32-06B | trimer | ITVFGLKSKLAPRREKLAEVIYSSLHLGLDIPKGKHA | I32-06B: |
| SEQ ID NO: 18 | | IRFLCLEKEDFYYPFDRSDDYTVIEINLMAGRSEETK | 24, 71, 73, 76, 77, 80, |
| | | MLLIFLLFIALERKLGIRAHDVEITIKEQPAHCWGFR | 81, 84, 85, 88, 114, |
| | | GRTGDSARDLDYDIYV | 118 |
| I32-19A | trimer | GSDLQKLQRFSTCDISDGLLNVYNIPTGGYFPNLTAI | I32-19A: |
| SEQ ID NO: 19 | | SPPQNSSIVGTAYTVLFAPIDDPRPAVNYIDSVPPNS | 208, 213, 218, 222, |
| | | ILVLALEPHLQSQFHPFIKITQAMYGGLMSTRAQYLK | 225, 226, 229, 233 |
| | | SNGTVVFGRIRDVDEHRTLNHPVFAYGVGSCAPKAVV | |
| | | KAVGTNVQLKILTSDGVTQTICPGDYIAGDNNGIVRI | |
| | | PVQETDISKLVTYIEKSIEVDRLVSEAIKNGLPAKAA | |
| | | QTARRMVLKDYI | |
| I32-19B | dimer | SGMRVYLGADHAGYELKQAIIAFLKMTGHEPIDCGAL | I32-19B: |
| SEQ ID NO: 20 | | RYDADDDYPAFCIAAATRTVADPGSLGIVLGGSGNGE | 20, 23, 24, 27, 117, |
| | | QIAANKVPGARCALAWSVQTAALAREHNNAQLIGIGG | 118, 122, 125 |
| | | RMHTLEEALRIVKAFVTTPWSKAQRHORRIDILAEYE | |
| | | RTHEAPPVPGAPA | |
| I32-28A | trimer | GDDARIAAIGDVDELNSQIGVLLAEPLPDDVRAALSA | I32-28A: |
| SEQ ID NO: 21 | | IQHDLFDLGGELCIPGHAAITEDHLLRLALWLVHYNG | 60, 61, 64, 67, 68, |
| | | QLPPLEEFILPGGARGAALAHVCRTVCRRAERSIKAL | 71, 110, 120, 123, 124, |
| | | GASEPLNIAPAAYVNLLSDLLFVLARVLNRAAGGADV | 128 |
| | | LWDRTRAH | |
| I32-28B | dimer | ILSAEQSFTLRHPHGQAAALAFVREPAAALAGVQRLR | I32-28B: |
| SEQ ID NO: 22 | | GLDSDGEQVWGELLVRVPLLGEVDLPFRSEIVRTPQG | 35, 36, 54, 122, 129, |
| | | AELRPLTLTGERAWVAVSGQATAAEGGEMAFAFQFQA | 137, 140, 141, 144, |
| | | HLATPEAEGEGGAAFEVMVQAAAGVTLLLVAMALPQG | 148 |
| | | LAAGLPPA | |
| I53-40A.1 | pentamer | TKKVGIVDTTFARVDMASAAILTLKMESPNIKIIRKT | I53-40A: |
| SEQ ID NO: 23 | | VPGIKDLPVACKKLLEEEGCDIVMALGMPGKKEKDKV | 20, 23, 24, 27, 28, |
| | | CAHEASLGLMLAQLMTNKHIIEVFVHEDEAKDDAELK | 109, 112, 113, 116, |
| | | ILAARRAIEHALNVYYLLFKPEYLTRMAGKGLRQGFE | 120, 124 |
| | | DAGPARE | |
| I53-40B.1 | trimer | DDINNQLKRLKVIPVIAIDNAEDIIPLGKVLAENGLP | I53-40B: |
| SEQ ID NO: 24 | | AAEITFRSSAAVKAIMLLRSAQPEMLIGAGTILNGVQ | 47, 51, 54, 58, 74, |
| | | ALAAKEAGADFVVSPGFNPNTVRACQIIGIDIVPGVN | 102 |
| | | NPSTVEQALEMGLTTLKFFPAEASGGISMVKSLVGPY | |
| | | GDIRLMPTGGITPDNIDNYLAIPQVLACGGTWMVDKK | |
| | | LVRNGEWDEIARLTREIVEQVNP | |
| I53-47A.1 | trimer | PIFTLNTNIKADDVPSDFLSLTSRLVGLILSKPGSYV | I53-47A: |
| SEQ ID NO: 25 | | AVHINTDQQLSFGGSTNPAAFGTLMSIGGIEPDKNRD | 22, 25, 29, 72, 79, |
| | | HSAVLFDHLNAMLGIPKNRMYIHFVNLNGDDVGWNGT | 86, 87 |
| | | TF | |
| I53- | trimer | PIFTLNTNIKADDVPSDFLSLTSRLVGLILSEPGSYV | I53-47A: |
| 47A.1NegT2 | | AVHINTDQQLSFGGSTNPAAFGTLMSIGGIEPDKNED | 22, 25, 29, 72, 79, |
| SEQ ID NO: 26 | | HSAVLFDHLNAMLGIPKNRMYIHFVDLDGDDVGWNGT | 86, 87 |
| | | TF | |
| I53-47B.1 | pentamer | NQHSHKDHETVRIAVVRARWHADIVDACVEAFEIAMA | I53-47B: |
| SEQ ID NO: 27 | | AIGGDRFAVDVEDVPGAYEIPLHARTLAETGRYGAVL | 28, 31, 35, 36, 39, |
| | | GTAFVVNGGIYRHEFVASAVIDGMMNVQLDTGVPVLS | 131, 132, 135, 139, |
| | | AVLTPHRYRDSDEHHRFFAAHFAVKGVEAARACIEIL | 146 |
| | | NAREKIAA | |
| 153-47B.1NegT2 | pentamer | NQHSHKDHETVRIAVVRARWHADIVDACVEAFEIAMA | I53-47B: |
| SEQ ID NO: 28 | | AIGGDRFAVDVFDVPGAYEIPLHARTLAETGRYGAVL | 28, 31, 35, 36, 39, |
| | | GTAFVVDGGIYDHEFVASAVIDGMMNVQLDTGVPVLS | 131, 132, 135, 139, |
| | | AVLTPHEYEDSDEDHEFFAAHFAVKGVEAARACIEIL | 146 |
| | | NAREKIAA | |
| I53-50A.1 | trimer | EELFKKHKIVAVLRANSVEEAIEKAVAVFAGGVHLIE | I53-50A: |
| SEQ ID NO: 29 | | ITFTVPDADTVIKALSVLKEKGAIIGAGTVTSVEQCR | 25, 29, 33, 54, 57 |
| | | KAVESGAEFIVSPHLDEEISQFCKEKGVFYMPGVMTP | |
| | | TELVKAMKLGHDILKLFPGEVVGPQFVKAMKGPFPNV | |
| | | KFVPTGGVNLDNVCEWFKAGVLAVGVGDALVKGDPDE | |
| | | VREKAKKFVEKIRGCTE | |
| I53- | trimer | EELFKKHKIVAVLRANSVEEAIEKAVAVFAGGVHLIE | I53-50A: |
| 50A.1NegT2 | | ITFTVPDADTVIKALSVLKEKGAIIGAGTVTSVEQCR | 25, 29, 33, 54, 57 |
| SEQ ID NO: 30 | | KAVESGAEFIVSPHLDEEISQFCKEKGVFYMPGVMTP | |
| | | TELVKAMKLGHDILKLFPGEVVGPEFVEAMKGPFPNV | |
| | | KFVPTGGVDLDDVCEWFDAGVLAVGVGDALVEGDPDE | |
| | | VREDAKEFVEEIRGCTE | |
| I53-50A.1PosT1 | trimer | EELFKKHKIVAVLRANSVEEAIEKAVAVFAGGVHLIE | I53-50A: |
| SEQ ID NO: 31 | | ITFTVPDADTVIKALSVLKEKGAIIGAGTVTSVEQCR | 25, 29, 33, 54, 57 |
| | | KAVESGAEFIVSPHLDEEISQFCKEKGVFYMPGVMTP | |
| | | TELVKAMKLGHDILKLFPGEVVGPQFVKAMKGPFPNV | |
| | | KFVPTGGVNLDNVCKWFKAGVLAVGVGKALVKGKPDE | |
| | | VREKAKKFVKKIRGCTE | |
| I53-50B.1 | pentamer | NQHSHKDHETVRIAVVRARWHAEIVDACVSAFEAAMR | I53-50B: |
| SEQ ID NO: 32 | | DIGGDRFAVDVFDVPGAYEIPLHARTLAETGRYGAVL | 24, 28, 36, 124, 125, |
| | | GTAFVVNGGIYRHEFVASAVIDGMMNVQLDTGVPVLS | 127, 128, 129, 131, |
| | | AVLTPHRYRDSDAHTLLFLALFAVKGMEAARACVEIL | 132, 133, 135, 139 |
| | | AAREKIAA | |
| I53-50B.1NegT2 | pentamer | NQHSHKDHETVRIAVVRARWHAEIVDACVSAFEAAMR | I53-50B: |
| SEQ ID NO: 33 | | DIGGDRFAVDVFDVPGAYEIPLHARTLAETGRYGAVL | 24, 28, 36, 124, 125, |
| | | GTAFVVDGGIYDHEFVASAVIDGMMNVQLDTGVPVLS | 127, 128, 129, 131, |
| | | AVLTPHEYEDSDADTLLFLALFAVKGMEAARACVEIL | 132, 133, 135, 139 |
| | | AAREKIAA | |
| I53-50B.4PosT1 | trimer | NQHSHKDHETVRIAVVRARWHAEIVDACVSAFEAAMR | I53-50B: |
| SEQ ID NO: 34 | | DIGGDRFAVDVFDVPGAYEIPLHARTLAETGRYGAVL | 24, 28, 36, 124, 125, |
| | | GTAFVVNGGIYRHEFVASAVINGMMNVQLNTGVPVLS | 127, 128, 129, 131, |
| | | AVLTPHNYDKSKAHTLLFLALFAVKGMEAARACVEIL | 132, 133, 135, 139 |
| | | AAREKIAA | |
| I53-40A genus | pentamer | TKKVGIVDTTFARVDMASAAILTLKMESPNIKIIRKT | |
| SEQ ID NO: 35 | | VPGIKDLPVACKKLLEEEGCDIVMALGMPGK(A/K)E | |
| | | KDKVCAHEASLGLMLAQLMTNKHIIEVFVHEDEAKDD | |
| | | AELKILAARRAIEHALNVYYLLFKPEYLTRMAGKGLR | |
| | | QGFEDAGPARE | |
| I53-40B genus | trimer | (S/D)(T/D)INNQLK(A/R)LKVIPVIAIDNAEDII | |
| SEQ ID NO: 36 | | PLGKVLAENGLPAAEITFRSSAAVKAIMLLRSAQPEM | |
| | | LIGAGTILNGVQALAAKEAGA(T/D)FVVSPGFNPNT | |
| | | VRACQIIGIDIVPGVNNPSTVE(A/Q)ALEMGLTTLK | |
| | | FFPAEASGGISMVKSLVGPYGDIRLMPTGGITP(S/D) | |
| | | NIDNYLAIPQVLACGGTWMVDKKLV(T/R)NGEWDE | |
| | | IARLTREIVEQVNP | |
| I53-47A genus | trimer | PIFTLNTNIKA(T/D)DVPSDFLSLTSRLVGLILS(K/ | |
| SEQ ID NO: 37 | | E)PGSYVAVHINTDQQLSFGGSTNPAAFGTLMSIGG | |
| | | IEP(S/D)KN(R/E)DHSAVLFDHLNAMLGIPKNRMY | |
| | | IHFV(N/D)L(N/D)GDDVGWNGTTF | |
| I53-47B genus | pentamer | NQHSHKD(Y/H)ETVRIAVVRARWHADIVDACVEAFE | |
| SEQ ID NO: 38 | | IAMAAIGGDRFAVDVFDVPGAYEIPLHARTLAETGRY | |
| | | GAVLGTAFVV(N/D)GGIY(R/D)HEFVASAVIDGMM | |
| | | NVQL(S/D)TGVPVLSAVLTPH(R/E)Y(R/E)DS(A/ | |
| | | D)E(H/D)H(R/E)FFAAHFAVKGVEAARACIEIL | |
| | | (A/N)AREKIAA | |
| I53-50A genus | trimer | EELFKKHKIVAVLRANSVEEAIEKAVAVFAGGVHLIE | |
| SEQ ID NO: 39 | | ITFTVPDADTVIKALSVLKEKGAIIGAGTVTSVEQCR | |
| | | KAVESGAEFIVSPHLDEEISQFCKEKGVFYMPGVMTP | |
| | | TELVKAMKLGH(T/D)ILKLFPGEVVGP(Q/E)FV(K/ | |
| | | E)AMKGPFPNVKFVPTGGV(N/D)LD(N/D)VC(E/ | |
| | | K)WF(K/D)AGVLAVGVG(S/K/D)ALV(K/E)G(T/ | |
| | | D/K)PDEVRE(K/D)AK(A/E/K)FV(E/K)(K/E)I | |
| | | RGCTE | |
| I53-50B genus | pentamer | NQHSHKD(Y/H)ETVRIAVVRARWHAEIVDACVSAFE | |
| SEQ ID NO: 40 | | AAM(A/R)DIGGDRFAVDVFDVPGAYEIPLHARTLAE | |
| | | TGRYGAVLGTAFVV(N/D)GGIY(R/D)HEFVASAVI | |
| | | (D/N)GMMNVQL(S/D/N)TGVPVLSAVLTPH(R/E/ | |
| | | N)Y(R/D/E)(D/K)S(D/K)A(H/D)TLLFLALFAV | |
| | | KGMEAARACVEILAAREKIAA | |
| T32-28A | dimer | GEVPIGDPKELNGMEIAAVYLOPIEMEPRGIDLAASL | |
| SEQ ID NO: 41 | | ADIHLEADIHALKNNPNGFPEGFWMPYLTIAYALANA | |
| | | DTGAIKTGTLMPMVADDGPHYGANIAMEKDKKGGFGV | |
| | | GTYALTFLISNPEKQGFGRHVDEETGVGKWFEPFVVT | |
| | | YFFKYTGTPK | |
| T32-28B | trimer | SQAIGILELTSIAKGMELGDAMLKSANVDLLVSKTIS | |
| SEQ ID NO: 42 | | PGKFLLMLGGDIGAIQQAIETGTSQAGEMLVDSLVLA | |
| | | NIHPSVLPAISGLNSVDKRQAVGIVETWSVAACISAA | |
| | | DLAVKGSNVTLVRVHMAFGIGGKCYMVVAGDVLDVAA | |
| | | AVATASLAAGAKGLLVYASIIPRPHEAMWRQMVEG | |
| T33-09A | timer | EEVVLITVPSALVAVKIAHALVEERLAACVNIVPGLT | |
| SEQ ID NO: 43 | | SIYRWQGSVVSDHELLLLVKTTTHAFPKLKERVKALH | |
| | | PYTVPEIVALPIAEGNREYLDWLRENTG | |
| T33-09B | timer | VRGIRGAITVEEDTPAAILAATIELLLKMLEANGIQS | |
| SEQ ID NO: 44 | | YEELAAVIFTVTEDLTSAFPAEAARLIGMHRVPLLSA | |
| | | REVPVPGSLPRVIRVLALWNTDTPQDRVRHVYLNEAV | |
| | | RLRPDLESAQ | |
| T33-15A | timer | SKAKIGIVTVSDRASAGITADISGKAIILALNLYLTS | |
| SEQ ID NO: 45 | | EWEPIYQVIPDEQDVIETTLIKMADEQDCCLIVTTGG | |
| | | TGPAKRDVTPEATEAVCDRMMPGFGELMRAESLKEVP | |
| | | TAILSRQTAGLRGDSLIVNLPGDPASISDCLLAVFPA | |
| | | IPYCIDLMEGPYLECNEAMIKPFRPKAK | |
| T33-15B | timer | VRGIRGAITVNSDTPTSIIIATILLLEKMLEANGIQS | |
| SEQ ID NO: 46 | | YEELAAVIFTVTEDLTSAFPAEAARQIGMHRVPLLSA | |
| | | REVPVPGSLPRVIRVLALWNTDTPQDRVRHVYLSEAV | |
| | | RLRPDLESAQ | |
| T33-21A | timer | RITTKVGDKGSTRLFGGEEVWKDSPIIEANGTLDELT | |
| SEQ ID NO: 47 | | SFIGEAKHYVDEEMKGILEEIQNDIYKIMGEIGSKGK | |
| | | IEGISEERIAWLLKLILRYMEMVNLKSFVLPGGTLES | |
| | | AKLDVCRTIARRALRKVLTVTREFGIGAEAAAYLLAL | |
| | | SDLLFLLARVIEIEKNKLKEVRS | |
| T33-21B | timer | PHLVIEATANLRLETSPGELLEQANKALFASGQFGEA | |
| SEQ ID NO: 48 | | DIKSRFVTLEAYRQGTAAVERAYLHACLSILDGRDIA | |
| | | TRTLLGASLCAVLAEAVAGGGEEGVOVSVEVREMERL | |
| | | SYAKRVVARQR | |
| T33-28A | timer | ESVNTSFLSPSLVTIRDFDNGQFAVLRIGRTGFPADK | |
| SEQ ID NO: 49 | | GDIDLCLDKMIGVRAAQIFLGDDTEDGFKGPHIRIRC | |
| | | VDIDDKHTYNAMVYVDLIVGTGASEVERETAEEEAKL | |
| | | ALRVALQVDIADEHSCVTQFEMKLREELLSSDSFHPD | |
| | | KDEYYKDFL | |
| T33-28B | timer | PVIQTFVSTPLDHHKRLLLAIIYRIVTRVVLGKPEDL | |
| SEQ ID NO: 50 | | VMMTFHDSTPMHFFGSTDPVACVRVEALGGYGPSEPE | |
| | | KVTSIVTAAITAVCGIVADRIFVLYFSPLHCGWNGTN | |
| | | F | |
| T33-31A | timer | EEVVLITVPSALVAVKIAHALVEERLAACVNIVPGLT | |
| SEQ ID NO: 51 | | SIYREEGSVVSDHELLLLVKTTTDAFPKLKERVKELH | |
| | | PYEVPEIVALPIAEGNREYLDWLRENTG | |
| T33-dn2A | trimer | NLAEKMYKAGNAMYRKGQYTIAIIAYTLALLKDPNNA | |
| SEQ ID NO: 68 | | EAWYNLGNAAYKKGEYDEAIEAYQKALELDPNNAEAW | |
| | | YNLGNAYYKQGDYDEAIEYYKKALRLDPRNVDAIENL | |
| | | IEAEEKQG | |
| T33-dn2B SEQ | trimer | EEAELAYLLGELAYKLGEYRIAIRAYRIALKRDPNNA | |
| ID NO: 69 | | EAWYNLGNAYYKQGDYREAIRYYLRALKLDPENAEAW | |
| | | YNLGNALYKQGKYDLAIIAYQAALEEDPNNAEAKQNL | |
| | | GNAKQKQG | |
| T33-dn5A SEQ | trimer | NSAEAMYKMGNAAYKQGDYILAIIAYLLALEKDPNNA | |
| ID NO: 70 | | EAWYNLGNAAYKQGDYDEAIEYYQKALELDPNNAEAW | |
| | | YNLGNAYYKQGDYDEAIEYYEKALELDPNNAEALKNL | |
| | | LEAIAEQD | |
| T33-dn5B SEQ | trimer | TDPLAVILYIAILKAEKSIARAKAAEALGKIGDERAV | |
| ID NO: 71 | | EPLIKALKDEDALVRAAAADALGQIGDERAVEPLIKA | |
| | | LKDEEGLVRASAAIALGQIGDERAVQPLIKALTDERD | |
| | | LVRVAAAVALGRIGDEKAVRPLIIVLKDEEGEVREAA | |
| | | AIALGSIGGERVRAAMEKLAERGTGFARKVAVNYLET | |
| | | HK | |
| T33-dn10A SEQ | trimer | EEAELAYLLGELAYKLGEYRIAIRAYRIALKRDPNNA | |
| ID NO: 72 | | EAWYNLGNAYYKQGDYDEAIEYYQKALELDPNNAEAW | |
| | | YNLGNAYYKQGDYDEAIEYYEKALELDPENLEALQNL | |
| | | LNAMDKQG | |
| T33-dn10B SEQ | trimer | IEEVVAEMIDILAESSKKSIEELARAADNKTTEKAVA | |
| ID NO: 73 | | EAIEEIARLATAAIQLIEALAKNLASEEFMARAISAI | |
| | | AELAKKAIEAIYRLADNHTTDTFMARAIAAIANLAVT | |
| | | AILAIAALASNHTTEEFMARAISAIAELAKKAIEAIY | |
| | | RLADNHTTDKFMAAAIEAIALLATLAILAIALLASNH | |
| | | TTEKFMARAIMAIAILAAKAIEAIYRLADNHTSPTYI | |
| | | EKAIEAIEKIARKAIKAIEMLAKNITTEEYKEKAKKI | |
| | | IDIIRKLAKMAIKKLEDNRT | |
| I53-dn5A SEQ | pentamer | KYDGSKLRIGILHARWNAEIILALVLGALKRLQEFGV | 31, 43, 45, 48, 49, |
| ID NO: 74 | | KRENIIIETVPGSFELPYGSKLFVEKQKRLGKPLDAI | 51, 52, 55, 56, 59, |
| | | IPIGVLIKGSTMHFEYICDSTTHOLMKLNFELGIPVI | 69, 74, 76 |
| | | FGVLTCLTDEQAEARAGLIEGKMHNHGEDWGAAAVEM | |
| | | ATKFN | |
| I53-dn5B SEQ | trimer | EEAELAYLLGELAYKLGEYRIAIRAYRIALKRDPNNA | [] |
| ID NO: 75 | | EAWYNLGNAYYKQGRYREAIEYYQKALELDPNNAEAW | |
| | | YNLGNAYYERGEYEEAIEYYRKALRLDPNNADAMQNL | |
| | | LNAKMREE | |
| I53_dn5A.1 SEQ | pentamer | KYDGSKLRIGILHARGNAEIILALVLGALKRLQEFGV | |
| ID NO: 171 | | KRENIIIETVPGSFELPYGSKLFVEKQKRLGKPLDAI | |
| | | IPIGVLIRGSTPHFDYIADSTTHQLMKLNFELGIPVI | |
| | | FGVITADTDEQAEARAGLIEGKMHNHGEDWGAAAVEM | |
| | | ATKFN | |
| I53_dn5A.2 SEQ | pentamer | KYDGSKLRIGILHARGNAEIILELVLGALKRLQEFGV | |
| ID NO: 172 | | KRENIIIETVPGSFELPYGSKLFVEKQKRLGKPLDAI | |
| | | IPIGVLIRGSTAHFDYIADSTTHQLMKLNFELGIPVI | |
| | | FGVLTTESDEQAEERAGTKAGNHGEDWGAAAVEMATK | |
| | | FN | |
| I3-01 | | MKMEELFKKHKIVAVLRANSVEEAKKKALAVFLGGVH | |
| SEQ ID NO: 173 | | LIEITFTVPDADTVIKELSFLKEMGAIIGAGTVTSVE | |
| | | QCRKAVESGAEFIVSPHLDEEISQFCKEKGVFYMPGV | |
| | | MTPTELVKAMKLGHTILKLFPGEVVGPQFVKAMKGPF | |
| | | PNVKFVPTGGVNLDNVCEWFKAGVLAVGVGSALVKGT | |
| | | PVEVAEKAKAFVEKIRGCTE | |
| I3-01 (M31) | | MKIEELFKKHKIVAVLRANSVEEAKKKALAVFLGGVH | |
| SEQ ID NO: 174 | | LIEITFTVPDADTVIKELSFLKEMGAIIGAGTVTSVE | |
| | | QCRKAVESGAEFIVSPHLDEEISQFCKEKGVEYMPGV | |
| | | MTPTELVKAMKLGHTILKLFPGEVVGPQFVKAMKGPF | |
| | | PNVKFVPTGGVNLDNVCEWFKAGVLAVGVGSALVKGT | |
| | | PVEVAEKAKAFVEKIRGCTE | |
| 1WA3-ref | | MKMEELFKKHKIVAVLRANSVEEAKEKALAVFEGGVH | |
| SEQ ID NO: 175 | | LIEITFTVPDADTVIKELSFLKEKGAIIGAGTVTSVE | |
| | | QCRKAVESGAEFIVSPHLDEEISQFCKEKGVFYMPGV | |
| | | MTPTELVKAMKLGHTILKLFPGEVVGPQFVKAMKGPF | |
| | | PNVKFVPTGGVNLDNVCEWFKAGVLAVGVGSALVKGT | |
| | | PDEVREKAKAFVEKIRGCTE | |
| 1WA3-1 | | (MK)MEELFKKHKIVAVLRANSVEEAKKKALAVFLGG | |
| SEQ ID NO: 176 | | VHLIEITFTVPDADTVIKELSFLKEMGAIIGAGTVTS | |
| | | VEQCRKAVESGAEFIVSPHLDEEISQFCKEKGVFYMP | |
| | | GVMTPTELVKAMKLGHTILKLFPGEVVGPQFVKAMKG | |
| | | PFPNVKFVPTGGVNLDNVCEWFKAGVLAVGVGSALVK | |
| | | GTPVEVAEKAKAFVEKIRGCTE | |
| 1WA3-2 | | (MK)IEELFKKHKIVAVLRANSVEEAKKKALAVFLGG | |
| SEQ ID NO: 177 | | VHLIEITFTVPDADTVIKELSFLKEMGAIIGAGTVTS | |
| | | VEQCRKAVESGAEFIVSPHLDEEISQFCKEKGVFYMP | |
| | | GVMTPTELVKAMKLGHTILKLFPGEVVGPQFVKAMKG | |
| | | PFPNVKFVPTGGVNLDNVCEWFKAGVLAVGVGSALVK | |
| | | GTPVEVAEKAKAFVEKIRGCTE | |
| 1WA3-3 | | (MK)MEELFKKHKIVAVLRANSVEEAKKKALAVFLGG | |
| SEQ ID NO: 178 | | VHLIEITFTVPDADTVIKELSFLKEMGAIIGAGTVTS | |
| | | VEQARKAVESGAEFIVSPHLDEEISQFAKEKGVFYMP | |
| | | GVMTPTELVKAMKLGHTILKLFPGEVVGPQFVKAMKG | |
| | | PFPNVKFVPTGGVNLDNVCEWFKAGVLAVGVGSALVK | |
| | | GTPVEVAEKAKAFVEKIRGCTE | |
| 1WA3-4 | | (MK)MEELFKKHKIVAVLRANSVEEAKMKALAVFVGG | |
| SEQ ID NO: 179 | | VHLIEITFTVPDADTVIKELSFLKELGAIIGAGTVTS | |
| | | VEQCRKAVESGAEFIVSPHLDEEISQFCKEKGVFYMP | |
| | | GVMTPTELVKAMKLGHTILKLFPGEVVGPQFVKAMKG | |
| | | PFPNVKFVPTGGVNLDNVCEWFKAGVLAVGVGSALVK | |
| | | GTIAEVAAKAAAFVEKIRGCTE | |
| 1WA3-5 | | (MK)MEELFKKHKIVAVLRANSVEEAKKKALAVFLGG | |
| SEQ ID NO: 180 | | VDLIEITFTVPDADTVIKELSFLKEMGAIIGAGTVTS | |
| | | VEQCRKAVESGAEFIVSPHLDEEISQFCKEKGVFYMP | |
| | | GVMTPTELVKAMKLGHTILKLFPGEVVGPQFVKAMKG | |
| | | PFPNVKFVPTGGVNLDNVCEWFKAGVQAVGVGEALNK | |
| | | GTPVEVAEKAKAFVEKIRGCTE | |
| 1WA3-6 | | (MK)MEELFKKHKIVAVLRANSVEEAKKKALAVFMGG | |
| SEQ ID NO: 181 | | VDLIEITFTVPDADTVIKELSFLKELGAIIGAGTVTS | |
| | | VEQCRKAVESGAEFIVSPHLDEEISQFCKEKGVFYMP | |
| | | GVMTPTELVKAMKLGHTILKLFPGEVVGPQFVKAMKG | |
| | | PFPNVKFVPTGGVNLDNVCEWFKAGVQAVGVGEALNK | |
| | | GTPAEVAEKAKAFVEKIRGCTE | |

Table 3 provides the amino acid sequence of the first and second multimerization domains. In each case, the pairs of sequences together form an 153 multimer with icosahedral symmetry. The right-hand column in Table 3 identifies the residue numbers in each exemplary polypeptide that were identified as present at the interface of resulting assembled virus-like particles (i.e., “identified interface residues”). As can be seen, the number of interface residues for the exemplary polypeptides of SEQ ID NO: 1-34 range from 4-13. In various embodiments, the first polypeptide and second polypeptide comprise an amino acid sequence that is at least 70%, at least 80%, at least 85%, at least 90%, at least 91%, at least 92%, at least 93%, at least 94%, at least 95%, at least 96%, at least 97%, at least 98%, at least 99%, or 100% identical over its length, and identical at least at 1, 2, 3, 4, 5, 6, 7, 8, 9, 10, 11, 12, or 13 identified interface positions (depending on the number of interface residues for a given polypeptide), to the amino acid sequence of a polypeptide selected from the group consisting of SEQ ID NOS: 1-34. SEQ ID NOs: 35-51 represent other amino acid sequences of the mutlimerization domains. In other embodiments, the first polypeptide and/or second polypeptide comprise an amino acid sequence that is at least 70%, at least 80%, at least 85%, at least 90%, at least 91%, at least 92%, at least 93%, at least 94%, at least 95%, at least 96%, at least 97%, at least 98%, at least 99%, or 100% identical over its length, and at least 20%, at least 25%, at least 30%, at least 33%, at least 35%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to the identified interface positions, to the amino acid sequence of a polypeptide selected from the group consisting of SEQ ID NOs: 1-51.

As is the case with proteins in general, the polypeptides are expected to tolerate some variation in the designed sequences without disrupting subsequent assembly into virus-like particles: particularly when such variation comprises conservative amino acid substitutions. As used here, “conservative amino acid substitution” means that: hydrophobic amino acids (Ala, Cys, Gly, Pro, Met, Val, Ile, Leu) can only be substituted with other hydrophobic amino acids; hydrophobic amino acids with bulky side chains (Phe, Tyr, Trp) can only be substituted with other hydrophobic amino acids with bulky side chains; amino acids with positively charged side chains (Arg, His, Lys) can only be substituted with other amino acids with positively charged side chains; amino acids with negatively charged side chains (Asp, Glu) can only be substituted with other amino acids with negatively charged side chains; and amino acids with polar uncharged side chains (Ser, Thr, Asn, Gln) can only be substituted with other amino acids with polar uncharged side chains.

In various embodiments of the VLPs of the invention, the first polypeptide and second polypeptide, or the vice versa, comprise polypeptides with the amino acid sequence selected from the following pairs, or modified versions thereof (i.e., permissible modifications as disclosed for the polypeptides of the invention: isolated polypeptides comprising an amino acid sequence that is at least 70%, at least 80%, at least 85%, at least 90%, at least 91%, at least 92%, at least 93%, at least 94%, at least 95%, at least 96%, at least 97%, at least 98%, at least 99%, or 100% over its length, and/or identical at least at one identified interface position, to the amino acid sequence indicated by the SEQ ID NO):
SEQ ID NO: 1 and SEQ ID NO: 2 (I53-34A and I53-34B);
SEQ ID NO: 3 and SEQ ID NO: 4 (I53-40A and I53-40B);
SEQ ID NO: 3 and SEQ ID NO: 24 (I53-40A and I53-40B.1);
SEQ ID NO: 23 and SEQ ID NO: 4 (I53-40A.1 and I53-40B);
SEQ ID NO: 35 and SEQ ID NO: 36 (I53-40A genus and I53-40B genus);
SEQ ID NO: 5 and SEQ ID NO: 6 (I53-47A and I53-47B);
SEQ ID NO: 5 and SEQ ID NO: 27 (I53-47A and I53-47B.1);
SEQ ID NO: 5 and SEQ ID NO: 28 (I53-47A and I53-47B.1NegT2);
SEQ ID NO: 25 and SEQ ID NO: 6 (I53-47A.1 and I53-47B);
SEQ ID NO: 25 and SEQ ID NO: 27 (I53-47A.1 and I53-47B.1);
SEQ ID NO: 25 and SEQ ID NO: 28 (I53-47A.1 and I53-47B.1NegT2);
SEQ ID NO: 26 and SEQ ID NO: 6 (I53-47A.1NegT2 and I53-47B);
SEQ ID NO: 26 and SEQ ID NO: 27 (I53-47A.1NegT2 and I53-47B.1);
SEQ ID NO: 26 and SEQ ID NO: 28 (I53-47A.1NegT2 and I53-47B.1NegT2);
SEQ ID NO: 37 and SEQ ID NO: 38 (I53-47A genus and I53-47B genus);
SEQ ID NO: 7 and SEQ ID NO: 8 (I53-50A and I53-50B);
SEQ ID NO: 7 and SEQ ID NO: 32 (I53-50A and I53-50B.1);
SEQ ID NO: 7 and SEQ ID NO: 33 (I53-50A and I53-50B.1NegT2);
SEQ ID NO: 7 and SEQ ID NO: 34 (I53-50A and I53-50B.4PosT1);
SEQ ID NO: 29 and SEQ ID NO: 8 (I53-50A.1 and I53-50B);
SEQ ID NO: 29 and SEQ ID NO: 32 (I53-50A.1 and I53-50B.1);
SEQ ID NO: 29 and SEQ ID NO: 33 (I53-50A.1 and I53-50B.1NegT2);
SEQ ID NO: 29 and SEQ ID NO: 34 (I53-50A.1 and I53-50B.4PosT1);
SEQ ID NO: 30 and SEQ ID NO: 8 (I53-50A.1NegT2 and I53-50B);
SEQ ID NO: 30 and SEQ ID NO: 32 (I53-50A.1NegT2 and I53-50B.1);
SEQ ID NO: 30 and SEQ ID NO: 33 (I53-50A.1NegT2 and I53-50B.1NegT2);
SEQ ID NO: 30 and SEQ ID NO: 34 (I53-50A.1NegT2 and I53-50B.4PosT1);
SEQ ID NO: 31 and SEQ ID NO: 8 (I53-50A.1PosT1 and I53-50B);
SEQ ID NO: 31 and SEQ ID NO: 32 (I53-50A.1PosT1 and I53-50B.1);
SEQ ID NO: 31 and SEQ ID NO: 33 (I53-50A.1PosT1 and I53-50B.1NegT2);
SEQ ID NO: 31 and SEQ ID NO: 34 (I53-50A.1PosT1 and I53-50B.4PosT1);
SEQ ID NO: 39 and SEQ ID NO: 40 (I53-50A genus and I53-50B genus);
SEQ ID NO: 9 and SEQ ID NO: 10 (I53-51A and I53-51B);
SEQ ID NO: 11 and SEQ ID NO: 12 (I52-03A and I52-03B);
SEQ ID NO: 13 and SEQ ID NO: 14 (I52-32A and I52-32B);
SEQ ID NO: 15 and SEQ ID NO: 16 (I52-33A and I52-33B)
SEQ ID NO: 17 and SEQ ID NO: 18 (I32-06A and I32-06B);
SEQ ID NO: 19 and SEQ ID NO: 20 (I32-19A and I32-19B);
SEQ ID NO: 21 and SEQ ID NO: 22 (I32-28A and I32-28B);
SEQ ID NO: 23 and SEQ ID NO: 24 (I53-40A.1 and I53-40B.1);
SEQ ID NO: 41 and SEQ ID NO: 42 (T32-28A and T32-28B);
SEQ ID NO: 43 and SEQ ID NO: 44 (T33-09A and T33-09B);
SEQ ID NO: 45 and SEQ ID NO: 46 (T33-15A and T33-15B);
SEQ ID NO: 47 and SEQ ID NO: 48 (T33-21A and T33-21B);
SEQ ID NO: 49 and SEQ ID NO: 50 (T33-28A and T32-28B); and
SEQ ID NO: 51 and SEQ ID NO: 44 (T33-31A and T33-09B (also referred to as T33-31B))

In some embodiments, the one or more rabies G proteins ectodomains are expressed as a fusion protein with the first multimerization domain.

Non-limiting examples of designed protein complexes useful in protein-based VLPs of the present disclosure include those disclosed in U.S. Pat. No. 9,630,994; Int'l Pat. Pub No. WO2018187325A1; U.S. Pat. Pub. No. US 2018/0137234 A1; U.S. Pat. Pub. No. US 2019/0155988 A2, each of which is incorporated herein in its entirety.

In various embodiments of the VLPs of the disclosure, the first multimerization domain and second multimerization domain, or the vice versa, comprise polypeptides with the amino acid sequence selected from the following pairs, or modified versions thereof (i.e., permissible modifications as disclosed for the polypeptides of the invention: isolated polypeptides comprising an amino acid sequence that is at least 70%, at least 80%, at least 85%, at least 90%, at least 91%, at least 92%, at least 93%, at least 94%, at least 95%, at least 96%, at least 97%, at least 98%, at least 99%, or 100% over its length, and/or identical at least at one identified interface position, to the amino acid sequence indicated by the SEQ ID NO):
SEQ ID NO: 68 and SEQ ID NO: 69 (T33-dn2A and T33-dn2B);
SEQ ID NO: 70 and SEQ ID NO: 71 (T33-dn5A and T33-dn5B);
SEQ ID NO: 72 and SEQ ID NO: 73 (T33-dn10A and T33-dn10B); or
SEQ ID NO: 74 and SEQ ID NO: 75 (I53-dn5A and I53-dn5B).

In some embodiments, the polypeptide comprises a multimerization domain that shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-dn5A (SEQ ID NO: 74).

For example, the second component may comprise I53-dn5A or a functional variant thereof.

In some embodiments, the antigen comprises a polypeptide that shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-dn5B (SEQ ID NO: 75).

For example, the first component may comprise I53-dn5B or a functional variant thereof, optionally N-terminally fused to the rabies G protein ectodomain.

Various protein nanostructures are known in the art and described, for example in U.S. Pat. Pub. Nos. US2015/0356240A1; US2016/0122392A1, US20180030429A1, US20190341124A1, and US2022/0072120A1, the contents of which are incorporated by reference herein. In some embodiments, the protein nanostructure comprises, as an assembly domain, a variant of KDPG aldolase (Protein Data Bank code 1WA3) engineered to self-assemble into a protein nanostructure. In its native form, 1WA3 non-covalently assembles to form a trimer via a first interface (the trimer interface). When 20 copies of the trimer (60 monomers) are computationally docked to form a one-component icosahedral protein nanostructure, sets of five monomers of 1WA3 contact one another via a second interface (the pentamer interface). By introducing amino acid substitutions, the pentamer interface may be stabilized such that the protein nanostructure will spontaneously self-assemble, e.g., within the expressing cell or when isolated trimers (or monomers) are mixed under suitable conditions.

In some embodiments, the assembly domain comprises amino acid substitutions that remove cysteine residues. In some embodiments, the assembly domain comprises C76A and/or C100A substitutions according to SEQ ID NOs: 175-181. In embodiments, the assembly domain comprises C76A, C100A, C165A, and/or C203A substitutions according to SEQ ID NOs: 175-181. Illustrative assembly domain sequences are provided in Table 3. In each case, the N-terminal MK is optional and not included when calculating sequence identity, but is shown only for numbering purposes, i.e., MK is included in the reference sequence but not necessarily in the assembly domain of the nanostructure.

In some embodiments, the assembly domain forms a trimer with other assembly domains to form a trimeric “component” of a protein nanostructure.

In some embodiments, the assembly domain is a ferritin polypeptide. In some embodiments, the assembly domain of a ferritin protein nanostructure comprises a polypeptide sequence at least 70% at least 80%, at least 85%, at least 90%, at least 91%, at least 92%, at least 93%, at least 94%, at least 95%, at least 96%, at least 97%, at least 98%, at least 99%, or 100% identity identical to any one of the following sequences:

(SEQ ID NO: 182)
MLSKDIIKLLNEQVNKEMNSSNLYMSMSSWCYTHSLDGAGLFLFDHAAEE
YEHAKKLIIFLNENNVPVQLTSISAPEHKFEGLTQIFQKAYEHEQHISES
INNIVDHAIKSKDHATFNFLQWYVAEQHEEEVLFKDILDKVELIGNENHG
LYLADQYVKGIAKSRKS.
(SEQ ID NO: 183)
MLKPEMIEKLNEQMNLELYSSLLYQQMSAWCSYHTFEGAAAFLRRHAQEE
MTHMQRLFDYLTDTGNLPRINTVESPFAEYSSLDELFQETYKHEQLITQK
INELAHAAMTNQDYPTFNFLQWYVSEQHEEEKLFKSIIDKLSLAGKSGEG
LYFIDKELSTLDAQN.
(SEQ ID NO: 184)
NFHQDCEAGLNRTVNLKFHSSYVYLSMASYFNRDDVALSNFAKFFRERSE
EEKEHAEKLIEYQNQRGGRVFLQSVEKPERDDWANGLEALQTALKLOKSV
NQALLDLHAVAADKSDPHMTDFLESPYLSESVETIKKLGDHITSLKKLWS
SHPGMAEYLFNKHTLG.
(SEQ ID NO: 185)
QFSKDIEKLLNEQVNKEMQSSNLYMSMSSWCYTHSLDGAGLFLFDHAAEE
YEHAKKLIIFLNENNVPVQLTSISAPEHKFEGLTQIFQKAYEHEQHISES
INNIVDHAIKSKDHATFNFLQWYVAEQHEEEVLFKDILDKIELIGNENHG
LYLADQYVKGIAKSRKSGS.
(SEQ ID NO: 186)
SGESQVRQNFKPEMEEKLNEQMNLELYSSLLYQQMSAWCSYHTFEGAAAF
LRRHAQEEMTHMQRLFDYLTDTGNLPRINTVESPFAEYSSLDELFQETYK
HEQLITQKINELAHAAMTNQDYPTFNFLQWYVSEQHEEEKLFKSIIDKLS
LAGKSGEGLYFIDKELSTLDGS.

In some embodiments, the first component and second component optionally include an additional trimerization tag (e.g., a FoldOn tag or GCN4 trimers). In some embodiments, the linker sequence comprises a foldon, wherein the foldon sequence is GYIPEAPRDG QAYVRKDGEWVLLSTFL (SEQ ID NO: 58). In some embodiments, the linker may comprise a Gly-Ser linker (i.e., a linker consisting of glycine and serine residues) of any suitable length. In some embodiments, the Gly-Ser linker may be 3, 4, 5, 6, 7, 8, 9, 10, 11, 12, 13, 14, 15, 16, 17, 18, 19, 20, or more amino acids in length.

In some embodiments, the VLP comprises a trimeric assembly of antigens comprising a first polypeptide comprising a first multimerization domain and a second polypeptide comprising a second multimerization domain. The first multimerization domain comprises a protein-protein interface that induces three copies of the first polypeptides to self-associate to form trimeric building blocks. In VLPs that have two or more components, each copy of the first multimerization domain further comprises a surface-exposed interface that interacts with a complementary surface-exposed interface on the second multimerization domain. Similarly stated, the second multimerization domain is adapted to multimerize with the first multimerization domain (of the first polypeptide of the first component). As described in King et al. (Nature 510, 103-108, 2014), Bale et al. (Science 353, 389-394, 2016), and patent publications WO2014124301 A1 and US20160122392 A1, the complementary protein-protein interface between the first multimerization domain and second multimerization domain drives the assembly of multiple copies of the trimeric assembly domain and second assembly domain into a target VLP. In some embodiments, each copy of the trimeric assembly domains of the VLP bears an antigenic protein, or antigenic fragment thereof, linked thereto (e.g., as a genetic fusion); these VLPs display the proteins at full valency. In other embodiments, the VLPs of the disclosure comprise one or more copies of first multimerization domains bearing antigenic proteins, or antigenic fragments thereof (e.g., as genetic fusions) as well as one or more first multimerization domains that do not bear antigenic proteins; these VLPs display the G proteins at partial valency. The first multimerization domains can be any polypeptide sequence that forms a trimer and interacts with second multimerization domains to drive assembly to a target VLP. In some embodiments, the VLP comprises a first polypeptide and a second polypeptide selected from those disclosed in US 20130274441 A1, US 2015/0356240 A1, US 2016/0122392 A1, WO 2018/187325 A1, each of which is incorporated by reference herein in its entirety.

In some embodiments of the VLPs of the present disclosure, the antigenic protein and the core of the VLP may be genetically fused such that they are both present in a single polypeptide. The linkage between the protein and the core allows the antigenic protein, or antigenic fragment thereof, to be displayed on the exterior of the VLP. As such, the point of connection to the core should be on the exterior of the core of the formed virus-like particle. A wide variety of polypeptide sequences can be used to link the proteins, or antigenic fragments thereof and the core of the virus-like particle. In some cases the linker comprises a polypeptide sequence. Any suitable linker polypeptide can be used. In some embodiments, the linker imposes a rigid relative orientation of the antigenic protein (e.g., ectodomain) or antigenic fragment thereof to the core. In some embodiments, the linker flexibly links the antigenic protein (e.g., ectodomain) or antigenic fragment thereof to the core. In some embodiments, the linker includes additional trimerization domains (e.g., the foldon domain of T4 fibritin) to assist in stabilizing the trimeric form of the G protein e.g., GYIPEAPRDGQAYVRKDGEWVLLSTFL (SEQ ID NO: 58) or a functional variant thereof.

In some embodiments, the linker may comprise a Gly-Ser linker (i.e., a linker consisting of glycine and serine residues) of any suitable length. In some embodiments, the Gly-Ser linker may be 3, 4, 5, 6, 7, 8, 9, 10, 11, 12, 13, 14, 15, 16, 17, 18, 19, 20, or more amino acids in length. In some embodiments, the Gly-Ser linker may comprise or consist of the amino acid sequence of GSGGSGSGSGGSGSG (SEQ ID NO: 59), GGSGGSGS (SEQ ID NO: 60) or GSGGSGSG (SEQ ID NO: 61). In some embodiments, the linker comprises the sequence GSGSGSG (SEQ ID NO: 62). In some embodiments, the linker comprises the sequence GSGSGSGSGSGSGSG (SEQ ID NO: 63).

Illustrative fusion proteins with I53-50A are provided in Table 4. In each case, the signal peptide at the N terminus or the purification tag at the C terminus may be replaced with known alternatives.

TABLE 4

| | SEQ ID | |
|---|---|---|
| Description | NO: | Sequence |
| Construct A | 82 | MVPQVLLFVPLLGFSLCFGKFPIYTIPDELGPWSPIDIHHLSCPNNLVVE |
| I53-50A | | DEGCTNLSEFSYMELKVGYISAIKVNGFTCTGVVTEAETYTNFVGYVTTT |
| | | FKRKHFRPTPDACRAAYNWKMAGDPRYEESLHNPYPDYHWLRTVRTTKES |
| | | LIIISPSVTDLDPYDKSLHSRGFPGGKCSGITVSSTYCSTNHDYTIWMPE |
| | | NPGPRTPCDIFTNSRGKRASKGNKTCGFVDERGLYKSLKGACRLKLCGVL |
| | | GLRLMDGTWVAMQTSDETKWCPPDQLVNLHDFRSDEIEHLVVEELVKKRE |
| | | ECLDALESIMTTKSVSFRRLSHLRKLVPGFGKAYTIFNKTLMEADAHYKS |
| | | VRTWNEIIPSKGCLKVGGRCHPHVNGVFFNGIILGPDGHVLIPEMQSSLL |
| | | QQHMELLKSSVIPLMHPLADPSTVFKEGDEAEDFVEVHLPDVYKQISGVD |
| | | LGLPGSGSGSGEKAAKAEEAARKMEELFKKHKIVAVLRANSVEEAIEKAV |
| | | AVFAGGVHLIEITFTVPDADTVIKALSVLKEKGAIIGAGTVTSVEQARKA |
| | | VESGAEFIVSPHLDEEISQFAKEKGVFYMPGVMTPTELVKAMKLGHTILK |
| | | LFPGEVVGPQFVKAMKGPFPNVKFVPTGGVNLDNVAEWFKAGVLAVGVGS |
| | | ALVKGTPDEVREKAKAFVEKIRGATELEHHHHHH |
| Construct B | 83 | MELLILKANAITTILTAVTFCFASGKFPIYTIPDELGPWSPIDIHHLSCP |
| I53-50A | | NNLVVEDEGCTNLSEFSYMELKVGYISAIKVNGFTCTGVVTSGSGRKHFR |
| | | PTPDACRAAYNWKMAGDPRYEESLSGSGESLIIISPSVTDLDPYDKSLHS |
| | | RGFPGGKCSGITVSSTYCSTNHDYTIWMPENPGPRTPCDIFTNSRGKRAS |
| | | KGNKTCGFVDERGLYKSLKGACRLKLCGVLGLRLMDGTWVAMQTSDETKW |
| | | CPPDQLVNLHDFRSDEIEHLVVEELVKKREECLDALESIMTTKSVSFRRL |
| | | SHLRKLVPGFGKAYTIFNKTLMEADAHYKSVRTWNEIIPSKGCLKVGGRC |
| | | HPHVNGVFFNGIILGPDGHVLIPEMQSSLLQQHMELLKSSVIPLMHPLAD |
| | | PSTVFKEGDEAEDFVEVHLPDVYKQISGVDLGLPGSGSGSGEKAAKAEEA |
| | | ARKMEELFKKHKIVAVLRANSVEEAIEKAVAVFAGGVHLIEITFTVPDAD |
| | | TVIKALSVLKEKGAIIGAGTVTSVEQARKAVESGAEFIVSPHLDEEISQF |
| | | AKEKGVFYMPGVMTPTELVKAMKLGHTILKLFPGEVVGPQFVKAMKGPFP |
| | | NVKFVPTGGVNLDNVAEWFKAGVLAVGVGSALVKGTPDEVREKAKAFVEK |
| | | IRGATELEHHHHHH |
| Construct B | 84 | ATGGAGTTGCTAATCCTCAAAGCAAATGCAATTACCACAATCCTCACTGC |
| I53-50A | | AGTCACATTTTGTTTTGCCTCAGGTAAATTCCCTATCTACACCATCCCTG |
| (polynucleotide) | | ATGAGCTGGGACCTTGGAGCCCCATCGACATCCACCACCTGAGCTGCCCC |
| | | AACAACCTGGTGGTCGAGGACGAGGGCTGTACTAACCTGAGCGAGTTCAG |
| | | CTACATGGAACTGAAGGTGGGCTACATCAGTGCCATTAAGGTGAACGGCT |
| | | TCACATGCACCGGCGTTGTGACCAGCGGCAGCGGCAGAAAGCACTTCCGC |
| | | CCCACACCTGACGCCTGCAGAGCCGCCTATAACTGGAAGATGGCCGGCGA |
| | | CCCCAGATACGAGGAATCTCTGAGCGGCAGCGGCGAGAGCCTGATCATCA |
| | | TCAGCCCCAGCGTGACAGACCTGGACCCTTACGACAAGAGCCTGCACAGC |
| | | AGAGGTTTTCCAGGCGGAAAGTGCAGTGGAATTACCGTCAGCAGCACCTA |
| | | CTGCTCCACAAACCACGATTACACCATCTGGATGCCCGAGAACCCTGGCC |
| | | CGCGGACACCGTGCGACATCTTCACCAATAGCAGAGGCAAGCGGGCCAGC |
| | | AAGGGCAACAAGACCTGTGGCTTCGTGGACGAAAGAGGCCTGTACAAGTC |
| | | ACTGAAGGGTGCTTGTAGACTGAAACTGTGCGGCGTGCTGGGCCTGAGAC |
| | | TGATGGACGGCACCTGGGTCGCCATGCAAACATCTGACGAGACAAAGTGG |
| | | TGCCCTCCTGACCAGCTGGTGAACCTGCACGACTTCAGAAGCGACGAGAT |
| | | CGAACACCTGGTCGTGGAAGAGCTGGTCAAGAAAAGAGAGGAGTGCCTGG |
| | | ATGCCCTCGAGTCTATCATGACCACCAAGTCTGTGTCCTTCCGGAGACTG |
| | | TCTCACCTGCGGAAGCTGGTGCCCGGATTCGGCAAGGCCTACACCATCTT |
| | | CAACAAGACACTGATGGAAGCCGATGCTCACTACAAGAGCGTTAGAACCT |
| | | GGAACGAGATCATTCCAAGCAAGGGTTGTCTGAAGGTGGGCGGCAGATGC |
| | | CACCCTCACGTGAACGGCGTCTTTTTCAACGGCATCATCCTGGGCCCCGA |
| | | TGGCCACGTGCTGATCCCTGAGATGCAGAGCAGCCTGCTGCAGCAGCACA |
| | | TGGAACTGCTGAAGTCCAGCGTCATCCCCCTGATGCACCCCCTTGCTGAT |
| | | CCTAGCACAGTGTTCAAGGAAGGCGACGAAGCTGAAGATTTCGTTGAGGT |
| | | GCACCTGCCTGATGTGTACAAGCAGATCAGCGGAGTGGACCTGGGCCTGC |
| | | CTGGCAGCGGCTCTGGATCTGGCGAGAAAGCTGCCAAGGCCGAGGAAGCC |
| | | GCTAGAAAGATGGAAGAGCTGTTTAAGAAGCACAAGATCGTGGCCGTGCT |
| | | GCGAGCTAATTCTGTCGAGGAAGCCATCGAGAAAGCCGTTGCCGTGTTCG |
| | | CCGGAGGCGTTCATCTGATCGAGATCACCTTCACCGTGCCCGATGCCGAC |
| | | ACCGTGATCAAGGCTCTGAGCGTGCTGAAAGAAAAGGGCGCCATCATCGG |
| | | CGCCGGCACAGTCACCAGCGTGGAACAGGCCAGAAAGGCCGTGGAAAGTG |
| | | GCGCCGAGTTCATCGTGTCTCCACACCTGGACGAGGAAATCTCACAGTTT |
| | | GCCAAGGAAAAGGGGGTGTTCTACATGCCTGGCGTGATGACCCCTACAGA |
| | | GCTGGTGAAAGCTATGAAACTGGGCCACACCATCCTGAAGCTGTTCCCAG |
| | | GCGAAGTGGTGGGCCCCCAATTTGTGAAGGCTATGAAGGGCCCATTCCCT |
| | | AACGTGAAGTTCGTGCCTACCGGCGGCGTGAATCTGGACAACGTGGCCGA |
| | | GTGGTTCAAGGCCGGCGTGCTGGCCGTGGGAGTGGGCAGCGCCCTGGTGA |
| | | AGGGCACCCCCGACGAGGTGCGGGAAAAGGCCAAAGCCTTCGTGGAAAAG |
| | | ATCAGAGGCGCTACAGAGCTCGAGCACCACCACCACCATCATTGA |
| Construct C | 85 | MVPQVLLFVPLLGFSLCFGKFPIYTIPDELGPWSPIDIHHLSCPNNLVVE |
| I53-50A | | DEGCTNLSEFSYMELGSGSGSGCDIFTNSRGKRASKGNKTCGFVDERGLY |
| | | KSLKGACRLKLCGVLGLRLMDGTWVAMQTSDETKWCPPDQLVNLHDFRSD |
| | | EIEPLVVEELVKKREECLDALESIMTTKSVSFRRLSHLRKLVPGFGKAYT |
| | | IFNKTLMEADAHYKSVRTWNEIIPSKGCLKVGGRCHPHVNGVFFNGIILG |
| | | PDGHVLIPEMQSSLLQQHMELLKSSVIPLMHPGSGSGSGEKAAKAEEAAR |
| | | KMEELFKKHKIVAVLRANSVEEAIEKAVAVFAGGVHLIEITFTVPDADTV |
| | | IKALSVLKEKGAIIGAGTVTSVEQARKAVESGAEFIVSPHLDEEISQFAK |
| | | EKGVFYMPGVMTPTELVKAMKLGHTILKLFPGEVVGPQFVKAMKGPFPNV |
| | | KFVPTGGVNLDNVAEWFKAGVLAVGVGSALVKGTPDEVREKAKAFVEKIR |
| | | GATELEHHHHHH |
| Construct C | 86 | ATGGTGCCTCAGGTGCTGCTGTTCGTGCCCCTGCTGGGCTTTAGCCTCTG |
| I53-50A | | CTTCGGAAAATTCCCTATCTACACCATCCCTGATGAGCTGGGACCTTGGA |
| (polynucleotide) | | GCCCCATCGACATCCACCACCTGAGCTGCCCCAACAACCTGGTGGTCGAG |
| | | GACGAGGGCTGTACTAACCTGAGCGAGTTCAGCTACATGGAACTGGGATC |
| | | TGGCTCTGGCAGCGGATGCGACATCTTCACCAATAGCAGAGGCAAGCGGG |
| | | CCAGCAAGGGCAACAAGACCTGTGGCTTCGTGGACGAAAGAGGCCTGTAC |
| | | AAGTCACTGAAGGGTGCTTGTAGACTGAAACTGTGCGGCGTGCTGGGCCT |
| | | GAGACTGATGGACGGCACCTGGGTCGCCATGCAAACATCTGACGAGACAA |
| | | AGTGGTGCCCTCCTGACCAGCTGGTGAACCTGCACGACTTCAGAAGCGAC |
| | | GAGATCGAACCTCTGGTCGTGGAAGAGCTGGTCAAGAAAAGAGAGGAGTG |
| | | CCTGGATGCCCTCGAGTCTATCATGACCACCAAGTCTGTGTCCTTCCGGA |
| | | GACTGTCTCACCTGCGGAAGCTGGTGCCCGGATTCGGCAAGGCCTACACC |
| | | ATCTTCAACAAGACACTGATGGAAGCCGATGCTCACTACAAGAGCGTTAG |
| | | AACCTGGAACGAGATCATTCCAAGCAAGGGTTGTCTGAAGGTGGGCGGCA |
| | | GATGCCACCCTCACGTGAACGGCGTCTTTTTCAACGGCATCATCCTGGGC |
| | | CCCGATGGCCACGTGCTGATCCCTGAGATGCAGAGCAGCCTGCTGCAGCA |
| | | GCACATGGAACTGCTGAAGTCCAGCGTCATCCCCCTGATGCACCCCGGCA |
| | | GCGGCTCTGGATCTGGCGAGAAAGCTGCCAAGGCCGAGGAAGCCGCTAGA |
| | | AAGATGGAAGAGCTGTTTAAGAAGCACAAGATCGTGGCCGTGCTGCGAGC |
| | | TAATTCTGTCGAGGAAGCCATCGAGAAAGCCGTTGCCGTGTTCGCCGGAG |
| | | GCGTTCATCTGATCGAGATCACCTTCACCGTGCCCGATGCCGACACCGTG |
| | | ATCAAGGCTCTGAGCGTGCTGAAAGAAAAGGGCGCCATCATCGGCGCCGG |
| | | CACAGTCACCAGCGTGGAACAGGCCAGAAAGGCCGTGGAAAGTGGCGCCG |
| | | AGTTCATCGTGTCTCCACACCTGGACGAGGAAATCTCACAGTTTGCCAAG |
| | | GAAAAGGGGGTGTTCTACATGCCTGGCGTGATGACCCCTACAGAGCTGGT |
| | | GAAAGCTATGAAACTGGGCCACACCATCCTGAAGCTGTTCCCAGGCGAAG |
| | | TGGTGGGCCCCCAATTTGTGAAGGCTATGAAGGGCCCATTCCCTAACGTG |
| | | AAGTTCGTGCCTACCGGCGGCGTGAATCTGGACAACGTGGCCGAGTGGTT |
| | | CAAGGCCGGCGTGCTGGCCGTGGGAGTGGGCAGCGCCCTGGTGAAGGGCA |
| | | CCCCCGACGAGGTGCGGGAAAAGGCCAAAGCCTTCGTGGAAAAGATCAGA |
| | | GGCGCTACAGAGCTCGAGCACCACCACCACCATCATTGA |
| Construct D | 87 | MVPQVLLFVPLLGFSLCFGKFPIYTIPDELGPWSPIDIHHLSCPNNLVVE |
| I53-50A | | DEGCTNLSEFSYMELKVGSSIGKCDIFTNSRGKRASKGNKTCGFVDERGL |
| | | YKSLKGACRLKLCGVLGLRLMDGTWVAMQTSDETKWCPPDQLVNLHDFRS |
| | | DEIEPLVVEELVKKREECLDALESIMTTKSVSFRRLSHLRKLVPGFGKAY |
| | | TIFNKTLMEADAHYKSVRTWNEIIPSKGCLKVGGRCHPHVNGVFFNGIIL |
| | | GPDGHVLIPEMQSSLLQQHMELLKSSVIPLMHPLADPSTVFKEGDEAEDF |
| | | VEVHLPDVYKQISGVDLGLPGSGSGSGEKAAKAEEAARKMEELFKKHKIV |
| | | AVLRANSVEEAIEKAVAVFAGGVHLIEITFTVPDADTVIKALSVLKEKGA |
| | | IIGAGTVTSVEQARKAVESGAEFIVSPHLDEEISQFAKEKGVFYMPGVMT |
| | | PTELVKAMKLGHTILKLFPGEVVGPQFVKAMKGPFPNVKFVPTGGVNLDN |
| | | VAEWFKAGVLAVGVGSALVKGTPDEVREKAKAFVEKIRGATELEHHHHHH |
| Construct D | 88 | ATGGTGCCTCAGGTGCTGCTGTTCGTGCCCCTGCTGGGCTTTAGCCTCTG |
| I53-50A | | CTTCGGAAAATTCCCTATCTACACCATCCCTGATGAGCTGGGACCTTGGA |
| (polynucleotide) | | GCCCCATCGACATCCACCACCTGAGCTGCCCCAACAACCTGGTGGTCGAG |
| | | GACGAGGGCTGTACTAACCTGAGCGAGTTCAGCTACATGGAACTGAAGGT |
| | | GGGCAGCTCTATCGGCAAGTGCGACATCTTCACCAATAGCAGAGGCAAGC |
| | | GGGCCAGCAAGGGCAACAAGACCTGTGGCTTCGTGGACGAAAGAGGCCTG |
| | | TACAAGTCACTGAAGGGTGCTTGTAGACTGAAACTGTGCGGCGTGCTGGG |
| | | CCTGAGACTGATGGACGGCACCTGGGTCGCCATGCAAACATCTGACGAGA |
| | | CAAAGTGGTGCCCTCCTGACCAGCTGGTGAACCTGCACGACTTCAGAAGC |
| | | GACGAGATCGAACCTCTGGTCGTGGAAGAGCTGGTCAAGAAAAGAGAGGA |
| | | GTGCCTGGATGCCCTCGAGTCTATCATGACCACCAAGTCTGTGTCCTTCC |
| | | GGAGACTGTCTCACCTGCGGAAGCTGGTGCCCGGATTCGGCAAGGCCTAC |
| | | ACCATCTTCAACAAGACACTGATGGAAGCCGATGCTCACTACAAGAGCGT |
| | | TAGAACCTGGAACGAGATCATTCCAAGCAAGGGTTGTCTGAAGGTGGGCG |
| | | GCAGATGCCACCCTCACGTGAACGGCGTCTTTTTCAACGGCATCATCCTG |
| | | GGCCCCGATGGCCACGTGCTGATCCCTGAGATGCAGAGCAGCCTGCTGCA |
| | | GCAGCACATGGAACTGCTGAAGTCCAGCGTCATCCCCCTGATGCACCCCC |
| | | TTGCTGATCCTAGCACAGTGTTCAAGGAAGGCGACGAAGCTGAAGATTTC |
| | | GTTGAGGTGCACCTGCCTGATGTGTACAAGCAGATCAGCGGAGTGGACCT |
| | | GGGCCTGCCTGGCAGCGGCTCTGGATCTGGCGAGAAAGCTGCCAAGGCCG |
| | | AGGAAGCCGCTAGAAAGATGGAAGAGCTGTTTAAGAAGCACAAGATCGTG |
| | | GCCGTGCTGCGAGCTAATTCTGTCGAGGAAGCCATCGAGAAAGCCGTTGC |
| | | CGTGTTCGCCGGAGGCGTTCATCTGATCGAGATCACCTTCACCGTGCCCG |
| | | ATGCCGACACCGTGATCAAGGCTCTGAGCGTGCTGAAAGAAAAGGGCGCC |
| | | ATCATCGGCGCCGGCACAGTCACCAGCGTGGAACAGGCCAGAAAGGCCGT |
| | | GGAAAGTGGCGCCGAGTTCATCGTGTCTCCACACCTGGACGAGGAAATCT |
| | | CACAGTTTGCCAAGGAAAAGGGGGTGTTCTACATGCCTGGCGTGATGACC |
| | | CCTACAGAGCTGGTGAAAGCTATGAAACTGGGCCACACCATCCTGAAGCT |
| | | GTTCCCAGGCGAAGTGGTGGGCCCCCAATTTGTGAAGGCTATGAAGGGCC |
| | | CATTCCCTAACGTGAAGTTCGTGCCTACCGGCGGCGTGAATCTGGACAAC |
| | | GTGGCCGAGTGGTTCAAGGCCGGCGTGCTGGCCGTGGGAGTGGGCAGCGC |
| | | CCTGGTGAAGGGCACCCCCGACGAGGTGCGGGAAAAGGCCAAAGCCTTCG |
| | | TGGAAAAGATCAGAGGCGCTACAGAGCTCGAGCACCACCACCACCATCAT |
| | | TGA |
| Construct E | 89 | MVPQVLLFVPLLGFSLCFGKFPIYTIPDELGPWSPIDIHHLSCPNNLVVE |
| I53-50A | | DEGCTNLSEFSYMELKVGIPGSNCDIFTNSRGKRASKGNKTCGFVDERGL |
| | | YKSLKGACRLKLCGVLGLRLMDGTWVAMQTSDETKWCPPDQLVNLHDFRS |
| | | DEIEPLVVEELVKKREECLDALESIMTTKSVSFRRLSHLRKLVPGFGKAY |
| | | TIFNKTLMEADAHYKSVRTWNEIIPSKGCLKVGGRCHPHVNGVFFNGIIL |
| | | GPDGHVLIPEMQSSLLQQHMELLKSSVIPLMHPGSGSGSGEKAAKAEEAA |
| | | RKMEELFKKHKIVAVLRANSVEEAIEKAVAVFAGGVHLIEITFTVPDADT |
| | | VIKALSVLKEKGAIIGAGTVTSVEQARKAVESGAEFIVSPHLDEEISQFA |
| | | KEKGVFYMPGVMTPTELVKAMKLGHTILKLFPGEVVGPQFVKAMKGPFPN |
| | | VKFVPTGGVNLDNVAEWFKAGVLAVGVGSALVKGTPDEVREKAKAFVEKI |
| | | RGATELEHHHHHH |
| Construct E | 90 | ATGGTGCCTCAGGTGCTGCTGTTCGTGCCCCTGCTGGGCTTTAGCCTCTG |
| I53-50A | | CTTCGGAAAATTCCCTATCTACACCATCCCTGATGAGCTGGGACCTTGGA |
| (polynucleotide) | | GCCCCATCGACATCCACCACCTGAGCTGCCCCAACAACCTGGTGGTCGAG |
| | | GACGAGGGCTGTACTAACCTGAGCGAGTTCAGCTACATGGAACTGAAGGT |
| | | GGGCATCCCCGGCTCTAACTGCGACATCTTCACCAATAGCAGAGGCAAGC |
| | | GGGCCAGCAAGGGCAACAAGACCTGTGGCTTCGTGGACGAAAGAGGCCTG |
| | | TACAAGTCACTGAAGGGTGCTTGTAGACTGAAACTGTGCGGCGTGCTGGG |
| | | CCTGAGACTGATGGACGGCACCTGGGTCGCCATGCAAACATCTGACGAGA |
| | | CAAAGTGGTGCCCTCCTGACCAGCTGGTGAACCTGCACGACTTCAGAAGC |
| | | GACGAGATCGAACCTCTGGTCGTGGAAGAGCTGGTCAAGAAAAGAGAGGA |
| | | GTGCCTGGATGCCCTCGAGTCTATCATGACCACCAAGTCTGTGTCCTTCC |
| | | GGAGACTGTCTCACCTGCGGAAGCTGGTGCCCGGATTCGGCAAGGCCTAC |
| | | ACCATCTTCAACAAGACACTGATGGAAGCCGATGCTCACTACAAGAGCGT |
| | | TAGAACCTGGAACGAGATCATTCCAAGCAAGGGTTGTCTGAAGGTGGGCG |
| | | GCAGATGCCACCCTCACGTGAACGGCGTCTTTTTCAACGGCATCATCCTG |
| | | GGCCCCGATGGCCACGTGCTGATCCCTGAGATGCAGAGCAGCCTGCTGCA |
| | | GCAGCACATGGAACTGCTGAAGTCCAGCGTCATCCCCCTGATGCACCCCG |
| | | GCAGCGGCTCTGGATCTGGCGAGAAAGCTGCCAAGGCCGAGGAAGCCGCT |
| | | AGAAAGATGGAAGAGCTGTTTAAGAAGCACAAGATCGTGGCCGTGCTGCG |
| | | AGCTAATTCTGTCGAGGAAGCCATCGAGAAAGCCGTTGCCGTGTTCGCCG |
| | | GAGGCGTTCATCTGATCGAGATCACCTTCACCGTGCCCGATGCCGACACC |
| | | GTGATCAAGGCTCTGAGCGTGCTGAAAGAAAAGGGCGCCATCATCGGCGC |
| | | CGGCACAGTCACCAGCGTGGAACAGGCCAGAAAGGCCGTGGAAAGTGGCG |
| | | CCGAGTTCATCGTGTCTCCACACCTGGACGAGGAAATCTCACAGTTTGCC |
| | | AAGGAAAAGGGGGTGTTCTACATGCCTGGCGTGATGACCCCTACAGAGCT |
| | | GGTGAAAGCTATGAAACTGGGCCACACCATCCTGAAGCTGTTCCCAGGCG |
| | | AAGTGGTGGGCCCCCAATTTGTGAAGGCTATGAAGGGCCCATTCCCTAAC |
| | | GTGAAGTTCGTGCCTACCGGCGGCGTGAATCTGGACAACGTGGCCGAGTG |
| | | GTTCAAGGCCGGCGTGCTGGCCGTGGGAGTGGGCAGCGCCCTGGTGAAGG |
| | | GCACCCCCGACGAGGTGCGGGAAAAGGCCAAAGCCTTCGTGGAAAAGATC |
| | | AGAGGCGCTACAGAGCTCGAGCACCACCACCACCATCATTGA |
| Construct F | 91 | MVPQVLLFVPLLGFSLCFGKFPIYTIPDELGPWSPIDIHHLSCPNNLVVE |
| I53-50A | | DEGCTNLSEFSYMELGSGSGSGCDIFTNSRGKRASKGNKTCGFVDERGLY |
| | | KSLKGACRLKLCGVLGLRLMDGTWVAMQTSDETKWCPPDQLVNLHDFRSD |
| | | EIEHLVVEELVKKREECLDALESIMTTKSVSFRRLSHLRKLVPGFGKAYT |
| | | IFNKTLMEADAHYKSVRTWNEIIPSKGCLKVGGRCHPHVNGVFFNGIILG |
| | | PDGHVLIPEMQSSLLQQHMELLKSSVIPLMHPGSGSGSGEKAAKAEEAAR |
| | | KMEELFKKHKIVAVLRANSVEEAIEKAVAVFAGGVHLIEITFTVPDADTV |
| | | IKALSVLKEKGAIIGAGTVTSVEQARKAVESGAEFIVSPHLDEEISQFAK |
| | | EKGVFYMPGVMTPTELVKAMKLGHTILKLFPGEVVGPQFVKAMKGPFPNV |
| | | KFVPTGGVNLDNVAEWFKAGVLAVGVGSALVKGTPDEVREKAKAFVEKIR |
| | | GATELEHHHHHH |
| Construct F | 92 | ATGGTGCCTCAGGTGCTGCTGTTCGTGCCCCTGCTGGGCTTTAGCCTCTG |
| I53-50A | | CTTCGGAAAATTCCCTATCTACACCATCCCTGATGAGCTGGGACCTTGGA |
| (polynucleotide) | | GCCCCATCGACATCCACCACCTGAGCTGCCCCAACAACCTGGTGGTCGAG |
| | | GACGAGGGCTGTACTAACCTGAGCGAGTTCAGCTACATGGAACTGGGATC |
| | | TGGCTCTGGCAGCGGATGCGACATCTTCACCAATAGCAGAGGCAAGCGGG |
| | | CCAGCAAGGGCAACAAGACCTGTGGCTTCGTGGACGAAAGAGGCCTGTAC |
| | | AAGTCACTGAAGGGTGCTTGTAGACTGAAACTGTGCGGCGTGCTGGGCCT |
| | | GAGACTGATGGACGGCACCTGGGTCGCCATGCAAACATCTGACGAGACAA |
| | | AGTGGTGCCCTCCTGACCAGCTGGTGAACCTGCACGACTTCAGAAGCGAC |
| | | GAGATCGAACACCTGGTCGTGGAAGAGCTGGTCAAGAAAAGAGAGGAGTG |
| | | CCTGGATGCCCTCGAGTCTATCATGACCACCAAGTCTGTGTCCTTCCGGA |
| | | GACTGTCTCACCTGCGGAAGCTGGTGCCCGGATTCGGCAAGGCCTACACC |
| | | ATCTTCAACAAGACACTGATGGAAGCCGATGCTCACTACAAGAGCGTTAG |
| | | AACCTGGAACGAGATCATTCCAAGCAAGGGTTGTCTGAAGGTGGGCGGCA |
| | | GATGCCACCCTCACGTGAACGGCGTCTTTTTCAACGGCATCATCCTGGGC |
| | | CCCGATGGCCACGTGCTGATCCCTGAGATGCAGAGCAGCCTGCTGCAGCA |
| | | GCACATGGAACTGCTGAAGTCCAGCGTCATCCCCCTGATGCACCCCGGCA |
| | | GCGGCTCTGGATCTGGCGAGAAAGCTGCCAAGGCCGAGGAAGCCGCTAGA |
| | | AAGATGGAAGAGCTGTTTAAGAAGCACAAGATCGTGGCCGTGCTGCGAGC |
| | | TAATTCTGTCGAGGAAGCCATCGAGAAAGCCGTTGCCGTGTTCGCCGGAG |
| | | GCGTTCATCTGATCGAGATCACCTTCACCGTGCCCGATGCCGACACCGTG |
| | | ATCAAGGCTCTGAGCGTGCTGAAAGAAAAGGGCGCCATCATCGGCGCCGG |
| | | CACAGTCACCAGCGTGGAACAGGCCAGAAAGGCCGTGGAAAGTGGCGCCG |
| | | AGTTCATCGTGTCTCCACACCTGGACGAGGAAATCTCACAGTTTGCCAAG |
| | | GAAAAGGGGGTGTTCTACATGCCTGGCGTGATGACCCCTACAGAGCTGGT |
| | | GAAAGCTATGAAACTGGGCCACACCATCCTGAAGCTGTTCCCAGGCGAAG |
| | | TGGTGGGCCCCCAATTTGTGAAGGCTATGAAGGGCCCATTCCCTAACGTG |
| | | AAGTTCGTGCCTACCGGCGGCGTGAATCTGGACAACGTGGCCGAGTGGTT |
| | | CAAGGCCGGCGTGCTGGCCGTGGGAGTGGGCAGCGCCCTGGTGAAGGGCA |
| | | CCCCCGACGAGGTGCGGGAAAAGGCCAAAGCCTTCGTGGAAAAGATCAGA |
| | | GGCGCTACAGAGCTCGAGCACCACCACCACCATCATTGA |
| Construct A | 93 | KFPIYTIPDELGPWSPIDIHHLSCPNNLVVEDEGCTNLSEFSYMELKVGY |
| I53-50A | | ISAIKVNGFTCTGVVTEAETYTNFVGYVTTTFKRKHFRPTPDACRAAYNW |
| without | | KMAGDPRYEESLHNPYPDYHWLRTVRTTKESLIIISPSVTDLDPYDKSLH |
| extensions | | SRGFPGGKCSGITVSSTYCSTNHDYTIWMPENPGPRTPCDIFTNSRGKRA |
| | | SKGNKTCGFVDERGLYKSLKGACRLKLCGVLGLRLMDGTWVAMQTSDETK |
| | | WCPPDQLVNLHDFRSDEIEHLVVEELVKKREECLDALESIMTTKSVSFRR |
| | | LSHLRKLVPGFGKAYTIFNKTLMEADAHYKSVRTWNEIIPSKGCLKVGGR |
| | | CHPHVNGVFFNGIILGPDGHVLIPEMQSSLLQQHMELLKSSVIPLMHPLA |
| | | DPSTVFKEGDEAEDFVEVHLPDVYKQISGVDLGLPGSGSGSGEKAAKAEE |
| | | AARKMEELFKKHKIVAVLRANSVEEAIEKAVAVFAGGVHLIEITFTVPDA |
| | | DTVIKALSVLKEKGAIIGAGTVTSVEQARKAVESGAEFIVSPHLDEEISQ |
| | | FAKEKGVFYMPGVMTPTELVKAMKLGHTILKLFPGEVVGPQFVKAMKGPF |
| | | PNVKFVPTGGVNLDNVAEWFKAGVLAVGVGSALVKGTPDEVREKAKAFVE |
| | | KIRGATE |
| Construct B | 94 | KFPIYTIPDELGPWSPIDIHHLSCPNNLVVEDEGCTNLSEFSYMELKVGY |
| I53-50A | | ISAIKVNGFTCTGVVTSGSGRKHFRPTPDACRAAYNWKMAGDPRYEESLS |
| without | | GSGESLIIISPSVTDLDPYDKSLHSRGFPGGKCSGITVSSTYCSTNHDYT |
| extensions | | IWMPENPGPRTPCDIFTNSRGKRASKGNKTCGFVDERGLYKSLKGACRLK |
| | | LCGVLGLRLMDGTWVAMQTSDETKWCPPDQLVNLHDFRSDEIEHLVVEEL |
| | | VKKREECLDALESIMTTKSVSFRRLSHLRKLVPGFGKAYTIFNKTLMEAD |
| | | AHYKSVRTWNEIIPSKGCLKVGGRCHPHVNGVFFNGIILGPDGHVLIPEM |
| | | QSSLLQQHMELLKSSVIPLMHPLADPSTVFKEGDEAEDFVEVHLPDVYKQ |
| | | ISGVDLGLPGSGSGSGEKAAKAEEAARKMEELFKKHKIVAVLRANSVEEA |
| | | IEKAVAVFAGGVHLIEITFTVPDADTVIKALSVLKEKGAIIGAGTVTSVE |
| | | QARKAVESGAEFIVSPHLDEEISQFAKEKGVFYMPGVMTPTELVKAMKLG |
| | | HTILKLFPGEVVGPQFVKAMKGPFPNVKFVPTGGVNLDNVAEWFKAGVLA |
| | | VGVGSALVKGTPDEVREKAKAFVEKIRGATE |
| Construct C | 95 | KFPIYTIPDELGPWSPIDIHHLSCPNNLVVEDEGCTNLSEFSYMELGSGS |
| I53-50A | | GSGCDIFTNSRGKRASKGNKTCGFVDERGLYKSLKGACRLKLCGVLGLRL |
| without | | MDGTWVAMQTSDETKWCPPDQLVNLHDFRSDEIEPLVVEELVKKREECLD |
| extensions | | ALESIMTTKSVSFRRLSHLRKLVPGFGKAYTIFNKTLMEADAHYKSVRTW |
| | | NEIIPSKGCLKVGGRCHPHVNGVFFNGIILGPDGHVLIPEMQSSLLQQHM |
| | | ELLKSSVIPLMHPGSGSGSGEKAAKAEEAARKMEELFKKHKIVAVLRANS |
| | | VEEAIEKAVAVFAGGVHLIEITFTVPDADTVIKALSVLKEKGAIIGAGTV |
| | | TSVEQARKAVESGAEFIVSPHLDEEISQFAKEKGVFYMPGVMTPTELVKA |
| | | MKLGHTILKLFPGEVVGPQFVKAMKGPFPNVKFVPTGGVNLDNVAEWFKA |
| | | GVLAVGVGSALVKGTPDEVREKAKAFVEKIRGATE |
| Construct F | 96 | KFPIYTIPDELGPWSPIDIHHLSCPNNLVVEDEGCTNLSEFSYMELGSGS |
| I53-50A | | GSGCDIFTNSRGKRASKGNKTCGFVDERGLYKSLKGACRLKLCGVLGLRL |
| without | | MDGTWVAMQTSDETKWCPPDQLVNLHDFRSDEIEHLVVEELVKKREECLD |
| extensions | | ALESIMTTKSVSFRRLSHLRKLVPGFGKAYTIFNKTLMEADAHYKSVRTW |
| | | NEIIPSKGCLKVGGRCHPHVNGVFFNGIILGPDGHVLIPEMQSSLLQQHM |
| | | ELLKSSVIPLMHPGSGSGSGEKAAKAEEAARKMEELFKKHKIVAVLRANS |
| | | VEEAIEKAVAVFAGGVHLIEITFTVPDADTVIKALSVLKEKGAIIGAGTV |
| | | TSVEQARKAVESGAEFIVSPHLDEEISQFAKEKGVFYMPGVMTPTELVKA |
| | | MKLGHTILKLFPGEVVGPQFVKAMKGPFPNVKFVPTGGVNLDNVAEWFKA |
| | | GVLAVGVGSALVKGTPDEVREKAKAFVEKIRGATE |

In some embodiments, the fusion protein shares at least 70%, at least 80%, at least 85%, at least 90%, at least 91%, at least 92%, at least 93%, at least 94%, at least 95%, at least 96%, at least 97%, at least 98%, at least 99%, or 100% identity to a polypeptide sequence selected from SEQ ID NOs: 82, 83, 85, 87, 89, 91, 93-96.

Illustrative fusion proteins with 153-dn5B are provided in Table 5. In each case, the signal peptide at the N terminus or the purification tag at the C terminus may be replaced with known alternatives.

TABLE 5

| | SEQ | |
|---|---|---|
| | ID | |
| Description | NO: | Sequence |
| Construct C | 97 | MVPQVLLFVPLLGFSLCFGKFPIYTIPDELGPWSPIDIHHLSCPNNLVVEDE |
| dn5B | | GCTNLSEFSYMELKVGYISAIKVNGFTCTGVVTSGSGRKHFRPTPDACRAAY |
| | | NWKMAGDPRYEESLSGSGESLIIISPSVTDLDPYDKSLHSRGFPGGKCSGIT |
| | | VSSTYCSTNHDYTIWMPENPGPRTPCDIFTNSRGKRASKGNKTCGFVDERGL |
| | | YKSLKGACRLKLCGVLGLRLMDGTWVAMQTSDETKWCPPDQLVNLHDFRSDE |
| | | IEPLVVEELVKKREECLDALESIMTTKSVSFRRLSHLRKLVPGFGKAYTIFN |
| | | KTLMEADAHYKSVRTWNEIIPSKGCLKVGGRCHPHVNGVFFNGIILGPDGHV |
| | | LIPEMQSSLLQQHMELLKSSVIPLMHPLADPSTVFKEGDEAEDFVEVHLPDV |
| | | YKQISGVDLGLPSAIGGSAEEAELAYLLGELAYKLGEYRIAIRAYRIALKRD |
| | | PNNAEAWYNLGNAYYKQGRYREAIEYYQKALELDPNNAEAWYNLGNAYYERG |
| | | EYEEAIEYYRKALRLDPNNADAMQNLLNAKMREEGSHHHHHH |
| Construct C | 98 | ATGGTGCCTCAGGTGCTGCTGTTCGTGCCCCTGCTGGGCTTTAGCCTCTGCT |
| dn5B | | TCGGAAAATTCCCTATCTACACCATCCCTGATGAGCTGGGACCTTGGAGCCC |
| (polynucleotide) | | CATCGACATCCACCACCTGAGCTGCCCCAACAACCTGGTGGTCGAGGACGAG |
| | | GGCTGTACTAACCTGAGCGAGTTCAGCTACATGGAACTGAAGGTGGGCTACA |
| | | TCAGTGCCATTAAGGTGAACGGCTTCACATGCACCGGCGTTGTGACCAGCGG |
| | | CAGCGGCAGAAAGCACTTCCGCCCCACACCTGACGCCTGCAGAGCCGCCTAT |
| | | AACTGGAAGATGGCCGGCGACCCCAGATACGAGGAATCTCTGAGCGGCAGCG |
| | | GCGAGAGCCTGATCATCATCAGCCCCAGCGTGACAGACCTGGACCCTTACGA |
| | | CAAGAGCCTGCACAGCAGAGGTTTTCCAGGCGGAAAGTGCAGTGGAATTACC |
| | | GTCAGCAGCACCTACTGCTCCACAAACCACGATTACACCATCTGGATGCCCG |
| | | AGAACCCTGGCCCGCGGACACCGTGCGACATCTTCACCAATAGCAGAGGCAA |
| | | GCGGGCCAGCAAGGGCAACAAGACCTGTGGCTTCGTGGACGAAAGAGGCCTG |
| | | TACAAGTCACTGAAGGGTGCTTGTAGACTGAAACTGTGCGGCGTGCTGGGCC |
| | | TGAGACTGATGGACGGCACCTGGGTCGCCATGCAAACATCTGACGAGACAAA |
| | | GTGGTGCCCTCCTGACCAGCTGGTGAACCTGCACGACTTCAGAAGCGACGAG |
| | | ATCGAACCTCTGGTCGTGGAAGAGCTGGTCAAGAAAAGAGAGGAGTGCCTGG |
| | | ATGCCCTCGAGTCTATCATGACCACCAAGTCTGTGTCCTTCCGGAGACTGTC |
| | | TCACCTGCGGAAGCTGGTGCCCGGATTCGGCAAGGCCTACACCATCTTCAAC |
| | | AAGACACTGATGGAAGCCGATGCTCACTACAAGAGCGTTAGAACCTGGAACG |
| | | AGATCATTCCAAGCAAGGGTTGTCTGAAGGTGGGCGGCAGATGCCACCCTCA |
| | | CGTGAACGGCGTCTTTTTCAACGGCATCATCCTGGGCCCCGATGGCCACGTG |
| | | CTGATCCCTGAGATGCAGAGCAGCCTGCTGCAGCAGCACATGGAACTGCTGA |
| | | AGTCCAGCGTCATCCCCCTGATGCACCCCCTTGCTGATCCTAGCACAGTGTT |
| | | CAAGGAAGGCGACGAAGCTGAAGATTTCGTTGAGGTGCACCTGCCTGATGTG |
| | | TACAAGCAGATCAGCGGAGTGGACCTGGGCCTGCCTTCTGCCATCGGAGGCT |
| | | CCGCCGAGGAGGCAGAGCTGGCCTACCTGCTGGGCGAGCTGGCCTACAAGCT |
| | | GGGCGAGTATAGGATCGCCATCCGGGCCTATAGAATCGCCCTGAAGCGCGAT |
| | | CCCAACAATGCCGAGGCCTGGTACAACCTGGGCAATGCCTACTATAAGCAGG |
| | | GCCGGTATAGAGAGGCCATCGAGTACTATCAGAAGGCCCTGGAGCTGGACCC |
| | | TAATAACGCTGAGGCCTGGTATAACCTGGGAAACGCCTACTATGAGCGGGGC |
| | | GAGTATGAGGAAGCTATCGAGTACTATAGGAAGGCCCTGCGCCTGGACCCAA |
| | | ACAATGCCGATGCCATGCAGAACCTGCTGAATGCCAAGATGAGAGAGGAGGG |
| | | CAGCCACCACCACCACCACCACTGA |
| Construct F | 99 | MVPQVLLFVPLLGFSLCFGKFPIYTIPDELGPWSPIDIHHLSCPNNLVVEDE |
| dn5B | | GCTNLSEFSYMELGSGSGSGCDIFTNSRGKRASKGNKTCGFVDERGLYKSLK |
| | | GACRLKLCGVLGLRLMDGTWVAMQTSDETKWCPPDQLVNLHDFRSDEIEPLV |
| | | VEELVKKREECLDALESIMTTKSVSFRRLSHLRKLVPGFGKAYTIFNKTLME |
| | | ADAHYKSVRTWNEIIPSKGCLKVGGRCHPHVNGVFFNGIILGPDGHVLIPEM |
| | | QSSLLQQHMELLKSSVIPLMHPSAIGGSAEEAELAYLLGELAYKLGEYRIAI |
| | | RAYRIALKRDPNNAEAWYNLGNAYYKQGRYREAIEYYQKALELDPNNAEAWY |
| | | NLGNAYYERGEYEEAIEYYRKALRLDPNNADAMONLLNAKMREEGSHHHHHH |
| Construct F | 100 | ATGGTGCCTCAGGTGCTGCTGTTCGTGCCCCTGCTGGGCTTTAGCCTCTGCT |
| dn5B | | TCGGAAAATTCCCTATCTACACCATCCCTGATGAGCTGGGACCTTGGAGCCC |
| (polynucleotide) | | CATCGACATCCACCACCTGAGCTGCCCCAACAACCTGGTGGTCGAGGACGAG |
| | | GGCTGTACTAACCTGAGCGAGTTCAGCTACATGGAACTGGGATCTGGCTCTG |
| | | GCAGCGGATGCGACATCTTCACCAATAGCAGAGGCAAGCGGGCCAGCAAGGG |
| | | CAACAAGACCTGTGGCTTCGTGGACGAAAGAGGCCTGTACAAGTCACTGAAG |
| | | GGTGCTTGTAGACTGAAACTGTGCGGCGTGCTGGGCCTGAGACTGATGGACG |
| | | GCACCTGGGTCGCCATGCAAACATCTGACGAGACAAAGTGGTGCCCTCCTGA |
| | | CCAGCTGGTGAACCTGCACGACTTCAGAAGCGACGAGATCGAACCTCTGGTC |
| | | GTGGAAGAGCTGGTCAAGAAAAGAGAGGAGTGCCTGGATGCCCTCGAGTCTA |
| | | TCATGACCACCAAGTCTGTGTCCTTCCGGAGACTGTCTCACCTGCGGAAGCT |
| | | GGTGCCCGGATTCGGCAAGGCCTACACCATCTTCAACAAGACACTGATGGAA |
| | | GCCGATGCTCACTACAAGAGCGTTAGAACCTGGAACGAGATCATTCCAAGCA |
| | | AGGGTTGTCTGAAGGTGGGCGGCAGATGCCACCCTCACGTGAACGGCGTCTT |
| | | TTTCAACGGCATCATCCTGGGCCCCGATGGCCACGTGCTGATCCCTGAGATG |
| | | CAGAGCAGCCTGCTGCAGCAGCACATGGAACTGCTGAAGTCCAGCGTCATCC |
| | | CCCTGATGCACCCCTCTGCCATCGGAGGCTCCGCCGAGGAGGCAGAGCTGGC |
| | | CTACCTGCTGGGCGAGCTGGCCTACAAGCTGGGCGAGTATAGGATCGCCATC |
| | | CGGGCCTATAGAATCGCCCTGAAGCGCGATCCCAACAATGCCGAGGCCTGGT |
| | | ACAACCTGGGCAATGCCTACTATAAGCAGGGCCGGTATAGAGAGGCCATCGA |
| | | GTACTATCAGAAGGCCCTGGAGCTGGACCCTAATAACGCTGAGGCCTGGTAT |
| | | AACCTGGGAAACGCCTACTATGAGCGGGGCGAGTATGAGGAAGCTATCGAGT |
| | | ACTATAGGAAGGCCCTGCGCCTGGACCCAAACAATGCCGATGCCATGCAGAA |
| | | CCTGCTGAATGCCAAGATGAGAGAGGAGGGCAGCCACCACCACCACCACCAC |
| | | TGA |
| Construct C | 101 | KFPIYTIPDELGPWSPIDIHHLSCPNNLVVEDEGCTNLSEFSYMELKVGYIS |
| dn5B without | | AIKVNGFTCTGVVTSGSGRKHFRPTPDACRAAYNWKMAGDPRYEESLSGSGE |
| extensions | | SLIIISPSVTDLDPYDKSLHSRGFPGGKCSGITVSSTYCSTNHDYTIWMPEN |
| | | PGPRTPCDIFTNSRGKRASKGNKTCGFVDERGLYKSLKGACRLKLCGVLGLR |
| | | LMDGTWVAMQTSDETKWCPPDQLVNLHDFRSDEIEPLVVEELVKKREECLDA |
| | | LESIMTTKSVSFRRLSHLRKLVPGFGKAYTIFNKTLMEADAHYKSVRTWNEI |
| | | IPSKGCLKVGGRCHPHVNGVFFNGIILGPDGHVLIPEMQSSLLQQHMELLKS |
| | | SVIPLMHPLADPSTVFKEGDEAEDFVEVHLPDVYKQISGVDLGLPSAIGGSA |
| | | EEAELAYLLGELAYKLGEYRIAIRAYRIALKRDPNNAEAWYNLGNAYYKQGR |
| | | YREAIEYYQKALELDPNNAEAWYNLGNAYYERGEYEEAIEYYRKALRLDPNN |
| | | ADAMQNLLNAKMREE |
| Construct F | 102 | KFPIYTIPDELGPWSPIDIHHLSCPNNLVVEDEGCTNLSEFSYMELGSGSGS |
| dn5B | | GCDIFTNSRGKRASKGNKTCGFVDERGLYKSLKGACRLKLCGVLGLRLMDGT |
| without | | WVAMQTSDETKWCPPDQLVNLHDFRSDEIEPLVVEELVKKREECLDALESIM |
| extensions | | TTKSVSFRRLSHLRKLVPGFGKAYTIFNKTLMEADAHYKSVRTWNEIIPSKG |
| | | CLKVGGRCHPHVNGVFFNGIILGPDGHVLIPEMQSSLLQQHMELLKSSVIPL |
| | | MHPSAIGGSAEEAELAYLLGELAYKLGEYRIAIRAYRIALKRDPNNAEAWYN |
| | | LGNAYYKQGRYREAIEYYQKALELDPNNAEAWYNLGNAYYERGEYEEAIEYY |
| | | RKALRLDPNNADAMQNLLNAKMREE |

In some embodiments, the fusion protein shares at least 70%, at least 80%, at least 85%, at least 90%, at least 91%, at least 92%, at least 93%, at least 94%, at least 95%, at least 96%, at least 97%, at least 98%, at least 99%, or 100% identity to a polypeptide sequence selected from SEQ ID NOs: 97, 99, 101, 102.

An illustrative fusion protein with ferritin is provided in Table 6. The signal peptide at the N terminus or the purification tag at the C terminus may be replaced with known alternatives.

TABLE 6

| | SEQ | |
|---|---|---|
| Descrip- | ID | |
| tion | NO: | Sequence |
| Construct | 103 | MVPQVLLFVPLLGFSLCFGKFPIYTIPDELGPWSPI |
| F | | DIHHLSCPNNLVVEDEGCTNLSEFSYMELGSGSGSG |
| ferritin | | CDIFTNSRGKRASKGNKTCGFVDERGLYKSLKGACR |
| | | LKLCGVLGLRLMDGTWVAMQTSDETKWCPPDQLVNL |
| | | HDFRSDEIEHLVVEELVKKREECLDALESIMTTKSV |
| | | SFRRLSHLRKLVPGFGKAYTIFNKTLMEADAHYKSV |
| | | RTWNEIIPSKGCLKVGGRCHPHVNGVFFNGIILGPD |
| | | GHVLIPEMQSSLLQQHMELLKSSVIPLMHPGGSGGS |
| | | GSGGSGGSGSDIIKLLNEQVNKEMQSSNLYMSMSSW |
| | | CYTHSLDGAGLFLFDHAAEEYEHAKKLIIFLNENNV |
| | | PVQLTSISAPEHKFEGLTQIFQKAYEHEQHISESIN |
| | | NIVDHAIKSKDHATFNFLQWYVAEQHEEEVLFKDIL |
| | | DKIELIGNENHGLYLADQYVKGIAKSRKS |

In some embodiments, the fusion protein shares at least 70%, at least 80%, at least 85%, at least 90%, at least 91%, at least 92%, at least 93%, at least 94%, at least 95%, at least 96%, at least 97%, at least 98%, at least 99%, or 100% identity to a polypeptide sequence selected from SEQ ID NO: 103.

### Assembly of VLPs

In some embodiments, a single component self-assembles into the VLP. In some embodiments, one or more purified samples of first and second components for use in forming a VLP are mixed in an approximately equimolar molar ratio in aqueous conditions (e.g., an I53-50A/B isosahedral VLP). The first and second components (through the multimerization domains and optionally through the ectodomains) interact with one another to drive assembly of the target VLP. Successful assembly of the target VLP can be confirmed by analyzing the in vitro assembly reaction by common biochemical or biophysical methods used to assess the physical size of proteins or protein assemblies, including but not limited to size exclusion chromatography, native (non-denaturing) gel electrophoresis, dynamic light scattering, multi-angle light scattering, analytical ultracentrifugation, negative stain electron microscopy, cryo-electron microscopy, or X-ray crystallography. If necessary, the assembled VLP can be purified from other species or molecules present in the in vitro assembly reaction using preparative techniques commonly used to isolate proteins by their physical size, including but not limited to size exclusion chromatography, preparative ultracentrifugation, tangential flow filtration, or preparative gel electrophoresis. The presence of the antigenic protein in the VLP can be assessed by techniques commonly used to determine the identity of protein molecules in aqueous solutions, including but not limited to SDS-PAGE, mass spectrometry, protein sequencing, ELISA, surface plasmon resonance, biolayer interferometry, or amino acid analysis. The accessibility of the protein on the exterior of the particle, as well as its conformation or antigenicity, can be assessed by techniques commonly used to detect the presence and conformation of an antigen, including but not limited to binding by monoclonal antibodies, conformation-specific monoclonal antibodies, surface plasmon resonance, biolayer interferometry, or antisera specific to the antigen.

In various embodiments, the VLPs of the disclosure comprise two or more distinct first polypeptides bearing different antigenic proteins as genetic fusions; these VLPs co-display multiple different proteins on the same VLP. These multi-antigen VLPs are produced by performing in vitro assembly with mixtures of two or more antigens each comprising a multimerization domain. The fraction of each antigen in the mixture determines the average valency of each antigenic protein in the resulting VLPs. The presence and average valency of each antigen in a given sample can be assessed by quantitative analysis using the techniques described above for evaluating the presence of antigenic proteins in full-valency VLPs.

In various embodiments, the VLPs are between about 20 nanometers (nm) to about 40 nm in diameter, with interior lumens between about 15 nm to about 32 nm across and pore sizes in the protein shells between about 1 nm to about 14 nm in their longest dimensions.

In some embodiments, the VLP has icosahedral symmetry. In such embodiment, the VLP may comprise 60 copies of a first component and 60 copies of a second component. In one such embodiment, the number of identical first polypeptides in each first assembly is different than the number of identical first polypeptides in each second assembly. For example, in some embodiments, the VLP comprises twelve first assemblies and twenty second assemblies; in such embodiments, each first assembly may, for example, comprise five copies of the identical first component, and each second assembly may, for example, comprise three copies of the identical second component. In other embodiments, the VLP comprises twelve first assemblies and thirty second assemblies; in such an embodiment, each first assembly may, for example, comprise five copies of the identical first component, and each second assembly may, for example, comprise two copies of the identical second component. In further embodiments, the VLP comprises twenty first assemblies and thirty second assemblies; in this embodiment, each first assembly may, for example, comprise three copies of the identical first component, and each second assembly may, for example, comprise two copies of the identical second component. All of these embodiments are capable of forming protein-based VLPs with regular icosahedral symmetry.

In various further embodiments, oligomeric states of the first and second multimerization domains are as follows:
I53-34A: trimer+I53-34B: pentamer;
I53-40A: pentamer+I53-40B: trimer;
I53-47A: trimer+I53-47B: pentamer;
I53-50A: trimer+I53-50B: pentamer;
I53-51A: trimer+I53-51B: pentamer;
I32-06A: dimer+I32-06B: trimer;
I32-19A: trimer+I32-19B: dimer;
I32-28A: trimer+I32-28B: dimer;
I52-03A: pentamer+I52-03B: dimer;
I52-32A: dimer+I52-32B: pentamer; and
I52-33A: pentamer+I52-33B: dimer.

In some embodiments, the first multimerization domain (of the first polypeptide) comprises a sequence that shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50A or a variant thereof:

### I53-50A

(SEQ ID NO: 7)

| | |
|---|---|
| 1 | MEELFKKHKI VAVLRANSVE EAIEKAVAVF AGGVHLIEIT |
| 41 | FTVPDADTVI KALSVLKEKG AIIGAGTVTS VEQCRKAVES |
| 81 | GAEFIVSPHL DEEISQFCKE KGVFYMPGVM TPTELVKAMK |
| 121 | LGHTILKLFP GEVVGPQFVK AMKGPFPNVK FVPTGGVNLD |
| 161 | NVCEWFKAGV LAVGVGSALV KGTPDEVREK AKAFVEKIRG |
| 201 | CTE |

In some embodiments, the second multimerization domain (of the second polypeptide) shares at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 7, or an antigenic fragment thereof.

The I53-50A protein sequence has two intra-monomer disulfide bonds. In some embodiments, the cysteine residues are mutated to residues that do not contain a thiol group (e.g., alanine or serine). Removal of the thiol group may promote correct protein folding while not impairing multimerization. In some embodiments, the multimerization domain of the first polypeptide comprises an amino acid substitution at one or more of positions 74, 97, 163, and 201 relative to SEQ ID NO: 7, as shown here:

### I53-50A

(SEQ ID NO: 7)

| | |
|---|---|
| 1 | MEELFKKHKI VAVLRANSVE EAIEKAVAVF AGGVHLIEIT |
| 41 | FTVPDADTVI KALSVLKEKG AIIGAGTVTS VEQCRKAVES |
| 81 | GAEFIVSPHL DEEISQFCKE KGVFYMPGVM TPTELVKAMK |
| 121 | LGHTILKLFP GEVVGPQFVK AMKGPFPNVK FVPTGGVNLD |
| 161 | NVCEWFKAGV LAVGVGSALV KGTPDEVREK AKAFVEKIRG |
| 201 | CTE |

In some embodiments, the multimerization domain of the first polypeptide comprises an amino acid substitution of one or more of C74A, C97A, C163A, and C201A relative to SEQ ID NO: 86. In some embodiments, the multimerization domain of the first polypeptide comprises SEQ ID NO: 86 or a variant thereof.

### I53-50A-ΔCys (Disulfide-Free Form)

(SEQ ID NO: 67)

| | |
|---|---|
| 1 | MEELFKKHKI VAVLRANSVE EAIEKAVAVF AGGVHLIEIT |
| 41 | FTVPDADTVI KALSVLKEKG AIIGAGTVTS VEQARKAVES |
| 81 | GAEFIVSPHL DEEISQFAKE KGVFYMPGVM TPTELVKAMK |
| 121 | LGHTILKLFP GEVVGPQFVK AMKGPFPNVK FVPTGGVNLD |
| 161 | NVAEWFKAGV LAVGVGSALV KGTPDEVREK AKAFVEKIRG |
| 201 | ATE |

In some embodiments, the multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 7 or SEQ ID NO: 86, or an antigenic fragment thereof, and comprises one, two, three or four amino acid substitutions selected from C74A, C97A, C163A, and C201A. Alternatively, the substitution may be of C to A, T, S, L, I, or any amino acid other than C.

A polypeptide provided herein may comprise one or more conservative amino acid substitutions. The terminology “conservative amino acid substitution” is well known in the art and relates to substitution of a particular amino acid by one having a similar characteristic (e.g., similar charge or hydrophobicity). Conservative mutations can include, without limitation, substitution of amino acid residues with e.g., similar charge or hydrophobicity but differing in size or bulkiness (e.g., to provide a cavity-filling function). A list of non-limiting exemplary conservative amino acid substitutions is given in the table below.

| For Amino Acid | Code | Replace With |
|---|---|---|
| Alanine | A | D-ala, Gly, Aib, β-Ala, Acp, L-Cys, D-Cys |
| Arginine | R | D-Arg, Lys, D-Lys, homo-Arg, D-homo-Arg, Met, |
| | | Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, β-Ala, Acp |
| Isoleucine | I | D-Ile, Val, D-Val, AdaA, AdaG, Leu, D-Leu, Met, D-Met |
| Leucine | L | D-Leu, Val, D-Val, AdaA, AdaG, Leu, D-Leu, Met, D-Met |
| Lysine | K | D-Lys, Arg, D-Arg, homo-Arg, D-homo-Arg, Met, |
| | | D-Met, Ile, D-Ile, Orn, D-Orn |
| Methionine | M | D-Met, S-Me-Cys, Ile, D-Ile, Leu, D-Leu, Val, D-Val |
| Phenylalanine | F | D-Phe, Tyr, D-Thr, L-Dopa, His, D-His, Trp, D-Trp, Trans-3,4 or 5- |
| | | phenylproline, AdaA, AdaG, cis-3,4 or 5-phenylproline, Bpa, D-Bpa |
| Proline | P | D-Pro, L-I-thioazolidine-4-carboxylic acid, D-or-L-1-oxazolidine-4- |
| | | carboxylic acid (Kauer, U.S. Pat. No. (4,511,390) |
| Serine | S | D-Ser, Thr, D-Thr, allo-Thr, Met, D-Met, Met (O), |
| | | D-Met (O), L-Cys, D-Cys |
| Threonine | T | D-Thr, Ser, D-Ser, allo-Thr, Met, D-Met, Met (O), |
| | | D-Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, AdaA, AdaG |

Alternatively, a non-conservative amino acid substitution may be preferred. For example, eradication of a flexible portion of the native rabies G protein secondary structure can be done by adding a cysteine residue (or vice versa). “Non-conservative substitution” refers to the substitution of an amino acid in one class with an amino acid from another class; for example, substitution of an Ala with Asp, Asn, Glu, or Gln. Additional non-limiting examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue. Substitutions of D-Cys for D-Ala, D-Ser, or D-Tyr (or another residue) may be used to remove intramolecular disulfide bonds, which may, in some cases improve protein stability or expression. Substitutions to D-Cys may be used to generate disulfide bonds that stabilize a protein or lock a protein into a desired conformation.

In some embodiments, the nanoparticle comprising the polypeptide comprises a multimerization domain. In some embodiments, the nanoparticle comprising the multimerization domain is a trimerization domain. In some embodiments, the nanoparticle comprising the multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to FoldOn (SEQ ID NO: 58). In some embodiments, the nanoparticle comprising the multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to a polypeptide sequence selected from SEQ ID NOs: 1, 4, 5, 7, 9, 18, 19, 21, 24, 25, 26, 29, 30, 31, 34, 36, 37, 39, 42, 43, 44, 45, 46, 47, 48, 49, 50, and 51, wherein the interface residues identified in Table 3 are conserved. In some embodiments, the nanoparticle comprising the multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50A (SEQ ID NO: 7). In some embodiments, the nanoparticle comprising the multimerization domain is I53-50A-Δcys (SEQ ID NO: 67). In some embodiments, the nanoparticle comprising the polypeptide shares at least 70%, at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs: 93-96. In some embodiments, the nanoparticle comprising the multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-dn5B (SEQ ID NO: 75). In some embodiments, the nanoparticle comprising the polypeptide shares at least 70%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 101 or SEQ ID NO: 102. In some embodiments, the nanoparticle comprising the multimerization domain is a ferritin polypeptide capable of forming a ferritin particle. In some embodiments, the nanoparticle comprising the polypeptide shares at least 70%, at least 80%, at least 85%, at least 90%, at least 91%, at least 92%, at least 93%, at least 94%, at least 95%, at least 96%, at least 97%, at least 98%, at least 99%, or 100% identity to Construct F-ferritin (SEQ ID NO: 103). In some embodiments, the nanoparticle comprising the multimerization domain is a particle-forming domain.

In another aspect, the disclosure provides a nanoparticle, comprising a polypeptide according to any embodiment; or a nanoparticle comprising a rabies G protein ectodomain. In some embodiments, the polypeptide comprises the rabies G protein ectodomain according to any embodiment.

In some embodiments of the nanoparticle, the nanoparticle is a protein-based virus-like particle (pbVLP) or nanostructure.

In some embodiments of the nanoparticle, the nanoparticle lacks a lipid component. In some embodiments of the nanoparticle, the nanoparticle comprises a lipid component. In some embodiments of the nanoparticle, the nanoparticle comprises a second polypeptide component.

In some embodiments of the nanoparticle, the nanoparticle comprises a second polypeptide component and the first polypeptide component, and wherein the nanoparticle is a self-assembling nanoparticle comprising the first and second polypeptide components symmetrically arranged with point group symmetry.

In some embodiments of the nanoparticle, (a) the first polypeptide component comprises any polypeptide of the disclosure, and the first polypeptide component forms a first homomeric complex via the multimerization domain of the polypetide; (b) the second polypeptide component comprises a second multimerization domain, and the second polypeptide component forms a second homomeric complex via the second multimerization domain of the polypetide; (c) the first homomeric complex and the second homomeric complex assemble to form the nanoparticle with point-group symmetry; (d) the nanoparticle lacks other polypeptide components; and/or (e) the nanoparticle lacks lipid components.

In some embodiments of the nanoparticle, the nanoparticle has icosahedral symmetry.

In some embodiments of the nanoparticle, the first multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50A (SEQ ID NO: 7) or I53-50A ΔCys (SEQ ID NO: 67); and the second multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50B (SEQ ID NO: 8) or to I53-50B.4PosT1 (SEQ ID NO: 34).

In some embodiments of the nanoparticle, the first multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-dn5B (SEQ ID NO: 75); and the second multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-dn5A (SEQ ID NO: 74).

In some embodiments of the nanoparticle, the first polypeptide component shares at least 70%, at least 80%, at least 85%, at least 90%, at least 91%, at least 92%, at least 93%, at least 94%, at least 95%, at least 96%, at least 97%, at least 98%, at least 99%, or 100% identity to a polypeptide sequence selected from SEQ ID NOs: 93-96; and the second polypeptide component shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50B (SEQ ID NO: 8) or to I53-50B.4PosT1 (SEQ ID NO: 34).

In some embodiments of the nanoparticle, the first polypeptide component shares at least 70%, at least 80%, at least 85%, at least 90%, at least 91%, at least 92%, at least 93%, at least 94%, at least 95%, at least 96%, at least 97%, at least 98%, at least 99%, or 100% identity to a polypeptide sequence selected from SEQ ID NOs: 101-102; and the second polypeptide component shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-dn5A (SEQ ID NO: 74).

### Delivery Vehicle

In some embodiments, polynucleotides (e.g., mRNA) encoding protein nanostructures including a component comprising a viral protein monomer of a trimeric viral antigen are formulated in a delivery vehicle. In some embodiments, the delivery vehicle is a non-viral vector. In some embodiments, the delivery vehicle is a lipid nanoparticle (LNP). In some embodiments, the delivery vehicle is a liposome. In some embodiments, the delivery vehicle is a polymeric-non-viral vector, such as spermine, polyetyleneimine, chitosan, or polyurethane. In some embodiments, the delivery vehicle is a polymer delivery system, such as poly-amido-amine (PAA), poly-beta aminoesters (PBAEs) or polyethylenimine (PEI). In some embodiments, the delivery vehicle is a ferritin nanoparticle. In some embodiments, the delivery vehicle is an encapsulin.

In some embodiments, polynucleotides (e.g., mRNA) encoding protein nanostructures including a component comprising a viral protein monomer of a trimeric viral antigen are formulated in a nanoparticle. In some embodiments, the nanoparticle is a lipid nanoparticle (LNP). In some embodiments, the polynucleotides are formulated in a lipid-polycation complex, referred to as a cationic LNP. As a non-limiting example, the polycation may include a cationic peptide or a polypeptide such as, but not limited to, polylysine, polyornithine and/or polyarginine. In some embodiments, the polynucleotides are formulated in a LNP that includes a non-cationic lipid such as, but not limited to, cholesterol or dioleoyl phosphatidylethanolamine (DOPE).

In various embodiments, the lipid nanoparticles have a mean diameter from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In embodiments, the LNPs are substantially non-toxic. In certain embodiments, polynucleotides, when present in the LNPs, are resistant in aqueous solution to degradation with a nuclease. Lipids and LNPs comprising polynucleotide and their method of preparation are described in, e.g., U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the contents of which are incorporated by reference herein.

Further exemplary lipids and LNPs and their manufacture are known in the art—for example in U.S. Pat. Appl. Pub. No. U.S. 2012/0276209, Semple et al., 2010, Nat Biotechnol., 28(2):172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tam et al., 2013, Nanomedicine, 9(5): 665-74, each of which are incorporated by reference herein. Lipids and their manufacture can be found, for example, in U.S. Pub. No. 2015/0376115 and 2016/0376224, the contents of which are incorporated by reference herein.

### Nucleic Acids, Vectors, and Cells

In another aspect, the present disclosure provides isolated nucleic acids encoding an antigen, a first component, and/or a second component, of the present disclosure. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, secretory signals, nuclear localization signals, and plasma membrane localization signals. It will be apparent to those of skill in the art, based on the teachings herein, which nucleic acid sequences will encode the proteins of the disclosure.

In a further aspect, the present disclosure provides recombinant expression vectors comprising the isolated nucleic acid of any embodiment or combination of embodiments of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters including, but not limited to, CMV, SV40, RSV, actin, EF) or inducible (driven by any of a number of inducible promoters including, but not limited to, tetracycline, ecdysone, steroid responsive). The construction of expression vectors for use in transfecting cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J., and the Ambion 1998 Catalog (Ambion, Austin, TX)). The expression vector must be replicable in the host organisms either as an episome or by integration into host chromosomal DNA.

In another aspect, the present disclosure provides host cells that have been transfected or transduced with the recombinant expression vectors disclosed herein, wherein the host cells can be either prokaryotic or eukaryotic. The cells can be transiently or stably transfected or transduced. Such transfection or transduction of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press); Culture of Animal Cells: A Manual of Basic Technique, 2nd Ed. (R. I. Freshney, 1987, Liss, Inc., New York, NY)).

In another aspect, the disclosure provides a method of producing an antigen, component, or VLP according to the disclosure. In some embodiments, the method comprises the steps of (a) culturing a host according to this aspect of the disclosure under conditions conducive to the expression of the polypeptide, and (b) optionally, recovering the expressed polypeptide.

In some embodiments, the disclosure provides a method of manufacturing a vaccine, comprising culturing a host cell comprising a polynucleotide comprising a sequence encoding the antigen of the disclosure in a culture medium so that the host cell secretes the antigen into the culture media; optionally purifying the antigen from the culture media; mixing the antigen with a second component, wherein the second component multimerizes with the antigen to form a VLP; and optionally purifying the VLP.

In some embodiments, the disclosure provides method of manufacturing a vaccine, comprising culturing a host cell comprising one or more polynucleotides comprising sequences encoding both components of the VLP of any one of disclosure so that the host cell secretes the first component and the second component into the culture media; and optionally purifying the VLP from the culture media.

Illustrative host cells include E. coli cells, 293 and 293F cells, HEK293 cells, Sf9 cells, Chinese hamster ovary (CHO) cells and any other cell line used in the production of recombinant proteins.

In various embodiments, the first component expresses at about 0.5 mg/L, about 1.0 mg/L, about 2.5 mg/L, about 5 mg/L, about 10 mg/L, about 25 mg/L, about 50 mg/L, about 250 mg/L, about 500 mg/L, about 1000 mg/L or greater in a method of manufacturing according to the disclosure (e.g., 293F or CHO cells grown in suspension). In various embodiments, the first component expresses at least 50%, at least 60%, at least 70%, at least 80%, at least 90%, or at least 95% of the expression level of a rabies G protein (optionally the same ectodomain as in the VLP) in the same or similar expression system. In various embodiments, the first component expresses at least 105%, at least 110%, at least 115%, at least 120%, at least 125%, at least 150%, at least 175%, or at least 200% of the expression level of a rabies G protein (optionally the same ectodomain as in the VLP) in the same or similar expression system.

In some embodiments, the rabies G protein ectodomain of the first component is in the prefusion conformation, or a substantial fraction of the rabies G protein ectodomain is in the prefusion conformation. In various embodiments, at least 50%, at least 60%, at least 70%, at least 80%, at least 90%, or at least 95% of the rabies G protein ectodomain of the first component is in the prefusion conformation. In some embodiments, the rabies G protein ectodomain of the VLP is in the prefusion conformation, or a substantial fraction of the rabies G protein ectodomain is in the prefusion conformation. In various embodiments, at least 50%, at least 60%, at least 70%, at least 80%, at least 90%, or at least 95% of the rabies G protein ectodomain of the VLP is in the prefusion conformation.

In some embodiments, the fraction of rabies G protein ectodomain in the prefusion state is determined by binding to a conformation-specific antibody (e.g., D1-25 and 1112-1). In some embodiments, the fraction of rabies G protein ectodomain in the first component in the prefusion conformation is at least 105%, at least 110%, at least 115%, at least 120%, at least 125%, at least 150%, at least 175%, or at least 200% greater than the fraction in a reference component, or than the fraction in a reference protein not linked to a multimerization domain. In some embodiments, the fraction of rabies G protein ectodomain in the first component in the prefusion conformation is at least 105%, at least 110%, at least 115%, at least 120%, at least 125%, at least 150%, at least 175%, or at least 200% greater than the fraction in a VLP (e.g., micelle VLP).

In another aspect, the disclosure provides a polynucleotide, comprising a polynucleotide sequence encoding any polypeptide or nanoparticle of the disclosure.

In some embodiments, the polynucleotide encoding the fusion protein shares at least 70%, at least 80%, at least 85%, at least 90%, at least 91%, at least 92%, at least 93%, at least 94%, at least 95%, at least 96%, at least 97%, at least 98%, at least 99%, or 100% identity to a polynucleotide sequence selected from SEQ ID NOs: 84, 86, 88, 90, 92, 98, 100.

In some embodiments, the polynucleotide is from about 500 nucleotides to about 10000 nucleotides. In some embodiments, the polynucleotide is from about 1000 nucleotides to about 10000 nucleotides. In some embodiments, the polynucleotide is from about 1000 nucleotides to about 9000 nucleotides. In some embodiments, the polynucleotide is from about 1000 nucleotides to about 8000 nucleotides. In some embodiments, the polynucleotide is from about 1000 nucleotides to about 7000 nucleotides. In some embodiments, the polynucleotide is from about 1000 nucleotides to about 6000 nucleotides. In some embodiments, the polynucleotide is from about 1000 nucleotides to about 5000 nucleotides. In some embodiments, the polynucleotide is from about 1000 nucleotides to about 4000 nucleotides. In some embodiments, the polynucleotide is from about 1000 nucleotides to about 3000 nucleotides. In some embodiments, the polynucleotide is from about 1000 nucleotides to about 2000 nucleotides. In some embodiments, the polynucleotide is from about 1000 nucleotides to about 1500 nucleotides. In some embodiments, the polynucleotide is from about 1000 nucleotides to about 1400 nucleotides. In some embodiments, the polynucleotide is from about 1000 nucleotides to about 1300 nucleotides. In some embodiments, the polynucleotide is from about 1000 nucleotides to about 1200 nucleotides. In some embodiments, the polynucleotide is from about 1000 nucleotides to about 1100 nucleotides. In some embodiments, the polynucleotide is from about 1100 nucleotides to about 1900 nucleotides.

In another aspect, the disclosure provides a vector, comprising a polynucleotide comprising a polynucleotide sequence encoding any polypeptide or nanoparticle of the disclosure.

In another aspect, the disclosure provides a kit, comprising a polypeptide, nanoparticle, polynucleotide, vector, or pharmaceutical composition of the disclosure.

### Vaccines and Administration

The disclosure also provides vaccines comprising the VLPs described herein. Such compositions can be used to raise antibodies in a mammal (e.g. a human). The composition of the vaccines of the disclosure typically include a pharmaceutically acceptable carrier, and a thorough discussion of such carriers is available in Remington: The Science and Practice of Pharmacy.

The pH of the composition is usually between about 4.5 to about 11, between about 5 to about 11, between about 5.5 to about 11, between about 6 to about 11, between about 5 to about 10.5, between about 5.5 to about 10.5, between about 6 to about 10.5, between about 5 to about 10, between about 5.5 to about 10, between about 6 to about 10, between about 5 to about 9.5, between about 5.5 to about 9.5, between about 6 to about 9.5, between about 5 to about 9, between about 5.5 to about 9, between about 6 to about 9, between about 5 to about 8.5, between about 5.5 to about 8.5, between about 6 to about 8.5, between about 5 to about 8, between about 5.5 to about 8, between about 6 to about 8, about 4.5, about 5, about 5.5, about 6, about 6.5, about 7, about 7.5, about 8, about 8.5, about 9, about 9.5, about 10, about 10.5, about 11, etc. Stable pH may be maintained by the use of a buffer e.g. a Tris buffer, a citrate buffer, a phosphate buffer, or a histidine buffer. Thus, a composition will generally include a buffer.

A composition may be sterile and/or pyrogen free. Compositions may be isotonic with respect to humans.

A pharmaceutical composition may comprise an effective amount of its antigen polypetide. An “effective amount” is an amount which, when administered to a subject, is effective for eliciting an antibody response against the antigen. This amount can vary depending upon the health and physical condition of the individual to be treated, their age, the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials. The antigen content of compositions of the disclosure will generally be expressed in terms of the mass of protein per dose. A dose of 10-500 μg (e.g., 50 μg) per antigen can be useful.

In another aspect, the disclosure provides a pharmaceutical composition for use as a vaccine, comprising any polypeptide or nanoparticle of the disclosure, and optionally one or more pharmaceutically acceptable excipients. In some embodiments of the pharmaceutical composition, the pharmaceutical composition comprises at least one adjuvant. In some embodiments of the pharmaceutical composition, the pharmaceutical composition comprises an oil-in-water emulsion. In some embodiments of the pharmaceutical composition, the pharmaceutical composition comprises a squalene-based oil-in-water emulsion. In some embodiments of the pharmaceutical composition, the pharmaceutical composition comprises a toll-like receptor (TLR) immunostimulant. In some embodiments of the pharmaceutical composition, the pharmaceutical composition comprises squalene, SLA, GLA, R848, IMQ, 3M-052, CpG, saponin (QS21), or combinations thereof.

The antigen polypeptide or nanoparticle may be the sole active agent in the composition, e.g., formulated as an aqueous vaccine, or the composition may further comprise one or more other agents suitable for an intended use, including but not limited to adjuvants to stimulate the immune system generally and improve immune responses overall.

Vaccine compositions may include an immunological adjuvant. Exemplary adjuvants include, but are not limited to, the following: 1. mineral-containing compositions; 2. oil emulsions; 3. saponin formulations; 4. virosomes and virus-like particles; 5. bacterial or microbial derivatives; 6. bioadhesives and mucoadhesives; 7. liposomes; 8. polyoxyethylene ether and polyoxyethylene ester formulations; 9. polyphosphazene (pcpp); 10. muramyl peptides; 11. imidazoquinolone compounds; 12. thiosemicarbazone compounds; 13. tryptanthrin compounds; 14. human immunomodulators; 15. lipopeptides; 16. benzonaphthyridines; 17. microparticles; 18. immunostimulatory polynucleotide (such as rna or dna; e.g., cpg-containing oligonucleotides).

In some embodiments, the adjuvant is an oil-in-water emulsion, such as a squalene-based oil-in-water emlusion, or an aluminum hydroxide adjuvant. In some embodiments, the adjuvant includes Allhydrogel. For example, the composition may include an aluminum salt adjuvant or an oil in water emulsion.

For example, the composition may include an aluminum salt adjuvant, an oil-in-water emulsion (e.g., an oil-in-water emulsion comprising squalene, such as MF59, SWE, or ASO3), a TLR9 agonist (such as CpG oligodeoxynucleotides), a TLR7 agonist (such as imidazoquinoline or imiquimod), or a combination thereof. In some embodiments, the adjuvant is a combination of an aluminum salt and CPG1018. Suitable aluminum salts include hydroxides (e.g., oxyhydroxides), phosphates (e.g., hydroxyphosphates, orthophosphates), aluminum (e.g., see chapters 8 & 9 of Vaccine Design. (1995) eds. Powell & Newman. ISBN: 030644867X. Plenum), or mixtures thereof. The salts can take any suitable form (e.g., gel, crystalline, amorphous, etc.), with adsorption of antigen to the salt being an example. The concentration of Al⁺⁺⁺ in a composition for administration to a patient may be less than 5 mg/ml, e.g. <4 mg/ml, <3 mg/ml, <2 mg/ml, <1 mg/ml, etc. In some embodiments, the range of Al⁺⁺⁺ is between 0.3 and 1 mg/ml, between 0.3 and 2 mg/ml, between 0.3 and 3 mg/ml, or between 0.3 and 4 mg/ml. In some embodiments, a maximum of 0.85 mg/dose is used. Aluminum hydroxide and aluminium phosphate adjuvants are suitable for use with the invention of the disclosure. In a preferred embodiment, a pharmaceutical composition provided herein comprises aluminum hydroxide as an adjuvant. In some embodiment, a pharmaceutical composition provided herein comprises 500 μg aluminium hydroxide.

Exemplary adjuvants include, but are not limited to, 3M-052, Adju-Phos™, Alhydrogel™, Adjumer™, albumin-heparin microparticles, Algal Glucan, Algammulin, Alum, Antigen Formulation, AS-2 adjuvant, ASO1, ASO3, autologous dendritic cells, autologous PBMC, Avridine™, B7-2, BAK, BAY R1005, BECC TLR-4 agonists, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CpG, CPG-1018, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod™, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, Keyhole Limpet Hemocyanin, Lipid-based Adjuvant, Liposomes, Loxoribine, LT(R192G), LT-OA or LT Oral Adjuvant, LT-R192G, LTK63, LTK72, Matrix-M™ adjuvant, MF59®, MONTANIDE ISA 51, MONTANIDE ISA 720, MPL.™., MPL-SE, MTP-PE, MTP-PE Liposomes, Murametide, Murapalmitine, NAGO, nCT native Cholera Toxin, Non-Ionic Surfactant Vesicles, non-toxic mutant E112K of Cholera Toxin mCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rehydragel HPA, Rehydragel LV, RIBI, Ribi like adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, SWE, Tetanus toxoid (TT), Theramide™, Threonyl muramyl dipeptide (TMDP), Ty Particles, and Walter Reed Liposomes. Selection of an adjuvant depends on the subject to be treated. Preferably, a pharmaceutically acceptable adjuvant is used. In preferred embodiments, the adjuvant is an aluminium hydroxide gel (e.g., Alhydrogel™). In preferred embodiments, the adjuvant is SWE. In preferred embodiments, the adjuvant is MF59.

In some embodiments, the adjuvant is a squalene emulsion.

In some embodiments, the adjuvant is a TLR4 immunostimulant (e.g., SLA, GLA), e.g., as described in Van Hoeven at al. PLoS One. 11(2):e0149610 (2016).

In some embodiments, the adjuvant is a TLR7/8 immunostimulant (e.g., R848, IMQ, 3M-052), e.g., as described in Dowling D. ImmunoHorizons (6):185-197 (2018).

In some embodiments, the adjuvant is a TLR9 immunostimulant (CpG), e.g., as described in Bode et al. Expert Rev Vaccines. 10(4):499-511 (2011).

In some embodiments, the adjuvant is saponin (QS21), e.g., as described in Zhu et al. Nat Prod Chem Res. 3(4):e113 (2016).

In some embodiments, the vaccine comprises a combination of two or more adjuvants (e.g., squalene emulsion and alum or a TLR4 immunostimulant).

One suitable immunological adjuvant comprises a compound of Formula (I) as defined in WO2011/027222, or a pharmaceutically acceptable salt thereof, adsorbed to an aluminum salt. Many further adjuvants can be used, including any of those disclosed in Powell & Newman (1995).

Compositions may include an antimicrobial, particularly when packaged in multiple dose format. Antimicrobials such as thiomersal and 2-phenoxyethanol are commonly found in vaccines, but sometimes it may be desirable to use either a mercury-free preservative or no preservative at all.

Compositions may comprise a detergent, e.g., a polysorbate, such as polysorbate 80. Detergents are generally present at low levels e.g. <0.01%.

Compositions may include sodium salts (e.g. sodium chloride) to give tonicity. A concentration of 10±2 mg/ml NaCl is typical, e.g., about 9 mg/ml.

In some embodiments, the buffer in the vaccine composition is a Tris buffer, a histidine buffer, a phosphate buffer, a citrate buffer or an acetate buffer. The composition may also include a lyoprotectant, e.g., sucrose, sorbitol or trehalose. In certain embodiments, the composition includes a preservative, e.g., benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant, e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the VLP, in lyophilized or liquid form. Exemplary stabilizers include, but are not limited to, sucrose, sorbitol, glycine, inositol, sodium chloride, methionine, arginine, and arginine hydrochloride.

In some embodiments, the disclosure provides a pharmaceutical composition comprising one or more pharmaceutically acceptable excipients.

In some embodiments, the pharmaceutical composition is a stable emulsion.

In some embodiments, the disclosure provides a pharmaceutical composition comprising one or more adjuvants. In some embodiments, the one or more adjuvants comprises a TLR4 immunostimulant, e.g., Monophosphoryl Lipid A (MPL), Glucopyranosyl Lipid A (GLA), and/or Soluble Leishmania Antigen (SLA).

Also provided herein are unit doses of the pharmaceutical composition described in the present disclosure. In some embodiments, the unit dose comprises about 1 μg to about 5 μg, about 5 μg to about 10 μg, about 10 μg to about 15 μg, about 15 μg to about 20 μg, about 20 μg to about 30 μg, about 30 μg to about 40 μg, about 40 μg to about 50 μg, about 50 μg to about 60 μg, about 60 μg to about 70 μg, about 70 μg to about 80 μg, about 80 μg to about 90 μg, about 90 μg to about 100 μg, about 100 μg to about 110 μg, about 110 μg to about 120 μg, about 120 μg to about 130 μg, about 130 μg to about 140 μg, about 140 μg to about 150 μg, about 150 μg to about 200 μg, about 200 μg to about 250 μg, about 250 μg to about 300 μg, about 300 μg to about 350 μg, about 350 μg to about 400 μg, about 400 μg to about 450 μg, or about 450 μg to about 500 μg of protein complex. In some embodiments, the unit dose comprises about 1 μg, about 2 μg, about 5 μg, about 10 μg, about 15 μg, about 25 μg, about 50 μg, about 75 μg, about 100 μg, about 125 μg, about 150 μg, about 200 μg, about 250 μg, or about 300 μg of the protein complex. In some embodiments, the unit dose comprises, 25 μg, 75 μg, 150 μg, 250 μg, or about 300 μg of the protein complex. The abbreviation μg may be used interchangeably with the abbreviation mcg to refer to micrograms of a substance.

The pH of the formulation can also vary. In general, it is between about pH 6.2 to about pH 8.0. In some embodiments, the pH is about 6.2, about 6.4, about 6.6, about 6.8, about 7.0, about 7.2, about 7.4, about 7.6, about 7.8, or about 8.0. The pH may also be within a range of values. Thus, in some embodiments the pH is between about 6.2 and about 8.0, between about 6.2 and about 7.8, between about 6.2 and about 7.6, between about 6.2 and about 7.4, between about 6.2 and about 7.2, between about 6.2 and about 7.0, between about 6.2 and about 6.8, between about 6.2 and about 6.6, or between about 6.2 and about 6.4. In other embodiments, the pH is between about 6.4 and about 8.0, between about 6.4 and about 7.8, between about 6.4 and about 7.6, between about 6.4 and about 7.4, between about 6.4 and about 7.2, between about 6.4 and about 7.0, between about 6.4 and about 6.8, or between about 6.4 and about 6.6. In still other embodiments, the pH is between about 6.6 and about 8.0, between about 6.6 and about 7.8, between about 6.6 and about 7.6, between about 6.6 and about 7.4, between about 6.6 and about 7.2, between about 6.6 and about 7.0, or between about 6.6 and about 6.8. In yet other embodiments, it is between about 6.8 and about 8.0, between about 6.8 and about 7.8, between about 6.8 and about 7.6, between about 6.8 and about 7.4, between about 6.8 and about 7.2, or between about 6.8 and about 7.0. In still other embodiments, it is between about 7.0 and about 8.0, between about 7.0 and about 7.8, between about 7.0 and about 7.6, between about 7.0 and about 7.4, between about 7.0 and about 7.2, between about 7.2 and about 8.0, between about 7.2 and about 7.8, between about 7.2 and about 7.6, between about 7.2 and about 7.4, between about 7.4 and about 8.0, between about 7.4 and about 7.8, about 7.4 and about about 7.6, between about 7.6 and about 8.0, or between about 7.6 and about 7.8.

In some embodiments, the pharmaceutical composition can include one or more salts, such as sodium chloride, sodium phosphate, or a combination thereof. In general, each salt is present in the formulation at about 10 mM to about 200 mM. Thus, in some embodiments, any salt that is present is present at about 10 mM to about 200 mM, about 20 mM to about 200 mM, about 25 mM to about 200 mM, at about 30 mM to about 200 mM, at about 40 mM to about 200 mM, at about 50 mM to about 200 mM, at about 75 mM to about 200 mM, at about 100 mM to about 200 mM, at about 125 mM to about 200 mM, at about 150 mM to about 200 mM, or at about 175 mM to about 200 mM. In other embodiments, any salt that is present is present at about 10 mM to about 175 mM, about 20 mM to about 175 mM, about 25 mM to about 175 mM, at about 30 mM to about 175 mM, at about 40 mM to about 175 mM, at about 50 mM to about 175 mM, at about 75 mM to about 175 mM, at about 100 mM to about 175 mM, at about 125 mM to about 175 mM, or at about 150 mM to about 175 mM. In still other embodiments, any salt that is present is present at about 10 mM to about 150 mM, about 20 mM to about 150 mM, about 25 mM to about 150 mM, at about 30 mM to about 150 mM, at about 40 mM to about 150 mM, at about 50 mM to about 150 mM, at about 75 mM to about 150 mM, at about 100 mM to about 150 mM, or at about 125 mM to about 150 mM. In yet other embodiments, any salt that is present is present at about 10 mM to about 125 mM, about 20 mM to about 125 mM, about 25 mM to about 125 mM, at about 30 mM to about 125 mM, at about 40 mM to about 125 mM, at about 50 mM to about 125 mM, at about 75 mM to about 125 mM, or at about 100 mM to about 125 mM. In some embodiments, any salt that is present is present at about 10 mM to about 100 mM, about 20 mM to about 100 mM, about 25 mM to about 100 mM, at about 30 mM to about 100 mM, at about 40 mM to about 100 mM, at about 50 mM to about 100 mM, or at about 75 mM to about 100 mM. In yet other embodiments, any salt that is present is present at about 10 mM to about 75 mM, about 20 mM to about 75 mM, about 25 mM to about 75 mM, at about 30 mM to about 75 mM, at about 40 mM to about 75 mM, or at about 50 mM to about 75 mM. In still other embodiments, any salt that is present is present at about 10 mM to about 50 mM, about 20 mM to about 50 mM, about 25 mM to about 50 mM, at about 30 mM to about 50 mM, or at about 40 mM to about 50 mM. In other embodiments, any salt that is present is present at about 10 mM to about 40 mM, about 20 mM to about 40 mM, about 25 mM to about 40 mM, at about 30 mM to about 40 mM, at about 10 mM to about 30 mM, at about 20 mM to about 30, at about 25 mM to about 30 mM, at about 10 mM to about 25 mM, at about 20 mM to about 25 mM, or at about 10 mM to about 20 mM. In some embodiments, the sodium chloride is present in the formulation at about 100 mM. In some embodiments, the sodium phosphate is present in the formulation at about 25 mM.

Formulations may further comprise a solubilizing agent such as a nonionic detergent. Such detergents include, but are not limited to polysorbate 80 (Tween® 80), Triton X-100 and polysorbate 20.

In some embodiments, the vaccine is a pediatric vaccine. Vaccines that may be co-formulated with the polypeptide, the virus-like particle, the nucleic acid, the expression vector, and/or the adjuvant. Without limitation, a vaccine against Hepatitis B, a vaccine against Rotavirus, a vaccine against diphtheria, tetanus and pertussis (“DTaP”), a vaccine against polio, a vaccine against influenza, and a vaccine against measles, mumps and rubella (“MMR”).

In another aspect, the disclosure provides a method of inducing an immune response against rabies, comprising administering to a subject in need thereof an immunologically effective amount of the immunogenic composition described herein, which comprises the VLP as described herein.

In certain embodiments, the immune response comprises the production of neutralizing antibodies against an infectious agent. In certain embodiments, the neutralizing antibodies are complement-independent.

The immune response can comprise a humoral immune response, a cell-mediated immune response, or both. In some embodiments an immune response is induced against each delivered antigenic protein. A cell-mediated immune response can comprise a Helper T-cell (Th) response, a CD8+ cytotoxic T-cell (CTL) response, or both. In some embodiments the immune response comprises a humoral immune response, and the antibodies are neutralizing antibodies. Neutralizing antibodies block viral infection of cells. Viruses infect epithelial cells and also fibroblast cells. In some embodiments the immune response reduces or prevents infection of both cell types. Neutralizing antibody responses can be complement-dependent or complement-independent. In some embodiments the neutralizing antibody response is complement-independent. In some embodiments the neutralizing antibody response is cross-neutralizing, i.e., wherein an antibody generated against an administered composition neutralizes a virus of a strain other than the strain used in the composition.

A useful measure of antibody potency in the art is “50% neutralization titer.” To determine 50% neutralizing titer, serum from immunized animals is diluted to assess how dilute serum can be yet retain the ability to block entry of 50% of viruses into cells. For example, a titer of 700 means that serum retained the ability to neutralize 50% of virus after being diluted 700-fold. Thus, higher titers indicate more potent neutralizing antibody responses. In some embodiments, the 50% neutralization titer is in a range having a lower limit of about 200, about 400, about 600, about 800, about 1000, about 1500, about 2000, about 2500, about 3000, about 3500, about 4000, about 4500, about 5000, about 5500, about 6000, about 6500, or about 7000. The 50% neutralization titer can have a range having an upper limit of about 400, about 600, about 800, about 1000, about 1500, about 2000, about 2500, about 3000, about 3500, about 4000, about 4500, about 5000, about 5500, about 6000, about 6500, about 7000, about 8000, about 9000, about 10000, about 11000, about 12000, about 13000, about 14000, about 15000, about 16000, about 17000, about 18000, about 19000, about 20000, about 21000, about 22000, about 23000, about 24000, about 25000, about 26000, about 27000, about 28000, about 29000, or about 30000. For example, the 50% neutralization titer can be about 3000 to about 25000. With respect to the disclosed titers, “about” means plus or minus 10% of the recited value.

The World Health Organization (WHO) has defined correlates of protection for rabies virus using rapid fluorescent focus inhibition test (RFFIT) or the fluorescent antibody virus neutralization test (FAVN) assays. RFFIT is a serum neutralization (inhibition) test. FAVN refers to a microtiter plate (e.g., 96 well) format adaption of the RFFIT method. The assay uses a constant amount of rabies virus incubated with a serial dilution of serum and then added to infect BHK-21 cells. The serum titer is the dilution at which 100% of the virus is neutralized in 50% of the wells. The titer is expressed in IU/ml by comparing it with the neutralizing dilution of a reference standard. In a illustrative method, serum from a vaccination subject is diluted fivefold (1 part serum in 4 parts diluent) and then further serially diluted in fivefold increments. The serially diluted samples are mixed with a standardized amount of live rabies virus and incubated. Each diluted sample is then used to infect a culture of target cells. The result of this test can be expressed as a Rabies virus neutralizing antibodies (RVNA) endpoint titer (e.g., 1:50) or as a value for RVNA potency (e.g., 0.5 IU). The IU stands for international unit and is calculated from the titer by comparing it against the titer of a standard reference serum: sample titer divided by the reference serum titer, multiplied by the IU/mL value of the reference serum. The 1978 Joint WHO/IABS symposium, Dev Biol Stand. 1978; 40:1-288, defined a correlate of protection for rabies-neutralizing titer against G protein of 0.5 IU/ml or higher based on data in dogs and cats that demonstrated 100% protection to virus challenge with neutralizing titers of 0.2 IU/ml and 0.1 IU/ml, respectively. A conservative value of 0.5 IU/ml was chosen as a minimum value for an adequate immunization titer.

In some embodiments, the virus-like particles of the disclosure generate an immune response of about 0.5 IU/mL, about 1.0 IU/mL, about 1.5 IU/mL, about 2.0 IU/mL, about 10 IU/mL, about 25 IU/mL, about 50 IU/mL, about 100 IU/mL or greater. In some embodiments, the virus-like particles of the disclosure generate an immune response of about 0.5 IU/mL or greater.

Compositions of the disclosure will generally be administered directly to a subject. Direct delivery may be accomplished by parenteral injection (e.g., subcutaneously, intraperitoneally, intravenously, intramuscularly, or to the interstitial space of a tissue), orally, intranasal, or by any other suitable route. For example, intramuscular administration to, for example, the thigh or upper arm may be used. Injection may be via a needle (e.g., a hypodermic needle), but a needle-free injection may alternatively be used. A typical intramuscular dosage volume is about 0.5 ml.

Dosage can be by a single dose schedule or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, etc. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, etc.). Multiple doses may be administered at least 1 month apart (e.g., about 2 months, about 3 months, about 4 months, about 6 months, about 8 months, about 10 months, about 12 months, about 16 months, etc.). A second or subsequent does may be administered over longer intervals, e.g., about 1 year or about 2 years after the previous dose.

Immunization may comprise between one and ten, or more administrations (e.g., injections) of the composition, such as 1, 2, 3, 4, 5, 6, 7, 8, 9, 10, or more administrations. The first administration may elicit no detectable immune response as generally each subsequent administration will boost the immune response generated by prior administrations.

Where the vaccine is for prophylactic use, the human is preferably a child (e.g., a toddler or infant), a teenager, or an adult; where the vaccine is for therapeutic use, the human is preferably a teenager or an adult. A vaccine intended for children may also be administered to adults, e.g., to assess safety, dosage, immunogenicity, etc.

Vaccines of the disclosure may be prophylactic (i.e., to prevent disease) or therapeutic (i.e., to reduce or eliminate the symptoms of a disease). The term prophylactic may be considered as reducing the severity of or preventing the onset of a particular condition. For the avoidance of doubt, the term prophylactic vaccine may also refer to vaccines that ameliorate the effects of a future infection, for example by reducing the severity or duration of such an infection.

Isolated and/or purified VLPs described herein can be administered alone or as either a prime or a boost in mixed-modality regimes, such as a, RNA primer or DNA primer followed by a protein boost. Benefits of the RNA-prime/protein-boost strategy, as compared to a protein-prime/protein-boost strategy, include, for example, increased antibody titers, a more balanced IgG1:IgG2a subtype profile, induction of TH1-type CD4+ T cell-mediated immune response similar to that of viral particles, and reduced production of non-neutralizing antibodies. The RNA prime can increase the immunogenicity of compositions regardless of whether they contain or do not contain an adjuvant.

The VLPs of the disclosure may be administered before, concurrently with, or after vaccination with rabies immune globulin (RIG), e.g., for post-exposure prophylaxis (PEP).

In the RNA-prime/protein boost-strategy, the RNA and the protein are directed to the same target antigen. Examples of suitable modes of delivering RNAs include virus-like replicon particles (VRPs), alphavirus RNA, replicons encapsulated in lipid nanoparticles (LNPs) or formulated RNAs, such as replicons formulated with cationic nanoemulsions (CNEs). Suitable cationic oil-in-water nanoemulsions are disclosed in WO2012/006380 e.g. comprising an oil core (e.g., comprising squalene) and a cationic lipid (e.g. DOTAP, DMTAP, DSTAP, DC-cholesterol, etc.).

Alternatively, two doses of the VLP may be administered at a predetermined interval to achieve a prime-boost effect. The predetermined interval may be 1, 2, 3, 4, 6, 7, 10, or 14 days; or 3-5 days, 7-10 days, or 10-14 days or the like. The predetermined interval may be 1, 2, 3, 4, or 6 weeks; or 2-3 weeks, 3-4 weeks, or 5-6 weeks or the like. The predetermined interval may be 1, 2, 3, or 4 months.

In some embodiments, the RNA molecule is encapsulated in, bound to or adsorbed on a cationic lipid, a liposome, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, a cationic nanoemulsion, or combinations thereof.

The disclosure further provides combination vaccines. The vaccines of the disclosure include vaccines comprising both a rabies VLP and vaccines for one or more of: Typhoid fever, Hepatitis A, Polio, Influenza, Hepatitis B, Yellow Fever, Japanese encephalitis, Parvovirus, Distemper, Adenovirus, Parainfluenza, Influenza, Measles, Lyme disease, Coronavirus, Vesicular stomatitis virus, Herpes simplex virus, Baculovirus, Thogotovirus, and Bornaviridae.

Also provided herein are kits for administration of nucleic acid (e.g., RNA), purified proteins, and purified VLPs described herein, and instructions for use. The disclosure also provides a delivery device pre-filled with a composition or a vaccine disclosed herein.

The pharmaceutical compositions described herein can be administered in combination with one or more additional therapeutic agents. The additional therapeutic agents may include, but are not limited to, antibiotics or antibacterial agents, antiemetic agents, antifungal agents, anti-inflammatory agents, antiviral agents, immunomodulatory agents, cytokines, antidepressants, hormones, alkylating agents, antimetabolites, antitumour antibiotics, antimitotic agents, topoisomerase inhibitors, cytostatic agents, anti-invasion agents, antiangiogenic agents, inhibitors of growth factor function inhibitors of viral replication, viral enzyme inhibitors, anticancer agents, α-interferons, β-interferon, ribavirin, hormones, and other toll-like receptor modulators, immunoglobulins (Igs), and antibodies modulating Ig function (such as anti-IgE (omalizumab)).

In certain embodiments, the compositions disclosed herein may be used as a medicament, e.g., for use in inducing or enhancing an immune response in a subject in need thereof, such as a mammal.

In certain embodiments, the compositions disclosed herein may be used in the manufacture of a medicament for inducing or enhancing an immune response in a subject in need thereof, such as a mammal.

One way of checking efficacy of therapeutic treatment involves monitoring infection by an infectious agent after administration of the compositions or vaccines disclosed herein. One way of checking efficacy of prophylactic treatment involves monitoring immune responses, systemically (such as monitoring the level of IgG1 and IgG2a production) and/or mucosally (such as monitoring the level of IgA production), against the antigen. Typically, antigen-specific serum antibody responses are determined post-immunization but pre-challenge whereas antigen-specific mucosal antibody responses are determined post-immunization and post-challenge.

### Method of Use

In another aspect, the disclosure provides a method of generating an immune response in a subject to a subject infected with rabies virus, comprising administering any polypeptide, nanoparticle, pharmaceutical composition, polynucleotide, or vector of the disclosure in an amount effective to generate an immune response.

In another aspect, the disclosure provides a method of immunizing a subject against infection by rabies to a subject infected with rabies virus, comprising administering any polypeptide, nanoparticle, pharmaceutical composition, polynucleotide, or vector of the disclosure in an amount effective to generate an immune response.

In another aspect, the disclosure provides a method of providing post-exposure prophylaxis to a subject infected with rabies virus, comprising administering any polypeptide, nanoparticle, pharmaceutical composition, polynucleotide, or vector of the disclosure in an amount effective to generate an immune response.

In some embodiments of the method, the immune response comprises a humoral immune response.

In some embodiments of the method, the immune response comprises a polyclonal antibody response against a rabies G protein.

In some embodiments of the method, the immune response comprises a neutralizing antibody response to rabies virus.

In some embodiments of the method, the method generates a protective immune response to rabies virus.

In some embodiments of the method, the method generates neutralizing antibodies to rabies virus.

In some embodiments of the method, the administering step comprises intramuscular injection or subcutaneous injection.

In some embodiments of the method, the method results in the production of Rabies-specific neutralizing antibodies in the subject in need thereof.

In some embodiments of the method, the method results in an increase in Rabies-specific neutralizing antibodies in the subject in need thereof, of at least about a 2-fold, at least about a 3-fold, at least about a 4-fold, at least about a 5-fold, at least about a 10-fold, at least about a 15-fold, at least about a 20-fold, or at least about a 25-fold increase compared to Rabies-specific neutralizing antibodies in the same subject prior to the administering step.

In some embodiments of the method, the method generates a neutralizing titer of at least 0.5 IU/mL in a rapid fluorescent focus inhibition test (RFFIT) and/or a fluorescent antibody virus neutralization (FAVN) test.

In some embodiments of the method, the subject is a non-human animal.

In some embodiments of the method, the subject is a companion animal.

In some embodiments of the method, the subject is a human.

In another aspect, the disclosure provides a host cell, comprising a polynucleotide comprising a polynucleotide sequence encoding any polypeptide or nanoparticle of the disclosure.

In another aspect, the disclosure provides a method of manufacturing a vaccine, comprising: culturing the host cell of the disclosure in a culture medium so that the host cell secretes a first polypeptide component into the culture media; purifying the first polypeptide component from the culture media; mixing the first polypeptide component with a second polypeptide component, wherein the first and second polypeptide components self-assemble to form a nanoparticle; and/or purifying the nanoparticle.

### Definitions

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation. Alternatively, “about” can mean plus or minus a range of, for example, up to 20%, up to 10%, or up to 5%. When used in conjunction with a range or series of values, the term “about” applies to the endpoints of the range or each of the values enumerated in the series, unless otherwise indicated. As used in this application, the terms “about” and “approximately” are used as equivalents. The term “about” when used herein to refer to a deletion of the fusion loop domain of the rabies G protein, may mean, +/−1, 2, 3, 4, 5, 6, 7, 8, 9, 10, 15, 20, 25, or 30 residues.

The term “antigen” refers to a polypeptide or polypeptide complex including at least one component designed to elicit an immune response.

The term “polypeptide” refers to a series of amino acid residues joined by peptide bonds and optionally one or more post-translational modifications (e.g., glycosylation) and/or other modifications (including but not limited to conjugation of the polypeptide moiety used as a marker—such as a fluorescent tag—or a colavently linked adjuvant).

The term “infection” refers to both symptomatic and asymptomatic infections.

The term “ectodomain” refers to the portion of a transmembrane protein or glycoprotein that, in the native state of the protein, is on the outside of the cellular or viral membrane.

The term “variant” refers to a polypeptide having one or more insertions, deletions, or amino acid substitutions relative to a reference polypeptide, but retains one or more properties of the reference protein. Where the term “variant” is used, the variant shares at least 90%, 91%, 92%, 93%, 94%, 95%, 96%, 97%, 98%, 99%, or 100% sequence identity to the reference protein.

The term “antigenic variant” refers to a variant that has one or more epitopes in common with a reference polypeptide and/or generates the same or similar immune response when administered to a subject as a reference polypeptide.

The term “functional variant” refers to a variant that exhibits the same or similar functional effect(s) as a reference polypeptide. For example, a functional variant of a multimerization domain is able to promote multimerization to the same extent, or to a similar extent, as a reference multimerization domain and/or is able to multimerize with the same cognate multimerization domains as a reference multimerization domain.

The term “linker” refers to chemical linkage (i.e., a covalent bond or series of covalent bonds with intervening chemical moieties). A “polypeptide linker” refers to a linker consisting of a polypeptide inserted between two other polypeptide chains.

The term “domain” refers to any portion of a polypeptide that adopts a tertiary structure.

The terms “multimerization domain” and “multimerize” refer to the ability of a polypeptide, or domain of a polypeptide, to form dimers, trimers, tetramers, pentamers, or hexamers and/or to form heteromers with other multimerization domains.

The term “trimerization domain” refers to a multimerization domain that forms trimers.

The term “VLP-forming domain” refers to a multimerization domain that, alone or with other multimerization domains, forms a symmetric protein complex.

The term “fragment” refers to a polypeptide having one or more N-terminal or C-terminal truncations compared to a reference polypeptide.

The term “functional fragment” refers to a functional variant of a fragment.

The term “amino acid substitution” refers to replacing a single amino acid residue in a sequence with another amino acid residue. The standard form of abbreviations for amino acid substitution are used. For example, V94R refers to substitution of valine (V) in a reference sequence with arginine (R). The abbreviation Arg94 refers to any sequence in which the 94th residue, relative to a reference sequence, is arginine (Arg).

The terms “helix” or “helical” refer to an α-helical secondary structure in a polypeptide that is known to occur, or predicted to occur. For example, a sequence may be described as helical when computational modeling suggests the sequence is likely to adopt a helical conformation.

The term “polypeptide component” refers to a polypeptide capable of assembly into a nanoparticle under appropriate conditions.

The term “vaccine” refers to a composition capable of use in producing an immune response in a subject.

The term “pharmaceutically acceptable excipients” means excipients biologically or pharmacologically compatible with in vivo use in animals or humans, and can mean excipients approved by a regulatory agency of the Federal or a state government or listed in the U.S. Pharmacopeia or other generally recognized pharmacopeia for use in animals, and more particularly in humans.

The term “excipient” as used herein refers to an inert substance which is commonly used as a diluent, vehicle, preservative, binder or stabilizing agent for drugs which imparts a beneficial physical property to a formulation, such as increased protein stability, increased protein solubility, and decreased viscosity. Examples of excipients include, but are not limited to, proteins (for example, but not limited to, serum albumin), amino acids (for example, but not limited to, aspartic acid, glutamic acid, lysine, arginine, glycine), surfactants (for example, but not limited to, SDS, Tween 20, Tween 80, polysorbate and nonionic surfactants), saccharides (for example, but not limited to, glucose, sucrose, maltose and trehalose), polyols (for example, but not limited to, mannitol and sorbitol), fatty acids and phospholipids (for example, but not limited to, alkyl sulfonates and caprylate).

The term “adjuvant” refers to a pharmaceutically acceptable substance that enhances the immune response to an antigen when co-administered with the antigen or administered before, during, or after administration of the antigen to a subject.

The term “TLR4 immunostimulant” refers to an adjuvant that stimulates Toll-like Receptor 4 (TLR4) in the immune cells of a subject to modulate an immune response e.g., Monophosphoryl Lipid A (MPL), Glucopyranosyl Lipid A (GLA), and/or Soluble Leishmania Antigen (SLA).

The term “effective amount” refers to the amount of a composition that, when administered to a subject for treating a state, disorder or condition, is sufficient to effect such treatment or when administered to a subject for generating an immune response is sufficient to generate such an immune response. The “effective amount” will vary depending on the active ingredient, the state, disorder, or condition to be treated and its severity, and the age, weight, physical condition and responsiveness of the subject to be treated.

The term “immune response” refers to elicitation of activity of one or more immune cell types in the subject. Immune responses include, for example, T cell and B cell responses.

The term “humoral immune response” refers to an immune response that generates plasma or serum antibodies (e.g., IgG).

The term “protective immune response” refers to an immune response that prevents and/or reduces the severity of infection with a pathogen when the subject is later challenged with the pathogen, or to an immune response that generates a level of immune response that correlates with protection. For example, vaccination may generate a protective immune response if it results in production, in the plasma or serum, of the subject (e.g., human, pet, or agricultural animal), of neutralizing antibodies that protect the subject against subsequent infection and/or are present in a quantity observed to confer protection upon test subjects (e.g., Syrian Golden hamsters (SGH)).

The term “polyclonal antibody response” refers to an antibody response comprising antibodies having more than one specificities and/or variation in their antibody sequences.

The term “neutralizing” (e.g., “neutralizing antibody response”) refers to antibodies that prevent infection and/or reduce the level of infection by a pathogen. A neutralizing antibody response can be measured either in in vitro assays (e.g., infection of cells in culture by a pathogen in the presence of the antibody) or in an in vivo assay (e.g., by determining a protective dose of an antibody through administering the antibody to a subject prior to challenge with an infective dose of a pathogen).

The term “predetermined time” refers to an interval of time selected as appropriate for observing a particular effect. A predetermined time may be selected before, or during, an experiment or procedure.

The term “post-exposure prophylaxis” refers to administering an antigenic composition (e.g., a vaccine) to a subject previously exposed to and/or infected with a pathogen in order to elicit an immune response to protect against infection by the pathogen and/or decrease the severity of one or more symptoms of infection by the pathogen.

The term “administering” refers to providing a composition to a subject in a manner that permits the composition to have its intended effect. Administration for vaccination or post-exposure prophylaxis may be performed by intramuscular injection, intravenous injection, intraperitoneal injection, subcutaneous injection, or any other suitable route.

The terms “immunization” and “immunizing” refer to administering a composition to a subject in an amount sufficient to elicit, after one or more administering steps, a desired immune response (e.g., a humoral immune response). The term “immunizing” as used herein includes post-exposure prophylaxis.

The term “subject” refers to a human or non-human animal to which a composition may be administered for vaccination, treatment, or other purpose. In some embodiments, the non-human animal is a non-human primate including, but not limited to, rabbit, hamster, gerbil, pig, cow, sheep, goat, guinea pig, rat, mouse, squirrel, wolf, fox, horse, zebra, giraffe, elephant, cat, dog, llama, or ferret.

The term “manufacturing” refers to production of a recombinant polypeptide or virus-like particle at any scale, including, but not limited to, at least 25-mL, 50-mL, 1-L, 2-L, 1,000-L, 50,000-L, or greater scale.

The terms “culturing” and “culture medium” refers to standard cell culture and recombinant protein expression techniques.

The term “host cell” refers to any cell capable of use in expression of a recombinant polypeptide.

The term “secretes” refers to the ability of host cells to secrete polypeptides into the media in which they are cultured.

The term “signal sequence” refers to a polypeptide sequence, typically at the N terminus of a polypeptide expressed in a host cell that directs the polypeptide to a particular cellular compartment. A signal sequence may be a secretion signal to cause the host cell to secrete the polypeptide into the media in which with host cell is cultured. Various signal sequences are known and it is within the skill of an ordinary artisan to select an appropriate signal sequence. A signal sequence is a short peptide (typically 16-30 amino acids in length) that is cleaved either during or after completion of translocation to generate a free signal peptide and mature protein.

The term “mixing” refers to placing two solutions into contact to permit the solutions to mix.

The term “purify” refers to separating a molecule from other substances present in a composition. Polypeptides may be purified by affinity (e.g., to an antibody or to a tag, e.g., using a His-tag capture resin), by charge (e.g., ion-exchange chromatography), by size (e.g., preparative ultracentrifugation, size exclusion chromatography), or otherwise.

The terms “polynucleotide” and “nucleic acid,” used interchangeably herein, refer to a polymeric form of nucleotides of more than about 100 nucleotides, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “identical” or percent “identity,” in the context of two or more nucleic acid or polypeptide sequences, refers to two or more sequences or subsequences that are the same or have a specified percentage of amino acid residues or nucleotides that are the same, when compared and aligned for maximum correspondence. Methods of alignment of sequences for comparison are well known in the art. Once aligned, the number of matches is determined by counting the number of positions where an identical nucleotide or amino acid residue is present in both sequences. The percent sequence identity is determined by dividing the number of matches in the alignment by the length of the reference sequence, followed by multiplying the resulting value by 100. For example, a peptide sequence that has 1166 matches when aligned with a reference sequence having 1554 amino acids is 75.0 percent identical to the test sequence (1166÷1554*100=75.0). As the terms are used herein, gaps in the alignment do not decrease the percent sequence identity. Unless otherwise specified, optimal alignment of sequences for comparison is conducted by the global alignment algorithm of Needleman and Wunsch, Mol. Biol. 48:443 (1970) as implemented by EMBOSS Needle (on the World Wide Web at ebi.ac.uk/Tools/psa/emboss_needle/) (Madeira et al. Nucleic Acids Res. 50(W1):W276-W279 (2022)). Other alignment methods may be used, including without limitation those described in Devereux, et al, Nucleic Acids Res. 12:387-95 (1984); Atschul et al. J. Mo. Biol. 215:403-10 (1990) (BLAST); Carrillo and Lipman Siam J. Appl. Math. 48(5) (1988); Computational Molecular Biology (Lesk, A M, ed., 1989); Biocomputing Informatics and Genome Projects, (Smith, DW, ed., 1993); Computer Analysis of Sequence Data, Part I, (Griffin and Griffin, eds., 1994); Sequence Analysis in Molecular Biology (von Heinje, 2012); Sequence Analysis Primer (Gribskov and Devereux, J., eds. 1993). Sequence identity is calculated using the implementation of the Needleman-Wunsch algorithm provided by the National Library of Medicine (on the World Wide Web at blast.ncbi.nlm.nih.gov/Blast.cgi?PAGE_TYPE=BlastSearch&BLAST_SPEC=GlobalAln).

The term “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

As used herein, the term “companion animal” refers to any animal for which an owner, breeder or caregiver controls the feeding habits. In some embodiments, a companion animal is an animal selected from the group consisting of dog, cat, rabbit, hamster, gerbil, ferret, and guinea pig. In some embodiments, a companion animal is a dog or a cat.

The description includes information that may be useful in understanding the present invention. No statement made herein should be construed as an admission that any of the information provided herein is prior art or relevant to the presently claimed inventions, or that any publication specifically or implicitly referenced is available as prior art.

### EXAMPLES

### Example 1

Various modification of rabies G protein were designed and tested as fusion proteins to the multimerization domain I53-50A. For many, expression was low and/or the protein aggregated. A subset of tested designs is listed in Table 7.

Human codon-optimized polynucleotide sequences were made by gene synthesis and cloned into expression vectors. Each expression vector was individually expressed by transient transfection in Expi293 cells. Supernatants were collected four days after transfection.

A Western blot using an anti-His₆ monoclonal antibody was performed. Conditioned media (12 μl) from transient transfections was mixed with 4 μl reduced sample loading buffer and heated at 95° C. for 5 minutes. Samples were loaded onto a NuPAGE 4-12% Bis-Tris protein gel and run for 2 hours at 120V. Proteins were transferred onto an Immuno-Blot PDVF membrane (Bio-Rad). Following transfer, the blot was blocked with 1× Casein buffer (Bio-Rad) for 1 hour at room temperature with shaking. Membrane was then incubated with an HRP-conjugated anti-His₆ monoclonal antibody for 2 hours at room temperature with shaking. Membrane was washed 3× for 15 minutes each in PBS with 0.1% Tween 20. Next the membrane was incubated with HRP substrate (Pierce ECL plus kit) for 2 minutes and imaged.

Expression levels were assessed by visual inspection of Western blots and designated as no expression (X) or low expression (*) to high expression (****), no expression to >10 mg/L.

TABLE 7
Antigen constructs and mutations

| | | Rabies G | Modification | Transient |
|---|---|---|---|---|
| Construct | Seq | protein | Numbering by | Expression |
| No. | ID | sequence | SEQ ID NO: 53 | Level |
| Construct A | 76 | 20-454 | N/A | * |
| Construct B | 77 | 20-454 | Substitution of two fusion loops | *** |
| | | | with polypeptide linkers: Residues | |
| | | | 85-102 and 132-148 each replaced | |
| | | | with Ser-Gly-Ser-Gly linker | |
| Construct C | 78 | 20-417 | Deletion of fusion loop domain: | **** |
| | | | Residues 66-207 replaced with Gly- | |
| | | | Ser-Gly-Ser-Gly-Ser-Gly linker | |
| | | | (construct included point mutation); | |
| | | | C-terminus at residue 417 | |
| Construct D | 79 | 20-454 | Deletion of fusion loop domain: | *** |
| | | | Residues 69-207 replaced with Ser- | |
| | | | Ser-Ile-Gly-Lys linker (construct | |
| | | | included point mutation); | |
| | | | C-terminus at residue 454 | |
| Construct E | 80 | 20-417 | Deletion of fusion loop domain: | *** |
| | | | Residues 69-207 replaced with Ile- | |
| | | | Pro-Gly-Ser-Asn linker (construct | |
| | | | included point mutation); | |
| | | | C-terminus at residue 417 | |
| Construct F | 81 | 20-417 | Deletion of fusion loop domain: | **** |
| | | | Residues 66-207 replaced with Gly- | |
| | | | Ser-Gly-Ser-Gly-Ser-Gly linker; | |
| | | | C-terminus at residue 417 | |

Construct A, the wild-type ectodomain sequence for rabies G fused to I53-50A, expressed at low levels (<2 mg/L). The resulting protein was severely aggregated.

Construct B which has two deletions within the fusion loop domain has greatly improved expression and did not aggregate like the wild-type sequence.

Deletions of the complete fusion loop domain was surprisingly well-tolerated as constructs with C-terminal trunctions to residue 417 (Construct C and Construct F) or residue 454 (Construct D and Construct E) all expressed well.

Rabies G protein have been reported to have toxicity in mammalian cells. To test the ability to stably express the selected rabies G proteins, polynucleotides encoding Construct B or Construct F were transfected into CHO-K1 and stable pools selected using glutamine synthetase as a selection marker. No Construct B expressing cells survived selection on glutamine for 14 days. Three other tested constructs also did not generate stable cell lines. A stable cell line for Construct F was successfully selected using a fed-batch system at 25-mL scale, expression of CompA-Construct F was over 400 mg/L.

### Example 2

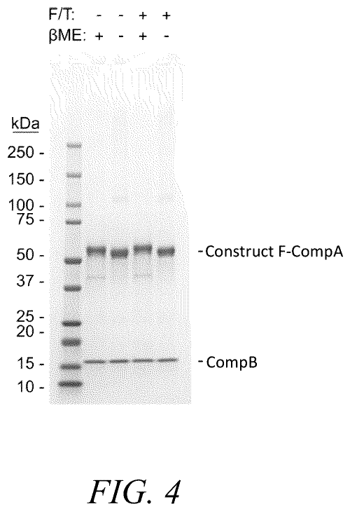

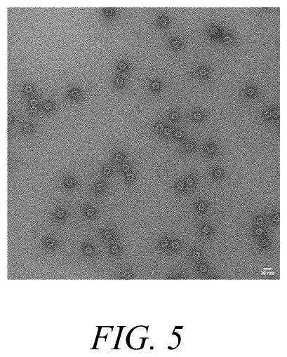

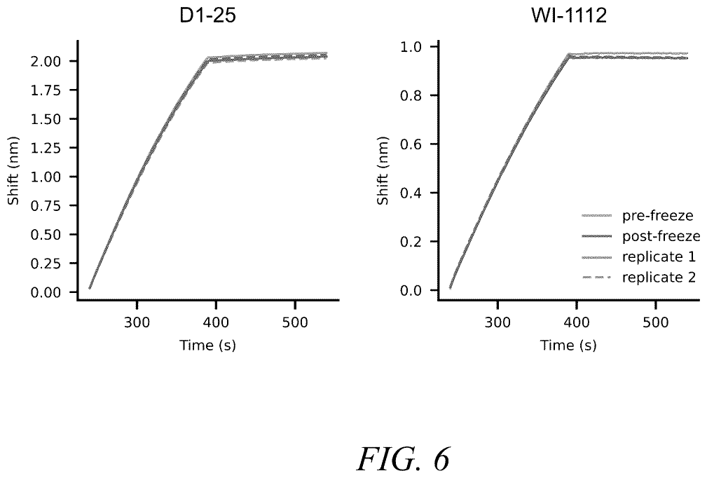

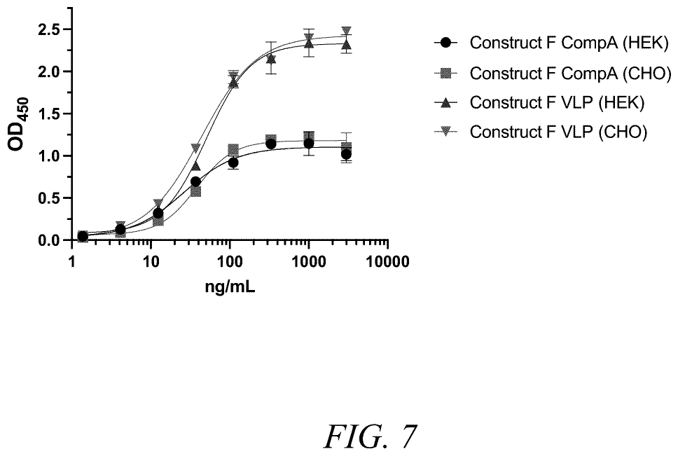

The I53-50A multimerization tag is capable of forming a trimer alone. When mixed with pentameric I53-50B, the I53-50A trimers are known to self-assemble to form an icosahedral particle. Construct B, Construct C, and Construct F (made by transient transfection) were assembled with I53-50B to generate VLPs. All assembled into monodisperse VLPs of the expected size. Representative characterization of Construct F VLPs is shown in FIG. 4 (SDS-PAGE), FIG. 5 (negative stain electron microscopy), FIG. 6 (BLI), and FIG. 7 (ELISA).

To characterize the rabies G protein on the resulting particles, binding of neutralizing antibodies D1-25 and 1112-1 (RD-Biotech) that recognize antigenic sites III and II, respectively, was tested by biolayer interferometry (BLI). Both neutralizing antibodies bind Construct F particles, whether stored at room temperature or subjected to freeze/thaw (FIG. 6).

Binding of particle (“VLP”) or soluble trimers (“CompA”) was also tested for samples made from transiently transfected Expi293 cells (“HEK”) or stable CHO-K1 cell lines (“CHO-K1”). The wells of a 96 well plate were coated with 2 μg/mL 1112-1 monoclonal antibody (100 μl) in carbonate-bicarbonate buffer, pH 9.6 at 4° C. overnight. Plates were washed 3× with 300 μl PBS with 0.05% Tween 20. After blocking with 100 μl PBS with 2% BSA for 1 hour at 37° C., the plates were washed 3× as above. Trimeric Construct F (Construct F compA) or Construct F particles (Construct F VLP) were added in a volume of 100 μl and the plates were gently shaken for 1 hour at 37° C. Plates were washed 3× with 200 μl PBS with 0.05% Tween 20. Biotinylated D1-25 (100 μl) in PBS with 0.05% Tween-20 and 0.1% BSA was added and the plates gently shaken for 1 hour at 37° C. After washing the plates 3× with 300 μl PBS with 0.05% Tween 20, 100 μl of a 1:5000 dilution of HRP-conjugated StreptAvidin was added to each well and gently shook for 1 hour at room temperature. Plates were washed 6× with 200 μl PBS with 0.05% Tween 20 and then 100 μl TMB substrate added and developed in the dark for 10 minutes. Reaction was stopped by addition of 100 μl 0.1M HCl and absorbance measured at 450 nm on a plate reader. The sandwich ELISA system would show a positive signal only if the sample binds both the 1112-1 capture antibody and the D1-25 detection antibody.

All CompA and VLP samples showed a positive signal in the sandwich ELISA (FIG. 7), indicating that the rabies G protein with deletion of the fusion loop domain is stably folded in an antigenic conformation, whether as a trimer or assembled into a particle. The source of sample (293F cells or CHO cells) did not impact binding; higher signal was observed for the particles than trimer (FIG. 7).

### Example 3

A study was undertaken in naïve BALB/c mice to explore the immunogenicity of a rabies G protein that includes a deletion of residues 66-207 displayed on a two-component virus-like particle (VLP). The experiments in Example 2 were performed using the I53-50A/I53-50B multimerization domain pair. For this Example, a different particle was used, in which the rabies G protein ectodomain was fused to the I53-dn5B multimerization domain, which forms a trimer, and then assembled with a pentamer I53-dn5A multimerization domain to form a particle.

The rabies G protein ectodomain fused to the I53-dn5B trimerization domain (SEQ ID NO: 97) (“Construct C-Component”) was expressed and purified, and then mixed with I53-dn5A to assemble a particle. The trimeric I53-dn5B protein complex was formulated with an oil-in-water adjuvant (Addavax™). The assembled I53-dn5B/dn5A particle was formulated with the oil-in-water adjuvant (Addavax™) or an aluminium hydroxide (alum) adjuvant (Alhydrogel™). The commercial adjuvanted rabies vaccine IMRAB® 3 (“ImRab3”) was used as a control.

A mouse immunogenicity study was performed that included four groups of eight female BALB/c mice immunized on days 0 and 21. Mice were immunized twice 2.5 μg of Construct C VLP, 4 μg of Construct C-dn5B (twice the antigen content of the VLPs), or one-tenth the commercial dosage of ImRab3. Blood samples were collected on days 0, 21, and 35 and processed to serum for analysis in a Rapid Fluorescent Foci Inhibition Test (RFFIT) test for neutralzing antibodies. Day 0 serum were pooled by group. Neutralizing antibody titer data was plotted displaying geometric mean with geometric standard deviation (SD). Group statistical analyses were performed using the nonparametric Mann-Whitney test (GraphPad Prism V.9.3.1).

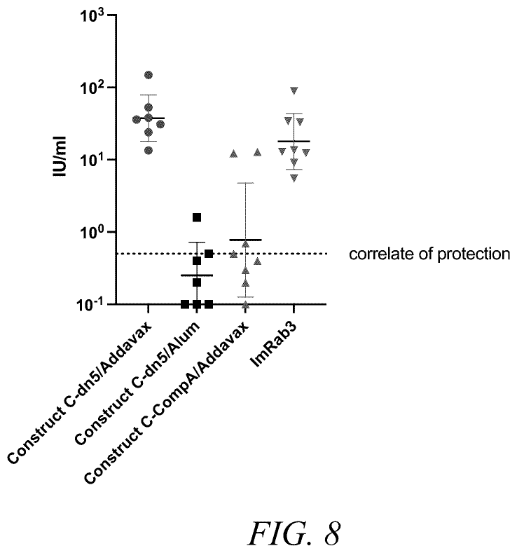

Day 35 sera showed robust neutralizing titers for the Construct C VLPs with oil-in-water adjuvant and ImRab3 control with geometric mean titers of 37.87 and 17.9 IU/ml, respectively (FIG. 8). Construct C VLPs (Construct C-dn5) formulated with alum or trimeric Construct C-dn5B component (Construct C-CompA) induced low but detectable titers with geometric mean titers of 0.25 and 0.78 IU/ml, respectively (FIG. 8). The recognized correlate of protection for rabies is 0.5 IU/ml.

### Example 4

A study was undertaken in naïve BALB/c mice to explore the immunogenicity of different rabies G antigens. Construct C displayed on either I53-50 or dn5 were included to compare the two VLP platforms. Construct F and Construct B (I53-50 VLPs) were also included. The objective of the study was to evaluate the induction of neutralizing titers by Construct B, Construct C, and Construct F. Mice were immunized twice with each VLP (0.1p g) formulated with Addavax, an oil in water adjuvant. One group was immunized with a commercial pet rabies vaccine MRAB® 3 (“ImRab3”) administered at a 1/10 canine dose. A RFFIT test (Rapid Fluorescent Foci Inhibition Test) was performed on serum samples to determine neutralizing antibody titers against rabies. Neutralizing antibody titer data was plotted displaying geometric mean with geometric SD. Group statistical analyses were performed using the nonparametric Mann-Whitney test (GraphPad Prism V.9.3.1).

Five groups of eight female BALB/c mice were immunized on days 0 and 21. Blood samples were collected on days 0, 21, and 35 and processed to serum for analysis in a RFFIT test for neutralization antibodies. Day 0 serum were pooled by group.

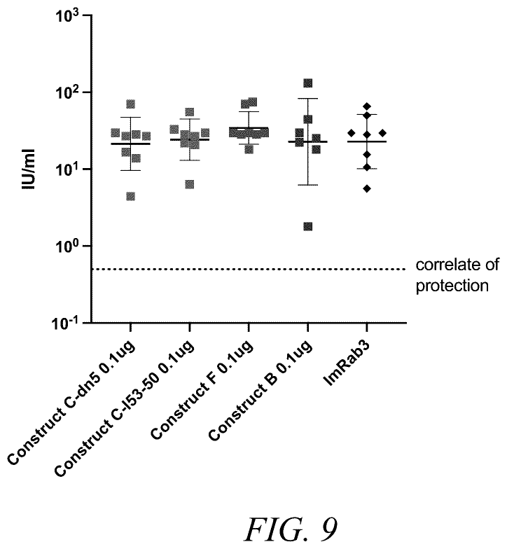

Day 35 sera showed neutralizing titers were induced by Construct C (I53-50 and dn5), Construct B, and Construct F at comparable levels (FIG. 9) and the titers of all animals were above the correlate of protection (0.5 IU/ml). The geometric mean titers induced by the VLPs ranged from 21.3 to 34.3 IU/ml and there were not statistically significant differences between the groups (Mann-Whitney test). The titers observed with the VLPs were comparable to those with ImRab3.

### Example 5

A study was undertaken in naïve ICR mice to explore the immunogenicity of two rabies G antigens displayed on a two-component virus-like particle (VLP). Construct C I53-50 particles or Construct F I53-50 particles, formulated with oil-in water adjuvant (Addavax™), were evaluated at 3 μg, 1 μg, 0.3 μg, and 0.1 μg doses, compared against ImRab3 control administered at a 1/10 canine dose. A RFFIT test (Rapid Fluorescent Foci Inhibition test) was performed to determine neutralizing antibody titers against rabies.

Nine groups of eight female out-breed ICR mice were immunized on days 0 and 21. Blood samples were collected on days 0, 21, and 35 and processed to serum for analysis. Day 0 serum were pooled by group. Neutralizing antibody titer data was plotted displaying geometric mean with geometric SD. Group statistical analyses were performed using the nonparametric Mann-Whitney test (GraphPad Prism V.9.3.1).

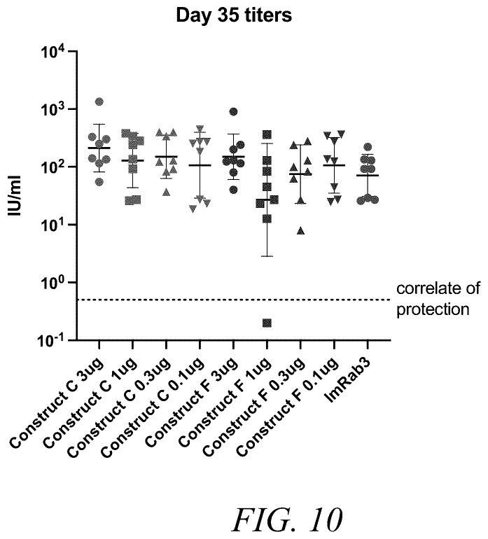

Day 0 serum samples for all groups resulted in titers below 0.2 IU/ml. Day 35 sera showed neutralizing geometric mean titers that range from 74.8 to 211 IU/ml for Construct C and Construct F (FIG. 10). As the dose titration resulted in comparable titers, the data demonstrates that 100 ng of rabies VLP formulated with Addavax induces a maximal humoral response in naïve ICR mice. All animals administered Construct C and Construct F had titers above the correlate of protection except for one animal in the 1 μg dose level for Construct F which was at background levels (<0.2 IU/ml). Titers observed with Construct C and Construct F were comparable to ImRab3 (geometric mean of 71.1) on day 35.

### Example 6

A study was undertaken in naïve Syrian Golden hamsters (SGH) to explore the immunogenicity of two rabies G antigens displayed on a two-component virus-like particle (VLP), essentially as described in Example 5 except for the subject animal. Nine groups of six female SGH were immunized on days 0 and 21. Blood samples were collected on days 0, 21, and 35 and processed to serum for analysis in a RFFIT test for neutralization antibodies. Day 0 serum were pooled by group. Neutralizing antibody titer data was plotted displaying geometric mean with geometric SD. Group statistical analyses were performed using the nonparametric Mann-Whitney test (GraphPad Prism V.9.3.1).

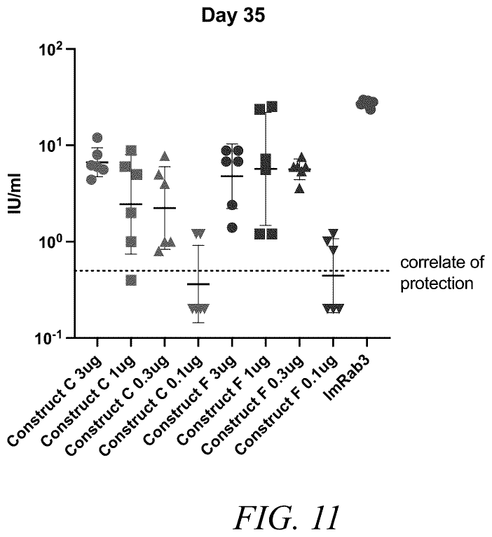

The pooled day 0 serum samples for all groups resulted in titers below 0.2 IU/ml. Day 35 sera showed neutralizing titers for Construct C were above the correlate of protection with the 3 μg, 1 μg and 0.3 μg dosage in 17/18 animals with geometric mean titers ranging from 2.2 to 6.7 IU/ml (FIG. 11). The lowest Construct C dosage group, 0.1 μg, resulted in 2/6 animals with titers above the correlate of protection. The three highest doses (3 μg, 1 μg, and 0.3 μg) of Construct F resulted in comparable titers with all animals above the correlate of protection (18 animals) with geometric mean titers ranging from 4.8 to 5.7 IU/ml and the lowest dose with 3/6 animals above the correlate of protection.

### Example 7

A study was undertaken in naïve BALB/c mice to explore the immunogenicity Construct F displayed on either I53-50 or dn5 as trimeric soluble proteins or VLPs. Construct F was fused to CompA (I53-50, SEQ ID NO: 95) or dn5B (dn5, SEQ ID NO: 97) and expressed as soluble trimeric proteins. The trimeric proteins were also combined with the appropriate pentamer subunits to assemble VLPs. Mice were immunized twice on days 0 and 21 with each trimeric protein or VLP at the dosages and formulations indicated in Table 8. The trimeric soluble proteins were administered at the equivalent antigen content as 0.5 μg and 0.1 μg VLPs. One group was immunized with a commercial pet rabies vaccine MRAB® 3 (“ImRab3”) administered at a 1/10 canine dose. A RFFIT test (Rapid Fluorescent Foci Inhibition Test) was performed on serum samples to determine neutralizing antibody titers against rabies. Neutralizing antibody titer data was plotted displaying geometric mean with geometric SD. Group statistical analyses were performed using the nonparametric Mann-Whitney test (GraphPad Prism V.10.0.0).

Eleven groups of eight female BALB/c mice were immunized on days 0 and 21. Blood samples were collected on days 0, 21, and 35 and processed to serum for analysis in a RFFIT test for neutralization antibodies. Day 0 serum were pooled by group.

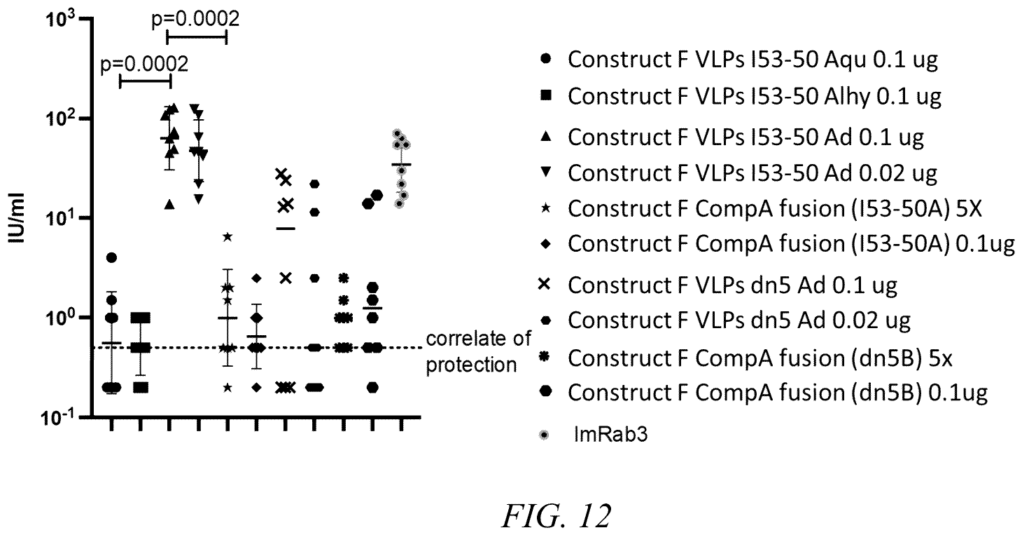

Day 35 sera showed neutralizing titers were induced to varying degrees above day 0 baseline titers (<0.2 IU/ml). Construct F I53-50 VLPs formulated with aqueous buffer or Alhydrogel induced low titers with only a subset of titers at or above the correlate of protection (0.5 IU/ml) with geometric mean titers of 0.56 and 0.52 IU/ml respectively (FIG. 12). Construct F I53-50 VLPs formulated with AddaVax induced significantly higher titers (p=0.0002, Mann-Whitney test) with geometric mean titers of 63.11 and 47.44 IU/ml for the 0.1 μg and 0.2 μg dosages respectively and all animals were above the correlate of protection (FIG. 12). The groups administered CompA-Construct F (soluble trimeric protein) had geometric mean titers of 1 and 0.65 IU/ml, significantly lower (p=0.0002, Mann-Whitney test) than the I53-50 VLPs with Construct F (FIG. 12). Construct F dn5 VLPs formulated with AddaVax induced titers with a wide distribution suggesting the antigen quality in the VLP preparation was suboptimal. The geometric mean titers were 2.65 and 1.03 IU/ml for the 0.1 μg and 0.2 μg dosages respectively and the majority of the animals was above the correlate of protection (FIG. 12). The groups administered dn5B-Construct F (soluble trimeric protein) had geometric mean titers of 0.91 and 1.56 IU/ml, similar to the dn5 VLPs with Construct F (FIG. 12). The titers observed with the I53-50 VLPs were comparable to those with ImRab3 (FIG. 12). The data demonstrates that presentation of the rabies G antigen is impacted by adjuvant and format with the VLP format superior to soluble protein.

TABLE 8
Animal enrollment and treatment groups

| Group | | Antigen per Dose | |
|---|---|---|---|
| No. | Experimental Group | (μg) | Adjuvant |
| 1 | Construct F VLP (I53-50) | 0.1 | Aqueous |
| 2 | Construct F VLP (I53-50) | 0.1 | Alhydrogel |
| 3 | Construct F VLP (I53-50) | 0.1 | Adda Vax |
| 4 | Construct F VLP (I53-50) | 0.02 | Adda Vax |
| 5 | Construct F-CompA fusion | 0.38 (5X antigen) | Adda Vax |
| | (I53-50A) | | |
| 6 | Construct F-CompA fusion | 0.076 (0.1 equi) | Adda Vax |
| | (I530-50A) | | |
| 7 | Construct F VLP (dn5) | 0.1 | Adda Vax |
| 8 | Construct F VLP (dn5) | 0.02 | Adda Vax |
| 9 | Construct F-CompA fusion | 0.37 (5X antigen) | Adda Vax |
| | (dn5B) | | |
| 10 | Construct F-CompA fusion | 0.074 (0.1 equiv) | Adda Vax |
| | (dn5B) | | |
| 11 | ImRab3 | 0.1 ml | N/A |

Each group n = 8 mice

### Example 8

A study was undertaken in Syrian Golden hamsters to evaluate the ability of Construct F VLPs to protect against a subsequent challenge with rabies virus. Five groups of ten female Syrian Golden hamsters were included in the study. Construct F VLPs (I53-50) were administered at dosages of 2 μg or 0.2 μg formulated with AddaVax on days 0 and 21. RabAvert, a commercial human rabies vaccine was administered on days 0, 7 and 21 at 1/20 the human dosage. Two groups were administered saline and one of the groups was not challenged with rabies virus. All other groups were challenged with an intramuscular injection of rabies virus on day 35. Blood samples were collected and processed to serum on days 0, 21, 34 and the day of sacrifice. The brain of each animal was collected on the day of sacrifice. A RFFIT test (Rapid Fluorescent Foci Inhibition Test) was performed on serum samples to determine neutralizing antibody titers against rabies. Neutralizing antibody titer data was plotted displaying geometric mean with geometric SD. Group statistical analyses were performed using the nonparametric Mann-Whitney test (GraphPad Prism V.10.0.0). A direct fluorescent antibody (DFA) test was performed on the brain specimens to detect the presence of the rabies virus. All animals post challenge with rabies virus were monitored for clinical signs of infection for 30 days and were euthanized upon reaching a humane endpoint (i.e., paralysis).

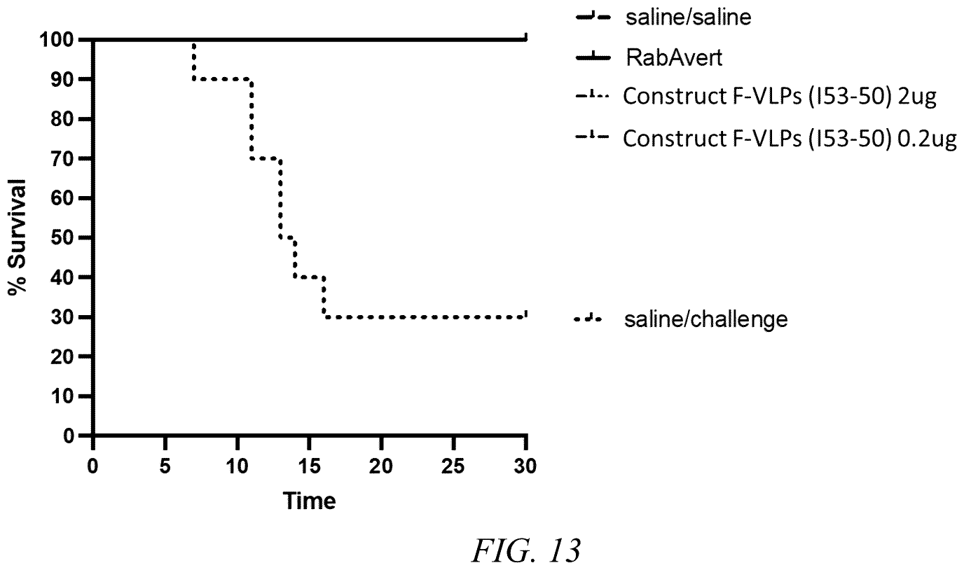

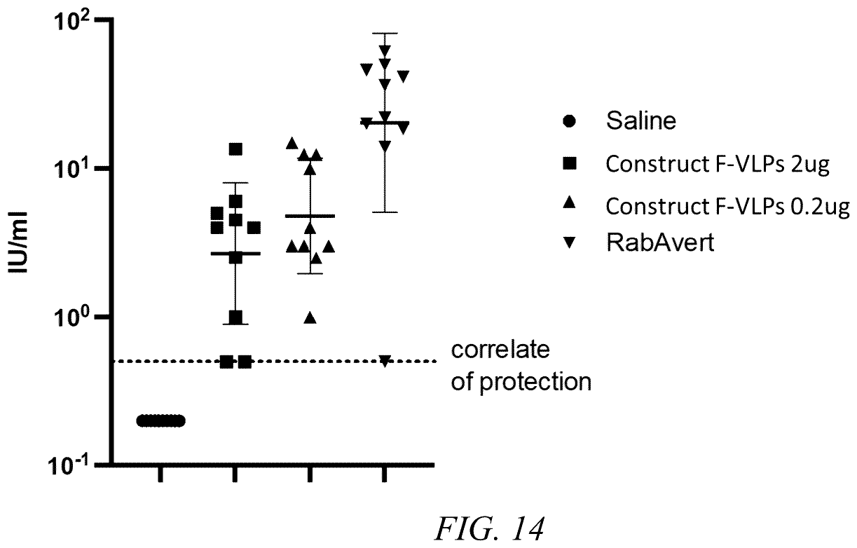

Of the animals administered saline and challenged with rabies virus, 7/10 animals were euthanized due to clinical signs of infection (FIG. 13) in the 30 days post challenge. Of the 20 animals immunized with Construct F VLPs, none were euthanized due to reaching a humane endpoint (FIG. 13). None of the RabAvert immunized animals reached a humane endpoint as well (FIG. 13). Results from the DFA test showed the presence of rabies virus only in the brains of the 7 animals in the control group that were euthanized due to reaching a humane endpoint. All other animals were negative for rabies virus. Neutralizing antibody titers from day 34 serum samples demonstrated that the saline injected animals had titers at the lower limit of detection for the assay (FIG. 14). Construct F VLP immunized animals (doses day 0 and 21) all had titers at or above the correlate of protection (FIG. 14). The RabAvert administered group (doses on days 0, 7 and 21) also had titers at or above the correlate of protection (FIG. 14). The study demonstrates that Construct F VLPs can fully protect animals from a rabies virus challenge.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A polypeptide, comprising a rabies G protein ectodomain, wherein the rabies G protein ectodomain comprises a deletion of a fusion loop domain of the rabies G protein ectodomain,
wherein the deleted fusion loop domain is about residue 70 to about residue 200 of the rabies G protein ectodomain, numbered according to SEQ ID NO: 53; or
wherein the deleted fusion loop domain is about residue 50 to about residue 180, about residue 70 to about residue 180, about residue 80 to about residue 180, about residue 90 to about residue 180, or about residue 100 to about residue 180; about residue 50 to about residue 190, about residue 70 to about residue 190, about residue 80 to about residue 190, about residue 90 to about residue 190, or about residue 100 to about residue 190; about residue 50 to about residue 200, about residue 70 to about residue 200, about residue 80 to about residue 200, about residue 90 to about residue 200, or about residue 100 to about residue 200; about residue 50 to about residue 210, about residue 70 to about residue 210, about residue 80 to about residue 210, about residue 90 to about residue 210, or about residue 100 to about residue 210; about residue 50 to about residue 220, about residue 70 to about residue 220, about residue 80 to about residue 220, about residue 90 to about residue 220, or about residue 100 to about residue 220 of the rabies G protein ectodomain, numbered according to SEQ ID NO: 53.

2. The polypeptide of claim 1, wherein the deleted fusion loop domain is residue 66 to residue 207 of the rabies G protein ectodomain, numbered according to SEQ ID NO: 53.

3. The polypeptide of claim 1 or claim 2, wherein the rabies G protein ectodomain comprises a first polypeptide segment linked to a second polypeptide segment,
wherein the first polypeptide segment shares at least 70% at least 80%, at least 85%, at least 90%, at least 91%, at least 92%, at least 93%, at least 94%, at least 95%, at least 96%, at least 97%, at least 98%, at least 99%, or 100% identity to residues 20-65 of SEQ ID NO: 53; and
wherein the second polypeptide segment shares at least 70% at least 80%, at least 85%, at least 90%, at least 91%, at least 92%, at least 93%, at least 94%, at least 95%, at least 96%, at least 97%, at least 98%, at least 99%, or 100% identity to residues 208-417 of SEQ ID NO: 53.

4. The polypeptide of claim 3, wherein the polypeptide is a fusion protein comprising, in N to C terminal order, the first polypeptide segment, a polypeptide linker, and the second polypeptide segment.

5. The polypeptide of claim 4, wherein the rabies G protein ectodomain shares at least 70% at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs: 77-81.

6. The polypeptide of any one of claims 1 to 5, wherein the polypeptide comprises a multimerization domain.

7. The polypeptide of claim 6, wherein the multimerization domain is a trimerization domain.

8. The polypeptide of claim 7, wherein the multimerization domain shares at least 70% at least 80%, at least 85%, at least 90%, at least 91%, at least 92%, at least 93%, at least 94%, at least 95%, at least 96%, at least 97%, at least 98%, at least 99%, or 100% identity to FoldOn (SEQ ID NO: 58).

9. The polypeptide of claim 6 or claim 7, wherein the multimerization domain shares at least 70% at least 80%, at least 85%, at least 90%, at least 91%, at least 92%, at least 93%, at least 94%, at least 95%, at least 96%, at least 97%, at least 98%, at least 99%, or 100% identity to a polypeptide sequence selected from SEQ ID NOs: 1, 4, 5, 7, 9, 18, 19, 21, 24, 25, 26, 29, 30, 31, 34, 36, 37, 39, 42, 43, 44, 45, 46, 47, 48, 49, 50, and 51, wherein the interface residues identified in Table 3 are conserved.

10. The polypeptide of claim 9, wherein the multimerization domain shares at least 70% at least 80%, at least 85%, at least 90%, at least 91%, at least 92%, at least 93%, at least 94%, at least 95%, at least 96%, at least 97%, at least 98%, at least 99%, or 100% identity to I53-50A (SEQ ID NO: 7).

11. The polypeptide of claim 10, wherein the multimerization domain is I53-50A-Δcys (SEQ ID NO: 67).

12. The polypeptide of claim 10 or claim 11, wherein the polypeptide shares at least 70% at least 80%, at least 85%, at least 90%, at least 91%, at least 92%, at least 93%, at least 94%, at least 95%, at least 96%, at least 97%, at least 98%, at least 99%, or 100% identity to any one of SEQ ID NOs: 93-96.

13. The polypeptide of claim 6 or claim 7, wherein the multimerization domain shares at least 70% at least 80%, at least 85%, at least 90%, at least 91%, at least 92%, at least 93%, at least 94%, at least 95%, at least 96%, at least 97%, at least 98%, at least 99%, or 100% identity to I53-dn5B (SEQ ID NO: 75).

14. The polypeptide of claim 10 or claim 11, wherein the polypeptide shares at least 70% at least 80%, at least 85%, at least 90%, at least 91%, at least 92%, at least 93%, at least 94%, at least 95%, at least 96%, at least 97%, at least 98%, at least 99%, or 100% identity to SEQ ID NO: 101 or SEQ ID NO: 102.

15. The polypeptide of claim 6 or claim 7, wherein the multimerization domain is a ferritin polypeptide capable of forming a ferritin particle.

16. The polypeptide of claim 10 or claim 11, wherein the polypeptide shares at least 70% at least 80%, at least 85%, at least 90%, at least 91%, at least 92%, at least 93%, at least 94%, at least 95%, at least 96%, at least 97%, at least 98%, at least 99%, or 100% identity to Construct F-ferritin (SEQ ID NO: 103).

17. The polypeptide of any one of claims 6 to 16, wherein the multimerization domain is a particle-forming domain.

18. A nanoparticle, comprising a polypeptide according to any one of claims 1 to 17.

19. The nanoparticle of claim 18, wherein the polypeptide comprises the rabies G protein ectodomain according to any on of claims 1 to 17.

20. The nanoparticle of claim 18 or claim 19, wherein the nanoparticle is a protein-based virus-like particle (pbVLP) or nanostructure.

21. The nanoparticle of any one of claims 18 to 20, wherein the nanoparticle lacks a lipid component.

22. The nanoparticle of claim 18, wherein the nanoparticle comprises a lipid component.

23. The nanoparticle of any one of claims 18 to 22, wherein the nanoparticle comprises a second polypeptide component.

24. The nanoparticle of any one of claims 18 to 21, wherein the nanoparticle comprises a second polypeptide component and the first polypeptide component, and wherein the nanoparticle is a self-assembling nanoparticle comprising the first and second polypeptide components symmetrically arranged with point group symmetry.

25. The nanoparticle of claim 23, wherein:
(a) the first polypeptide component comprises the polypeptide according to any one of claims 1 to 17, and the first polypeptide component forms a first homomeric complex via the multimerization domain of the polypetide;
(b) the second polypeptide component comprises a second multimerization domain, and the second polypeptide component forms a second homomeric complex via the second multimerization domain of the polypetide;
(c) the first homomeric complex and the second homomeric complex assemble to form the nanoparticle with point-group symmetry;
(d) the nanoparticle lacks other polypeptide components; and/or
(e) the nanoparticle lacks lipid components.

26. The nanoparticle of claim 25, wherein the nanoparticle has icosahedral symmetry.

27. The nanoparticle of claim 26, wherein:
the first multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50A (SEQ ID NO: 7) or I53-50A ΔCys (SEQ ID NO: 67); and
the second multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50B (SEQ ID NO: 8) or to I53-50B.4PosT1 (SEQ ID NO: 34).

28. The nanoparticle of claim 26, wherein:
the first multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-dn5B (SEQ ID NO: 75); and
the second multimerization domain shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-dn5A (SEQ ID NO: 74).

29. The nanoparticle of any one of claims 18 to 26, wherein:
the first polypeptide component shares at least 70%, at least 80%, at least 85%, at least 90%, at least 91%, at least 92%, at least 93%, at least 94%, at least 95%, at least 96%, at least 97%, at least 98%, at least 99%, or 100% identity to a polypeptide sequence selected from SEQ ID NOs: 93-96; and
the second polypeptide component shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-50B (SEQ ID NO: 8) or to I53-50B.4PosT1 (SEQ ID NO: 34).

30. The nanoparticle of any one of claims 18 to 26, wherein:
the first polypeptide component shares at least 70%, at least 80%, at least 85%, at least 90%, at least 91%, at least 92%, at least 93%, at least 94%, at least 95%, at least 96%, at least 97%, at least 98%, at least 99%, or 100% identity to a polypeptide sequence selected from SEQ ID NOs: 101-102; and
the second polypeptide component shares at least 70%, at least 80%, at least 85%, at least 90%, at least 91%, at least 92%, at least 93%, at least 94%, at least 95%, at least 96%, at least 97%, at least 98%, at least 99%, or 100% identity to I53-dn5A (SEQ ID NO: 74).

31. A pharmaceutical composition for use as a vaccine, comprising the polypeptide of any one of claims 1 to 17 or the nanoparticle of any one of claims 18 to 30, and optionally one or more pharmaceutically acceptable excipients.

32. The pharmaceutical composition of claim 31, wherein the pharmaceutical composition comprises at least one adjuvant.

33. The pharmaceutical composition of claim 32, wherein the pharmaceutical composition comprises an oil-in-water emulsion.

34. The pharmaceutical composition of claim 33, wherein the pharmaceutical composition comprises a squalene-based oil-in-water emulsion.

35. The pharmaceutical composition of claim 32, wherein the pharmaceutical composition comprises a toll-like receptor (TLR) immunostimulant.

36. The pharmaceutical composition of claim 32, wherein the pharmaceutical composition comprises squalene, SLA, GLA, R848, IMQ, 3M-052, CpG, saponin (QS21), or combinations thereof.

37. A polynucleotide, comprising a polynucleotide sequence encoding the polypeptide of any one of claims 1 to 17 or the nanoparticle of any one of claims 18 to 30.

38. A vector, comprising a polynucleotide comprising a polynucleotide sequence encoding any polypeptide of any one of claims 1 to 17 or the nanoparticle of any one of claims 18 to 30.

39. A method of generating an immune response in a subject to a subject infected with rabies virus, comprising:
administering the polypeptide of any one of claims 1 to 17, the nanoparticle of any one of claims 18 to 29, the pharmaceutical composition of any one of claims 30 to 35, the polynucleotide of claim 36, or the vector of claim 37 in an amount effective to generate an immune response.

40. A method of immunizing a subject against infection by rabies to a subject infected with rabies virus, comprising:
administering the polypeptide of any one of claims 1 to 17, the nanoparticle of any one of claims 18 to 29, the pharmaceutical composition of any one of claims 30 to 35, the polynucleotide of claim 36, or the vector of claim 37 in an amount effective to generate an immune response.

41. A method of providing post-exposure prophylaxis to a subject infected with rabies virus, comprising:
administering the polypeptide of any one of claims 1 to 17, the nanoparticle of any one of claims 18 to 29, the pharmaceutical composition of any one of claims 30 to 35, the polynucleotide of claim 36, or the vector of claim 37 in an amount effective to generate an immune response.

42. The method of any one of claims 39 to 41, wherein the immune response comprises a humoral immune response.

43. The method of any one of claims 39 to 42, wherein the immune response comprises a polyclonal antibody response against a rabies G protein.

44. The method of any one of claims 39 to 43, wherein the immune response comprises a neutralizing antibody response to rabies virus.

45. The method of any one of claims 39 to 44, wherein the method generates a protective immune response to rabies virus.

46. The method of any one of claims 39 to 45, wherein the method generates neutralizing antibodies to rabies virus.

47. The method of any one of claims 39 to 46, wherein the administering step comprises intramuscular injection or subcutaneous injection.

48. The method of any one of claims 39 to 47, wherein the method results in the production of rabies-specific neutralizing antibodies in the subject in need thereof.

49. The method of any one of claims 39 to 48, wherein the method results in an increase in Rabies-specific neutralizing antibodies in the subject in need thereof, of at least about 2-fold, at least about 3-fold, at least about 4-fold, at least about 5-fold, at least about 10-fold, at least about 15-fold, at least about 20-fold, or at least about 25-fold increase compared to Rabies-specific neutralizing antibodies in the same subject prior to the administering step.

50. The method of any one of claims 39 to 49, wherein the method generates a neutralizing titer of at least 0.5 IU/mL in a rapid fluorescent focus inhibition test (RFFIT) and/or a fluorescent virus antibody neutralization (FAVN) test.

51. The method of any one of claims 39 to 50, wherein the subject is a non-human animal.

52. The method of any one of claims 39 to 51, wherein the subject is a companion animal.

53. The method of any one of claims 39 to 52, wherein the subject is a human.

54. A host cell, comprising a polynucleotide comprising a polynucleotide sequence encoding of any one of claims 1 to 17 or the nanoparticle of any one of claims 18 to 30.

55. A method of manufacturing a vaccine, comprising:
culturing the host cell of claim 51 in a culture medium so that the host cell secretes a first polypeptide component into the culture media;
purifying the first polypeptide component from the culture media;
mixing the first polypeptide component with a second polypeptide component, wherein the first and second polypeptide components self-assemble to form a nanoparticle; and/or
purifying the nanoparticle.

56. A kit, comprising the polypeptide of any one of claims 1 to 17, the nanoparticle of any one of claims 18 to 30, the pharmaceutical composition of any one of claims 31 to 36, the polynucleotide of claim 37, or the vector of claim 38.


SEQUENCE LISTING (186 sequences)

SEQ ID NO: 1 - protein, 206 aa, synthetic construct
EGMDPLAVLAESRLLPLLTVRGGEDLAGLATVLELMGVGALEITLRTEKGLEALKALRKSGLLLGAGTVRSPKEAEAALEAGAAFLVSPGLLEEVAALAQARGVPYLPGVLTPTEVERALALGLSALKFFPAEPFQGVRVLRAYAEVFPEVRFLPTGGIKEEHLPHYAALPNLLAVGGSWLLQGDLAAVMKKVKAAKALLSPQAPG

SEQ ID NO: 2 - protein, 155 aa, synthetic construct
TKKVGIVDTTFARVDMAEAAIRTLKALSPNIKIIRKTVPGIKDLPVACKKLLEEEGCDIVMALGMPGKAEKDKVCAHEASLGLMLAQLMTNKHIIEVFVHEDEAKDDDELDILALVRAIEHAANVYYLLFKPEYLTRMAGKGLRQGREDAGPARE

SEQ ID NO: 3 - protein, 155 aa, synthetic construct
TKKVGIVDTTFARVDMASAAILTLKMESPNIKIIRKTVPGIKDLPVACKKLLEEEGCDIVMALGMPGKAEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 4 - protein, 208 aa, synthetic construct
STINNQLKALKVIPVIAIDNAEDIIPLGKVLAENGLPAAEITFRSSAAVKAIMLLRSAQPEMLIGAGTILNGVQALAAKEAGATFVVSPGFNPNTVRACQIIGIDIVPGVNNPSTVEAALEMGLTTLKFFPAEASGGISMVKSLVGPYGDIRLMPTGGITPSNIDNYLAIPQVLACGGTWMVDKKLVTNGEWDEIARLTREIVEQVNP

SEQ ID NO: 5 - protein, 113 aa, synthetic construct
PIFTLNTNIKATDVPSDFLSLTSRLVGLILSKPGSYVAVHINTDQQLSFGGSTNPAAFGTLMSIGGIEPSKNRDHSAVLFDHLNAMLGIPKNRMYIHFVNLNGDDVGWNGTTF

SEQ ID NO: 6 - protein, 156 aa, synthetic construct
NQHSHKDYETVRIAVVRARWHADIVDACVEAFEIAMAAIGGDRFAVDVFDVPGAYEIPLHARTLAETGRYGAVLGTAFVVNGGIYRHEFVASAVIDGMMNVQLSTGVPVLSAVLTPHRYRDSAEHHRFFAAHFAVKGVEAARACIEILAAREKIAA

SEQ ID NO: 7 - protein, 203 aa, synthetic construct
MEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 8 - protein, 156 aa, synthetic construct
NQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 9 - protein, 176 aa, synthetic construct
FTKSGDDGNTNVINKRVGKDSPLVNFLGDLDELNSFIGFAISKIPWEDMKKDLERVQVELFEIGEDLSTQSSKKKIDESYVLWLLAATAIYRIESGPVKLFVIPGGSEEASVLHVTRSVARRVERNAVKYTKELPEINRMIIVYLNRLSSLLFAMALVANKRRNQSEKIYEIGKSW

SEQ ID NO: 10 - protein, 156 aa, synthetic construct
NQHSHKDYETVRIAVVRARWHADIVDQCVRAFEEAMADAGGDRFAVDVFDVPGAYEIPLHARTLAETGRYGAVLGTAFVVNGGIYRHEFVASAVIDGMMNVQLSTGVPVLSAVLTPHRYRSSREHHEFFREHFMVKGVEAAAACITILAAREKIAA

SEQ ID NO: 11 - protein, 200 aa, synthetic construct
GHTKGPTPQQHDGSALRIGIVHARWNKTIIMPLLIGTIAKLLECGVKASNIVVQSVPGSWELPIAVQRLYSASQLQTPSSGPSLSAGDLLGSSTTDLTALPTTTASSTGPFDALIAIGVLIKGETMHFEYIADSVSHGLMRVQLDTGVPVIFGVLTVLTDDQAKARAGVIEGSHNHGEDWGLAAVEMGVRRRDWAAGKTE

SEQ ID NO: 12 - protein, 236 aa, synthetic construct
YEVDHADVYDLFYLGRGKDYAAEASDIADLVRSRTPEASSLLDVACGTGTHLEHFTKEFGDTAGLELSEDMLTHARKRLPDATLHQGDMRDFQLGRKFSAVVSMFSSVGYLKTVAELGAAVASFAEHLEPGGVVVVEPWWFPETFADGWVSADVVRRDGRTVARVSHSVREGNATRMEVHFTVADPGKGVRHFSDVHLITLFHQREYEAAFMAAGLRVEYLEGGPSGRGLFVGVPA

SEQ ID NO: 13 - protein, 137 aa, synthetic construct
GMKEKFVLIITHGDFGKGLLSGAEVIIGKQENVHTVGLNLGDNIEKVAKEVMRIIIAKLAEDKEIIIVVDLFGGSPFNIALEMMKTFDVKVITGINMPMLVELLTSINVYDTTELLENISKIGKDGIKVIEKSSLKM

SEQ ID NO: 14 - protein, 153 aa, synthetic construct
KYDGSKLRIGILHARWNLEIIAALVAGAIKRLQEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 15 - protein, 163 aa, synthetic construct
AVKGLGEVDQKYDGSKLRIGILHARWNRKIILALVAGAVLRLLEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 16 - protein, 174 aa, synthetic construct
GANWYLDNESSRLSFTSTKNADIAEVHRFLVLHGKVDPKGLAEVEVETESISTGIPLRDMLLRVLVFQVSKFPVAQINAQLDMRPINNLAPGAQLELRLPLTVSLRGKSHSYNAELLATRLDERRFQVVTLEPLVIHAQDFDMVRAFNALRLVAGLSAVSLSVPVGAVLIFTAR

SEQ ID NO: 17 - protein, 207 aa, synthetic construct
TDYIRDGSAIKALSFAIILAEADLRHIPQDLQRLAVRVIHACGMVDVANDLAFSEGAGKAGRNALLAGAPILCDARMVAEGITRSRLPADNRVIYTLSDPSVPELAKKIGNTRSAAALDLWLPHIEGSIVAIGNAPTALFRLFELLDAGAPKPALIIGMPVGFVGAAESKDELAANSRGVPYVIVRGRRGGSAMTAAAVNALASERE

SEQ ID NO: 18 - protein, 127 aa, synthetic construct
ITVFGLKSKLAPRREKLAEVIYSSLHLGLDIPKGKHAIRFLCLEKEDFYYPFDRSDDYTVIEINLMAGRSEETKMLLIFLLFIALERKLGIRAHDVEITIKEQPAHCWGFRGRTGDSARDLDYDIYV

SEQ ID NO: 19 - protein, 234 aa, synthetic construct
GSDLQKLQRFSTCDISDGLLNVYNIPTGGYFPNLTAISPPQNSSIVGTAYTVLFAPIDDPRPAVNYIDSVPPNSILVLALEPHLQSQFHPFIKITQAMYGGLMSTRAQYLKSNGTVVFGRIRDVDEHRTLNHPVFAYGVGSCAPKAVVKAVGTNVQLKILTSDGVTQTICPGDYIAGDNNGIVRIPVQETDISKLVTYIEKSIEVDRLVSEAIKNGLPAKAAQTARRMVLKDYI

SEQ ID NO: 20 - protein, 161 aa, synthetic construct
SGMRVYLGADHAGYELKQAIIAFLKMTGHEPIDCGALRYDADDDYPAFCIAAATRTVADPGSLGIVLGGSGNGEQIAANKVPGARCALAWSVQTAALAREHNNAQLIGIGGRMHTLEEALRIVKAFVTTPWSKAQRHQRRIDILAEYERTHEAPPVPGAPA

SEQ ID NO: 21 - protein, 156 aa, synthetic construct
GDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 22 - protein, 156 aa, synthetic construct
ILSAEQSFTLRHPHGQAAALAFVREPAAALAGVQRLRGLDSDGEQVWGELLVRVPLLGEVDLPFRSEIVRTPQGAELRPLTLTGERAWVAVSGQATAAEGGEMAFAFQFQAHLATPEAEGEGGAAFEVMVQAAAGVTLLLVAMALPQGLAAGLPPA

SEQ ID NO: 23 - protein, 155 aa, synthetic construct
TKKVGIVDTTFARVDMASAAILTLKMESPNIKIIRKTVPGIKDLPVACKKLLEEEGCDIVMALGMPGKKEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 24 - protein, 208 aa, synthetic construct
DDINNQLKRLKVIPVIAIDNAEDIIPLGKVLAENGLPAAEITFRSSAAVKAIMLLRSAQPEMLIGAGTILNGVQALAAKEAGADFVVSPGFNPNTVRACQIIGIDIVPGVNNPSTVEQALEMGLTTLKFFPAEASGGISMVKSLVGPYGDIRLMPTGGITPDNIDNYLAIPQVLACGGTWMVDKKLVRNGEWDEIARLTREIVEQVNP

SEQ ID NO: 25 - protein, 113 aa, synthetic construct
PIFTLNTNIKADDVPSDFLSLTSRLVGLILSKPGSYVAVHINTDQQLSFGGSTNPAAFGTLMSIGGIEPDKNRDHSAVLFDHLNAMLGIPKNRMYIHFVNLNGDDVGWNGTTF

SEQ ID NO: 26 - protein, 113 aa, synthetic construct
PIFTLNTNIKADDVPSDFLSLTSRLVGLILSEPGSYVAVHINTDQQLSFGGSTNPAAFGTLMSIGGIEPDKNEDHSAVLFDHLNAMLGIPKNRMYIHFVDLDGDDVGWNGTTF

SEQ ID NO: 27 - protein, 156 aa, synthetic construct
NQHSHKDHETVRIAVVRARWHADIVDACVEAFEIAMAAIGGDRFAVDVFDVPGAYEIPLHARTLAETGRYGAVLGTAFVVNGGIYRHEFVASAVIDGMMNVQLDTGVPVLSAVLTPHRYRDSDEHHRFFAAHFAVKGVEAARACIEILNAREKIAA

SEQ ID NO: 28 - protein, 156 aa, synthetic construct
NQHSHKDHETVRIAVVRARWHADIVDACVEAFEIAMAAIGGDRFAVDVFDVPGAYEIPLHARTLAETGRYGAVLGTAFVVDGGIYDHEFVASAVIDGMMNVQLDTGVPVLSAVLTPHEYEDSDEDHEFFAAHFAVKGVEAARACIEILNAREKIAA

SEQ ID NO: 29 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCEWFKAGVLAVGVGDALVKGDPDEVREKAKKFVEKIRGCTE

SEQ ID NO: 30 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVEAMKGPFPNVKFVPTGGVDLDDVCEWFDAGVLAVGVGDALVEGDPDEVREDAKEFVEEIRGCTE

SEQ ID NO: 31 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTE

SEQ ID NO: 32 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 33 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 34 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 35 - protein, 155 aa, synthetic construct
  VARIANT 69: note = X represents A or K
TKKVGIVDTTFARVDMASAAILTLKMESPNIKIIRKTVPGIKDLPVACKKLLEEEGCDIVMALGMPGKXEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 36 - protein, 208 aa, synthetic construct
  VARIANT 1: note = X represents S or D
  VARIANT 2: note = X represents T or D
  VARIANT 9: note = X represents A or R
  VARIANT 84: note = X represents T or D
  VARIANT 118: note = X represents A or Q
  VARIANT 162: note = X represents S or D
  VARIANT 188: note = X represents T or R
XXINNQLKXLKVIPVIAIDNAEDIIPLGKVLAENGLPAAEITFRSSAAVKAIMLLRSAQPEMLIGAGTILNGVQALAAKEAGAXFVVSPGFNPNTVRACQIIGIDIVPGVNNPSTVEXALEMGLTTLKFFPAEASGGISMVKSLVGPYGDIRLMPTGGITPXNIDNYLAIPQVLACGGTWMVDKKLVXNGEWDEIARLTREIVEQVNP

SEQ ID NO: 37 - protein, 113 aa, synthetic construct
  VARIANT 12: note = X represents T or D
  VARIANT 32: note = X represents K or E
  VARIANT 70: note = X represents S or D
  VARIANT 73: note = X represents R or E
  VARIANT 100: note = X represents N or D
  VARIANT 102: note = X represents N or D
PIFTLNTNIKAXDVPSDFLSLTSRLVGLILSXPGSYVAVHINTDQQLSFGGSTNPAAFGTLMSIGGIEPXKNXDHSAVLFDHLNAMLGIPKNRMYIHFVXLXGDDVGWNGTTF

SEQ ID NO: 38 - protein, 156 aa, synthetic construct
  VARIANT 8: note = X represents Y or H
  VARIANT 81: note = X represents N or D
  VARIANT 86: note = X represents R or D
  VARIANT 104: note = X represents S or D
  VARIANT 118: note = X represents R or E
  VARIANT 120: note = X represents R or E
  VARIANT 123: note = X represents A or D
  VARIANT 125: note = X represents H or D
  VARIANT 127: note = X represents R or E
  VARIANT 149: note = X represents A or N
NQHSHKDXETVRIAVVRARWHADIVDACVEAFEIAMAAIGGDRFAVDVFDVPGAYEIPLHARTLAETGRYGAVLGTAFVVXGGIYXHEFVASAVIDGMMNVQLXTGVPVLSAVLTPHXYXDSXEXHXFFAAHFAVKGVEAARACIEILXAREKIAA

SEQ ID NO: 39 - protein, 202 aa, synthetic construct
  VARIANT 123: note = X represents T or D
  VARIANT 136: note = X represents Q or E
  VARIANT 139: note = X represents K or E
  VARIANT 157: note = X represents N or D
  VARIANT 160: note = X represents N or D
  VARIANT 163: note = X represents E or K
  VARIANT 166: note = X represents K or D
  VARIANT 176: note = X represents S, K or D
  VARIANT 180: note = X represents K or E
  VARIANT 182: note = X represents T, D or K
  VARIANT 189: note = X represents K or D
  VARIANT 192: note = X represents A, E or K
  VARIANT 195: note = X represents E or K
  VARIANT 196: note = X represents K or E
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHXILKLFPGEVVGPXFVXAMKGPFPNVKFVPTGGVXLDXVCXWFXAGVLAVGVGXALVXGXPDEVREXAKXFVXXIRGCTE

SEQ ID NO: 40 - protein, 156 aa, synthetic construct
  VARIANT 8: note = X represents Y or H
  VARIANT 37: note = X represents A or R
  VARIANT 81: note = X represents N or D
  VARIANT 86: note = X represents R or D
  VARIANT 96: note = X represents D or N
  VARIANT 104: note = X represents S, D or N
  VARIANT 118: note = X represents R, E or N
  VARIANT 120: note = X represents R, D or E
  VARIANT 121: note = X represents D or K
  VARIANT 123: note = X represents D or K
  VARIANT 125: note = X represents H or D
NQHSHKDXETVRIAVVRARWHAEIVDACVSAFEAAMXDIGGDRFAVDVFDVPGAYEIPLHARTLAETGRYGAVLGTAFVVXGGIYXHEFVASAVIXGMMNVQLXTGVPVLSAVLTPHXYXXSXAXTLLFLALFAVKGMEAARACVEILAAREKIAA

SEQ ID NO: 41 - protein, 158 aa, synthetic construct
GEVPIGDPKELNGMEIAAVYLQPIEMEPRGIDLAASLADIHLEADIHALKNNPNGFPEGFWMPYLTIAYALANADTGAIKTGTLMPMVADDGPHYGANIAMEKDKKGGFGVGTYALTFLISNPEKQGFGRHVDEETGVGKWFEPFVVTYFFKYTGTPK

SEQ ID NO: 42 - protein, 183 aa, synthetic construct
SQAIGILELTSIAKGMELGDAMLKSANVDLLVSKTISPGKFLLMLGGDIGAIQQAIETGTSQAGEMLVDSLVLANIHPSVLPAISGLNSVDKRQAVGIVETWSVAACISAADLAVKGSNVTLVRVHMAFGIGGKCYMVVAGDVLDVAAAVATASLAAGAKGLLVYASIIPRPHEAMWRQMVEG

SEQ ID NO: 43 - protein, 102 aa, synthetic construct
EEVVLITVPSALVAVKIAHALVEERLAACVNIVPGLTSIYRWQGSVVSDHELLLLVKTTTHAFPKLKERVKALHPYTVPEIVALPIAEGNREYLDWLRENTG

SEQ ID NO: 44 - protein, 121 aa, synthetic construct
VRGIRGAITVEEDTPAAILAATIELLLKMLEANGIQSYEELAAVIFTVTEDLTSAFPAEAARLIGMHRVPLLSAREVPVPGSLPRVIRVLALWNTDTPQDRVRHVYLNEAVRLRPDLESAQ

SEQ ID NO: 45 - protein, 176 aa, synthetic construct
SKAKIGIVTVSDRASAGITADISGKAIILALNLYLTSEWEPIYQVIPDEQDVIETTLIKMADEQDCCLIVTTGGTGPAKRDVTPEATEAVCDRMMPGFGELMRAESLKEVPTAILSRQTAGLRGDSLIVNLPGDPASISDCLLAVFPAIPYCIDLMEGPYLECNEAMIKPFRPKAK

SEQ ID NO: 46 - protein, 121 aa, synthetic construct
VRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 47 - protein, 171 aa, synthetic construct
RITTKVGDKGSTRLFGGEEVWKDSPIIEANGTLDELTSFIGEAKHYVDEEMKGILEEIQNDIYKIMGEIGSKGKIEGISEERIAWLLKLILRYMEMVNLKSFVLPGGTLESAKLDVCRTIARRALRKVLTVTREFGIGAEAAAYLLALSDLLFLLARVIEIEKNKLKEVRS

SEQ ID NO: 48 - protein, 122 aa, synthetic construct
PHLVIEATANLRLETSPGELLEQANKALFASGQFGEADIKSRFVTLEAYRQGTAAVERAYLHACLSILDGRDIATRTLLGASLCAVLAEAVAGGGEEGVQVSVEVREMERLSYAKRVVARQR

SEQ ID NO: 49 - protein, 157 aa, synthetic construct
ESVNTSFLSPSLVTIRDFDNGQFAVLRIGRTGFPADKGDIDLCLDKMIGVRAAQIFLGDDTEDGFKGPHIRIRCVDIDDKHTYNAMVYVDLIVGTGASEVERETAEEEAKLALRVALQVDIADEHSCVTQFEMKLREELLSSDSFHPDKDEYYKDFL

SEQ ID NO: 50 - protein, 112 aa, synthetic construct
PVIQTFVSTPLDHHKRLLLAIIYRIVTRVVLGKPEDLVMMTFHDSTPMHFFGSTDPVACVRVEALGGYGPSEPEKVTSIVTAAITAVCGIVADRIFVLYFSPLHCGWNGTNF

SEQ ID NO: 51 - protein, 102 aa, synthetic construct
EEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 52 - protein, 19 aa, synthetic construct
MVPQVLLFVPLLGFSLCFG

SEQ ID NO: 53 - protein, 524 aa, Lyssavirus rabies
MVPQVLLFVPLLGFSLCFGKFPIYTIPDELGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRGFPGGKCSGITVSSTYCSTNHDYTIWMPENPGPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPNWGKYVLMTAGAMIGLVLIFSLMTWCRRANRPESKQRSFGGTGRNVSVTSQSGKVIPSWESYRSGGEIRL

SEQ ID NO: 54 - protein, 440 aa, synthetic construct
KFPIYTIPDELGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRGFPGGKCSGITVSSTYCSTNHDYTIWMPENPGPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPNWGKY

SEQ ID NO: 55 - protein, 398 aa, synthetic construct
  UNSURE 47..188: note = X represents any amino acid or is absent
KFPIYTIPDELGPWSPIDIHHLSCPNNLVVEDEGCTNLSEFSYMELXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHP

SEQ ID NO: 56 - protein, 46 aa, synthetic construct
KFPIYTIPDELGPWSPIDIHHLSCPNNLVVEDEGCTNLSEFSYMEL

SEQ ID NO: 57 - protein, 210 aa, synthetic construct
CDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHP

SEQ ID NO: 58 - protein, 27 aa, synthetic construct
GYIPEAPRDGQAYVRKDGEWVLLSTFL

SEQ ID NO: 59 - protein, 15 aa, synthetic construct
GSGGSGSGSGGSGSG

SEQ ID NO: 60 - protein, 8 aa, synthetic construct
GGSGGSGS

SEQ ID NO: 61 - protein, 8 aa, synthetic construct
GSGGSGSG

SEQ ID NO: 62 - protein, 7 aa, synthetic construct
GSGSGSG

SEQ ID NO: 63 - protein, 15 aa, synthetic construct
GSGSGSGSGSGSGSG

SEQ ID NO: 64 - protein, 19 aa, synthetic construct
TVFKEGDEAEDFVEVHLPD

SEQ ID NO: 65 - protein, 32 aa, synthetic construct
TVFKEGDEAEDFVEVHLPDVYKQISGVDLGLP

SEQ ID NO: 66 - protein, 37 aa, synthetic construct
TVFKEGDEAEDFVEVHLPDVYKQISGVDLGLPNWGKY

SEQ ID NO: 67 - protein, 203 aa, synthetic construct
MEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 68 - protein, 119 aa, synthetic construct
NLAEKMYKAGNAMYRKGQYTIAIIAYTLALLKDPNNAEAWYNLGNAAYKKGEYDEAIEAYQKALELDPNNAEAWYNLGNAYYKQGDYDEAIEYYKKALRLDPRNVDAIENLIEAEEKQG

SEQ ID NO: 69 - protein, 119 aa, synthetic construct
EEAELAYLLGELAYKLGEYRIAIRAYRIALKRDPNNAEAWYNLGNAYYKQGDYREAIRYYLRALKLDPENAEAWYNLGNALYKQGKYDLAIIAYQAALEEDPNNAEAKQNLGNAKQKQG

SEQ ID NO: 70 - protein, 119 aa, synthetic construct
NSAEAMYKMGNAAYKQGDYILAIIAYLLALEKDPNNAEAWYNLGNAAYKQGDYDEAIEYYQKALELDPNNAEAWYNLGNAYYKQGDYDEAIEYYEKALELDPNNAEALKNLLEAIAEQD

SEQ ID NO: 71 - protein, 187 aa, synthetic construct
TDPLAVILYIAILKAEKSIARAKAAEALGKIGDERAVEPLIKALKDEDALVRAAAADALGQIGDERAVEPLIKALKDEEGLVRASAAIALGQIGDERAVQPLIKALTDERDLVRVAAAVALGRIGDEKAVRPLIIVLKDEEGEVREAAAIALGSIGGERVRAAMEKLAERGTGFARKVAVNYLETHK

SEQ ID NO: 72 - protein, 119 aa, synthetic construct
EEAELAYLLGELAYKLGEYRIAIRAYRIALKRDPNNAEAWYNLGNAYYKQGDYDEAIEYYQKALELDPNNAEAWYNLGNAYYKQGDYDEAIEYYEKALELDPENLEALQNLLNAMDKQG

SEQ ID NO: 73 - protein, 279 aa, synthetic construct
IEEVVAEMIDILAESSKKSIEELARAADNKTTEKAVAEAIEEIARLATAAIQLIEALAKNLASEEFMARAISAIAELAKKAIEAIYRLADNHTTDTFMARAIAAIANLAVTAILAIAALASNHTTEEFMARAISAIAELAKKAIEAIYRLADNHTTDKFMAAAIEAIALLATLAILAIALLASNHTTEKFMARAIMAIAILAAKAIEAIYRLADNHTSPTYIEKAIEAIEKIARKAIKAIEMLAKNITTEEYKEKAKKIIDIIRKLAKMAIKKLEDNRT

SEQ ID NO: 74 - protein, 153 aa, synthetic construct
KYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 75 - protein, 119 aa, synthetic construct
EEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 76 - protein, 454 aa, synthetic construct
MVPQVLLFVPLLGFSLCFGKFPIYTIPDELGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRGFPGGKCSGITVSSTYCSTNHDYTIWMPENPGPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLP

SEQ ID NO: 77 - protein, 434 aa, synthetic construct
MELLILKANAITTILTAVTFCFASGKFPIYTIPDELGPWSPIDIHHLSCPNNLVVEDEGCTNLSEFSYMELKVGYISAIKVNGFTCTGVVTSGSGRKHFRPTPDACRAAYNWKMAGDPRYEESLSGSGESLIIISPSVTDLDPYDKSLHSRGFPGGKCSGITVSSTYCSTNHDYTIWMPENPGPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLP

SEQ ID NO: 78 - protein, 282 aa, synthetic construct
MVPQVLLFVPLLGFSLCFGKFPIYTIPDELGPWSPIDIHHLSCPNNLVVEDEGCTNLSEFSYMELGSGSGSGCDIFTNSRGKRASKGNKTCGFVDERGLYKSLKGACRLKLCGVLGLRLMDGTWVAMQTSDETKWCPPDQLVNLHDFRSDEIEPLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHP

SEQ ID NO: 79 - protein, 320 aa, synthetic construct
MVPQVLLFVPLLGFSLCFGKFPIYTIPDELGPWSPIDIHHLSCPNNLVVEDEGCTNLSEFSYMELKVGSSIGKCDIFTNSRGKRASKGNKTCGFVDERGLYKSLKGACRLKLCGVLGLRLMDGTWVAMQTSDETKWCPPDQLVNLHDFRSDEIEPLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLP

SEQ ID NO: 80 - protein, 283 aa, synthetic construct
MVPQVLLFVPLLGFSLCFGKFPIYTIPDELGPWSPIDIHHLSCPNNLVVEDEGCTNLSEFSYMELKVGIPGSNCDIFTNSRGKRASKGNKTCGFVDERGLYKSLKGACRLKLCGVLGLRLMDGTWVAMQTSDETKWCPPDQLVNLHDFRSDEIEPLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHP

SEQ ID NO: 81 - protein, 282 aa, synthetic construct
MVPQVLLFVPLLGFSLCFGKFPIYTIPDELGPWSPIDIHHLSCPNNLVVEDEGCTNLSEFSYMELGSGSGSGCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHP

SEQ ID NO: 82 - protein, 684 aa, synthetic construct
MVPQVLLFVPLLGFSLCFGKFPIYTIPDELGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRGFPGGKCSGITVSSTYCSTNHDYTIWMPENPGPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPGSGSGSGEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 83 - protein, 664 aa, synthetic construct
MELLILKANAITTILTAVTFCFASGKFPIYTIPDELGPWSPIDIHHLSCPNNLVVEDEGCTNLSEFSYMELKVGYISAIKVNGFTCTGVVTSGSGRKHFRPTPDACRAAYNWKMAGDPRYEESLSGSGESLIIISPSVTDLDPYDKSLHSRGFPGGKCSGITVSSTYCSTNHDYTIWMPENPGPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPGSGSGSGEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 84 - DNA, 1995 bp, synthetic construct
  source 1..1995: mol_type = other DNA
atggagttgctaatcctcaaagcaaatgcaattaccacaatcctcactgcagtcacattttgttttgcctcaggtaaattccctatctacaccatccctgatgagctgggaccttggagccccatcgacatccaccacctgagctgccccaacaacctggtggtcgaggacgagggctgtactaacctgagcgagttcagctacatggaactgaaggtgggctacatcagtgccattaaggtgaacggcttcacatgcaccggcgttgtgaccagcggcagcggcagaaagcacttccgccccacacctgacgcctgcagagccgcctataactggaagatggccggcgaccccagatacgaggaatctctgagcggcagcggcgagagcctgatcatcatcagccccagcgtgacagacctggacccttacgacaagagcctgcacagcagaggttttccaggcggaaagtgcagtggaattaccgtcagcagcacctactgctccacaaaccacgattacaccatctggatgcccgagaaccctggcccgcggacaccgtgcgacatcttcaccaatagcagaggcaagcgggccagcaagggcaacaagacctgtggcttcgtggacgaaagaggcctgtacaagtcactgaagggtgcttgtagactgaaactgtgcggcgtgctgggcctgagactgatggacggcacctgggtcgccatgcaaacatctgacgagacaaagtggtgccctcctgaccagctggtgaacctgcacgacttcagaagcgacgagatcgaacacctggtcgtggaagagctggtcaagaaaagagaggagtgcctggatgccctcgagtctatcatgaccaccaagtctgtgtccttccggagactgtctcacctgcggaagctggtgcccggattcggcaaggcctacaccatcttcaacaagacactgatggaagccgatgctcactacaagagcgttagaacctggaacgagatcattccaagcaagggttgtctgaaggtgggcggcagatgccaccctcacgtgaacggcgtctttttcaacggcatcatcctgggccccgatggccacgtgctgatccctgagatgcagagcagcctgctgcagcagcacatggaactgctgaagtccagcgtcatccccctgatgcacccccttgctgatcctagcacagtgttcaaggaaggcgacgaagctgaagatttcgttgaggtgcacctgcctgatgtgtacaagcagatcagcggagtggacctgggcctgcctggcagcggctctggatctggcgagaaagctgccaaggccgaggaagccgctagaaagatggaagagctgtttaagaagcacaagatcgtggccgtgctgcgagctaattctgtcgaggaagccatcgagaaagccgttgccgtgttcgccggaggcgttcatctgatcgagatcaccttcaccgtgcccgatgccgacaccgtgatcaaggctctgagcgtgctgaaagaaaagggcgccatcatcggcgccggcacagtcaccagcgtggaacaggccagaaaggccgtggaaagtggcgccgagttcatcgtgtctccacacctggacgaggaaatctcacagtttgccaaggaaaagggggtgttctacatgcctggcgtgatgacccctacagagctggtgaaagctatgaaactgggccacaccatcctgaagctgttcccaggcgaagtggtgggcccccaatttgtgaaggctatgaagggcccattccctaacgtgaagttcgtgcctaccggcggcgtgaatctggacaacgtggccgagtggttcaaggccggcgtgctggccgtgggagtgggcagcgccctggtgaagggcacccccgacgaggtgcgggaaaaggccaaagccttcgtggaaaagatcagaggcgctacagagctcgagcaccaccaccaccatcattga

SEQ ID NO: 85 - protein, 512 aa, synthetic construct
MVPQVLLFVPLLGFSLCFGKFPIYTIPDELGPWSPIDIHHLSCPNNLVVEDEGCTNLSEFSYMELGSGSGSGCDIFTNSRGKRASKGNKTCGFVDERGLYKSLKGACRLKLCGVLGLRLMDGTWVAMQTSDETKWCPPDQLVNLHDFRSDEIEPLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPGSGSGSGEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 86 - DNA, 1539 bp, synthetic construct
  source 1..1539: mol_type = other DNA
atggtgcctcaggtgctgctgttcgtgcccctgctgggctttagcctctgcttcggaaaattccctatctacaccatccctgatgagctgggaccttggagccccatcgacatccaccacctgagctgccccaacaacctggtggtcgaggacgagggctgtactaacctgagcgagttcagctacatggaactgggatctggctctggcagcggatgcgacatcttcaccaatagcagaggcaagcgggccagcaagggcaacaagacctgtggcttcgtggacgaaagaggcctgtacaagtcactgaagggtgcttgtagactgaaactgtgcggcgtgctgggcctgagactgatggacggcacctgggtcgccatgcaaacatctgacgagacaaagtggtgccctcctgaccagctggtgaacctgcacgacttcagaagcgacgagatcgaacctctggtcgtggaagagctggtcaagaaaagagaggagtgcctggatgccctcgagtctatcatgaccaccaagtctgtgtccttccggagactgtctcacctgcggaagctggtgcccggattcggcaaggcctacaccatcttcaacaagacactgatggaagccgatgctcactacaagagcgttagaacctggaacgagatcattccaagcaagggttgtctgaaggtgggcggcagatgccaccctcacgtgaacggcgtctttttcaacggcatcatcctgggccccgatggccacgtgctgatccctgagatgcagagcagcctgctgcagcagcacatggaactgctgaagtccagcgtcatccccctgatgcaccccggcagcggctctggatctggcgagaaagctgccaaggccgaggaagccgctagaaagatggaagagctgtttaagaagcacaagatcgtggccgtgctgcgagctaattctgtcgaggaagccatcgagaaagccgttgccgtgttcgccggaggcgttcatctgatcgagatcaccttcaccgtgcccgatgccgacaccgtgatcaaggctctgagcgtgctgaaagaaaagggcgccatcatcggcgccggcacagtcaccagcgtggaacaggccagaaaggccgtggaaagtggcgccgagttcatcgtgtctccacacctggacgaggaaatctcacagtttgccaaggaaaagggggtgttctacatgcctggcgtgatgacccctacagagctggtgaaagctatgaaactgggccacaccatcctgaagctgttcccaggcgaagtggtgggcccccaatttgtgaaggctatgaagggcccattccctaacgtgaagttcgtgcctaccggcggcgtgaatctggacaacgtggccgagtggttcaaggccggcgtgctggccgtgggagtgggcagcgccctggtgaagggcacccccgacgaggtgcgggaaaaggccaaagccttcgtggaaaagatcagaggcgctacagagctcgagcaccaccaccaccatcattga

SEQ ID NO: 87 - protein, 550 aa, synthetic construct
MVPQVLLFVPLLGFSLCFGKFPIYTIPDELGPWSPIDIHHLSCPNNLVVEDEGCTNLSEFSYMELKVGSSIGKCDIFTNSRGKRASKGNKTCGFVDERGLYKSLKGACRLKLCGVLGLRLMDGTWVAMQTSDETKWCPPDQLVNLHDFRSDEIEPLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPGSGSGSGEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 88 - DNA, 1653 bp, synthetic construct
  source 1..1653: mol_type = other DNA
atggtgcctcaggtgctgctgttcgtgcccctgctgggctttagcctctgcttcggaaaattccctatctacaccatccctgatgagctgggaccttggagccccatcgacatccaccacctgagctgccccaacaacctggtggtcgaggacgagggctgtactaacctgagcgagttcagctacatggaactgaaggtgggcagctctatcggcaagtgcgacatcttcaccaatagcagaggcaagcgggccagcaagggcaacaagacctgtggcttcgtggacgaaagaggcctgtacaagtcactgaagggtgcttgtagactgaaactgtgcggcgtgctgggcctgagactgatggacggcacctgggtcgccatgcaaacatctgacgagacaaagtggtgccctcctgaccagctggtgaacctgcacgacttcagaagcgacgagatcgaacctctggtcgtggaagagctggtcaagaaaagagaggagtgcctggatgccctcgagtctatcatgaccaccaagtctgtgtccttccggagactgtctcacctgcggaagctggtgcccggattcggcaaggcctacaccatcttcaacaagacactgatggaagccgatgctcactacaagagcgttagaacctggaacgagatcattccaagcaagggttgtctgaaggtgggcggcagatgccaccctcacgtgaacggcgtctttttcaacggcatcatcctgggccccgatggccacgtgctgatccctgagatgcagagcagcctgctgcagcagcacatggaactgctgaagtccagcgtcatccccctgatgcacccccttgctgatcctagcacagtgttcaaggaaggcgacgaagctgaagatttcgttgaggtgcacctgcctgatgtgtacaagcagatcagcggagtggacctgggcctgcctggcagcggctctggatctggcgagaaagctgccaaggccgaggaagccgctagaaagatggaagagctgtttaagaagcacaagatcgtggccgtgctgcgagctaattctgtcgaggaagccatcgagaaagccgttgccgtgttcgccggaggcgttcatctgatcgagatcaccttcaccgtgcccgatgccgacaccgtgatcaaggctctgagcgtgctgaaagaaaagggcgccatcatcggcgccggcacagtcaccagcgtggaacaggccagaaaggccgtggaaagtggcgccgagttcatcgtgtctccacacctggacgaggaaatctcacagtttgccaaggaaaagggggtgttctacatgcctggcgtgatgacccctacagagctggtgaaagctatgaaactgggccacaccatcctgaagctgttcccaggcgaagtggtgggcccccaatttgtgaaggctatgaagggcccattccctaacgtgaagttcgtgcctaccggcggcgtgaatctggacaacgtggccgagtggttcaaggccggcgtgctggccgtgggagtgggcagcgccctggtgaagggcacccccgacgaggtgcgggaaaaggccaaagccttcgtggaaaagatcagaggcgctacagagctcgagcaccaccaccaccatcattga

SEQ ID NO: 89 - protein, 513 aa, synthetic construct
MVPQVLLFVPLLGFSLCFGKFPIYTIPDELGPWSPIDIHHLSCPNNLVVEDEGCTNLSEFSYMELKVGIPGSNCDIFTNSRGKRASKGNKTCGFVDERGLYKSLKGACRLKLCGVLGLRLMDGTWVAMQTSDETKWCPPDQLVNLHDFRSDEIEPLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPGSGSGSGEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 90 - DNA, 1542 bp, synthetic construct
  source 1..1542: mol_type = other DNA
atggtgcctcaggtgctgctgttcgtgcccctgctgggctttagcctctgcttcggaaaattccctatctacaccatccctgatgagctgggaccttggagccccatcgacatccaccacctgagctgccccaacaacctggtggtcgaggacgagggctgtactaacctgagcgagttcagctacatggaactgaaggtgggcatccccggctctaactgcgacatcttcaccaatagcagaggcaagcgggccagcaagggcaacaagacctgtggcttcgtggacgaaagaggcctgtacaagtcactgaagggtgcttgtagactgaaactgtgcggcgtgctgggcctgagactgatggacggcacctgggtcgccatgcaaacatctgacgagacaaagtggtgccctcctgaccagctggtgaacctgcacgacttcagaagcgacgagatcgaacctctggtcgtggaagagctggtcaagaaaagagaggagtgcctggatgccctcgagtctatcatgaccaccaagtctgtgtccttccggagactgtctcacctgcggaagctggtgcccggattcggcaaggcctacaccatcttcaacaagacactgatggaagccgatgctcactacaagagcgttagaacctggaacgagatcattccaagcaagggttgtctgaaggtgggcggcagatgccaccctcacgtgaacggcgtctttttcaacggcatcatcctgggccccgatggccacgtgctgatccctgagatgcagagcagcctgctgcagcagcacatggaactgctgaagtccagcgtcatccccctgatgcaccccggcagcggctctggatctggcgagaaagctgccaaggccgaggaagccgctagaaagatggaagagctgtttaagaagcacaagatcgtggccgtgctgcgagctaattctgtcgaggaagccatcgagaaagccgttgccgtgttcgccggaggcgttcatctgatcgagatcaccttcaccgtgcccgatgccgacaccgtgatcaaggctctgagcgtgctgaaagaaaagggcgccatcatcggcgccggcacagtcaccagcgtggaacaggccagaaaggccgtggaaagtggcgccgagttcatcgtgtctccacacctggacgaggaaatctcacagtttgccaaggaaaagggggtgttctacatgcctggcgtgatgacccctacagagctggtgaaagctatgaaactgggccacaccatcctgaagctgttcccaggcgaagtggtgggcccccaatttgtgaaggctatgaagggcccattccctaacgtgaagttcgtgcctaccggcggcgtgaatctggacaacgtggccgagtggttcaaggccggcgtgctggccgtgggagtgggcagcgccctggtgaagggcacccccgacgaggtgcgggaaaaggccaaagccttcgtggaaaagatcagaggcgctacagagctcgagcaccaccaccaccatcattga

SEQ ID NO: 91 - protein, 512 aa, synthetic construct
MVPQVLLFVPLLGFSLCFGKFPIYTIPDELGPWSPIDIHHLSCPNNLVVEDEGCTNLSEFSYMELGSGSGSGCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPGSGSGSGEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 92 - DNA, 1539 bp, synthetic construct
  source 1..1539: mol_type = other DNA
atggtgcctcaggtgctgctgttcgtgcccctgctgggctttagcctctgcttcggaaaattccctatctacaccatccctgatgagctgggaccttggagccccatcgacatccaccacctgagctgccccaacaacctggtggtcgaggacgagggctgtactaacctgagcgagttcagctacatggaactgggatctggctctggcagcggatgcgacatcttcaccaatagcagaggcaagcgggccagcaagggcaacaagacctgtggcttcgtggacgaaagaggcctgtacaagtcactgaagggtgcttgtagactgaaactgtgcggcgtgctgggcctgagactgatggacggcacctgggtcgccatgcaaacatctgacgagacaaagtggtgccctcctgaccagctggtgaacctgcacgacttcagaagcgacgagatcgaacacctggtcgtggaagagctggtcaagaaaagagaggagtgcctggatgccctcgagtctatcatgaccaccaagtctgtgtccttccggagactgtctcacctgcggaagctggtgcccggattcggcaaggcctacaccatcttcaacaagacactgatggaagccgatgctcactacaagagcgttagaacctggaacgagatcattccaagcaagggttgtctgaaggtgggcggcagatgccaccctcacgtgaacggcgtctttttcaacggcatcatcctgggccccgatggccacgtgctgatccctgagatgcagagcagcctgctgcagcagcacatggaactgctgaagtccagcgtcatccccctgatgcaccccggcagcggctctggatctggcgagaaagctgccaaggccgaggaagccgctagaaagatggaagagctgtttaagaagcacaagatcgtggccgtgctgcgagctaattctgtcgaggaagccatcgagaaagccgttgccgtgttcgccggaggcgttcatctgatcgagatcaccttcaccgtgcccgatgccgacaccgtgatcaaggctctgagcgtgctgaaagaaaagggcgccatcatcggcgccggcacagtcaccagcgtggaacaggccagaaaggccgtggaaagtggcgccgagttcatcgtgtctccacacctggacgaggaaatctcacagtttgccaaggaaaagggggtgttctacatgcctggcgtgatgacccctacagagctggtgaaagctatgaaactgggccacaccatcctgaagctgttcccaggcgaagtggtgggcccccaatttgtgaaggctatgaagggcccattccctaacgtgaagttcgtgcctaccggcggcgtgaatctggacaacgtggccgagtggttcaaggccggcgtgctggccgtgggagtgggcagcgccctggtgaagggcacccccgacgaggtgcgggaaaaggccaaagccttcgtggaaaagatcagaggcgctacagagctcgagcaccaccaccaccatcattga

SEQ ID NO: 93 - protein, 657 aa, synthetic construct
KFPIYTIPDELGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRGFPGGKCSGITVSSTYCSTNHDYTIWMPENPGPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPGSGSGSGEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 94 - protein, 631 aa, synthetic construct
KFPIYTIPDELGPWSPIDIHHLSCPNNLVVEDEGCTNLSEFSYMELKVGYISAIKVNGFTCTGVVTSGSGRKHFRPTPDACRAAYNWKMAGDPRYEESLSGSGESLIIISPSVTDLDPYDKSLHSRGFPGGKCSGITVSSTYCSTNHDYTIWMPENPGPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPGSGSGSGEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 95 - protein, 485 aa, synthetic construct
KFPIYTIPDELGPWSPIDIHHLSCPNNLVVEDEGCTNLSEFSYMELGSGSGSGCDIFTNSRGKRASKGNKTCGFVDERGLYKSLKGACRLKLCGVLGLRLMDGTWVAMQTSDETKWCPPDQLVNLHDFRSDEIEPLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPGSGSGSGEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 96 - protein, 485 aa, synthetic construct
KFPIYTIPDELGPWSPIDIHHLSCPNNLVVEDEGCTNLSEFSYMELGSGSGSGCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPGSGSGSGEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 97 - protein, 562 aa, synthetic construct
MVPQVLLFVPLLGFSLCFGKFPIYTIPDELGPWSPIDIHHLSCPNNLVVEDEGCTNLSEFSYMELKVGYISAIKVNGFTCTGVVTSGSGRKHFRPTPDACRAAYNWKMAGDPRYEESLSGSGESLIIISPSVTDLDPYDKSLHSRGFPGGKCSGITVSSTYCSTNHDYTIWMPENPGPRTPCDIFTNSRGKRASKGNKTCGFVDERGLYKSLKGACRLKLCGVLGLRLMDGTWVAMQTSDETKWCPPDQLVNLHDFRSDEIEPLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPSAIGGSAEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGSHHHHHH

SEQ ID NO: 98 - DNA, 1689 bp, synthetic construct
  source 1..1689: mol_type = other DNA
atggtgcctcaggtgctgctgttcgtgcccctgctgggctttagcctctgcttcggaaaattccctatctacaccatccctgatgagctgggaccttggagccccatcgacatccaccacctgagctgccccaacaacctggtggtcgaggacgagggctgtactaacctgagcgagttcagctacatggaactgaaggtgggctacatcagtgccattaaggtgaacggcttcacatgcaccggcgttgtgaccagcggcagcggcagaaagcacttccgccccacacctgacgcctgcagagccgcctataactggaagatggccggcgaccccagatacgaggaatctctgagcggcagcggcgagagcctgatcatcatcagccccagcgtgacagacctggacccttacgacaagagcctgcacagcagaggttttccaggcggaaagtgcagtggaattaccgtcagcagcacctactgctccacaaaccacgattacaccatctggatgcccgagaaccctggcccgcggacaccgtgcgacatcttcaccaatagcagaggcaagcgggccagcaagggcaacaagacctgtggcttcgtggacgaaagaggcctgtacaagtcactgaagggtgcttgtagactgaaactgtgcggcgtgctgggcctgagactgatggacggcacctgggtcgccatgcaaacatctgacgagacaaagtggtgccctcctgaccagctggtgaacctgcacgacttcagaagcgacgagatcgaacctctggtcgtggaagagctggtcaagaaaagagaggagtgcctggatgccctcgagtctatcatgaccaccaagtctgtgtccttccggagactgtctcacctgcggaagctggtgcccggattcggcaaggcctacaccatcttcaacaagacactgatggaagccgatgctcactacaagagcgttagaacctggaacgagatcattccaagcaagggttgtctgaaggtgggcggcagatgccaccctcacgtgaacggcgtctttttcaacggcatcatcctgggccccgatggccacgtgctgatccctgagatgcagagcagcctgctgcagcagcacatggaactgctgaagtccagcgtcatccccctgatgcacccccttgctgatcctagcacagtgttcaaggaaggcgacgaagctgaagatttcgttgaggtgcacctgcctgatgtgtacaagcagatcagcggagtggacctgggcctgccttctgccatcggaggctccgccgaggaggcagagctggcctacctgctgggcgagctggcctacaagctgggcgagtataggatcgccatccgggcctatagaatcgccctgaagcgcgatcccaacaatgccgaggcctggtacaacctgggcaatgcctactataagcagggccggtatagagaggccatcgagtactatcagaaggccctggagctggaccctaataacgctgaggcctggtataacctgggaaacgcctactatgagcggggcgagtatgaggaagctatcgagtactataggaaggccctgcgcctggacccaaacaatgccgatgccatgcagaacctgctgaatgccaagatgagagaggagggcagccaccaccaccaccaccactga

SEQ ID NO: 99 - protein, 416 aa, synthetic construct
MVPQVLLFVPLLGFSLCFGKFPIYTIPDELGPWSPIDIHHLSCPNNLVVEDEGCTNLSEFSYMELGSGSGSGCDIFTNSRGKRASKGNKTCGFVDERGLYKSLKGACRLKLCGVLGLRLMDGTWVAMQTSDETKWCPPDQLVNLHDFRSDEIEPLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPSAIGGSAEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGSHHHHHH

SEQ ID NO: 100 - DNA, 1251 bp, synthetic construct
  source 1..1251: mol_type = other DNA
atggtgcctcaggtgctgctgttcgtgcccctgctgggctttagcctctgcttcggaaaattccctatctacaccatccctgatgagctgggaccttggagccccatcgacatccaccacctgagctgccccaacaacctggtggtcgaggacgagggctgtactaacctgagcgagttcagctacatggaactgggatctggctctggcagcggatgcgacatcttcaccaatagcagaggcaagcgggccagcaagggcaacaagacctgtggcttcgtggacgaaagaggcctgtacaagtcactgaagggtgcttgtagactgaaactgtgcggcgtgctgggcctgagactgatggacggcacctgggtcgccatgcaaacatctgacgagacaaagtggtgccctcctgaccagctggtgaacctgcacgacttcagaagcgacgagatcgaacctctggtcgtggaagagctggtcaagaaaagagaggagtgcctggatgccctcgagtctatcatgaccaccaagtctgtgtccttccggagactgtctcacctgcggaagctggtgcccggattcggcaaggcctacaccatcttcaacaagacactgatggaagccgatgctcactacaagagcgttagaacctggaacgagatcattccaagcaagggttgtctgaaggtgggcggcagatgccaccctcacgtgaacggcgtctttttcaacggcatcatcctgggccccgatggccacgtgctgatccctgagatgcagagcagcctgctgcagcagcacatggaactgctgaagtccagcgtcatccccctgatgcacccctctgccatcggaggctccgccgaggaggcagagctggcctacctgctgggcgagctggcctacaagctgggcgagtataggatcgccatccgggcctatagaatcgccctgaagcgcgatcccaacaatgccgaggcctggtacaacctgggcaatgcctactataagcagggccggtatagagaggccatcgagtactatcagaaggccctggagctggaccctaataacgctgaggcctggtataacctgggaaacgcctactatgagcggggcgagtatgaggaagctatcgagtactataggaaggccctgcgcctggacccaaacaatgccgatgccatgcagaacctgctgaatgccaagatgagagaggagggcagccaccaccaccaccaccactga

SEQ ID NO: 101 - protein, 535 aa, synthetic construct
KFPIYTIPDELGPWSPIDIHHLSCPNNLVVEDEGCTNLSEFSYMELKVGYISAIKVNGFTCTGVVTSGSGRKHFRPTPDACRAAYNWKMAGDPRYEESLSGSGESLIIISPSVTDLDPYDKSLHSRGFPGGKCSGITVSSTYCSTNHDYTIWMPENPGPRTPCDIFTNSRGKRASKGNKTCGFVDERGLYKSLKGACRLKLCGVLGLRLMDGTWVAMQTSDETKWCPPDQLVNLHDFRSDEIEPLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPSAIGGSAEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 102 - protein, 389 aa, synthetic construct
KFPIYTIPDELGPWSPIDIHHLSCPNNLVVEDEGCTNLSEFSYMELGSGSGSGCDIFTNSRGKRASKGNKTCGFVDERGLYKSLKGACRLKLCGVLGLRLMDGTWVAMQTSDETKWCPPDQLVNLHDFRSDEIEPLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPSAIGGSAEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 103 - protein, 461 aa, synthetic construct
MVPQVLLFVPLLGFSLCFGKFPIYTIPDELGPWSPIDIHHLSCPNNLVVEDEGCTNLSEFSYMELGSGSGSGCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPGGSGGSGSGGSGGSGSDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 104 - protein, 403 aa, synthetic construct
KFPIYTIPDKLGPWSPIDIHHLSCPNNLVVEDEGCTNLSGFSYMELKVGYISAIKVNGFTCTGVVTEAETYTNFVGYVTTTFKRKHFRPIPDACRAAYNWKMAGDPRYEESLQNPYPDYHWLRTVKTTKESLIIISPSVADLDPYDKSLHSRVFPGGKCLGITVSSTYCSTNHDYTIWMPEEPRLGTSCDIFTSSKGKKASKGGKTCGFVDERGLYKSLKGACKLKLCGVLGLRLMDGTWVSIQTSDDTKWCPSDQLVNLHDFHSDEIEHLVVEELVKKREECLDALESIMTTKSVSFRRLSYLRKLVPGFGKAYTIFNKTLMEADAHYKSVRTWNEIIPSKGCLKVRERCHPPVDGVFFNGIILGPDGNVLIPEMQSSLLQQHMELLESSVIPLMHPLADPS

SEQ ID NO: 105 - protein, 403 aa, Synthetic construct
KFPIYTIPDTLGPWSPIDIHHLSCPNNLVVEDEGCTNLSGFSYMELKVGHISAIKVNGFTCTGVVTEAETYTNFVGYVTTTFKRKHFRPTPDACRAAYNWKMAGDPRYEESLHSPYPDYHWLRTVKTTKESLVIISPSVADLDPYDNSLHSRVFPSGKCSGITVSSVYCSTNHDYTVWMPESLRLGTSCDIFTNSRGKRVSKGSKTCGFVDERGLYKSLKGACKLKLCGVRGLRLMDGTWVAMQTSNETKWCPPDQLVNLHDLRSDEIEHLVIEELVKKREECLDALESIITTKSVSFRRLSYLRKLVPGFGKAYTIFNKTLMEAEAHYKSVRTWNEIIPSKGCLRVGGRCHPHVNGVFFNGIILGPDGHVLIPEMQSSLLQQHIELLESSVIPLMHPLADPF

SEQ ID NO: 106 - protein, 403 aa, Synthetic construct
KFPIYTIPDKLGPWSPIDIHHLSCPNNLVVEDEGCTNLSGFSYMELKVGYISAIKVNGFTCTGVVTEAETYTNFVGYVTTTFKRKHFRPTPDACRAAYNWKMAGDPRYEESLHNPYPDYHWLRTVKTTKESLVIISPSVADLDPYDKSLHSRVFPSGKCLGITISSTYCSTNHDYTIWMPENPRLGTSCDIFTNSRGKRASKGSKTCGFVDERGLYKSLKGACKLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGHVLIPEMQSSLLQQHMELLESSVIPLMHPLADPS

SEQ ID NO: 107 - protein, 403 aa, Synthetic construct
KFPIYTIPDKLGPWSPIDIHHLSCPNNLVVEDEGCTNLSGFSYMELKVGYISAIKTNGFTCTGVVTEAETYTNFVGYVTTTFKRKHFRPTPDACRAAYNWKMAGDPRYEESLHNPYPDYHWLRTVKTTKESLVIISPSVADLDPYDRSLHSRVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPMADPS

SEQ ID NO: 108 - protein, 403 aa, Synthetic construct
KFPIYTIPTKLGPWSPIDIHHLSCPNNLVVEDEGCTNLSGFSYMELKVGRISAIKVNGFTCTGVVTEAETYTNFVGYVTTTFKRKHFRPMPGCMYSRVQLEDGRSPQIEESLHNPYPDYHWLRTVRTTKESLIIISPSVTDLDPYDKSLHSRVFPGRKCSGITVSSTYCSTNHDYTVWMPEILRLGTSCDIFTNSRGKRASKGSKTCGFVDERGLYKSLKGACKLKLCGVPGLRLMDGTWVAMQTSNETKWCPPGQLVNLHDLHSDEIEHLVVEELVKKREECLDALESITTTKSVSFRRLSHLRKLVPGFGKAYTIFNKTLMEAEAHYKSVRTWNEIIPSKGCLRVGGGCHPHVNGVFFNGIILGPDGHVLIPEMQSSLLQQHIELLESSVIPLMHPLADPF

SEQ ID NO: 109 - protein, 403 aa, Synthetic construct
KFPIYTIPDKLGPWSPIDIHHLSCPNNLVVEDEGCNSLSGFSYMELKVGYISAIKVNGFTCTGVVTEAETYTNFVGYVTTTFKRKHFRPMPDACRSAYNWKLAGDPRYEESLHNPYPEYHWLRTVKTTKESLIVISPSVADLDPYDKSLHSRIFPNGKCSGMTVSSTYCSTNHEYTIWMPEKVGLGTSCDIFTTSKGKRASKGGRTCGFVDERGLYKSLKGACKLKLCGVSGLRLMDGTWVAIQTSDETKWCSPDQLVNLHDLHSDEIEHLVVEELVRKREECLDALESIMTTKSVSFRRLSHLRKLVPGFGKAYTIFNKTLMEADAHYKSVRTWNEIIPSKGCLRVGGKCHPHVNGVFFNGIILGPDGHVLIPEMQSSLLQQHIELLESSVIPLMHPLADLS

SEQ ID NO: 110 - protein, 403 aa, Synthetic construct
KFPIYTIPDKLGPWSPIDIHHLSCPNNLVVEDEGCTNLSGFSYMELKVGYISAIKVNGFTCTGVVTEAETYTNFVGYVTTTFKRKHFRPTPDACRSAYNWKMAGDPRYEESLHNPYPDYHWLRTVKTTKESVVIISPSVADLDPYDKSLHSRVFPRGKCSGITVSSAYCSTNHDYTIWMPENPRLGTSCDIFTNSRGKRASKGSKTCGFVDERGLYKSLKGACKLKLCGVLGLRLMDGTWVAIQTSNETKWCPPDQLVNLHDFHSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGHVLIPEMQSSLLQQHMELLKSSVIPLMHPLADPS

SEQ ID NO: 111 - protein, 403 aa, Synthetic construct
KFPIYTIPDKLGPWSPIDIHHLSCPNNLVVEDEGCTNLSGFSYMELKVGYISAIKVNGFTCTGVVTEAETYTNFVGYVTTTFKRKHFRPMPDACRAAYNWKMAGDPRYEESLHNPYPDYHWLRTVKTSKESLVIISPSVADLDPYDKSLHSRVFPGGKCSGITVSSTCCSTNHDYTIWMPENPRLGTSCDIFTNSRGKRASKGGKTCGFVDERGLYKSLKGACKMKLCGVLGLRLMDGTWVAIQTSDEIKWCSPDQLVNLHDFHSDEIEHLVVEELVKKREECLDALETIMTTKSVSFRRLSHLRKLVPGFGKAYTIFNKTLMEADAHYKSIRTWNEIIPSKGCLRVGGRCHPHVNGVFFNGIILGPDGHVLIPEMQSSLLHQHMELLESSVIPLMHPLADPS

SEQ ID NO: 112 - protein, 403 aa, Synthetic construct
KFPIYTIPDKLGPWSPIDIHHLSCPNNLVVEDEGCTNLSGFSYMELKVGYISAIKVNGFTCTGVVTEAETYTNFVGYVTTTFKRKHFRPTPDECRAAYNWKMAGDPRYEESLHNPYPDYHWLRTVKTTKESLVIISPSVADLDPYDKSLHSRVFPSGKCSGITVSSTYCSTNHDYTIWMPENPRLGTSCDIFTNSRGKRASKGSKTCGFVDERGLYKSLKGACKLKLCGVLGLKLMDGTWVAMQTSDETKWCPPDQLVNLHDLRSDEIEHLVVEELVKKREECLDALESIMTTKSVSFRRLSHLRKLVPGFGKAYTIFNKTLMEADAHYKSVRTWNEIIPSKGCLKVEGRCHPHVNGVFFNGVILGPDGHVLIPEMQSSLLQQHMELLESSVIPLMHPLADPS

SEQ ID NO: 113 - protein, 403 aa, Synthetic construct
KFPIYTIPDKLGPWSPIDIHHLSCPNNLVVEDEGCNSLSGFSYMELKVGYISAIKVNGFTCTGVVTEAETYTNFVGYVTTTFKRKHFCPMPDACRSAYNWKMAGDPRYEESLHNPYPDYHWLRTVKTTKESLIIISPSVAELDPYDKSLHSRIFPGGKCTGLTVSSTYCSTNHDYTIWMPEKAGLGTSCDIFTNSKGKRASKGGKTCGFVDERGLYKSLKGACKLKLCGVSGLRLMDGTWVAIQTLDETKWCSPDQLVNLHDFHSDELEHLVVEELVRKREECLDALESIMTTKSVSFRRLSHLRKLVPGFGKAYTIFNKTLMEADAHYKSVRTWSEIIPSKGCLRVGGRCHPHVNGVFFNGIILGPDGHVLIPEMQSSLLQQHVELLESSVIPLMHPLADPS

SEQ ID NO: 114 - protein, 403 aa, Synthetic construct
KFPIYTIPDKLGPWSPIDIHHLSCPNNLVVEDEGCTSLSGFSYMELKVGYISAIKVNGFTCTGVVTEAETYTNFVGYVTTTFKRKHFRPMSDACRAAYNWKVAGDPRYEESLQNPYPDYHWLRTVKTTKESLIIISPSVADLDPYDKSLRSRIFPGGKCLGITVSSTYCSTNHDYTIWMPEEARLGTSCDIFTNSKGKRASKGGKICGFVDERGLYKSLKGACRLKLCGVSGLRLMDGTWVAIQTSEETKWCPSDQLVNLHDFHSDEIEHLVVEELVKKREECLDTLESIMTTKSVSFRRLSHLRKLVPGFGKAYTIFNKTLMEADAHYNSVRTWNEIIPSKGCLKVGGRCHPHVDGVFFNGIILGPDGNVLIPEMQSSLLQQHMELLESSVIPLMHPLADPS

SEQ ID NO: 115 - protein, 403 aa, Synthetic construct
KFPIYTIPDKLGPWSPIDIHHLSCPNNLVVEDEGCTNLSGFSYMELKVGYISAIKVNGFTCTGVVTEAETYTNFVGYVTTTFRRKHFRPTPDACRTAYNWKMAGDPRYEESLHNPYPDYHWLRTVKTTKESLVIISPSVADLDPYDRSLHSRVFPNGKCSGITVSSTYCPTNHDYTIWMPENPRLGTSCDIFVNSRGKRASKGSKTCGFVDERGLYKSLKGACKLKLCGVLGLRLMDGTWVAMRTSDETKWCPPDQLVNIHDFHSDEIEHLVVEELVKKREECLDALESIMTTKSVSFRRLSHLRKLVPGFGKAYTIFNKTLMEADAHYKSVRTWDEIIPSKGCLRVGGRCHPHVHGVFFNGIILGPDGHVLIPEMQSSLLQQHMELLESSVIPLMHPLADPS

SEQ ID NO: 116 - protein, 403 aa, Synthetic construct
KFPIYTIPDKLGPWSPIDIHHLSCPNNLVVEDEECTSLSGFSYMELKVGYISAIKVNGFTCTGVVTEAETYTNFVGYVTTTFKRKHFRPTPDACRAAYNWKMAGDPRYEESLHNPYPDYHWLRTVKTTKESLIIISPSVADLDPYDKSLHSRVFPGGKCLGITISSTYCSTNHDYTIWMPEEARLGTSCDIFANSRGKRASKGGRTCGFVDERGLYKSLKGACKLKLCGVPGLRLMDGTWVTIQTSNETKWCPPDRLVNLHDFHSDEIEHLVVEELVKKREECLDALESIMTTKSVSFRRLSYLRKLVPGFGKAYTIFNKTLMEADAHYKSVRTWNEIIPSKGCLKVGGRCHPHVDGVFFNGIILGPDGNVLIPEMQSSLLQQHMELLESSVIPLMHPLADPS

SEQ ID NO: 117 - protein, 403 aa, Synthetic construct
GFPIYTIPDKLGPWSPIDIHHLSCPNNLVVEDEGCTSLSGFSYMELKVGYISAIKVNGFTCTGVVTEAETYTNFVGYVTTTFKRKHFRPMPDSCRAAYNWKTAGDPRYEESLQNPYPDYHWLRTVKTTKESLIIISPSVADLDPYDKSLHSRVFPGGKCLGITVSSTYCSTNHDYTIWMPEEARLGTSCDIFTNSRGKRASKGGRTCGFVDERGLYKSLKGACKLKLCGVLGLRLMDGTWVTIHTSNETKWCPSDQLVNLHDFHSDEIEHLVVEELVKKREECLDALESIMTTKSVSFRRLSHLRKLVPGFGKAYTIFNKTLMEADAHYKSIRTWNEIIPSKGCLKVGGRCHPHVDGVFFNGIILGPDGNVLIPEMQSSLLQQHVELLESSVIPLMHPLADPS

SEQ ID NO: 118 - protein, 403 aa, Synthetic construct
KFPIYPIPDKLGPWSPIDIHHLSCPNNLVGEDEGCISLSGFSYMELKVGYISAIKVNGFTCTGVVTEAETYTNFVGYVTTTFKRKHFRPIPDACRAAYNWKIAGDPRYEESLQNPYPDYHWLRTVKTTKESLIIISPSVADLDPYDKSLHSRLFPGGKCLGITISSTYCSTNHDYTIWMPEEPRLGTSCDIFTNSKGKKASKGGKVCGFVDERGLYKSLKGACKLKLCGVSGLRLMDGTWVTIQTSDETKWCPPDQLVNLHDFHSDEIEHLVVEELVKKREECLDALESIMTTKSVSFRRLSYLRKLVPGFGKAYTIFNKTLMEADAHYKSVRTWNEIIPSKGCLKVSDRCHPHVDGVFFNGIILGPDGNVLIPEMQSSLLQQHMELLESSVIPLMHPLADPS

SEQ ID NO: 119 - protein, 403 aa, Synthetic construct
KFPIYTIPDKLGPWSPIDIHHLSCPNNLVVEDEGCTSLSGFSYMELKVGYISAIKVNGFTCTGVVTEAETYTNFVGYVTTTFKRKHFRPMPDACRAAHDWKIAGDPRYEDSLQNPYPDYHWLRTVKTTKESLVIISPSVAELDPYDKSLHSRVFPSGKCLGITVSSTYCSTNHDYTIWMPVEARLGTSCDIFTNSRGKKASKGGRTCGFVDERGLYKSLKGACKLKLCGVPGLRLMDGTWVSIQTSDDIKWCPPDQLVNLHDFHSDEIEHLVVEELIKKREGCLDALESIMTTKSVSFRRLSHLRKLVPGFGKAYTIFNNTLMEADAHYKSVRTWNEVIPSKGCLKVGGRCHPPVNGVFFNGIILGPDGKVLIPEMQSSLLQQHMELLESSVIPLMHPLADPS

SEQ ID NO: 120 - protein, 403 aa, Synthetic construct
KFPIYTIPEKLGPWSPIDIHHLSCPNNLVVEDEGCTTLSGFSYIELKVGYISAIKVNGFTCTGVVTEAETYTNFVGYVTTTFKRKHFRPMPDACRAAYNWKIAGDPRYEESLHNPYPDYHWLRTVKTTKESLVIISPSVADLDPYDKSLHSRVFPGGKCLGITISSTYCSTNHDYTIWMPEEARLGTSCDIFTNSRGKRASKGGRTCGFVDERGLYKSLKGACKLKLCGVPGLRLMDGTWVSIQASDETKWCSPDQLVNVHDFHSDEIEHLVVEELVKRREECLDALESIVTTKSISFRRLSHLRKLVPGFGKAYTIFNKTLMEADAHYKSVRTWNEIIPSKGCLSVGGRCHPHVNGVFFNGIILGPDGNVLIPEMQSSLLQQHMELLESSVIPLMHPLADPS

SEQ ID NO: 121 - protein, 403 aa, Synthetic construct
KFPIYTIPEKLGPWSPIDIHHLGCPNNLVVEDEGCDSLSGFSYMELKVGYISAIKVNGFTCTGVVTEAETYTNFVGYVTTTFKRKHFRPMPDACRAAYNWKTAGDPRYEESLHNPYPDYHWLRTVKTTKESLIIISPSVADLDPYDKSLHSRVFPGGKCLGITVSSTYCSTNHDYTIWMPEETRLGTSCDIFTNSRGKRASKGGKTCGFVDERGLYKSLKGACKLKLCGVPGLRLMDGTWVSIQTPDETKWCSPDQLVNLHDFHSDEIEHLVVEELVKKREECLDALESIMTTKSVSFRRLSHLRKLVPGFGKAYTIFNKTLMEADAHYKSVRTWDEIIPSKGCLRVGGRCHPHVNGVFFNGIILGPDGHVLIPEMQSSLLQQHMELLESSVIPLMHPVANPS

SEQ ID NO: 122 - protein, 403 aa, Synthetic construct
EFPIYTIPEKLGPWSPIDIHHLSCPNNLVVEDEGCNSLSGFSYMELKVGYISSIKVNGFTCTGVVTEAETYTNFVGYVTTTFKRKHFRPMPDACRAAYNWKMSGDPRYEESLHNPYPDYHWLRTVKTTKESLVIISPSVADLDPYDKSLHSRVFSGGKCLGITISSTYCSTNHDYTLWMPEEARLGTSCDIFTNSKGKRASKGGKTCGFVDERGLYKSLKGACKLKLCGVPGLRLMDGTWVSIQTSDETKWCPPDQLVNIHDFHSDEIEHLVVEELVKKREECLDALESIMTTKSVSFRRLSHLRKLVPGFGKAYTIFNKTLMEADAHYKSVRTWNEVIPSKGCLSVGGRCHPHVDGVFFNGIILGPDGSVLIPEMQSSLLQQHMELLESSVIPLMHPLANPS

SEQ ID NO: 123 - protein, 403 aa, Synthetic construct
KFPIYTIPDKIGPWSPIDIHHLSCPNNLVVEDEGCTSLSGFSYMELKVGYISAIKVNGFTCTGVVTEAETYTNFVGYVTTTFKRKHFRPTPDACRAAYNWKMAGDPRYEDSLQNPYPDYHWLRTVKTTKESLIIISPSVADLDPYDKSLHSRVFPSGKCLGITVSSTYCSTNHDYTIWMPEEARLGTSCDIFTNSKGKRASKGGKTCGFVDERGLYKSLRGACKLKLCGVPGLRLMDGTWVSIQTTDETKWCPSDQLVNLHDFHSDEIEHLVVEELVKKREECLDALESIMTTKSVSFRRLSHLRKLVPGFGKAYTIFNKTLMEADAHYKSVQTWNEIIPSKGCLKVGGRCHPHVDGVFFNGIILGPDGNVLIPEMQSSLLQQHMELLESSVIPLMHPLADPS

SEQ ID NO: 124 - protein, 403 aa, Synthetic construct
EFPIYTIPDKLGPWSPIDIHHLSCPNNLVAENDGCTSLSGFSYMELKVGYISAIKVNGFTCTGVMTEAETYTNFVGYVTTTFKRKHFRPMPDACRAAHDWKIAGDPRYEDSLQNPYPDYHWLRTVKTTKESLVIISPSVADLDPYDKSLHSRVFPSGKCLGITVSSTYCSTNHDYTIWMPVEPRLGTSCDIFTNSRGKRASKGGRVCGFVDERGLYKSLKGACKLKLCGVPGIRLMDGTWVSIQTSEEIKWCPPDRLVNLHDFHSDEIEHLVVEELIKRRENCLDALESIMTTKSVSFRRLSHLRKLVPGFGKAYTIFNKTLMEADAHYKSVKTWNEVIPSKGCLEVGGRCHPPVNGVFFNGIILGPDGNVLIPEMQSSLLQQHMELLESSVIPLMHPLADPS

SEQ ID NO: 125 - protein, 403 aa, Synthetic construct
KFPIYTIPDKLGPWSPIDIHHLSCPNNLVVEDEGCTSLSGFSYMELKVGYISAIKVNGFTCTGVVTEAETYTNFVGYVTTTFKRKHFRPMPDACRAAHEWKMAGDPRYEDSLQNPYPDYHWLRTVKTTKESLIIISPSVADLDPYDKSLHSRVFPGGNCLGITISSTYCSTNHDYTIWMPEEARLGTSCDIFTSSRGKRASKGGKICGFVDERGLYKSLKGACKLKLCGVPGLRLMDGTWVSIQTSDETKWCPPDQLVNVHDFHSDEIEHLIVEELVKKREECLDALESIMTTKSVSFRRLSHLRKLVPGFGKAYTIFNKTLMEADAHYKSVRTWNEIIPSKGCLKVGGMCHPHVGGVFFNGIILGPDGTVLIPEMQSSLLQQHMELLESSVIPLMHPLADPS

SEQ ID NO: 126 - protein, 403 aa, Synthetic construct
KFPIYTIPEKLGPWSPIDIHHLSCPNNLVVEDEGCDSLSGFSYMELKVGYISAIKVNGFTCTGVVTEAETYTNFVGYVTTTFKRKHFRPMPDACRAAYNWKTAGDPRYEESLHNPYPDYHWLRTVKTTKESLIIISPSVADLDPYDKSLHSRVFPGGKCLGITVSSTYCSTNHDYTIWMPEEARLGTSCDIFTNSKGKRASKGGRTCGFVDERGLYKSLKGACKLKLCGVPGLRLMDGTWVAIQTPGETKWCSPDQLVNLHDFRSDEIEHLVVEELIKKREECLDALESIMTTKSVSFRRLSNLRKLVPGFGKAYTIFNKTLMEADAHYKSVRTWDEIIPSKGCLRVGGRCHPHVNGVFFNGIILGPDGHVLIPEMQSSLLQQHMELLESSVIPLIHPLADPS

SEQ ID NO: 127 - protein, 403 aa, Synthetic construct
KFPIYTIPEKLGPWSPIDIHHLSCPNNLVVEDEGCNSLSGFSYMELKVGYISAIKVNGFTCTGVVTEAETYTNFVGYVTTTFKRKHFRPMPDACRAAYNWKTAGDPRYEESLHNPYPDYHWLRTVKTTKESLIIISPSVADLDPYDKSLHSRIFPGGKCLGITISSTYCSTNHDYTIWMPEEARLGTSCDIFTNSKGKRASKGGRTCGFVDERGLYKSLKGACKLKLCGVPGLRLMDGTWVAIQTPNETKWCSPDQLVNVHDFRSDEIEHLVVEELIRKREECLDALESIMTTKSVSFRRLSNLRKLVPGFGKAYTIFNKTLMEADAHYKSVRTWNEIIPSRGCLKVGGRCHPHVNGVFFNGIILGPDGHILIPEMQSSLLQQHMELLESSVIPLIHPLADPS

SEQ ID NO: 128 - protein, 403 aa, Synthetic construct
KFPIYTIPDKLGPWSPIDIHHLSCPNNLVVEDEGCNSLSGISYMELKVGYISAIKVNGFTCTGVVTEAETYTNFVGYVTTTFKRKHFHPMPDACRSAYNWKLAGDPRYEESLHNPYPDYHWLRTVKTTKESLIIISPSVADLDPYDKSLHSRIFPSGKCSGMTISSTYCSTNHEYTIWMPEKAGLGTSCDIFTTSKGKRASKGGKTCGFVDERGLYKSLKGACKLKLCGVSGLRLMDGTWVAIQTSEETKWCPPDQLVNLHDLHSDEIEHLVVEELVKKREECLDALESIMTTKSVSFRRLSHLRKLVPGFGKAYTIFNKTLMEADAHYKSIRTWNEIIPSKGCLRVGGKCHPHVDGVFFNGIILGPDGHVLIPEMQSSLLQQHMELLESSVIPLMHPLADLS

SEQ ID NO: 129 - protein, 403 aa, Synthetic construct
KFPIYTIPDKLGPWSPIDIHHLSCPNNLVVEDEGCNSLSGFSYMELKVGYISAIKVNGFTCTGVVTEAETYTNFVGYVTTTFKRKHFRPVPDACRSAYNWKLAGDPRYEESLHNPYPDYHWLRTVKTTKESLIIISPSVADLDPYDKSLHSRIFPDGKCSGITVSSTHCSTNHDYTIWMPEKAGLGTSCDIFINSRGKRASKGGRTCGFVDERGLYKSLKGACKLKLCGVPGLRLMDGTWAAIQTADETKWCSPDQLVNLHDFHSDEIEHLVVEDLVKKREECLDALESIMTTKSVSFRRLSHLRKLVPGFGKAYTIFNKTLMEADAHYKSVRTWNEIIPSKGCLRVGGRCHPHVNGVFFNGIILGPDGHVLIPEMQSSFLQQHMELLESSVIPLMHPLADPS

SEQ ID NO: 130 - protein, 403 aa, Synthetic construct
KFPIYTIPDKLGPWSPIDIHHLSCPNNLVVEDEGCTNLSGFSYMELKVGYISAIKVNGFTCTGVVTEAETYTNFVGYVTTTFRRKHFRPTPDACRAAYNWKMAGDPRYEESLHNPYPDYHWLRTVKTTKESLVIISPSVADLDPYDKSLHSRVFPGGKCSGITVSSTYCSTNHDYTIWMPENPRLGTSCDIFVNSRGKRASKGGKTCGFVDERGLYKSLKGACKLKLCGVPGLRLMDGTWVAIQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGHVLIPEMQSSLLQQHMELLESSVIPLMHPLADPS

SEQ ID NO: 131 - protein, 403 aa, Synthetic construct
KFPIYTIPDKLGPWSPIDIHHLSCPNNLVAEDEGCTTLSGFSYMELKVGYISAIKVNGFTCTGVVTEAETYTNFIGYVTTTFKRKHFSPRPDACRTAYNWKITGDPRYEESLQNPYPDYSWLRTVKTTKESLVIISPSVADLDPYDKSLHSRVFPGGRCLGITIYSTYCSTNHDYTVWLPEEARLGTSCDIFVKSRGKRASKGGRTCGFVDERGLYRSLKGACKLKLCGVPGLRLVDGTWVTIQTSDETKWCPSEQLVNLHNFHSDEIEHLVVEELVKKREECLDALESIMTTKSVSFRRLSHLRKLVPGFGKAYTIFNKTLMEADAHYTSVRTWNEIIPSKGCLKVGGKCHPHVGGVFFNGIILGPDGNILIPEMQSSLLQQHMELLESSVIPLMHPLADPS

SEQ ID NO: 132 - protein, 403 aa, Synthetic construct
KFPIYTIPDKLGPWSPIDIHHLSCPNNLVVEDEGCNNLSGFSYMELKVGYISAIKVNGFTCTGVVTEAETYTNFVGYVTTTFKRKHFRPTPDACRAAYNWKIAGDPRYEESLHNPYPDYHWLRTVKTTKESLVIISPSVADLDPYDKSLHSRVFPSGKCSGMTVSSTCCSTNHDYTIWMPENPRLGTSCDIFTNSRGKRASKGSKTCGFVDERGLYKSLKGACKLKLCGVLGLRLMDGTWVAMQTSDEIKWCPPDQLVNLHDFRSDEIEHLVVEELVKKREECLDALESIMTTKSVSFRRLSHLRKLVPGFGKAYTIFNRTLMEADAHYKSVRTWNEVIPSKGCLRVGGRCHPHVNGVFFNGIILGPDGHVLIPEMQSSLLQQHMELLESSVIPLMHPLANPS

SEQ ID NO: 133 - protein, 403 aa, Synthetic construct
KFPIYTIPDKLGPWSPIDIHHLSCPNNLVIEDEGCNNLSGFSYMELKVGYISAIKVNGFTCTGVVTEAETYTNFVGYVTTTFKRKHFRPMPDACRSAYNWKLAGDPRYEESLHNPYPDYHWLRTVKTTKESLIVISPSVADLDPYDKSLHSRIFPGGKCSGMTVSSTYCSTNHEYTIWMPEKPGLGTSCDIFATSKGKRASKGGRTCGFVDERGLYKSLKGACKLKLCGVSGLRLMDGTWVAIQTSDETKWCSPDQLVNLHDLHSDEIEHLVVEELVRKREECLDALESIMTTKSVSFRRLSHLRKLVPGFGKAYTIFNKTLMEADAHYKSVRTWNEIIPSKGCLRVGGKCHPPVNGVFFNGIILGPDGHVLIPEMQSSLLQQHMELLESSVIPLMHPLADPS

SEQ ID NO: 134 - protein, 403 aa, Synthetic construct
KFPIYTIPDKIGPWSPIDIHHLSCPNNLVVEDEGCTSLSGFSYMELKVGYISAIKVNGFTCTGVVTEAETYTNFVGYVTTTFKRKHFRPMPDACRAAYNWKMAGDPRYEESLQNPYPDYNWLRTVKTTKESLIIISPSVADLDPYDKSLHSRVFPNGKCLGITVSSTYCSTNHDYTIWMPEEARLGTSCDIFTNSKGKRASKGGKTCGFVDERGLYKSLRGACKLKLCGVPGLRLMDGTWVSIQTSDETKWCPPDQLVNVHDFHSDEIEHLVVEELVRKREECLDALESIMTTKSVSFRRLSHLRKLVPGFGKAYTIFNKTLMEADAHYKSIQTWNEIIPSKGCLKVGGMCHPHLDGVFFNGIILGPDGNVLIPEMQSSLLQQHMELLESSVIPLMHPLADPS

SEQ ID NO: 135 - protein, 403 aa, Synthetic construct
KFPLYTIPDKLGPWSPIDIHHLSCPNNLVVEDEGCTNLSGFSYMELKVGYISAIKVSGFTCTGVVTEAETYTNFVGYVTTTFKRRHFRPTPDACRAAYNWKMAGDPRYEESLHNPYPDYHWLRTVKTTKESLVIISPSVTDLDPYDKSLHSRVFPGGKCSGITVSSTYCSTNHDYTIWMPESPRLGTSCDIFTNSRGKRASKGSKTCGFVDERGLYKSLKGACKLKLCGVLGLRLMDGTWVAMQTSDETKWCLPDQLVNLHDFRSDEIEHLVVEELVRKREECLDALESIMTTKSVSFRRLSHLRKLVPGFGKAYTIFNKTLMEADAHYKSIRTWNEIVPSKGCLRVGGRCHPHVNGVFFNGIILGPDGQVLIPEMQSSLLQQHMELLESSVIPLMHPLADPS

SEQ ID NO: 136 - protein, 403 aa, Synthetic construct
KFPIYTIPDKLGPWSPIDIHHLSCPNNLVVEDEGCTNLSGFSYMELKVGYISAIKVNGFTCTGVVTEAETYTNFVGYVTTTFKRKHLRPAPDACRNAFNWKMAGDPRYEESLHNPYPDYHWLRTVKTTKESLVIISPSVADLDPYDKSLHSRVFPSGKCSGITVSSTYCSTNHDYTVWMPENPRLGTSCDIFTNSKGKRASKGSRTCGFVDERGLYKSLKGACKLKLCGVPGLRLTDGTWVAMQTSEETKWCPHDQLVNLHDFRLDEIEHLVVEELVKKREECLDALESIMTTKSVSFRRLSHLRKLVPGFGKAYTIFNKTLMEADAHYKSVRTWNEIIPSKGCLKVGGSCHPHVNGVFFNGIILGPDGHVLIPEMQSSLLQQHMELLESSVIPLMHPLADPS

SEQ ID NO: 137 - protein, 403 aa, Synthetic construct
KFPIYTIPDKLGPWSPIDIHHLSCPNNLVVEDEGCTNLSGFSYMELKVGYISAIKVNGFTCTGVVTEAETYTNFVGYVTTTFKRKHFRPTPDACRAAYNWKMAGDPRYEESLHNPYPDYHWLRTVKTTKESLVVISPSVADLDPYDKSLHSRVFPSGKCSGITVSSTYCSTNHDYTIWMPENPRLGTSCDIFVNSRGKRASKGSKTCGFVDERGLYKSLKGACKLKLCGVLGLRLMDGTWVAIQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGHVLIPEMQSSLLQQHMELLESSVIPLMHPLADPS

SEQ ID NO: 138 - protein, 403 aa, Synthetic construct
KFPIYTIPEKLGPWSPIDIHHLSCPNNLVAEDEGCNSLSGFSYMELKVGYISAIKVNGFTCTGVVTEAETYTNFVGYVTTTFKRKHFRPTPDACRAAYNWKMAGDPRYEESLHNPYPDYHWLRTVKTTKESLVIISPSVADLDPYDKSLHSRVFPDGKCLGITISSTYCSTNHDYTIWMPEEARLGTSCDIFTTSKGKRASKGDKICGFVDERGLYKSLKGACKLKLCGVLGLRLMDGTWVSIQTPGETKWCSPDQLVNLHDFHSDEIEHLVAEELVKKREECLDALESIMTTKSVSFRRLSHLRKLVPGFGKAYTIFNKTLMEADAHYKSVRTWNEVIPSKGCLSVGGRCHPHVNGVFFNGIILGPDGSVLIPEMQSSLLQQHMELLESSVIPLMHPLADPS

SEQ ID NO: 139 - protein, 403 aa, Synthetic construct
KFPIYTIPEKLGPWSPIDIHHLSCPNNLVVEDEGCNSLSGFSYMELKVGYISAIKVNGFTCTGVVTEAETYTNFVGYVTTTFKRKHFRPMPDACRAAYNWKMAGDPRYEESLHNPYPDYHWLRTVKTTKESLVIISPSVADLDPYDKSLHSRVFPGGKCLGITISSTYCSTNHDYTIWMPEEARLGTSCDIFTNSKGKRASKGGRTCGFVDERGLYKSLKGACKLKLCGVPGLRLMDGTWVSIQTSEETKWCSSDQLVNIHDFRSDEIEHLVVEELVKKREECLDALESIMTTKSVSFRRLSHLRKLVPGFGKAYTIFNNTLMEADAHYKSVKTWNEIIPSKGCLSVGGRCHPNVNGVFFNGIILGPDGSVLIPEMQSSLLQQHMELLESSVIPLMHPLANPS

SEQ ID NO: 140 - protein, 403 aa, Synthetic construct
EFPIYTIPDKLGPWSPIDIHHLSCPNNLVVEDEGCTNLSGFSYMELKVGYISAIKVNGFTCTGVVTEAETYTNFVGYVTTTFKRKHFRPTPDACRAAYNWKMAGDPRYEESLHNPYPDYHWLRTVKTTKESLVIISPSVADLDPYDKSLHSRVFPSGKCSGITVSSTYCLTNHDYTIWMPENLRLGTSCDIFTNSRGKRASRGSKTCGFVDERGLYKSLKGACKLKLCGVLGLRLMDGTWVAMQTSDETKWCPSDQLVNLHDFRSDEIEHLVVEELVKKREECLDAIESIMTTKSVSFRRLSHLRKLVPGFGKAYTIFNKTLMEADAHYKSVRTWNEIIPSKGCLRVGGRCHPHVNGVFFNGIILGPDGHVLIPEMQSSLLQQHVELLESSVIPLMHPLADPS

SEQ ID NO: 141 - protein, 403 aa, Synthetic construct
KFPIYTIPDKLGPWSPIDIHHLSCPNNLVVEDEGCSNLSGFSYMELKVGYISAIKVDGFTCTGVVTEAETYTNFIGYVTTTFKRKHFRPMPDACRSAYNWKIAGDPRYEESLHTPYPDYHWLRTVKTTKESLVIISPSVADLDPYDKSLHSMVFPNGKCSGITVYSTYCSTNHDYTIWMPEKAGLGTSCDIFTNSKGKRASKGGRTCGFVDERGLYKSLRGACKLKLCGVPGLRLMDGTWATIQTPEETKWCSPDQLVNLHDFHSDEIEHLVVEDLVKKREECLDTLESIMTTKSVSFRRLSHLRKLVPGFGKAYTIFNKTLMEADAHYKSVRTWNEIIPSKGCLRVGGRCHPHVNGVFFNGVIMGPDGHVLIPEMQSSLLQQHMELLESSVIPLIHPLADPS

SEQ ID NO: 142 - protein, 403 aa, Synthetic construct
DFPIYTIPEKLGPWSPIDIHHLSCPNNLVVEDEGCSSLSGFSYMELKVGYISAIKVNGFTCTGVVTEAETYTNFVGYVTTTFKRKHFRPMPDACRAAYNWKTAGDPRYEESLQNPYPDYHWLRTVKTTKESLVIISPSVADLDPYDKSLHSRVFSDGKCLGITISSTYCSTNHDYTIWMPEEARLGTSCDIFTTSRGKRASKGGKTCGFVDERGLYKSLKGACKLKLCGVSGLRLMDGTWVSIQAPDEAKWCSTDQLVNIHDIRSDEIEHLVVEELVKKREECLDALESIMTTKSVSFRRLSHLRKLVPGFGKAYTIFNKTLMEADAHYKSVRTWSEVIPSKGCLSVGGRCHPHENGVFFNGIILGPDGSVLIPEMQSSLLQQHMELLESSVIPLMHPLANPS

SEQ ID NO: 143 - protein, 403 aa, Synthetic construct
KFPIYTIPDKLGPWSPIDIHHLSCPNNLVVEDEGCTNLSGFSYMELKVGYISAIKVNGFTCTGVVTEAETYTNFIGYVTTVFKRKHSRPTPDACRAAYNWKMAGDPRYEESLHNPYPDYHWLRTVKTTKESLVIISPSVADLDPYDKSLHSRVFPSGKCSGITVSSTYCLTNHEYTIWMPENLRLGTSCDIFTNSRGKRASRGSKTCGFVDERGLYKSLKGACKLKLCGVLGLRLMDGTWVAMQTSDETKWCPPDQLVNLHDFRSDEIEHLVVEELVKKREECLDAIESIMTTKSVSFRRLSHLRKLVPGFGKAYTIFNKTLMEADAHYKSVRTWNEIIPSKGCLRVGGRCHPHVKGVFFNGIILGPDGHVLIPEMQSSLLQQHMELLESSIIPLMHPLADPS

SEQ ID NO: 144 - protein, 403 aa, Synthetic construct
KFPIYTIPDKLGPWSPIDIHHLSCPNNLVVEDEGCTNLSGFSYMELKVGYISAIKVNGFTCTGVVTEAETYTNFVGYVTTTFKRKHFRPTPDACRAAYNWKMAGDPRYEESLHNPYPDYHWLRTVKTTKESLVIISPSVSDLDPYDKSLHSRVFPSGKCSGITVSSTYCPTNHDYTIWMPENPRLGTSCDIFTNSRGKRASKGSKTCGFVDERGLYKSLKGACKLKLCGVLGLRLMDGTWAAIQTSDEAKWCPPDQLVNIHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGHVLIPEMQSSLLQQHMELLESSVIPLMLPLADPS

SEQ ID NO: 145 - protein, 403 aa, Synthetic construct
KFPIYTIPDKLGPWSPIDIHHLSCPNNLVVEDEGCTSLSGFSYMELKVGYISAIKVNGFTCTGVVTEAETYTNFVGYVTTTFKRKHFRPTPDACRAAYNWKVAGDPRYEESLQNPYPDYHWLRTVKTTKESLIIISPSVADLDPYDKSLHSRVFPSGKCLGITISSTYCSTNHDYTIWMPEEARLGTSCDIFTNSKGKKASKGGRTCGFVDERGLYKSLKGACKLKLCGVPGLRLMDGTWVTIQTSDETKWCPPDQLVNLHDFHSDEIEHLVVEELVKKREECLDALESIMTTKSVSFRRLSHLRKLVPGFGKAYTIFNKTLMEADAHYKSVRTWNEIIPSKGCLKVGGRCHPHVDGVFFNGIILGPDGNVLIPEMQSSLLQQHMELLESSVIPLMHPLADPS

SEQ ID NO: 146 - protein, 403 aa, Synthetic construct
KFPIYTIPDKLGPWSPIDIHHLSCPNNLVVEDEGCTNLSGFSYMELKVGYISAIKVNGFTCTGVVTEAETYTNFVGYVTTTFKRKHLRPTPDACRAAYNWKMAGDPRYEESLHNPYPDYHWLRTVKTTKESLVIISPSVADLDPYDKSLHSRVFPSGKCSGMTVSSTYCSTNHDYTIWMPENPRLGTSCDIFTNSKGKRASKGSKICGFVDERGLYKSLKGACKLKLCGVTGLRLMDGTWVAMQTSDETKWCPPDQLVNLHDFRSDEIEHLVVEELVKKREECLDALESIMITKSVSFRRLSHLRKLVPGFGKAYTIFNKTLMEADAHYKSVRTWNEIIPSKGCLRVGGRCHPHVNGVFFNGIILGPDGHVLIPEMQSSLLQQHMELLESSVIPLMHPLADPS

SEQ ID NO: 147 - protein, 403 aa, Synthetic construct
KFPIYTIPDKLGPWSPIDIHHLSCPNNLVVEDEGCTNLSGFSYMELKVGYISAIKVNGFTCTGVVTEAETYTNFVGYVTTTFRRKHFRPTPDACRSAYNWKMAGDPRYEESLHNPYPDYHWLRTVKTTKESLVIISPSVADLDPYDKSLHSRVFPNGKCSGLTVSSTYCSTNHDYTIWMPENPRLGTSCDIFTNSRGKRASRGNKTCGFVDERGLYKSLKGACKLKLCGVLGLRLIDGTWVAMHTSDETKWCPPDQLVNLHDFRSDEIEHLVVEELVKKREECLDALESIMTTKSVSFRRLSHLRKLVPGFGKAYTIFNKTLMEADAHYKSIRTWDEIVPSKGCLRVGGRCHPHVNGVFFNGIILGSDGHVLIPEMQSSLLQQHVELLESSVVPLMHPLADPS

SEQ ID NO: 148 - protein, 403 aa, Synthetic construct
KFPIYTIPDKLGPWSPIDIHHLSCPNNLVIEDEGCTNLSGFSYMELKVGYISAIKVNGFTCTGVVTEAETYTNFVGYVTTTFRRKHFRPTSDACRAAYNWKMAGDPRYEESLHNPYPDYHWLRTVRTTKESLVIISPSVADLDPYDKSLHSRVFPNGRCSGLTVSSTFCSTNHDYTIWMPENPRLGTSCDIFTNSRGKRASKGNRTCGFVDERGLYKSLKGACKLKLCGVLGLRLMDGTWVAMQTSEETKWCPPDQLVNLHDFRSDEIEHLVVEELVKKREECLDALESIMTTKSVSFRRLSHLRKLVPGFGKAYTIFNKTLMEADAHYKSIRTWNEIIPSKGCLRVGGRCHPHVNGVFFNGIILGSDGHVLIPEMQSSLLQQHVELLESSVVPLMHPLADPS

SEQ ID NO: 149 - protein, 403 aa, Synthetic construct
KFPIYTIPDKLGPWSPIDIHHLSCPNNLVVEDEGCTNLSEFSYVELKVGYILAIKMNGFTCTGVVTEAETYTNFVGYVTTTFKRKHFRPTPDACRAAYNWKMAGDPRYEESLHNPYPDYHWLRTVRTTKESLVIISPSVADLDPYDRSLHSRVFPSGKCSGVAVSSTYCSTNHDYTIWMPENPRLGMSCDIFTKSRGKRASKGSETCGFVDERGLYKSLKGACKLKLCGVPGLRLMDGTWVAMQTSNETKWCPPDQLVNLHDFRSDEIEHLVVEELVRKREECLDALESIMTTKSVSFRRLSLLRKLVPGFGKAYTIFNKTLMEADAHYKSVRTWNEILPSKGCLRVGGRCHPHVNGVFFNGIILGPDGNVLIPEMQSSLLQQHMELLESSVIPLVHPLADPS

SEQ ID NO: 150 - protein, 403 aa, Synthetic construct
KFPIYTIPDKLGPWSPIDIHHLSCPNNLVLEDEGCTNLSGFSYMELKVGYISAIKVSGFTCTGVVTEAETYTNFVGYVTTTFKRKHFRPTPDACRAAYNWKIAGDPRYEESLHNPYPDYHWLRTVKTTKESLVIISPSVADLDPYDKSLHSRVFSREKCLGMTVSSTYCSTNHDYTIWMPENPRLGTSCDIFTNSKGKRASRGSKTCGFVDERGLYKSLKGACKLKLCGVLGLRLVDGTWIAMQTSDEAKWCPSDQLVNLHDFRSDEIEHLVVEELVKKREECLDALESIMTTKSVSFRRLSHLRKLVPGFGKAYTIFNKTLMEADAHYKSIRTWNEIIPSKGCLKVGGRCHPHVNGVFFNGIILGPDGHVLIPEMQSSLLQQHMELLESSVIPLMHPLADPS

SEQ ID NO: 151 - protein, 403 aa, Synthetic construct
KFPIYTIPDKLGPWSPIDIHHLSCPNNLIVEDEGCSSLSGFSYMELKVGYISAIKVNGFTCTGVVTEAETYTNFVGYVTTTFKRKHFRPMPDACRAAYNWKMAGDPRYEESLHNPYPDYHWLRTVKTTKESLIIISPSVADLDPYDKSLHSRVFPGGKCLGITISSTYCSTNHDYTIWMPEEPRLRTSCDIFTNSKGKRASKGGKMCGFVDERGLYKSLKGACKLKLCGVPGLRLMDGTWVSVQTPDETKWCSPDQLVNLHDFHSDEIEHLVVEELVKKREECLDTLESIMTTKSVSFRRLSHLRKLVPGFGKAYTIFNKTLMEADAHYKSIQTWNEVIPSKGCLKVGGRCHPHVNGVFLNGIILGPDGNVLIPEMQSSLLQQHMELLESSVIPLMHPLSDPS

SEQ ID NO: 152 - protein, 403 aa, Synthetic construct
KFPIYTIPDKLGPWSPIDIHHLSCPNNLIVEDEGCTSLSGLSYMELKVGYISAIKVNGFTCTGVVTEAETYTNFVGYVTTTFKRKHFRPMPDACRAAYNWKIAGDPRYEESLQNPYPDSHWLRTVKTTKESLIIISPSVADLDPYDKSLRSRVFPGGKCLGITISSTYCSTNHDYTIWMPEEARLGTSCDIFTNSKGKRASKGGKICGFVDERGLYKSLKGACKLKLCGVSGLRLMDGTWVSIQIPDEIKWCPSDQLVNLHDFHSDEIEHLVVEELVKKREECLDALESIMTTKSVSFRRLSHLRKLVPGFGKAYTIFNKTLMEADAHYNSVRTWNEIIPSKGCLKVGGRCHPHVDGVFFNGIIMGPDGSVLIPEMQSSLLQQHMELLESSVIPLMHPLADPS

SEQ ID NO: 153 - protein, 403 aa, Synthetic construct
KFPVYTIPEKLGPWSPIDIHHLSCPNNLAVEDEGCNSLSGFSYMELKVGYISAIKVNGFTCTGVVTEAETYTNFVGYVTTTFKRKHFRPMPDACRAAYNWKMAGDPRYEESLHNPYPDYHWLRTVTTTKESLIIISPSVANLDPYDKSLHSRVFPGGKCMGITVSSTFCSTNHDYTIWMPENARLGTSCDIFTTSKGKKASKGGKICGFVDERGLYKSLKGACKLKLCGVSGLRLLDGTWVTIQTSDEIKWCPPDKLVNLHDFHSDEIEHLVVEELVRKREECLDTLESIMTTKSVSFRRLSHLRKLVPGFGKAYTIFNKTLMEADAHYKSVNTWNEIIPSKGCLTVGGRCHPHVNGVFFNGIILGPDGHVLIPEMQSSLLQQHMELLESSVIPLMHPLADPS

SEQ ID NO: 154 - protein, 403 aa, Synthetic construct
KFPIYTIPDKLGPWSPIDIHHLSCPNNLVVEDEGCTSLSGFSYMELKVGYISAIKVNGFTCTGVVTEAETYTNFVGYVTTTFKRKHFRPMPDACRAAHEWKTAGDPRYEDSLQNPYPDYHWLRTVKTTKESLVIISPSVADLDPYDKSLHSRVFPGGKCLGVTISSTYCSTNHDYTIWMPEEAGLGTSCDIFTNSRGKRASKGGRICGFVDERGLYKSLKGACKLKLCGVPGLRLMDGTWVSIQAPDETKWCPPDQVVNLHDFHSDEIEHLVVEELIKKREECLDALESIMTTKSVSFRRLSHLRKLVPGFGKAYTIFNKTLMEADAHYKSVRTWNEVVPSKGCLKVGGRCHSDVDGVFFNGIILGPDGNVLIPEMQSSLLQQHMELLESSVIPLMHPLADPS

SEQ ID NO: 155 - protein, 403 aa, Synthetic construct
KFPIYTIPDKLGPWSPIDIHHLSCPNNLVVEDEGCTSLSGFSYMELKVGYISAIKVNGFTCTGVVTEAETYTNFVGYVTTTFKRRHFRPMPDACRAAHNWKIAGDPRYEDSLQNPYPDYHWLRTVKTTKESLIIISPSVADLDPYDKSLHSRVFPGGKCLGVTISSTYCSTNHDYTIWMPEEAGLGTSCDIFTNSRGKRASKGGKTCGFVDERGLYKSLKGSCKLKLCGVPGLRLIDGTWVSIQTPDETKWCPPDQVVNLHDFHSDEIEHLVVEELIKKREECLDALESIMTTKSVSFRRLSHLRKLVPGFGKAYTIHNKTLMEADAHYKSVRTWNEIIPSKGCLRVGGRCHPNVDGVFFNGIILGPDGNVLIPEMQSSLLQQHMELLESSVIPLMHPLADPS

SEQ ID NO: 156 - protein, 403 aa, Synthetic construct
KFPIYTIPDKLGPWSPIDIHHLCCPNNLVAEEEGCTSLSGFSYMELKVGYISAIKVNGFTCTGVVTEAETYTNFVGYVTTTFKRKHFRPMPDACRAAYDWKMAGDPRYEDSLQNPYPDYHWLRTVKTTKESLIIISPSVADLDPYDKSLHSRVFPGGKCLGITISSTYCSTNHDYTIWMPEEARLGTSCNIFTNSRGKKASKGGRTCGFVDERGLYKSLKGACKLKLCGVPGLRLMDGTWVSIQTSNETKWCPPDQLVNLHDFHSDEIEHLVVEELVKKREECLDALESIMTTKSVSFRRLSHLRKLVPGFGKAYTIFNKTLMEADAHYKSVRTWNEIIPSKGCLKVGGRCHPHVDGVFFNGIILGPDGHVLIPEMQSSLLQQHMELLESSVIPLIHPLADPS

SEQ ID NO: 157 - protein, 403 aa, Synthetic construct
KFPIYTIPDKLGPWSPIDIHHLSCPNNLVVEDEGCTNLSGFSYMELKVGYISAIKVNGFTCTGVVTEAETYTNFVGYVTTTFKRKHFRPTPDACRAAYNWKMAGDPRYEESLHNPYPDYHWLRTVKTTKESLVIISPSVADLDPYDKSLHSRVFPSGKCLGITVSSTYCSTNHDYTVWRPENPRLGTSCDIFTNSRGKRASKGSKTCGFVDERGWYKSWKGACKLKLCGVLGLRLIDGTWIAMQTSEETKWCPPDQLVNLHDFRSDEIEHLVVEELVKKREECLDALESIMTTKSVSFRRLSHLRKLVPGFGKAYTIFNKTLMEADAHYKSIRTWNEIIPSKGCLRVGGKCHPHVNGVFFNGIILGPDGHVLIPEMQSSLLQQHMELLESSVIPLMHPLADPY

SEQ ID NO: 158 - protein, 403 aa, Synthetic construct
KFPIYTIPDKLGPWSPIDLHHLSCPNNLVVEDEGCTNLSGFSYMELKVGYISAIKVNGFTCTGVVTEAETYTNFVGYVTTTFKRKHFRPTPDACRAAYNWKMAGDPRYEESLHNPYPDYHWLRTVKTTKESLVIISPSVTDLDPYDKSLHSRVFPGGNCSGITVSSTYCSTNHDYTIWMPENLRLGTSCDIFTHSRGKRASKGDKTCGFVDERGLYKSLKGACKLKLCGVLGLRLMDGTWVAMQTSDETKWCPPGQLVNLHDFRSDEIEHLVVEELVKKREECLDALESIMTTKSVSFRRLSHLRKLVPGFGKAYTIFNKTLMEADAHYKSVQTWNEIIPSKGCLRVGERCHPHVNGVFFNGIILGSDGHVLIPEMQSSLLQQHMELLESSVIPLMHPLADPS

SEQ ID NO: 159 - protein, 403 aa, Synthetic construct
KFPIYTIPDKLGPWSPIDIHHLSCPNNLVVEDEGCTNLSGFSYLELKVGHISAIKVNGFTCTGVVTEAETYTNFVGYVTTTFKRKHFRPTPDACRAAYNWKTAGDPRYEESLQNPYPDYQWLRTVRTTKESLVIISPSAADLDPYDKSLHSRVFPSGKCSGITVSSVYCSTNHDYTIWMPENPRQGMSCDIFTNSRGKRASKERKTCGFVDERGLYKSLRGSCKLKLCGVLGLRLMDGTWVAMQTSNETKWCSPDQLVNLHDFHSDEIEHLVVEELVKKREECLDALESIMTTKSVSFRRLSHLRKLVPGFGKAYTIINKTLMEAEAHYKSVRTWNEIVPSKGCLRVEGRCHPHVNGVFFNGIILGPDGHVLIPEMQSSLLQQHMELLESSVIPLMHPLADPS

SEQ ID NO: 160 - protein, 403 aa, Synthetic construct
KFPIYTIPDKLGPWSPIDIHHLSCPNNLVVEDEGCTNLSGFSYMELKVGYISAIKVNGFTCTGVVTEAETYTNFVGYVTTTFRRKHFRPTPDACRAAYNWKMAGDPRYEESLHNPYPDYHWLRTVKTTKESLVIISPSVADLDPYDKSLHSRVFLNGKCSGLTVSSTYCSTNHDYSIWMPENPRLGTSCDIFVTSRGKRASKGSKTCGFVDERGLYKSLKGACKLKLCGVLGLRLIDGTWVAMQTSEEPKWCPPDQLVNLHNFRSDEIEHLVVEELVKKREECLDALESIMTTKSVSFRRLSHLRKLVPGFGKAYTIFNKTLMEADAHYKSIRTWDEIIPSKGCLRVGGRCHPHVNGVFFNGIILGSDGHVLIPEMQSSLLQQHVELLESSVIPLMHPLADPS

SEQ ID NO: 161 - protein, 403 aa, Synthetic construct
DFPLYTIPEKIGPWTPIDLTHLSCPNNLLSEDDGCSSSSTFSYIELRTGYLTHQKVSGFTCTGVINEAVTYTNFVGYVTTTFKRKHFKPTASACRDAYHWKISGDPRYEESLHTPYPDNSWLRTVTTTKESLLIISPSIVEMDIYSRSLHSPMFPTGRCYDFYKSTPSCLTNHDYTIWLPDDANVRLTCDIFVTSTGKKSMNGSKMCGFTDERGLYRTLKGACKLTLCGKPGLRLFDGTWISITRPEIVMWCSPNQLVNVHNNRVDEIEHLIVGDLIRRREECLDTLETVLMSKSVSFRRLSHFRKLVPGFGKAYTIANGSLMETNVHYKRVDRWEEILPSKGCLKLNDKCLNPENGVFFNGIIKGPDGQVLIPEMQSSLLKQHMDLLKASVFPLRHPLIDQT

SEQ ID NO: 162 - protein, 403 aa, Synthetic construct
KFPIYTIPDKLGPWSPIDIHHLSCPNNLVVEDEGCTTLTPFSYMELKVGYITSIKVSGFTCTGVVTEAETYTNFVGYVTTTFRRRHFRPSVNSCRDAYNWKIAGDPRYEESLHNPYPDSHWLRTVKTTKESLLIISPSVADMDAYDKKLYSKMFPNGKCSEISPGSPFCPTNHEYTIWMPESSNPGISCDIFTRSMGKKATKDGQLCGFVDERGLYKSLKGACRLRLCGISGLRLMDGSWVSLPQVNNSEWCSPDQLVNIHDFHSDEIEHLVADELVKKREDCLDALETIIFTKSISFRRLSHLRKLVPGFGKAYTIINRTLMEAEAHYKSVREWKEIIPSKGCLKAGGRCYPHHNGIFFNGIILGPGGEILIPEMQSALLQQHIELLESSVVPLKHPLADPS

SEQ ID NO: 163 - protein, 403 aa, Synthetic construct
DFPLYTIPEKIDSWTPIDMIHLSCPNNLLSGDDGCSTSTSFSYVELKTGYLTHQKVSGFTCTGVISEAVTYTNFVGYVTTTFKRKHFKPTVSACRDAFHWKISGDPRYEESLHTPYPDSSWLRTVTTTKESLLIISPSIVEMDIYSRTLHSPMFPTGTCSRFYPSVPSCATNHDYTLWLPEDSNLSLICDIFVTSTGRKAMNGSKMCGFTDERGFYRTLRGACKLTLCGKPGLRLYDGTWVSFTRPEVHVWCSPNQLVNVHNNRIDELEHLIVDDLIQKREACLDTLETILMSKSISFRRLSHFRKLVPGYGKAYTILNGSLMETNVHYRRVDNWTDILPSKGCLKVGNKCLDPHKGVYFNGIIKGPDGHILIPEMQSGMLKQHIDLLKAAVFPLRHPLIDQS

SEQ ID NO: 164 - protein, 403 aa, Synthetic construct
DFPLYTIPERLNPWTPIDLIHLSCPNNLLSDAEGCSETSSFTYVELKTGFLAHQKVPGFTCTGVINEAVTYTNFVGYVTTTFKRKHFKPTVSACRDAFNWKVSGDPRYEESLHTPYPDNSWLRTVTTTKESLLIISPSIVEMDVYGRTLHSPMFPTGICSKFYPSIPSCATNHDYTLWLPEDPNLSLICDIFVTSTGRKAMNGSRMCGFTDERGFYRTLKGACKLTLCGKPGLRLYDGTWVSFTRPEIHVWCSPDQLVNVHNNRIDEIEHLIVDDLIRKREECLDTLETVLMSKSLSFRRLSHFRKLVPGYGKAYTILNGSLMETNVHYKRVDNWVDILPSKGCLKVNNKCMESDTGVFFNGIIKGPDGRILIPEMQSGLLKQHMDLLKAAVFPLRHPLIDKA

SEQ ID NO: 165 - protein, 403 aa, Synthetic construct
EFPLYTIPEKIEKWTPIDMIHLSCPNNLLSEEEGCNTESPLTYFELKSGYLAHQKVPGFTCTGVVTEAETYTNFVGYVTTTFKRKHFGPTVAACRDAYNWKVSGDPRYEESLHTPYPDSSWLRTVTTTKESLLIISPSIVEMDVYGRTLHSPMFPSGICSKLYPSVPSCKTNHDYTLWLPEDPSLSLICDIFTSGSGRKAMNGSRICGFKDERGFYRSLKGACKLTLCGKPGIRLFDGTWVSFTRPEVHVWCTPNQLVNIHNDRLDEIEHLIVEDIVKRREECLDTLETIFMSQSISFRRLSHFRKLVPGYGKAYTILNGSLMEANVYYKRVDKWADILPSKGCLKVGQQCMDPVNGVFFNGIIKGPDGQILIPEMQSEQLKQHMDLLKAAVFPLRHPLISQE

SEQ ID NO: 166 - protein, 403 aa, Synthetic construct
DFPLYTIPEKIGTWTPIDLIHLTCPNNLLSGDDGCSDTATFGYIELKTGYLTHQKVSGFTCTGVVNEAVTYTNFVGYVTTTFKRKHFKPTALACRDAFHWKISGDPRYEESLHTPYPDNSWLRTVTTTKESLLIISPSIVEMDVYSRTLHSPMFPGGVCSKFYPSSPSCPTNHDYTLWLPEDANLSMACDIFVTSTGKKSMNGSRMCGFTDERGFYRTLKGACKLTLCGKPGLRLYDGTWVSFTRPEINVWCSPNQLVNVHNNRLDEIEHLIVGDLIRKREECLDTLETILMSKSISFRRLSHFRKLVPGYGKAYTIINGSLMETNVHYLRVDSWNDILPSKGCLKMNKQCVDSYKGVFFNGIIKGLDGHILIPEMQSSLLKQHMDLLKAAVFPLRHPLIDQN

SEQ ID NO: 167 - protein, 403 aa, Synthetic construct
EFPLYTIPEKIEKWTPIDMIHLSCPNNMLSEEEGCNTESPFTYFELKSGYLAHQKVPGFTCTGVVNEAETYTNFVGYVTTTFKRKHFKPTVAACRDAYNWKVSGDPRYEESLHTPYPDSSWLRTVTTTKEALLIISPSIVEMDIYGRTLHSPMFPSGKCSKLYPSVPSCTTNHDYTLWLPEDSSLSLICDIFTSSSGQKAMNGSRICGFKDERGFYRSLKGSCKLTLCGKPGIRLFDGTWVSFTKPDVHVWCTPNQLVNIHNDRLDEVEHLIVDDIIKKREECLDTLETILMSQSVSFRRLSHFRKLVPGYGKAYTILNGSLMETNVYYKRVDRWADILPSRGCLKVGQQCMDPVKGVLFNGIIKGPDGQILIPEMQSEQLKQHMDLLKAAMFPLRHPLINRE

SEQ ID NO: 168 - protein, 403 aa, Synthetic construct
EFPLYTIPEKIEKWTPIDMIHLSCPNNLLSEEEGCNTESPFTYFELKSGYLAHQKVPGFTCTGVVNEAETYTNFVGYVTTTFKRKHFMPTVAACRDAYNWKVSGDPRYEESLHTPYPDSSWLRTVTTTKESLLIISPSIVEMDVYGRTLHSPMFPSGICSKLYPSVPSCKTNHDYTLWLPEDPSLSLICDIFTSSSGQKVMNGSRICGFRDERGFYRSLKGACKLTLCGRPGIRLFDGTWVSFTKPDIHVWCTPNQLVNIHNDRLDEIEHLIVEDIIKKREECLDTLETILMSQSISFRRLSHFRKLVPGYGKAYTILNGSLMETNVYYKRVDKWTDILPSKGCLKVGQQCMEPVNGVLFNGIIKGPDGQILIPEMQSEQLKQHMDLLKAAVFPLRHPLIKRE

SEQ ID NO: 169 - protein, 403 aa, Synthetic construct
DFPLYTIPEKIGPWTPIDLIHLSCPNNLQSEDEGCGTSSVFSYVELKTGYLTHQKVSGFTCTGVVNEAVTYTNFVGYVTTTFKRKHFKPTALACRDAYHWKISGDPRYEESLHTPYPDNSWLRTVTTTKESLVIISPSIVEMDVYSRTLHSPMFPTGTCSRFYPSSPSCATNHDYTLWLPDDPNLSLACDIFVTSTGKKSMNGSRMCGFTDERGYYRTIKGACKLTLCGKPGLRLFDGTWISFTRPEVTTRCLPNQLVNIHNNRIDEVEHLIVEDLIRKREECLDTLETVLMSKSISFRRLSHFRKLVPGYGKAYTILNGSLMETNVHYLKVDNWSEILPSKGCLKINNQCVAHYKGVFFNGIIKGPDGHILIPEMQSSLLKQHMDLLKAAVFPLRHPLIEPG

SEQ ID NO: 170 - protein, 403 aa, Synthetic construct
KFPIYTIPDKLGPWSPIDIHHLSCPNNMVVEDEGCTNLSGFSYMELKVGYISAIKVNGFTCTGVVTEAETYTNFVGYVTTTFKRKHFRPVPDACRDAHNWKIAGDPRYEESLHNPYPDYHWLRTVKTTKESLVIISPSVADLDPYDKSLHSSVFPGGKCSGITVSSTYCSTNHDYTIWMPENPRLGTSCDIFTYSKGKRASKESKTCGFVDERGLYKSLKGACKLKLCGVPGLRLMDGTWVAIQTSDEVKWCPPDQLVNLHDLHSDELEHRVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGHVLIPEMQSSLLQQHMELLESSVIPLMHPLADPS

SEQ ID NO: 171 - protein, 153 aa, synthetic construct
KYDGSKLRIGILHARGNAEIILALVLGALKRLQEFGVKRENIIIETVPGSFELPYGSKLFVEKQKRLGKPLDAIIPIGVLIRGSTPHFDYIADSTTHQLMKLNFELGIPVIFGVITADTDEQAEARAGLIEGKMHNHGEDWGAAAVEMATKFN

SEQ ID NO: 172 - protein, 150 aa, synthetic construct
KYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESDEQAEERAGTKAGNHGEDWGAAAVEMATKFN

SEQ ID NO: 173 - protein, 205 aa, synthetic construct
MKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 174 - protein, 205 aa, synthetic construct
MKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVEYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 175 - protein, 205 aa, synthetic construct
MKMEELFKKHKIVAVLRANSVEEAKEKALAVFEGGVHLIEITFTVPDADTVIKELSFLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 176 - protein, 205 aa, synthetic construct; identical to SEQ ID NO: 173

SEQ ID NO: 177 - protein, 205 aa, synthetic construct
MKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 178 - protein, 205 aa, synthetic construct
MKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 179 - protein, 205 aa, synthetic construct
MKMEELFKKHKIVAVLRANSVEEAKMKALAVFVGGVHLIEITFTVPDADTVIKELSFLKELGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTIAEVAAKAAAFVEKIRGCTE

SEQ ID NO: 180 - protein, 205 aa, synthetic construct
MKMEELFKKHKIVAVLRANSVEEAKKKALAVFLGGVDLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGEALNKGTPVEVAEKAKAFVEKIRGCTE

SEQ ID NO: 181 - protein, 205 aa, synthetic construct
MKMEELFKKHKIVAVLRANSVEEAKKKALAVFMGGVDLIEITFTVPDADTVIKELSFLKELGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGEALNKGTPAEVAEKAKAFVEKIRGCTE

SEQ ID NO: 182 - protein, 167 aa, synthetic construct
MLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKVELIGNENHGLYLADQYVKGIAKSRKS

SEQ ID NO: 183 - protein, 165 aa, synthetic construct
MLKPEMIEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDAQN

SEQ ID NO: 184 - protein, 166 aa, synthetic construct
NFHQDCEAGLNRTVNLKFHSSYVYLSMASYFNRDDVALSNFAKFFRERSEEEKEHAEKLIEYQNQRGGRVFLQSVEKPERDDWANGLEALQTALKLQKSVNQALLDLHAVAADKSDPHMTDFLESPYLSESVETIKKLGDHITSLKKLWSSHPGMAEYLFNKHTLG

SEQ ID NO: 185 - protein, 169 aa, synthetic construct
QFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 186 - protein, 172 aa, synthetic construct
SGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS